(19) United States
(12) Patent Application Publication
(10) Pub. No.: US 2025/0059204 A1
(43) Pub. Date: Feb. 20, 2025

(54) FUSED BENZOISOXAZOLYL COMPOUNDS AS KAT6A INHIBITORS

(71) Applicants: Aurigene Oncology Limited, Bangalore, Karnataka (IN); Olema Pharmaceuticals, Inc., San Francisco, CA (US)

(72) Inventors: Chandregowda Venkateshappa, Bangalore (IN); Kalisankar Bera, Bangalore (IN); Chandrasekhar Abbineni, Hyderabad (IN); Susanta Samajdar, Bangalore (IN); David C. Myles, Berkeley, CA (US); Brian R. Hearn, Moraga, CA (US)

(21) Appl. No.: 18/718,391

(22) PCT Filed: Dec. 12, 2022
(86) PCT No.: PCT/US22/81352

§ 371 Date: Jun. 10, 2024

(30) Foreign Application Priority Data
Dec. 13, 2021 (IN) 202141058005

(51) Int. Cl. C07D 498/14; A61K 31/424; A61P 35/00

(52) U.S. Cl. CPC C07D 498/14; A61K 31/424; A61P 35/00

(57) ABSTRACT

The present invention provides fused benzoisoxazolyl compounds represented by compound of formula (I), which are therapeutically useful as KAT6A inhibitors particularly in the treatment and/or prevention of the diseases or disorders mediated by KAT6A in a subject. The present invention also provides preparation of the compounds and pharmaceutical compositions comprising at least one of the compounds of formula (I) or a pharmaceutically acceptable salt or a stereoisomer or a tautomer thereof.

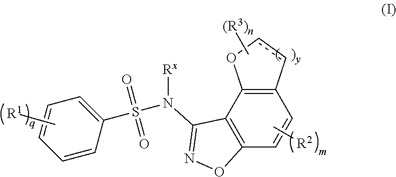

### CROSS-REFERENCES TO RELATED APPLICATIONS

This application claims priority to India Application No. 202141058005, filed Dec. 13, 2021, which is incorporated herein in its entirety for all purposes.

### FIELD OF THE INVENTION

The present invention relates to fused benzoisoxazolyl compounds represented by compound of formula (I), a pharmaceutically acceptable salt or a stereoisomer or a tautomer thereof which are useful as KAT6A inhibitors for the treatment of diseases or disorders mediated by KAT6A. The present invention also relates to a method of preparation of the said compounds and pharmaceutical compositions comprising the said compounds.

### BACKGROUND OF THE INVENTION

The MYST family of histone acetyltransferases (HATs) is named after its four founding members MOZ, Ybf2 (Sas3), Sas2, and Tip60. Presence of zinc fingers and chromodomains is characteristic feature of these HATs. MYST acetylates lysine residues on histones H2A, H3, and H4. Several MYST family proteins contain zinc fingers as well as the highly conserved motif A found among GNATs that facilitate acetyl-CoA binding. MYST HATs are involved in several key nuclear processes and play critical roles in gene-specific transcription regulation, DNA damage response, repair and replication. The anomalous activity of these HATs or their associated complexes can easily lead to severe cellular malfunction, resulting in cell death or uncontrolled growth and malignancy. Indeed, the MYST family HATs have been implicated in several forms of human cancer. (Avvakumov, N. et al. “The MYST family of histone acetyltransferases and their intimate links to cancer.” Oncogene 26.37 (2007): 5395-5407.)

MOZ (monocytic leukaemia zinc finger protein) is known as oncogene in human. MOZ plays a key role as transcriptional coactivator and epigenetic regulator in the process of proliferation and the differentiation of hematopoietic progenitor and stem cells. The insights into the deregulation of these processes indicate that MOZ fusion proteins are related to the formation of leukemic stem cells and interfere with the activities of key proteins such as transcription factors, which render MOZ as a promising target for acute myeloid leukaemia therapy. Targeting MOZ by small molecules will hold promise for acute myeloid leukaemia therapy. (Zhou C. et al. “MOZ/KAT6A: a promising target for acute myeloid leukaemia therapy.” (2020): 759-761).

Cellular senescence plays a key role to restrict tumour growth. KAT6A represses cellular senescence in mouse embryonic fibroblasts (MEFs) while not affecting apoptosis or DNA damage. MOZ directly binds to genes that inhibit senescence including Cdc6, E2f2, Ezh2 and Melk, and in its absence, H3K9ac and H3K27ac at the TSS of these loci is reduced. (Sheikh, B. N., et al. “MOZ (MYST3, KAT6A) inhibits senescence via the INK4A-ARF pathway.” Oncogene 34.47 (2015): 5807-5820).

Histone acetyltransferase KAT6A-upregulated PI3K/AKT signalling through TRIM24 binding is critical for cell proliferation and tumour growth in gliomas. KAT6A promotes H3K23 acetylation and association with TRIM24, leading to increased PIK3 CA expression and PI3K/Akt signalling activation, resulting in enhanced glioma tumorigenesis. KAT6A functions as an oncogene in gliomas. (Lv, D., et al. “Histone acetyltransferase KAT6A upregulates PI3K/AKT signalling through TRIM24 binding.” Cancer research 77.22 (2017): 6190-6201).

Many patent publications disclose several small molecule compounds and their derivatives capable of targeting KAT target proteins. Considering the established role of KATs in diseases such as cancer and because there are currently no FDA-approved targeted therapeutics for specific KAT6A or KAT6B target protein, there is a need for the development of compounds, compositions, and methods for treating KAT6A- or KAT6B-activated proliferative disorders and autoimmune diseases.

### BRIEF SUMMARY OF THE INVENTION

In some embodiments, a compound of the present invention is a compound of Formula (I):

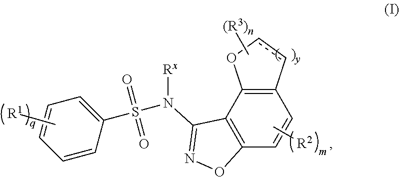

or a pharmaceutically acceptable salt or a stereoisomer or a tautomer thereof; wherein, dotted line [symbol] represents a single bond or is absent;
each R¹ is independently hydrogen, (C₁-C₆)alkyl, (C₂-C₆)alkenyl, (C₂-C₆)alkynyl, (C₁-C₆)alkoxy, (C₂-C₆)alkoxyalkyl, (C₁-C₆)hydroxyalkyl, (C₁-C₆)aminoalkyl, halogen, (C₁-C₆)haloalkyl, (C₁-C₆)haloalkoxy, —C(O)R^{1h}, —C(O)OR^{1h}, —OC(O)R^{1h}, —C(O)N(R^{1h})(R^{1j}), —N(R^{1h})C(O)(R^{1j}), —S(O)₂R^{1h}, S(O)₂N(R^{1h})(R^{1j}), hydroxy, —CN, —NO₂, —SF₅, (C₃-C₈)cycloalkyl, 3- to 8-membered heterocycloalkyl, (C₆-C₁₀)aryl, or 5- to 10-membered heteroaryl, wherein each alkoxy is substituted with 0, 1, 2, or 3 R^{1f}, and each cycloalkyl, heterocycloalkyl, aryl, and heteroaryl is substituted with 0, 1, 2, or 3 R^{1g};
each R^{1h} and R¹ⁱ is independently hydrogen or (C₁-C₆)alkyl;
each R^{1f} is independently 5- to 10-membered heteroaryl;
each R^{1g} is independently (C₁-C₆)alkyl, (C₁-C₆)alkoxy, halogen, (C₁-C₆)haloalkyl, or (C₁-C₆)haloalkoxy;
alternatively, two R¹ groups on adjacent ring atoms are combined to form (C₅-C₈)cycloalkyl or a 5- to 8-membered heterocycloalkyl;
each R² is independently hydrogen, (C₁-C₆)alkyl, (C₂-C₆)alkenyl, (C₂-C₆)alkynyl, (C₁-C₆)alkoxy, (C₁-C₆)hydroxyalkyl, halogen, (C₁-C₆)haloalkoxy, hydroxy, —OR^{4c}, —NHR^{4c}, —SR^{4c}, —C(O)R^{4c}, —S(O)R^{4c}, —S(O)₂R^{4c}, (C₃-C₈)cycloalkyl, (C₆-C₁₀)aryl, 3- to 8-membered heterocycloalkyl, or 5- to 10-membered heteroaryl; wherein each alkyl group is substituted with 0, 1, 2 or 3 R^{4a}; and wherein each cycloalkyl, heterocycloalkyl, aryl, and heteroaryl is substituted with 0, 1, 2 or 3 R^{4b};
each R³ is independently hydrogen, (C₁-C₆)alkyl, (C₁-C₆)alkoxy, halogen, (C₁-C₆)haloalkoxy, or hydroxy; wherein each alkyl is substituted with 0, 1, 2, or 3 R^{3a}; each R^{3a} is independently hydroxy, (C₁-C₆)alkoxy or halogen;
each R^{4a} is independently (C₁-C₆)alkoxy, halogen, C₁-C₆ haloalkoxy, —NH₂, —CN, —NO₂, C₃-C₈ cycloalkyl, (C₆-C₁₀)aryl, 3- to 8-membered heterocycloalkyl, or 5- to 10-membered heteroaryl, wherein each cycloalkyl, heterocycloalkyl, aryl, and heteroaryl is substituted with 0, 1, 2, or 3 R^{4a1};
each R^{4a1} is independently (C₁-C₆)alkyl, (C₂-C₆)alkenyl, (C₂-C₆)alkynyl, (C₁-C₆)alkoxy, (C₂-C₆)alkoxyalkyl, (C₁-C₆)hydroxyalkyl, halogen, (C₁-C₆)haloalkyl, (C₁-C₆)haloalkoxy, hydroxy, —N(R^{4a2})(R^{4a3}), —C(O)R^{4a2}, —C(O)OR^{4a2}, —OC(O)R^{4a2}, —C(O)N(R^{4a2})(R^{4a3}), —N(R^{4a2})C(O)(R^{4a3}), —S(O)₂R^{4a2}, —S(O)₂N(R^{4a2})(R^{4a3}), —CN, or —NO₂; each R^{4a2} and R^{4a3} is independently hydrogen or (C₁-C₆)alkyl;
each R^{4b} is independently (C₁-C₆)alkyl, (C₂-C₆)alkenyl, (C₂-C₆)alkynyl, (C₁-C₆)alkoxy, (C₁-C₆)hydroxyalkyl, halogen, (C₁-C₆)haloalkyl, (C₁-C₆)haloalkoxy, hydroxy, —NH₂, —CN, —NO₂, (C₃-C₈)cycloalkyl, (C₆-C₁₀)aryl, 3- to 8-membered heterocycloalkyl, or 5- to 10-membered heteroaryl;
each R⁴, is (C₃-C₈)cycloalkyl, (C₆-C₁₀)aryl, 3- to 8-membered heterocycloalkyl, or 5- to 10-membered heteroaryl; R^{x} is hydrogen or (C₁-C₆)alkyl;
subscript ‘m’ is an integer of 1 or 2;
subscript ‘y’ is an integer of 1 or 2;
subscript ‘n’ is an integer of 1, 2 or 3; and
subscript ‘q’ is an integer of from 1 to 5,
wherein each heterocycloalkyl and heteroaryl includes 1 to 4 heteroatoms each independently N, O or S.

In some embodiments, a composition of the present invention is a pharmaceutical composition comprising a compound of the present invention, or a pharmaceutically acceptable salt or stereoisomer or a tautomer thereof and a pharmaceutically acceptable carrier or excipient.

In some embodiments, a compound of the present invention is a compound or a pharmaceutically acceptable salt or stereoisomer or a tautomer thereof for use as a medicament.

In some embodiments, a compound of the present invention is a compound for use in the treatment of a disease or disorder mediated by KAT6A.

In some embodiments, a method of the present invention is a method of modulating KAT6A in a subject comprising administering to the subject in need thereof, a therapeutically effective amount of compound of the present invention or a pharmaceutically acceptable salt or a stereoisomer or a tautomer thereof.

In some embodiments, a method of the present invention is a method of treating a disease or disorder mediated by KAT6A in a subject comprising administering to the subject in need thereof a therapeutically effective amount of a compound of the present invention or a pharmaceutically acceptable salt or a stereoisomer or a tautomer thereof.

### DETAILED DESCRIPTION OF THE INVENTION

### I. General

Provided herein are fused benzoisoxazolyl compounds represented by a compound of formula (I) and, a pharmaceutically acceptable salt or a stereoisomer or a tautomer thereof.

Also, provided herein are pharmaceutical compositions comprising the said compounds which are useful as KAT6A inhibitors for the treatment of diseases or disorders dependent on or mediated by KAT6A. The present invention also provides a preparation of compound of formula (I) or a pharmaceutically acceptable salt or a stereoisomer or a tautomer thereof.

### II. Definitions

Unless defined otherwise, all technical and scientific terms used herein have the same meaning as is commonly understood by one of skill in art to which the subject matter herein belongs. As used in the specification and the appended claims, unless specified to the contrary, the following terms have the meaning indicated in order to facilitate the understanding of the present invention.

The singular forms “a”, “an” and “the” encompass plural references unless the context clearly indicates otherwise.

As used herein, the terms “optional” or “optionally” mean that the subsequently described event or circumstance may occur or may not occur and that the description includes instances where the event or circumstance occurs as well as instances in which it does not. For example, “optionally substituted alkyl” refers to an event or circumstance in which the said alkyl may be substituted as well as the event or circumstance in which the alkyl is not substituted. The phrase “optionally substituted” can also be interchangeably used as an unsubstituted or substituted group. For example, “optionally substituted alkyl” can be used as ‘unsubstituted or substituted alkyl’ group. In some embodiments, optionally substituted refers to a group that has 0, 1, 2, or 3 substituents defined therein.

The term “substituted” refers to moieties having substituents replacing hydrogen on one or more carbons of the backbone. It will be understood that “substitution” or “substituted with” includes the implicit proviso that such substitution is in accordance with permitted valence of the substituted atom and the substituent and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Unless specifically stated, the substituents can include any substituents described herein, for example, a halogen, a hydroxyl, a carbonyl (such as a carboxyl, an alkoxycarbonyl, a formyl or an acyl), a thiocarbonyl (such as a thioester, a thioacetate or a thioformate), an alkoxyl, an oxo, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heteroaryl, a heterocycloalkyl, an aralkyl or an aromatic or heteroaromatic moiety. It will be understood by those skilled in the art that substituents can themselves be substituted, if appropriate.

As used herein, the term “alkyl” refers to saturated aliphatic groups, including but not limited to C₁-C₁₀ straight-chain alkyl groups or C₃-C₁₀ branched-chain alkyl groups. Preferably, the “alkyl” group refers to C₁-C₆ straight-chain alkyl groups or C₃-C₈ branched-chain alkyl groups. Most preferably, the “alkyl” group refers to C₁-C₄ straight-chain alkyl groups or C₃-C₆ branched-chain alkyl groups. Examples of “alkyl” include, but are not limited to, methyl, ethyl, 1-propyl, 2-propyl, isopropyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl and 4-octyl. The “alkyl” group may be optionally substituted.

“Alkenyl” refers to a straight chain or branched hydrocarbon having at least 2 carbon atoms and at least one double bond. Alkenyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. Alkenyl groups can be substituted or unsubstituted.

“Alkynyl” refers to either a straight chain or branched hydrocarbon having at least 2 carbon atoms and at least one triple bond. Alkynyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Examples of alkynyl groups include, but are not limited to, acetylenyl, propynyl, 1-butynyl, 2-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatrienyl. Alkynyl groups can be substituted or unsubstituted.

As used herein, the term “halo” or “halogen” alone or in combination with other term(s) means fluorine, chlorine, bromine or iodine.

As used herein, the term “haloalkyl” refers to alkyl substituted with one or more halogen atoms, wherein the ‘halogen’ and ‘alkyl’ groups are as defined above. Examples of “haloalkyl” include but are not limited to fluoromethyl, difluoromethyl, chloromethyl, trifluoromethyl and 2,2,2-trifluoroethyl.

As used herein, the term “alkoxy” refers to the group —O-alkyl, where alkyl groups are as defined above. Preferably, the “alkoxy” group refers to C₁-C₆ straight-chain alkoxy groups or C₃-C₈ branched-chain alkoxy groups. Most preferably, the “alkoxy” group refers to C₁-C₄ straight-chain alkoxy groups or C₃-C₆ branched-chain alkoxy groups. Exemplary C₁-C₁₀ alkoxy group include but are not limited to methoxy, ethoxy, n-propoxy, n-butoxy or t-butoxy. An alkoxy group can be optionally substituted with one or more suitable groups, as described herein.

As used herein, the term “alkoxyalkyl” refers to an alkyl group substituted with one or more alkoxy groups, wherein the alkyl and alkoxy groups are as defined above. In some embodiments, alkoxyalkyl represents (C₁-C₆)alkyl(C₁-C₆)alkoxy and preferably (C₁-C₄)alkyl(C₁-C₄)alkoxy. Exemplary alkoxyalkyl group include, but are not limited to methoxymethyl, ethoxymethyl and ethoxyethyl.

As used herein, the term “aryl” is optionally substituted monocyclic, bicyclic or polycyclic aromatic hydrocarbon ring system of about 6 to 14 carbon atoms. Preferably, aryl includes, 6- to 10-membered aromatic hydrocarbon ring systems. Examples of a C₆-C₁₄ aryl groups include, but are not limited to phenyl, naphthyl, biphenyl, anthryl, tetrahydronaphthyl, fluorenyl, indanyl, biphenylenyl and acenaphthyl. Aryl group can be optionally substituted with one or more suitable groups, described herein.

“Cycloalkyl” refers to a single saturated or partially unsaturated all carbon ring having 3 to 20 annular carbon atoms (i.e., C₃-20 cycloalkyl), for example from 3 to 12 annular atoms, for example from 3 to 10 annular atoms, or 3 to 8 annular atoms, or 3 to 6 annular atoms, or 3 to 5 annular atoms, or 3 to 4 annular atoms. The term “cycloalkyl” also includes multiple condensed, saturated and partially unsaturated all carbon ring systems (e.g., ring systems comprising 2, 3 or 4 carbocyclic rings). Accordingly, cycloalkyl includes multicyclic carbocycles such as a bicyclic carbocycles (e.g., bicyclic carbocycles having about 6 to 12 annular carbon atoms such as bicyclo[3.1.0]hexane and bicyclo[2.1.1]hexane), and polycyclic carbocycles (e.g. tricyclic and tetracyclic carbocycles with up to about 20 annular carbon atoms). The rings of a multiple condensed ring system can be connected to each other via fused, spiro and bridged bonds when allowed by valency requirements. Non-limiting examples of monocyclic cycloalkyl include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl and 1-cyclohex-3-enyl.

As used herein, the term “heterocyclyl” or “heterocyclic” alone or in combination with other term(s) includes both “heterocycloalkyl” and “heteroaryl” groups which are as described herein. As used herein, the term “heterocycloalkyl” refers to a non-aromatic, saturated or partially saturated, bridged bicyclic, monocyclic or polycyclic ring system of 3- to 15-member, unless the ring size is specifically mentioned, having at least one heteroatom or hetero group selected from O, N, S, S(O), S(O)₂, NH or C(O) with the remaining ring atoms being independently selected from the group consisting of carbon, oxygen, nitrogen and sulfur. The term “heterocycloalkyl” also refers to the bridged bicyclic ring system having at least one heteroatom or hetero group selected from O, N, S, S(O), S(O)₂, NH or C(O). Examples of “heterocycloalkyl” include, but not limited to, aziridinyl, oxiranyl, thiiranyl, azetidinyl, oxetanyl, imidazolidinyl, pyrrolidinyl, oxazolidinyl, thiazolidinyl, pyrazolidinyl, tetrahydrofuranyl, piperidinyl, dihydropyridinyl, piperazinyl, tetrahydropyranyl, morpholinyl, thiomorpholinyl, 1,4-dioxanyl, dioxidothiomorpholinyl, oxopiperazinyl, oxopiperidinyl, tetrahydrofuryl, tetrahydropyranyl, tetrahydrothiophenyl, dihydropyranyl, indolinyl, indolinylmethyl, isoindolinyl, oxoisoindolinyl, dioxoisoindolinyl, aza-bicyclooctanyl, diazabicyclooctanyl, azocinyl, chromanyl, isochromanyl and xanthenyl. Attachment of a heterocycloalkyl substituent can occur via either a carbon atom or a heteroatom. A heterocycloalkyl group can be optionally substituted with one or more suitable groups by one or more aforesaid groups. Preferably “heterocycloalkyl” refers to 4- to 6-membered ring (unless the ring size is specifically mentioned) selected from the group consisting of azetidinyl, oxetanyl, imidazolidinyl, pyrrolidinyl, oxazolidinyl, thiazolidinyl, pyrazolidinyl, tetrahydrofuranyl, piperidinyl, piperazinyl, tetrahydropyranyl, morpholinyl and thiomorpholinyl. All heterocycloalkyl are optionally substituted by one or more groups described herein.

As used herein, the term “heteroaryl” refers to a completely unsaturated and aromatic ring system containing a total of 5 to 14 ring atoms, unless the ring size is specifically mentioned. At least one of the ring atoms is a heteroatom (i.e., O, N or S), with the remaining ring atoms/groups being independently selected from C, N, O or S. A heteroaryl may be a single-ring (monocyclic) or multiple rings (bicyclic, tricyclic or polycyclic) fused together or linked covalently. Preferably, “heteroaryl” is a 5- to 6-membered ring, unless the ring size is specifically mentioned. The rings may contain from 1 to 4 additional heteroatoms selected from N, O and S, wherein the N atom is optionally quarternized. Any suitable ring position of the heteroaryl moiety may be covalently linked to the defined chemical structure. Examples of “heteroaryl” include but not limited to furanyl, thienyl, pyrrolyl, pyrazolyl, imidazolyl, oxazolyl, cinnolinyl, isoxazolyl, thiazolyl, isothiazolyl, 1H-tetrazolyl, oxadiazolyl, triazolyl, pyridyl (pyridinyl), 3-fluoropyridyl, pyrimidinyl, pyrazinyl, pyridazinyl, benzoxazolyl, benzisoxazolyl, benzothiazolyl, benzofuranyl, benzothienyl, benzotriazinyl, phthalazinyl, thianthrene, dibenzofuranyl, dibenzothienyl, benzimidazolyl, indolyl, isoindolyl, indazolyl, quinolinyl, isoquinolinyl, quinazolinyl, quinoxalinyl, purinyl, pteridinyl, 9H-carbazolyl, α-carbolinyl, indolizinyl, benzoisothiazolyl, benzoxazolyl, pyrrolopyridyl, purinyl, benzothiadiazolyl, benzoxadiazolyl, benzotriazolyl, benzothiadiazolyl, carbazolyl, dibenzothienyl, acridinyl and the like. Heteroaryl group may be optionally further substituted.

As used herein, the term “amino” refers to an —NH₂ group.

As used herein, the term “aminoalkyl” refers to an alkyl group substituted with an amino group, wherein the amino and alkyl groups are as defined above. Examples of “aminoalkyl” include but are not limited to —CH₃—NH₂, —CH₂—CH₂—NH₂, —CH₂—CH₂—CH₂—NH₂, —CH(CH₃)—CH₂—NH₂, —CH₂—CH₂—NH (CH₃) and —CH₂—CH₂—N(CH₃)₂.

As used herein, the term “hydroxy” or “hydroxyl” alone or in combination with other term(s) means —OH.

The term “hydroxyalkyl” refers to an alkyl group substituted with one or more hydroxyls.

As used herein, the term “haloalkoxy” refers to an alkoxy group substituted with one or more halogen atoms (i.e., haloC₁₋₈alkoxy). Examples of “haloalkoxy” include but are not limited to fluoromethoxy, difluoromethoxy, trifluoromethoxy, 2,2,2-trifluoroethoxy, pentafluoroethoxy, pentachloroethoxy, chloromethoxy, dichloromethoxy, trichloromethoxy and 1-bromoethoxy.

The term “heteroatom” as used herein designates a sulfur, nitrogen or oxygen atom.

As used herein, the term ‘compound(s)’ comprises the compound(s) disclosed in the present invention.

As used herein, the term “comprise” or “comprising” is generally used in the sense of include, that is to say permitting the presence of one or more features or components.

As used herein, the term “or” means “and/or” unless stated otherwise.

As used herein, the term “including” as well as other forms, such as “include”, “includes” and “included” is not limiting.

As used herein, the term “composition” is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

As used herein, the term “pharmaceutical composition” refers to a composition(s) containing a therapeutically effective amount of at least one compound of formula (I) or a pharmaceutically acceptable salt thereof; and a pharmaceutically acceptable carrier.

The pharmaceutical composition(s) usually contain(s) about 1% to 99%, for example, about 5% to 75% or from about 10% to about 30% by weight of the compound of formula (I) or (II) or pharmaceutically acceptable salts thereof. The amount of the compound of formula (I) or pharmaceutically acceptable salts thereof in the pharmaceutical composition(s) can range from about 1 mg to about 1000 mg or from about 2.5 mg to about 500 mg or from about 5 mg to about 250 mg or in any range falling within the broader range of 1 mg to 1000 mg or higher or lower than the aforementioned range.

As used herein, “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant or emulsifier that has been approved by the United States Food and Drug Administration as being acceptable for use in humans or domestic animals.

The term “administer,” “administering,” or “administration” as used in this disclosure refers to either directly administering one or more disclosed compounds or a pharmaceutically acceptable salt of one or more disclosed compounds or a composition comprising one or more disclosed compounds to a subject or analog of the compound or a pharmaceutically acceptable salt of the compound or composition to the subject, which can form an equivalent amount of active compound within the subject's body.

The term “carrier” as used in this disclosure, encompasses carriers, excipients and diluents and means a material, composition or vehicle, such as a liquid or solid filler, diluent, excipient, solvent or encapsulating material, involved in carrying or transporting a pharmaceutical agent from one organ or portion of the body to another organ or portion of the body of a subject.

As used herein, the term “treat”, “treating” and “treatment” refer to a method of alleviating or abrogating a disease and/or its attendant symptoms.

As used herein, the term “prevent”, “preventing” and “prevention” refer to a method of preventing the onset of a disease and/or its attendant symptoms or barring a subject from acquiring a disease. As used herein, “prevent”, “preventing” and “prevention” also include delaying the onset of a disease and/or its attendant symptoms and reducing a subject's risk of acquiring a disease.

As used herein, the term “subject” that may be interchangeable with ‘patient’, refers to an animal, preferably a mammal and most preferably a human. In some embodiments, the subject is a mammal, such as a mouse, a rat, a dog, a cat, another veterinary animal, such as a goat, a pig, a horse, a cow, or a donkey; or a primate, such as a non-human primate, e.g., a cynomolgous monkey, rhesus monkey, or chimpanzee, or a human. In some embodiments, the subject is a human.

As used herein, the term, “therapeutically effective amount” refers to an amount of a compound of formula (I) or a pharmaceutically acceptable salt or a stereoisomer or a tautomer thereof; or a composition comprising the compound of formula (I) or a pharmaceutically acceptable salt or a stereoisomer or a tautomer thereof, effective in producing the desired therapeutic response in a particular patient suffering from a diseases or disorder, in particular their use in diseases or disorder associated with cancer. Particularly, the term “therapeutically effective amount” includes the amount of the compound of formula (I) or a pharmaceutically acceptable salt or a stereoisomer or a tautomer thereof, when administered, that induces a positive modification in the disease or disorder to be treated or is sufficient to prevent development of or alleviate to some extent, one or more of the symptoms of the disease or disorder being treated in a subject. In respect of the therapeutic amount of the compound, the amount of the compound used for the treatment of a subject is low enough to avoid undue or severe side effects, within the scope of sound medical judgment can also be considered. The therapeutically effective amount of the compound or composition will be varied with the particular condition being treated, the severity of the condition being treated or prevented, the duration of the treatment, the nature of concurrent therapy, the age and physical condition of the end user, the specific compound or composition employed the particular pharmaceutically acceptable carrier utilized.

“Pharmaceutically acceptable” means that, which is useful in preparing a pharmaceutical composition that is generally safe, non-toxic and neither biologically nor otherwise undesirable and includes that which is acceptable for veterinary as well as human pharmaceutical use.

The term “pharmaceutically acceptable salt” refers to a product obtained by reaction of the compound of the present invention with a suitable acid or a base. In some cases, a medicament can be present in the form of a pharmaceutically acceptable salt. In some instances, a pharmaceutically acceptable salt can be a salt described in Berge et al, J. Pharm. Sci, 1977. In some instances, a pharmaceutically acceptable salts can include those salts derived from a mineral, organic acid or inorganic base. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the non-toxic salts of the parent compound formed, e.g., from non-toxic inorganic or organic acids.

The pharmaceutically acceptable salts of the present invention can be prepared from a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20^{th} ed., Mack Publishing Company, Easton, Pa., (1985); and in “Handbook of Pharmaceutical Salts: Properties, Selection and Use” by Stahl and Wermuth (Wiley-VCH, Weinheim, Germany, 2002).

The present invention also provides methods for formulating the disclosed compounds as for pharmaceutical administration.

In a preferred embodiment, when such pharmaceutical compositions are for human administration, particularly for invasive routes of administration (i.e., routes, such as injection or implantation, that circumvent transport or diffusion through an epithelial barrier), the aqueous solution is pyrogen-free or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

The term “stereoisomers” refers to any enantiomers, diastereoisomers or geometrical isomers of the compound of formula (I), wherever they are chiral or when they bear one or more double bonds. When the compounds of the formula (I) and related formulae are chiral, they can exist in racemic or in optically active form. It should be understood that the invention encompasses all stereochemical isomeric forms, including diastereomeric, enantiomeric and epimeric forms, as well as d-Isomers and l-Isomers and mixtures thereof. Individual stereoisomers of compounds can be prepared synthetically from commercially available starting materials which contain chiral centres or by preparation of mixtures of enantiomeric products followed by separation such as conversion to a mixture of diastereomers followed by separation or recrystallization, chromatographic techniques, direct separation of enantiomers on chiral chromatographic columns or any other appropriate method known in the art. Starting compounds of particular stereochemistry are either commercially available or can be made and resolved by techniques known in the art. Additionally, the compounds of the present invention may exist as geometric Isomers. The present invention includes all cis, trans, syn, anti, entgegen (E) and zusammen (Z) Isomers as well as the appropriate mixtures thereof.

The term “enantiomers” refers to a pair of stereoisomers which are non-superimposable mirror images of one another. The term “enantiomer” refers to a single member of this pair of stereoisomers. The term “racemic” refers to a 1:1 mixture of a pair of enantiomers. The disclosure includes enantiomers of the compounds described herein. Each compound herein disclosed includes all the enantiomers that conform to the general structure of the compound. The compounds may be in a racemic or enantiomerically pure form or any other form in terms of stereochemistry. In some embodiments the compounds are the (R, S)-enantiomer.

The term “diastereomers” refers to the set of stereoisomers which cannot be made superimposable by rotation around single bonds. For example, cis- and trans-double bonds, endo- and exo-substitution on bicyclic ring systems and compounds containing multiple stereogenic centres with different relative configurations are considered to be diastereomers. The term “diastereomer” refers to any member of this set of compounds. In some examples presented, the synthetic route may produce a single diastereomer or a mixture of diastereomers. The disclosure includes diastereomers of the compounds described herein.

The term “tautomer” refers to compounds in which hydrogen atoms are transposed to other parts of the molecules and the chemical bonds between the atoms of the molecules are consequently rearranged. Compounds of the present invention, free form and salts thereof, may exist in multiple tautomeric forms. It is understood that all tautomeric forms, insofar as they may exist, are included within the invention.

Each embodiment is provided by way of explanation of the invention and not by way of limitation of the invention. In fact, it will be apparent to those skilled in the art that various modifications and variations can be made to the compounds, compositions and methods described herein without departing from the scope or spirit of the invention. For instance, features illustrated or described herein as part of an embodiment can be applied to another embodiment to yield a still further embodiment. Thus, it is intended that the present invention includes such modifications and variations and their equivalents. Other objects, features and aspects of the present invention are disclosed in or are obvious from, the following detailed description. It is to be understood by one of ordinary skill in the art that the present specification is just a description of exemplary embodiments only and not to be construed as limiting the broader aspects of the present invention.

All publications and patents mentioned herein are hereby incorporated by reference in their entirety as if each individual publication or patent were specifically and individually indicated to be incorporated by reference. In case of conflict, the present application, including any definitions herein, will control.

### III. Compounds

The present invention provides fused benzoisoxazolyl compounds, referred as a compound of formula (I), which are useful as KAT6A inhibitors for the treatment of diseases or disorders dependent on or mediated by KAT6A. The present invention further provides pharmaceutical compositions comprising the said compounds or a pharmaceutically acceptable salt, or a stereoisomer or a tautomer thereof as therapeutic agents.

Each embodiment is provided by way of explanation of the invention and not by way of limitation of the invention. In fact, it will be apparent to those skilled in the art that various modifications and variations can be made to the compounds, compositions and methods described herein without departing from the scope or spirit of the invention. For instance, features illustrated or described herein as part of one embodiment can be applied to another embodiment to yield a still further embodiment. Thus, it is intended that the present invention includes such modifications and variations and their equivalents. Other objects, features and aspects of the present invention are disclosed in or are obvious from, the following detailed description. It is to be understood by one of ordinary skill in the art that the present specification is just a description of exemplary embodiments only and not to be construed as limiting the broader aspects of the present invention.

In some embodiments, the present invention provides a compound of Formula (I):

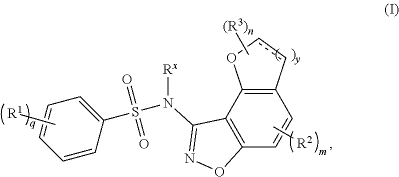

or a pharmaceutically acceptable salt or a stereoisomer or a tautomer thereof; wherein, dotted line [symbol] represents a single bond or is absent;
each R¹ is independently hydrogen, (C₁-C₆)alkyl, (C₂-C₆)alkenyl, (C₂-C₆)alkynyl, (C₁-C₆)alkoxy, (C₂-C₆)alkoxyalkyl, (C₁-C₆)hydroxyalkyl, (C₁-C₆)aminoalkyl, halogen, (C₁-C₆)haloalkyl, (C₁-C₆)haloalkoxy, —C(O)R^{1h}, C(O)OR^{1h}—OC(O)R^{1h}, —C(O)N(R^{1h})(R^{1j}), —N(R^{1h})C(O)(R^{1j}), —S(O)₂Rh, —S(O)₂N(R^{1h})(R^{1j}), hydroxy, —CN, —NO₂, —SF₅, (C₃-C₈)cycloalkyl, 3- to 8-membered heterocycloalkyl, (C₆-C₁₀)aryl, or 5- to 10-membered heteroaryl, wherein each alkoxy is substituted with 0, 1, 2, or 3 R^{1f}, and each cycloalkyl, heterocycloalkyl, aryl, and heteroaryl is substituted with 0, 1, 2, or 3 R^{1g};
each R^{1h} and R¹ⁱ is independently hydrogen or (C₁-C₆)alkyl;
each R^{1f} is independently 5- to 10-membered heteroaryl;
each R^{1g} is independently (C₁-C₆)alkyl, (C₁-C₆)alkoxy, halogen, (C₁-C₆)haloalkyl, or (C₁-C₆)haloalkoxy;
alternatively, two R¹ groups on adjacent ring atoms are combined to form (C₅-C₈)cycloalkyl or a 5- to 8-membered heterocycloalkyl;
each R² is independently hydrogen, (C₁-C₆)alkyl, (C₂-C₆)alkenyl, (C₂-C₆)alkynyl, (C₁-C₆)alkoxy, (C₁-C₆)hydroxyalkyl, halogen, (C₁-C₆)haloalkoxy, hydroxy, —OR^{4c}, —NHR^{4c}, —SR^{4c}, —C(O)R^{4c}, —S(O)R^{4c}, —S(O)₂R^{4c}, (C₃-C₈)cycloalkyl, (C₆-C₁₀)aryl, 3- to 8-membered heterocycloalkyl, or 5- to 10-membered heteroaryl; wherein each alkyl group is substituted with 0, 1, 2 or 3 R^{4a}; and wherein each cycloalkyl, heterocycloalkyl, aryl, and heteroaryl is substituted with 0, 1, 2 or 3 R^{4b};
each R³ is independently hydrogen, (C₁-C₆)alkyl, (C₁-C₆)alkoxy, halogen, (C₁-C₆)haloalkoxy, or hydroxy; wherein each alkyl is substituted with 0, 1, 2, or 3 R^{3a};
each R^{3a} is independently hydroxy, (C₁-C₆)alkoxy or halogen;
each R^{4a} is independently (C₁-C₆)alkoxy, halogen, C₁-C₆ haloalkoxy, —NH₂, —CN, —NO₂, C₃-C₈ cycloalkyl, (C₆-C₁₀)aryl, 3- to 8-membered heterocycloalkyl, or 5- to 10-membered heteroaryl, wherein each cycloalkyl, heterocycloalkyl, aryl, and heteroaryl is substituted with 0, 1, 2, or 3 R^{4a1};
each R^{4a1} is independently (C₁-C₆)alkyl, (C₂-C₆)alkenyl, (C₂-C₆)alkynyl, (C₁-C₆)alkoxy, (C₂-C₆)alkoxyalkyl, (C₁-C₆)hydroxyalkyl, halogen, (C₁-C₆)haloalkyl, (C₁-C₆)haloalkoxy, hydroxy, —N(R^{4a2})(R^{4a3}), —C(O)R^{4a2}, —C(O)OR^{4a2}, —OC(O)R^{4a2}, —C(O)N(R^{4a2})(R^{4a3}), —N(R^{4a2})C(O)(R^{4a3}), —S(O)₂R^{4a2}, —S(O)₂N(R^{4a2})(R^{4a3}), —CN, or —NO₂;
each R^{4a2} and R^{4a3} is independently hydrogen or (C₁-C₆)alkyl;
each R^{4b} is independently (C₁-C₆)alkyl, (C₂-C₆)alkenyl, (C₂-C₆)alkynyl, (C₁-C₆)alkoxy, (C₁-C₆)hydroxyalkyl, halogen, (C₁-C₆)haloalkyl, (C₁-C₆)haloalkoxy, hydroxy, —NH₂, —CN, —NO₂, (C₃-C₈)cycloalkyl, (C₆-C₁₀)aryl, 3- to 8-membered heterocycloalkyl, or 5- to 10-membered heteroaryl;
each R^{4c} is (C₃-C₈)cycloalkyl, (C₆-C₁₀)aryl, 3- to 8-membered heterocycloalkyl, or 5- to 10-membered heteroaryl; R^{x} is hydrogen or (C₁-C₆)alkyl;
subscript ‘m’ is an integer of 1 or 2;
subscript ‘y’ is an integer of 1 or 2;
subscript ‘n’ is an integer of 1, 2 or 3; and
subscript ‘q’ is an integer of from 1 to 5,
wherein each heterocycloalkyl and heteroaryl includes 1 to 4 heteroatoms each independently N, O or S.

In some embodiments, the present invention provides a compound of Formula (I):

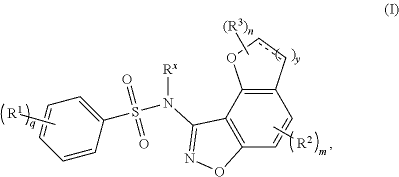

or a pharmaceutically acceptable salt or a stereoisomer or a tautomer thereof; wherein, dotted line [symbol] represents a single bond or is absent;
each R¹ is independently hydrogen, halogen, alkyl, haloalkyl, aminoalkyl, alkoxy or haloalkoxy;
each R² is independently hydrogen, alkyl, alkoxy, halogen, haloalkoxy, aryl or heterocyclyl; wherein the alkyl group is substituted with 0, 1, 2 or 3 R^{4a}; and the aryl and heterocyclyl groups are substituted with 0, 1, 2 or 3 R^{4b};
each R³ is independently hydrogen, alkyl, halogen, hydroxy or alkoxy; wherein the alkyl group is substituted with 0, 1, or more substituent(s) selected from hydroxy, alkoxy and halogen;
each R^{4a} is independently halogen, haloalkoxy, amino, alkoxy, aryl or heterocyclyl;
each R^{4b} is independently halogen, haloalkoxy, amino, alkyl, alkoxy, aryl or heterocyclyl;
R^{x} is hydrogen or alkyl;
subscript ‘m’ is an integer of 1 or 2;
subscript ‘y’ is an integer of 1 or 2;
subscript ‘n’ is an integer of 1, 2 or 3; and
subscript ‘q’ is an integer of from 1 to 5.

In some embodiments, the compound of Formula (I), or pharmaceutically acceptable salt thereof, is the compound wherein dotted line [symbol] represents a single bond or is absent;
each R¹ is independently hydrogen, halogen, (C₁-C₆)alkyl, (C₁-C₆)haloalkyl, (C₁-C₆)aminoalkyl, (C₁-C₆)alkoxy or (C₁-C₆)haloalkoxy;
each R² is independently hydrogen, (C₁-C₆)alkyl, (C₁-C₆)alkoxy, halogen, (C₁-C₆)haloalkoxy, (C₆-C₁₀)aryl, 3- to 8-membered heterocycloalkyl, or 5- to 10-membered heteroaryl; wherein the alkyl group is substituted with 0, 1, 2 or 3 R^{4a}; and the aryl, heterocycloalkyl and heteroaryl groups are substituted with 0, 1, 2 or 3 R^{4b};
each R³ is independently hydrogen, (C₁-C₆)alkyl, halogen, hydroxy or (C₁-C₆)alkoxy; wherein the alkyl group is substituted with 0, 1, 2, or 3 substituent(s) selected from hydroxy, (C₁-C₆)alkoxy and halogen;
each R^{4a} is independently halogen, haloalkoxy, amino, (C₁-C₆)alkoxy, (C₆-C₁₀)aryl or 3- to 8-membered heterocyclyl;
each R^{4b} is independently halogen, (C₁-C₆)haloalkoxy, amino, (C₁-C₆)alkyl, (C₁-C₆)alkoxy, (C₆-C₁₀)aryl, 3- to 8-heterocycloalkyl or 5- to 10-heteroaryl;
R^{x} is hydrogen or (C₁-C₆)alkyl;
subscript ‘m’ is an integer of 1 or 2;
subscript ‘y’ is an integer of 1 or 2;
subscript ‘n’ is an integer of 1, 2 or 3; and
subscript ‘q’ is an integer of from 1 to 5,
wherein each heterocycloalkyl and heteroaryl includes 1 to 4 heteroatoms each independently N, O or S.

In some embodiments, the compound of Formula (I), (IA-1), (IB-1), (IB-2), (IB-3), (IB-4), or (ID), or pharmaceutically acceptable salt thereof, is the compound wherein
each R¹ is independently (C₁-C₆)alkyl, (C₂-C₆)alkenyl, (C₂-C₆)alkynyl, (C₁-C₆)alkoxy, (C₂-C₆)alkoxyalkyl, (C₁-C₆)hydroxyalkyl, (C₁-C₆)aminoalkyl, halogen, (C₁-C₆)haloalkyl, (C₁-C₆)haloalkoxy, —C(O)R^{1h}, —C(O)OR^{1h}, (C₃-C₈)cycloalkyl, 3- to 8-membered heterocycloalkyl, (C₆-C₁₀)aryl, or 5- to 10-membered heteroaryl, wherein each alkoxy is substituted with 0, 1, 2, or 3 R^{1f}, and each cycloalkyl, heterocycloalkyl, aryl, and heteroaryl is substituted with 0, 1, 2, or 3 R^{1g};
each R^{1h} is independently hydrogen or (C₁-C₆)alkyl;
each R^{1f} is independently 5- to 10-membered heteroaryl; and
each R^{1g} is independently (C₁-C₆)alkyl, (C₁-C₆)alkoxy, or (C₁-C₆)haloalkyl; alternatively, two R¹ groups on adjacent ring atoms are combined to form (C₅-C₈)cycloalkyl or a 5- to 8-membered heterocycloalkyl.

In some embodiments, the compound of Formula (I), (IA-1), (IB-1), (IB-2), (IB-3), (IB-4), or (ID), or pharmaceutically acceptable salt thereof, is the compound wherein
each R¹ is independently (C₁-C₆)alkyl, (C₁-C₆)alkoxy, halogen, (C₁-C₆)haloalkyl, (C₁-C₆)haloalkoxy, —C(O)OR^{1h}, (C₃-C₈)cycloalkyl, (C₆-C₁₀)aryl, or 5- to 6-membered heteroaryl having 1 to 3 heteroatoms each N, wherein each alkoxy is substituted with 0, 1, or 2 Rif, and each cycloalkyl, aryl, and heteroaryl is substituted with 0, 1, or 2 R^{1g};
each R^{1h} is independently hydrogen or (C₁-C₆)alkyl;
each R^{1f} is independently 5- to 6-membered heteroaryl having 1 to 3 heteroatoms each N; and
each R^{1g} is independently (C₁-C₆)haloalkyl;
alternatively, two R¹ groups on adjacent ring atoms are combined to form (C₅-C₈)cycloalkyl.

In some embodiments, the compound of Formula (I), (IA-1), (IB-1), (IB-2), (IB-3), (IB-4), or (ID), or pharmaceutically acceptable salt thereof, is the compound wherein
each R¹ is independently (C₁-C₄)alkyl, (C₁-C₃)alkoxy, halogen, (C₁-C₃)haloalkyl, (C₁-C₃)haloalkoxy, —C(O)OR^{1h}, (C₃-C₈)cycloalkyl, (C₆-C₁₀)aryl, or 5- to 6-membered heteroaryl having 1 to 3 heteroatoms each N, wherein each alkoxy is substituted with 0 or 1 R^{1f};
each R^{1h} is independently hydrogen or (C₁-C₃)alkyl; and
each R^{1f} is independently 5- to 6-membered heteroaryl having 1 to 3 heteroatoms each N;
alternatively, two R¹ groups on adjacent ring atoms are combined to form (C₅-C₈)cycloalkyl.

In some embodiments, the compound of Formula (I), (IA-1), (IB-1), (IB-2), (IB-3), (IB-4), or (ID), or pharmaceutically acceptable salt thereof, is the compound wherein each R¹ is independently hydrogen, halogen, alkyl, haloalkyl, alkoxy or haloalkoxy.

In some embodiments, the compound of Formula (I), (IA-1), (IB-1), (IB-2), (IB-3), (IB-4), or (ID), or pharmaceutically acceptable salt thereof, is the compound wherein each R¹ is independently hydrogen, halogen, alkyl, alkoxy or haloalkoxy. In some embodiments, the compound of Formula (I), (IA-1), (IB-1), (IB-2), (IB-3), (IB-4), or (ID), or pharmaceutically acceptable salt thereof, is the compound wherein each R¹ is independently hydrogen, alkyl or alkoxy. In some embodiments, each R¹ is independently hydrogen or alkoxy.

In some embodiments, the compound of Formula (I), (IA-1), (IB-1), (IB-2), (IB-3), (IB-4), or (ID), or pharmaceutically acceptable salt thereof, is the compound wherein each R¹ is independently Et, t-Bu, OMe, OEt, OCH₂-pyrid-2-yl, F, Cl, CF₃, OCF₃, COOH, COOMe, cyclopropyl, phenyl, or

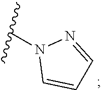 [SMILES: *n1cccn1]

alternatively, two R¹ on adjacent ring atoms are combined to form cyclopentyl or cyclohexyl.

In some embodiments, the compound of Formula (I), (IA-1), (IB-1), (IB-2), (IB-3), (IB-4), or (ID), or pharmaceutically acceptable salt thereof, is the compound wherein
each R¹ is independently hydrogen, halogen, (C₁-C₆)alkyl or (C₁-C₆)alkoxy;
each R² is independently

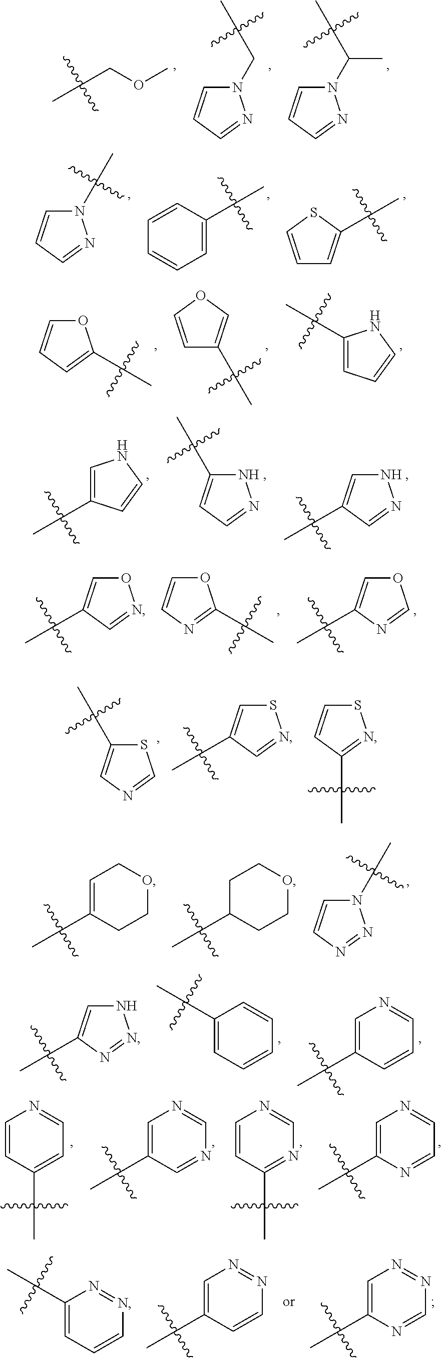

wherein each group is substituted with 0 or 1 (C₁-C₆)alkyl or (C₁-C₆)alkoxy;
each R³ is independently hydrogen, (C₁-C₆)alkyl or halogen; wherein the alkyl group is substituted with 0 to 3 substituents selected from hydroxy, (C₁-C₆)alkoxy and halogen;
subscript ‘m’ is 1;
subscript ‘n’ is 1 or 2; and
subscript ‘q’ is 1 or 2.

In some embodiments, the compound of Formula (I), (IA), (IA-1), (IB), (IB-1), (IB-2), (IB-3), (IB-4), or (IC), or pharmaceutically acceptable salt thereof, is the compound wherein each R² is independently hydrogen, alkyl, alkoxy, aryl or heterocyclyl; wherein the alkyl group is substituted with 0 or 1 R^{4a}; and the aryl and heterocyclyl groups are substituted with 0 or 1 R^{4b}.

In some embodiments, the compound of Formula (I), (IA), (IA-1), (IB), (IB-1), (IB-2), (IB-3), (IB-4), or (IC), or pharmaceutically acceptable salt thereof, is the compound wherein each R² is independently heterocyclyl comprising heteroaryl or heterocycloalkyl.

In some embodiments, the compound of Formula (I), (IA), (IA-1), (IB), (IB-1), (IB-2), (IB-3), (IB-4), or (IC), or pharmaceutically acceptable salt thereof, is the compound wherein each R² is independently an alkyl substituted with 0, 1, 2 or 3 R^{4a}. In some embodiments, the compound of Formula (I), (IA), (IA-1), (IB), (IB-1), (IB-2), (IB-3), (IB-4), or (IC), or pharmaceutically acceptable salt thereof, is the compound wherein each R² is independently an alkyl substituted with R^{4a}. In some embodiments, the compound of Formula (I), (IA), (IA-1), (IB), (IB-1), (IB-2), (IB-3), (IB-4), or (IC), or pharmaceutically acceptable salt thereof, is the compound wherein each R² is independently an alkyl substituted with alkoxy or heteroaryl.

In some embodiments, the compound of Formula (I), (IA), (IA-1), (IB), (IB-1), (IB-2), (IB-3), (IB-4), or (IC), or pharmaceutically acceptable salt thereof, is the compound wherein each R^{4a} is independently halogen, amino, alkoxy, aryl or heterocyclyl (heteroaryl and heterocycloalkyl). In some embodiments, the compound of Formula (I), (IA), (IA-1), (IB), (IB-1), (IB-2), (IB-3), (IB-4), or (IC), or pharmaceutically acceptable salt thereof, is the compound wherein each R^{4a} is independently 6-membered aryl or 5- to 6-membered heterocyclyl. In some embodiments, the compound of Formula (I), (IA), (IA-1), (IB), (IB-1), (IB-2), (IB-3), (IB-4), or (IC), or pharmaceutically acceptable salt thereof, is the compound wherein each R^{4a} is independently 5- to 6-membered heterocyclyl comprising 5- to 6-membered heteroaryl or 5- to 6-membered heterocycloalkyl.

In some embodiments, the compound of Formula (I), (IA), (IA-1), (IB), (IB-1), (IB-2), (IB-3), (IB-4), or (IC), or pharmaceutically acceptable salt thereof, is the compound wherein each R² is independently aryl, heteroaryl or heterocycloalkyl; wherein each group is substituted with 0, 1, 2 or 3 R^{4b}. In some embodiments, the compound of Formula (I), (IA), (IA-1), (IB), (IB-1), (IB-2), (IB-3), (IB-4), or (IC), or pharmaceutically acceptable salt thereof, is the compound wherein R^{4b} represents halogen, haloalkoxy, amino, alkyl, alkoxy or heterocyclyl (heteroaryl and heterocycloalkyl).

In some embodiments, the compound of Formula (I), (IA), (IA-1), (IB), (IB-1), (IB-2), (IB-3), (IB-4), or (IC), or pharmaceutically acceptable salt thereof, is the compound wherein each R² is independently aryl, heteroaryl or heterocycloalkyl; wherein each group is substituted with 0 or 1 alkyl or alkoxy.

In some embodiments, the compound of Formula (I), (IA), (IA-1), (IB), (IB-1), (IB-2), (IB-3), (IB-4), or (IC), or pharmaceutically acceptable salt thereof, is the compound wherein each R² is independently hydrogen, alkyl, aryl, 5- to 6-membered heteroaryl or 5- to 6-membered heterocycloalkyl.

In some embodiments, the compound of Formula (I), (IA), (IA-1), (IB), (IB-1), (IB-2), (IB-3), (IB-4), or (IC), or pharmaceutically acceptable salt thereof, is the compound wherein each R² is independently

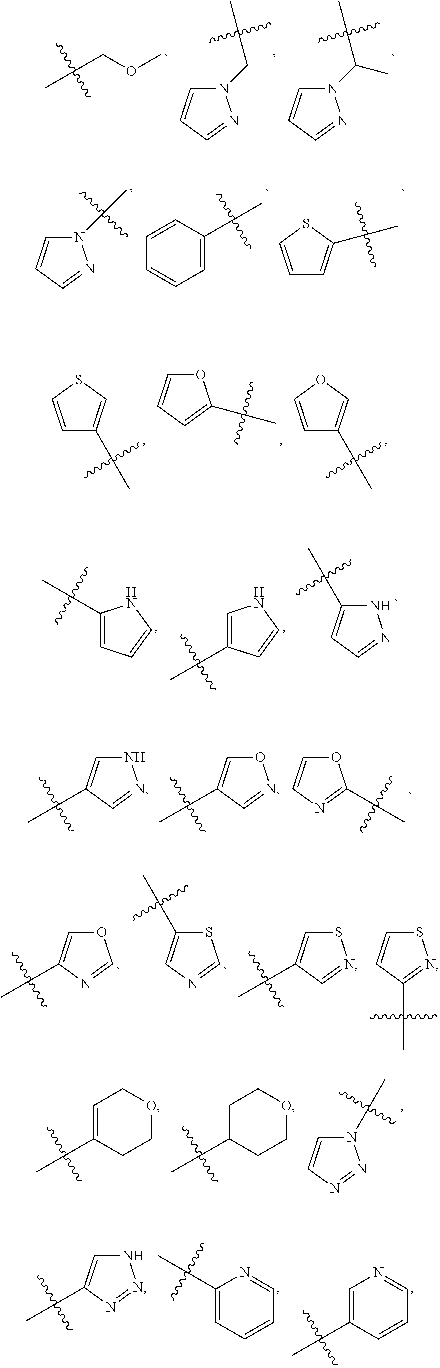

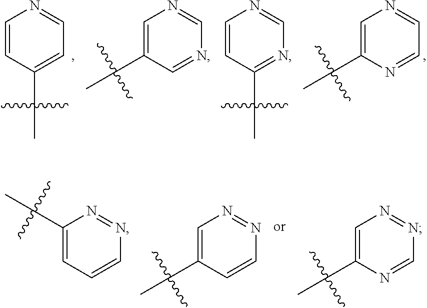

wherein [symbol] represents points of attachment and each group is optionally substituted with alkyl or alkoxy.

In some embodiments, the compound of Formula (I), (IA), (IA-1), (IB), (IB-1), (IB-2), (IB-3), (IB-4), (IC), or (ID), or pharmaceutically acceptable salt thereof, is the compound wherein each R³ is independently hydrogen, alkyl or halogen; wherein the alkyl group is substituted with 0 to 3 substituent(s) selected from hydroxy, alkoxy and halogen. In some embodiments, the compound of Formula (I), (IA), (IA-1), (IB), (IB-1), (IB-2), (IB-3), (IB-4), (IC), or (ID), or pharmaceutically acceptable salt thereof, is the compound wherein each R³ is independently hydrogen or alkyl. In some embodiments, the compound of Formula (I), (IA), (IA-1), (IB), (IB-1), (IB-2), (IB-3), (IB-4), (IC), or (ID), or pharmaceutically acceptable salt thereof, is the compound wherein each R³ is independently hydrogen, (C₁-C₆)alkyl or halogen; wherein the alkyl group is substituted with 0 to 3 substituent(s) selected from hydroxy, (C₁-C₆)alkoxy and halogen.

In some embodiments, the compound of Formula (I), or pharmaceutically acceptable salt thereof, is the compound wherein R^{x} is hydrogen.

In some embodiments, the compound of Formula (I), or pharmaceutically acceptable salt thereof, is the compound wherein subscript ‘m’ is 1. In some embodiments, the compound of Formula (I), or pharmaceutically acceptable salt thereof, is the compound wherein subscript ‘n’ is an integer of 1 to 2. In some embodiments, the compound of Formula (I), or pharmaceutically acceptable salt thereof, is the compound wherein subscript ‘q’ is an integer of from 1 to 3. In some embodiments, the compound of Formula (I), or pharmaceutically acceptable salt thereof, is the compound wherein subscript ‘y’ is 1. In some embodiments, the compound of Formula (I), or pharmaceutically acceptable salt thereof, is the compound wherein subscript ‘y’ is 2.

In some embodiments, the compound of Formula (I), or pharmaceutically acceptable salt thereof, is the compound wherein the formula

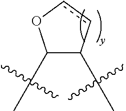

represents

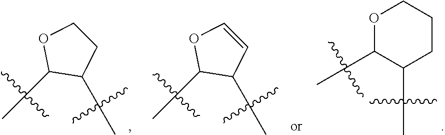 [SMILES: *C1CCOC1* ; *C1C=COC1* ; *C1CCCOC1*]

In some embodiments, the present invention provides a compound of Formula (I), wherein
dotted lines [symbol] represents a single bond or is absent;
each R¹ is independently hydrogen, halogen, alkyl or alkoxy;
each R² is independently hydrogen, alkyl, alkoxy, aryl or heterocyclyl; wherein the alkyl group is substituted with 0, 1, 2 or 3 R^{4a}; and the aryl and heterocyclyl groups are substituted with 0, 1, 2 or 3 R^{4b};
each R^{4a} is independently halogen, amino, alkoxy, aryl, heteroaryl or heterocycloalkyl;
each R^{4b} is independently halogen, haloalkoxy, amino, alkyl, alkoxy, heteroaryl or heterocycloalkyl;
each R³ is independently hydrogen, alkyl or halogen; wherein the alkyl group is substituted with 0 to 3 substituent(s) selected from hydroxy, alkoxy or halogen;
R^{x} represents hydrogen;
subscript ‘m’ is 1;
subscript ‘y’ is an integer of 1 or 2;
subscript ‘n’ is an integer of 1 or 2; and
subscript ‘q’ is an integer of from 1 to 3.

In some embodiments, the present invention provides a compound of Formula (I), wherein
dotted lines [symbol] represents a single bond or is absent;
each R¹ is independently hydrogen, halogen, (C₁-C₆)alkyl or (C₁-C₆)alkoxy;
each R² is independently

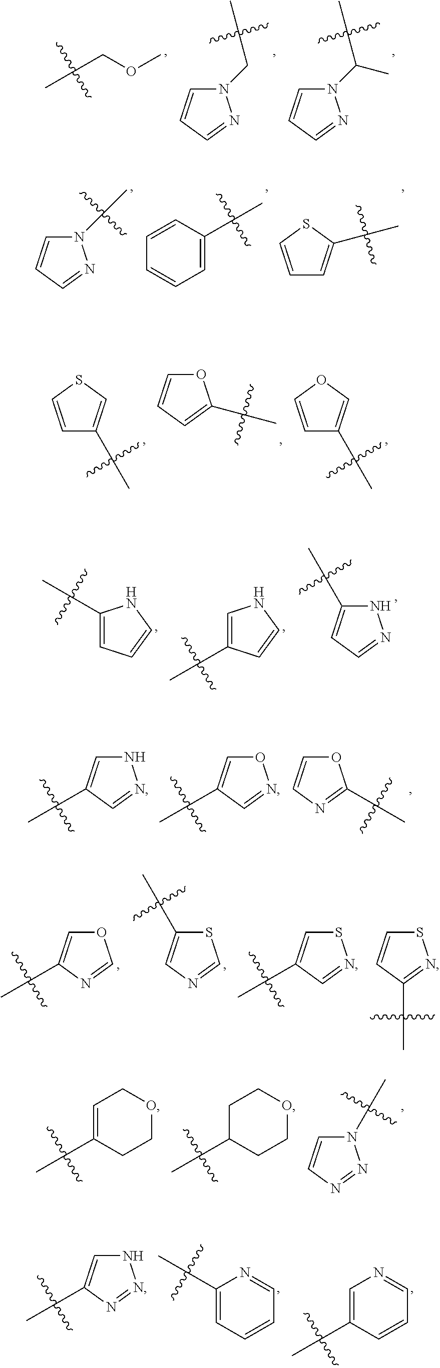

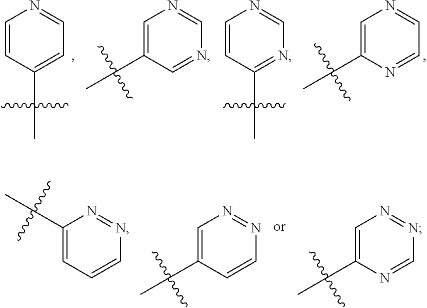

wherein each group is substituted with 0 or 1 (C₁-C₆)alkyl or (C₁-C₆)alkoxy;
each R³ is independently hydrogen, (C₁-C₆)alkyl or halogen; wherein the alkyl group is substituted with 0 to 3 substituent(s) selected from hydroxy, (C₁-C₆)alkoxy and halogen;
subscript ‘m’ is 1;
subscript ‘y’ is an integer of 1 or 2;
subscript ‘n’ is an integer of 1 or 2; and
subscript ‘q’ is an integer of 1 or 2.

In some embodiments, the present invention provides a compound of Formula (IA):

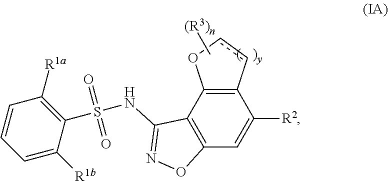

or a pharmaceutically acceptable salt or a stereoisomer or a tautomer thereof,
wherein each R^{1a} and R^{1b} is independently hydrogen, halogen, alkyl or alkoxy; and R², R³, subscript ‘n’ and subscript ‘y’ are as defined in compound of Formula (I).

In some embodiments, the compound of Formula (IA), (IB) or (IC), or pharmaceutically acceptable salt thereof, is the compound wherein R^{1a} and R^{1b} independently represent halogen, alkyl or alkoxy. In some embodiments, the compound of Formula (IA), (IB) or (IC), or pharmaceutically acceptable salt thereof, is the compound wherein R¹ and R^{1b} independently represent halogen, (C₁-C₆)alkyl or (C₁-C₆)alkoxy.

In some embodiments, the compound of Formula (I), (IA), (IA-1), (IB), (IB-1), (IB-2), (IB-3), (IB-4), or (IC), or pharmaceutically acceptable salt thereof, is the compound wherein each R² is independently (C₁-C₆)alkyl, (C₁-C₆)alkoxy, 6-membered aryl or 5- to 6-membered heterocyclyl; wherein the alkyl group is substituted with 0 or 1 R^{4a}; and the aryl and heterocyclyl groups are substituted with 0 or 1 R^{4b}.

In some embodiments, the compound of Formula (I), (IA), (IA-1), (IB), (IB-1), (IB-2), (IB-3), (IB-4), or (IC), or pharmaceutically acceptable salt thereof, is the compound wherein each R² is independently (C₁-C₆)alkyl, 6-membered aryl or 5- to 6-membered heterocyclyl.

In some embodiments, the compound of Formula (I), (IA), (IA-1), (IB), (IB-1), (IB-2), (IB-3), (IB-4), or (IC), or pharmaceutically acceptable salt thereof, is the compound wherein each R² is independently 5- to 6-membered heterocyclyl comprising 5- to 6-membered heteroaryl and 5- to 6-membered heterocycloalkyl.

In some embodiments, the compound of Formula (I), (IA), (IA-1), (IB), (IB-1), (IB-2), (IB-3), (IB-4), or (IC), or pharmaceutically acceptable salt thereof, is the compound wherein each R² is independently 6-membered aryl, 5- to 6-membered heteroaryl or 5- to 6-membered heterocycloalkyl; wherein each group is substituted with 0, 1, 2 or 3 occurrence(s) of R^{4b}.

In some embodiments, the compound of Formula (I), (IA), (IA-1), (IB), (IB-1), (IB-2), (IB-3), (IB-4), or (IC), or pharmaceutically acceptable salt thereof, is the compound wherein each R² is independently (C₁-C₆)alkyl substituted with R^{4a}.

In some embodiments, the compound of Formula (I), (IA), (IA-1), (IB), (IB-1), (IB-2), (IB-3), (IB-4), or (IC), or pharmaceutically acceptable salt thereof, is the compound wherein each R^{4a} is independently halogen, (C₁-C₆)alkoxy or 5- to 6-membered heterocyclyl.

In some embodiments, the compound of Formula (I), (IA), (IA-1), (IB), (IB-1), (IB-2), (IB-3), (IB-4), or (IC), or pharmaceutically acceptable salt thereof, is the compound wherein each R^{4a} is independently (C₁-C₆)alkoxy or 5- to 6-membered heteroaryl.

In some embodiments, the compound of Formula (I), (IA), (IA-1), (IB), (IB-1), (IB-2), (IB-3), (IB-4), or (IC), or pharmaceutically acceptable salt thereof, is the compound wherein R^{4b} represents halogen, halo(C₁-C₆)alkoxy, amino, (C₁-C₆)alkyl, (C₁-C₆)alkoxy or 5- to 6-membered heterocyclyl.

In some embodiments, the compound of Formula (I), (IA), (IA-1), (IB), (IB-1), (IB-2), (IB-3), (IB-4), or (IC), or pharmaceutically acceptable salt thereof, is the compound wherein R^{4b} represents halogen, halo(C₁-C₆)alkoxy, amino, (C₁-C₆)alkyl, (C₁-C₆)alkoxy, 5- to 6-membered heteroaryl or 5- to 6-membered heterocycloalkyl.

In some embodiments, the compound of Formula (I), (IA), (IA-1), (IB), (IB-1), (IB-2), (IB-3), (IB-4), or (IC), or pharmaceutically acceptable salt thereof, is the compound wherein R^{4b} represents halogen, (C₁-C₆)alkyl or (C₁-C₆)alkoxy.

In some embodiments, the compound of Formula (I), (IA), (IA-1), (IB), (IB-1), (IB-2), (IB-3), (IB-4), or (IC), or pharmaceutically acceptable salt thereof, is the compound wherein each R^{4a} is independently (C₁-C₆)alkoxy or 5- to 6-membered heteroaryl; and R^{4b} represents halogen, halo(C₁-C₆)alkoxy, amino, (C₁-C₆)alkyl, (C₁-C₆)alkoxy, 5- to 6-membered heteroaryl or 5- to 6-membered heterocycloalkyl.

In some embodiments, the compound of Formula (I), (IA), (IA-1), (IB), (IB-1), (IB-2), (IB-3), (IB-4), or (IC), or pharmaceutically acceptable salt thereof, is the compound wherein each R² is independently an alkyl substituted with (C₁-C₆)alkoxy or 5- to 6-membered heteroaryl.

In some embodiments, the compound of Formula (I), (IA), (IA-1), (IB), (IB-1), (IB-2), (IB-3), (IB-4), or (IC), or pharmaceutically acceptable salt thereof, is the compound wherein each R² is independently aryl, heteroaryl or heterocycloalkyl; wherein each group is substituted with 0 or 1 (C₁-C₆)alkyl or (C₁-C₆)alkoxy.

In some embodiments, the compound of Formula (I), (IA), (IA-1), (IB), (IB-1), (IB-2), (IB-3), (IB-4), or (IC), or pharmaceutically acceptable salt thereof, is the compound wherein each R² is independently

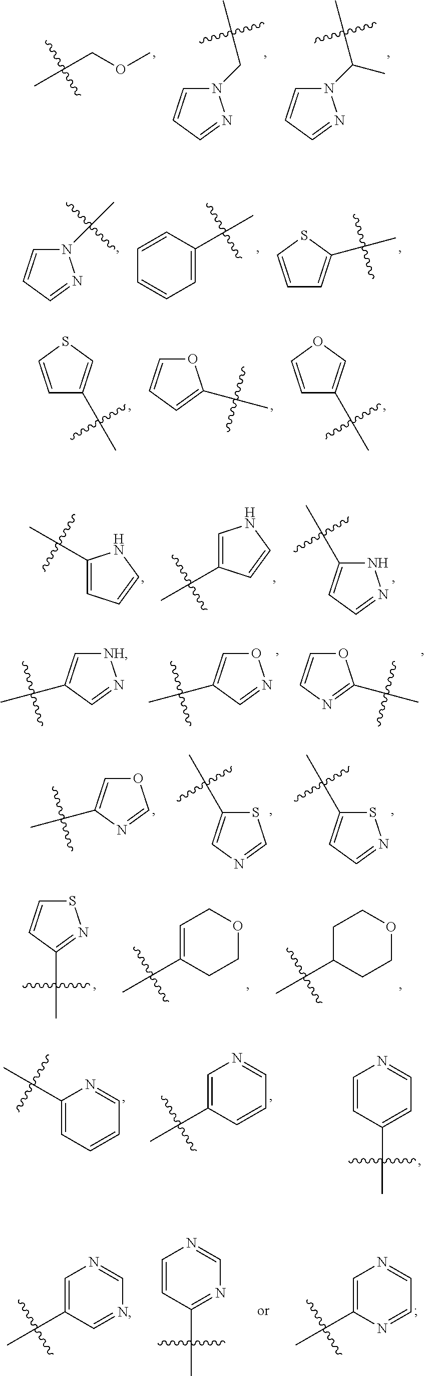

wherein each group is substituted with 0 or 1 (C₁-C₆)alkyl or (C₁-C₆)alkoxy.

In some embodiments, the compound of Formula (I), (IA), (IA-1), (IB), (IB-1), (IB-2), (IB-3), or (IB-4), or pharmaceutically acceptable salt thereof, is the compound wherein subscript ‘n’ is an integer of 1 or 2. In some embodiments, the compound of Formula (I), (IA) or (IA-1), or pharmaceutically acceptable salt thereof, is the compound wherein subscript ‘y’ is 1. In some embodiments, the compound of Formula (I), (IA) or (IA-1), or pharmaceutically acceptable salt thereof, is the compound wherein subscript ‘y’ is 2.

In some embodiments, the compound of Formula (I), (IA), (IA-1), (IB), (IB-1), (IB-2), (IB-3), (IB-4), (IC), or (ID), or pharmaceutically acceptable salt thereof, is the compound wherein each R³ is independently hydrogen, (C₁-C₆)alkyl or halogen; wherein the alkyl group is substituted with 0 to 3 substituent(s) selected from hydroxy, (C₁-C₆)alkoxy or halogen.

In some embodiments, the compound of Formula (I), (IA), (IA-1), (IB), (IB-1), (IB-2), (IB-3), (IB-4), (IC), or (ID), or pharmaceutically acceptable salt thereof, is the compound wherein each R³ is independently (C₁-C₆)alkyl substituted with (C₁-C₆)alkoxy.

In some embodiments, the present invention provides a compound of Formula (IA), wherein
dotted lines [symbol] represents a single bond or is absent;
R^{1a} and R^{1b} independently represent (C₁-C₆)alkyl or (C₁-C₆)alkoxy;
each R² is independently

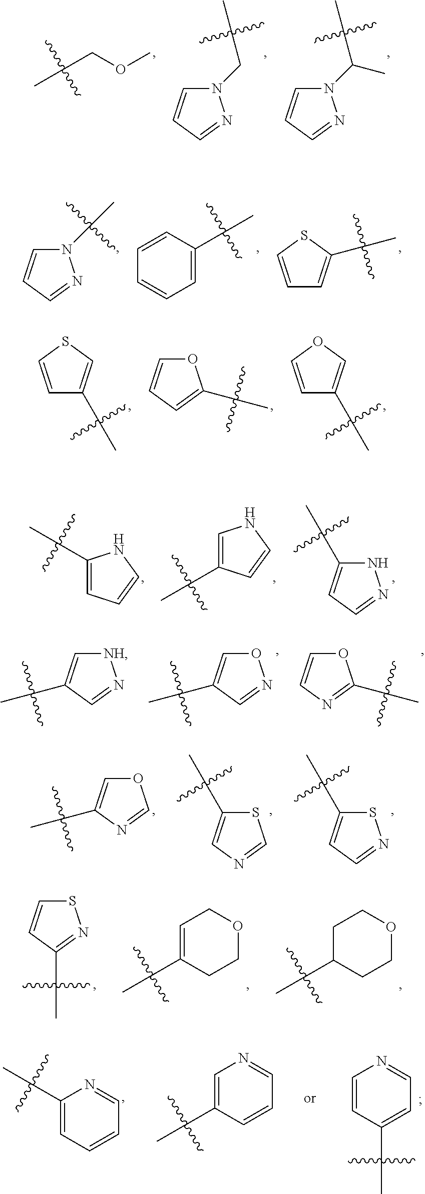

wherein each group is substituted with 0 or 1 (C₁-C₆)alkyl or (C₁-C₆)alkoxy;
each R³ is independently hydrogen, (C₁-C₆)alkyl or halogen; wherein the alkyl group is substituted with 0 to 3 substituent(s) selected from hydroxy, (C₁-C₆)alkoxy and halogen;
subscript ‘y’ is an integer of 1 or 2; and
subscript ‘n’ is an integer of 1 or 2.

In some embodiments, the compound of Formula (I), or pharmaceutically acceptable salt thereof has the structure of Formula (IA-1):

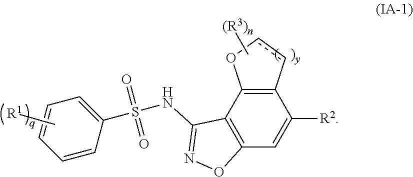

In some embodiments, the compound of Formula (I), (IA) or (IA-1), or the pharmaceutically acceptable salt thereof, is the compound wherein the dotted line [symbol] is absent.

In some embodiments, the compound of Formula (I), (IA) or (IA-1), or the pharmaceutically acceptable salt thereof, is the compound wherein subscript ‘y’ is 1. In some embodiments, the compound of Formula (I), (IA) or (IA-1), or the pharmaceutically acceptable salt thereof, is the compound wherein subscript ‘y’ is 2.

In some embodiments, the present invention provides a compound of Formula (IB):

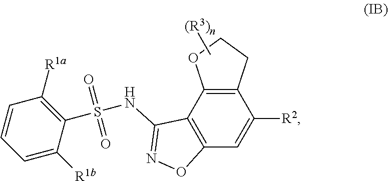

or a pharmaceutically acceptable salt or a stereoisomer or a tautomer thereof, wherein, each R^{1a} and R^{1b} is independently hydrogen, halogen, alkyl or alkoxy; and R², R³ and subscript ‘n’ are as defined in compound of Formula (I).

In some embodiments, the compound of Formula (IA), (IB), or (IC), or pharmaceutically acceptable salt thereof, is the compound wherein each R^{1a} and R^{1b} is independently hydrogen, halogen, (C₁-C₆)alkyl or (C₁-C₆)alkoxy.

In some embodiments, the compound of Formula (IA), (IB), or (IC), or pharmaceutically acceptable salt thereof, is the compound wherein R^{1a} and R^{1b} are each independently (C₁-C₆)alkyl or (C₁-C₆)alkoxy.

In some embodiments, the compound of Formula (I), (IA), (IA-1), (IB), (IB-1), (IB-2), (IB-3), (IB-4), or (IC), or pharmaceutically acceptable salt thereof, is the compound wherein each R² is independently

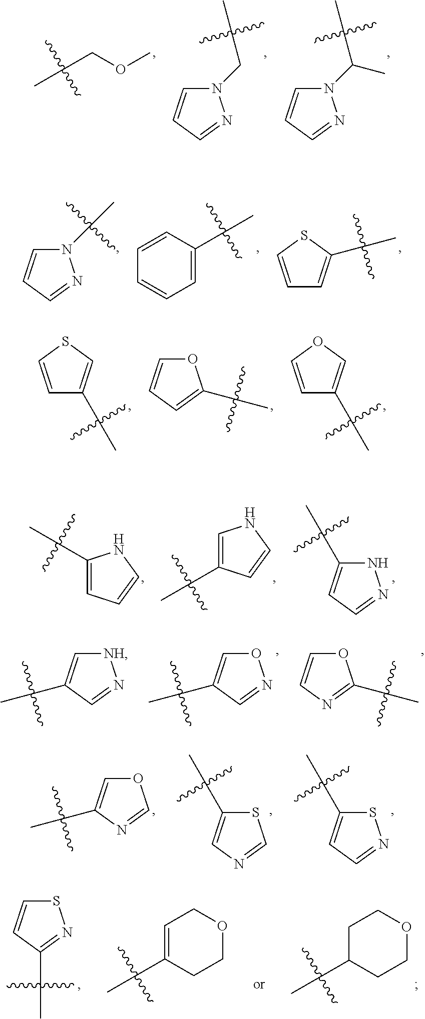

wherein each group is substituted with 0 or 1 (C₁-C₆)alkyl or (C₁-C₆)alkoxy.

In some embodiments, the compound of Formula (I), (IA), (IA-1), (IB), (IB-1), (IB-2), (IB-3), (IB-4), (IC), or (ID), or pharmaceutically acceptable salt thereof, is the compound wherein each R³ is independently hydrogen, (C₁-C₆)alkyl or halogen; wherein the alkyl group is substituted with 0 to 3 substituent(s) selected from hydroxy, (C₁-C₆)alkoxy and halogen.

In some embodiments, the compound of Formula (IA), (IB), or (IC), or pharmaceutically acceptable salt thereof, is the compound wherein each R^{1a} and R^{1b} is independently (C₁-C₆)alkyl or (C₁-C₆)alkoxy;
R² is

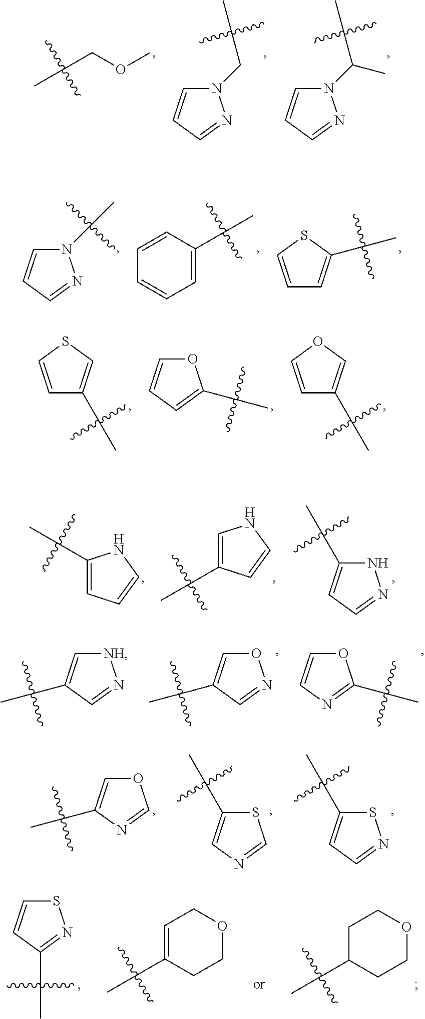

which is substituted with 0 or 1 (C₁-C₆)alkyl or (C₁-C₆)alkoxy; and
each R³ is independently hydrogen, (C₁-C₆)alkyl or halogen; wherein the alkyl group is substituted with 0 to 3 substituent(s) selected from hydroxy, (C₁-C₆)alkoxy and halogen.

In some embodiments, the present invention provides a compound of Formula (IB), wherein
each R^{1a} and R^{1b} is independently (C₁-C₆)alkyl or (C₁-C₆)alkoxy;
R² is

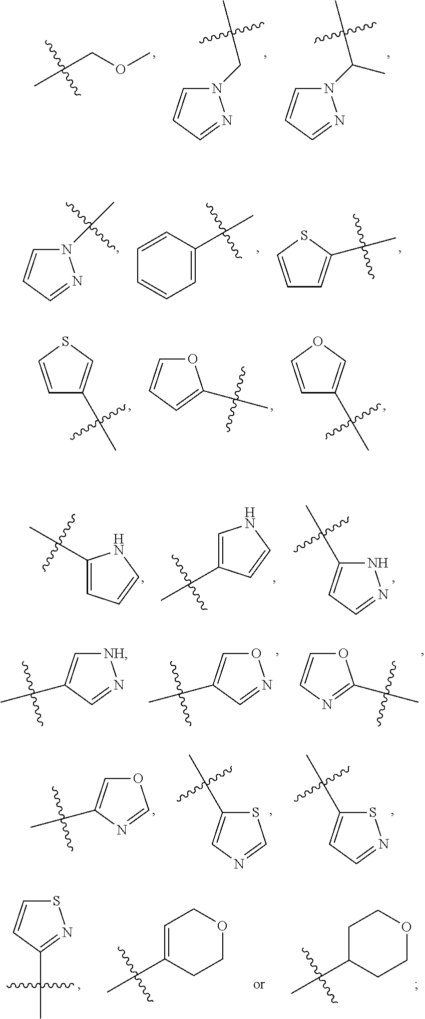

which is substituted with 0 or 1 (C₁-C₆)alkyl or (C₁-C₆)alkoxy;
each R³ is independently hydrogen, (C₁-C₆)alkyl or halogen; wherein the alkyl group is substituted with 0 to 3 substituent(s) selected from hydroxy, (C₁-C₆)alkoxy and halogen; and subscript ‘n’ is 1 or 2.

In some embodiments, the compound of Formula (I) or (IA-1), or pharmaceutically acceptable salt thereof, is the compound having the structure of Formula (IB-1):

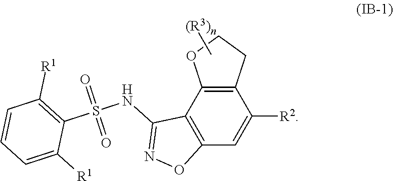

In some embodiments, the compound of Formula (I) or (IA-1), or pharmaceutically acceptable salt thereof, is the compound having the structure of Formula (IB-2):

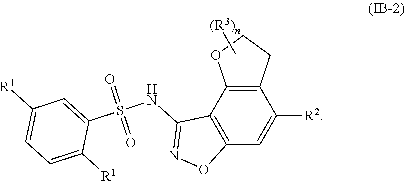

In some embodiments, the compound of Formula (I) or (IA-1), or pharmaceutically acceptable salt thereof, is the compound having the structure of Formula (IB-3):

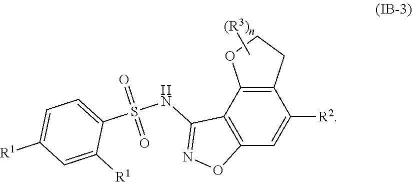

In some embodiments, the compound of Formula (IB-1), (IB-2), or (IB-3), or pharmaceutically acceptable salt thereof, is the compound wherein
each R¹ is independently (C₁-C₆)alkyl, (C₁-C₆)alkoxy, halogen, (C₁-C₆)haloalkyl, (C₁-C₆)haloalkoxy, —C(O)OR^{1h}, (C₃-C₈)cycloalkyl, (C₆-C₁₀)aryl, or 5- to 10-membered heteroaryl having 1 to 3 heteroatoms each N, wherein each alkoxy is substituted with 0, 1, 2, or 3 R^{1f}, and each aryl, and heteroaryl is substituted with 0, 1, 2, or 3 R^{1g};
each R^{1h} is independently hydrogen or (C₁-C₆)alkyl;
each R^{1f} is independently 5- to 6-membered heteroaryl having 1 to 3 heteroatoms each N; and
each R^{1g} is independently (C₁-C₆)haloalkyl.

In some embodiments, the compound of Formula (IB-1), (IB-2), or (IB-3), or pharmaceutically acceptable salt thereof, is the compound wherein each R¹ is independently (C₁-C₃)alkyl, (C₁-C₃)alkoxy, halogen, or (C₁-C₃)haloalkyl.

In some embodiments, the compound of Formula (I) or (IA-1), or pharmaceutically acceptable salt thereof, is the compound having the structure of Formula (IB-4):

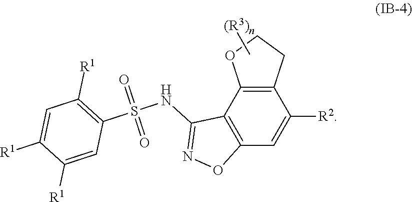

In some embodiments, the compound of Formula (IB-4), or pharmaceutically acceptable salt thereof, is the compound wherein each R¹ is independently (C₁-C₆)alkyl, (C₁-C₆)alkoxy, halogen, (C₁-C₆)haloalkyl, (C₁-C₆)haloalkoxy, —C(O)OR^{1h}, (C₃-C₈)cycloalkyl, (C₆-C₁₀) aryl, or 5- to 10-membered heteroaryl having 1 to 3 heteroatoms each N, wherein each alkoxy is substituted with 0, 1, 2, or 3 R^{1f}, and each aryl, and heteroaryl is substituted with 0, 1, 2, or 3 R^{1g};
each R^{1h} is independently hydrogen or (C₁-C₆)alkyl;
each R^{1f} is independently 5- to 6-membered heteroaryl having 1 to 3 heteroatoms each N; and
each R^{1g} is independently (C₁-C₆)haloalkyl;
alternatively, two R¹ groups on adjacent ring atoms are combined to form (C₅-C₈)cycloalkyl.

In some embodiments, the present invention provides a compound of Formula (IC):

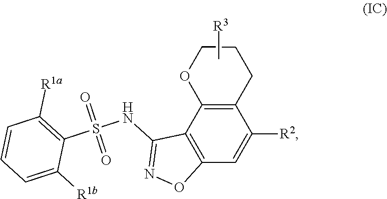

or a pharmaceutically acceptable salt or a stereoisomer or a tautomer thereof, wherein each R^{1a} and R^{1b} is independently hydrogen, halogen, alkyl or alkoxy; and R² and R³ are as defined in compound of Formula (I).

In some embodiments, the compound of Formula (IC), or pharmaceutically acceptable salt thereof, is the compound wherein each R^{1a} and R^{1b} is independently hydrogen, halogen, (C₁-C₆)alkyl or (C₁-C₆)alkoxy.

In some embodiments, the compound of Formula (IA), (IB), or (IC), or pharmaceutically acceptable salt thereof, is the compound wherein each R^{1a} and R^{1b} is independently (C₁-C₆)alkoxy.

In some embodiments, the compound of Formula (I), (IA), (IA-1), (IB), (IB-1), (IB-2), (IB-3), (IB-4), or (IC), or pharmaceutically acceptable salt thereof, is the compound wherein
each R² is independently (C₁-C₆)alkyl, (C₂-C₆)alkenyl, (C₂-C₆)alkynyl, (C₁-C₆)alkoxy, (C₁-C₆)hydroxyalkyl, halogen, (C₁-C₆)haloalkoxy, hydroxy, (C₃-C₈)cycloalkyl, (C₆-C₁₀)aryl, 3- to 8-membered heterocycloalkyl, or 5- to 10-membered heteroaryl; wherein each alkyl group is substituted with 0, 1, 2 or 3 R^{4a}; and each cycloalkyl, heterocycloalkyl, aryl, and heteroaryl is substituted with 0, 1, 2 or 3 R^{4b};
each R^{4a} is independently (C₁-C₆)alkoxy, halogen, C₁-C₆ haloalkoxy, —NH₂, —CN, —NO₂, C₃-C₈ cycloalkyl, (C₆-C₁₀)aryl, 3- to 8-membered heterocycloalkyl, or 5- to 10-membered heteroaryl, wherein each cycloalkyl, heterocycloalkyl, aryl, and heteroaryl is substituted with 0, 1, 2, or 3 R^{4a1};
each R^{4a1} is independently (C₁-C₆)alkyl, (C₂-C₆)alkenyl, (C₂-C₆)alkynyl, (C₁-C₆)alkoxy, (C₂-C₆)alkoxyalkyl, (C₁-C₆)hydroxyalkyl, halogen, (C₁-C₆)haloalkyl, (C₁-C₆)haloalkoxy, or —CN; and
each R^{4b} is independently (C₁-C₆)alkyl, (C₂-C₆)alkenyl, (C₂-C₆)alkynyl, (C₁-C₆)alkoxy, (C₁-C₆)hydroxyalkyl, halogen, (C₁-C₆)haloalkyl, (C₁-C₆)haloalkoxy, (C₃-C₈)cycloalkyl, (C₆-C₁₀)aryl, 3- to 8-membered heterocycloalkyl, or 5- to 10-membered heteroaryl.

In some embodiments, the compound of Formula (I), (IA), (IA-1), (IB), (IB-1), (IB-2), (IB-3), (IB-4), or (IC), or pharmaceutically acceptable salt thereof, is the compound wherein
each R² is independently (C₁-C₆)alkyl, (C₂-C₆)alkenyl, (C₂-C₆)alkynyl, (C₃-C₈)cycloalkyl, (C₆-C₁₀)aryl, 3- to 8-membered heterocycloalkyl, or 5- to 10-membered heteroaryl; wherein each alkyl group is substituted with 0, 1, 2 or 3 R^{4a}; and each cycloalkyl, heterocycloalkyl, aryl, and heteroaryl is substituted with 0, 1, 2 or 3 R^{4b};
each R^{4a} is independently C₃-C₈ cycloalkyl, (C₆-C₁₀)aryl, 3- to 8-membered heterocycloalkyl, or 5- to 10-membered heteroaryl, wherein each cycloalkyl, heterocycloalkyl, aryl, and heteroaryl is substituted with 0, 1, 2, or 3 R^{4a1};
each R^{4a1} is independently halogen, (C₁-C₆)haloalkyl, (C₁-C₆)haloalkoxy, or —CN; and each R^{4b} is independently (C₁-C₆)alkyl, (C₂-C₆)alkenyl, (C₂-C₆)alkynyl, (C₁-C₆)alkoxy, or (C₁-C₆)hydroxyalkyl.

In some embodiments, the compound of Formula (I), (IA), (IA-1), (IB), (IB-1), (IB-2), (IB-3), (IB-4), or (IC), or pharmaceutically acceptable salt thereof, is the compound wherein
each R² is independently (C₁-C₆)alkyl, (C₆-C₁₀)aryl, 5- to 6-membered heterocycloalkyl including 1 to 2 heteroatoms each independently O, or 5- to 6-membered heteroaryl including 1 to 3 heteroatoms each independently N, O or S; wherein each alkyl group is substituted with 0, 1, 2 or 3 R^{4a}; and each heterocycloalkyl, aryl, and heteroaryl is substituted with 0, 1, 2 or 3 R^{4b};
each R^{4a} is independently 5- to 6-membered heteroaryl including 1 to 3 heteroatoms each independently N, substituted with 0, 1, 2, or 3 R^{4a1};
each R^{4a1} is independently halogen, (C₁-C₆)haloalkyl, or —CN; and
each R^{4b} is independently (C₁-C₆)alkyl, or (C₁-C₆)alkoxy.

In some embodiments, the compound of Formula (I), (IA), (IA-1), (IB), (IB-1), (IB-2), (IB-3), (IB-4), or (IC), or pharmaceutically acceptable salt thereof, is the compound wherein
each R² is independently (C₁-C₃)alkyl, phenyl, 5- to 6-membered heterocycloalkyl including 1 to 2 heteroatoms each independently O, or 5- to 6-membered heteroaryl including 1 to 3 heteroatoms each independently N, O or S; wherein each alkyl group is substituted with 0, 1, 2 or 3 R^{4a}; and each heterocycloalkyl, aryl, and heteroaryl is substituted with 0, 1, 2 or 3 R^{4b};
each R^{4a} is independently 5- to 6-membered heteroaryl including 1 to 3 heteroatoms each independently N, substituted with 0, 1, 2, or 3 R^{4a1};
each R^{4a1} is independently halogen, (C₁-C₃)haloalkyl, or —CN; and each R^{4b} is independently (C₁-C₃)alkyl, or (C₁-C₃)alkoxy.

In some embodiments, the compound of Formula (I), (IA), (IA-1), (IB), (IB-1), (IB-2), (IB-3), (IB-4), or (IC), or pharmaceutically acceptable salt thereof, is the compound wherein
each R² is independently (C₁-C₆)alkyl; wherein each alkyl group is substituted with 1 or 2 R^{4a};
each R^{4a} is independently 5- to 6-membered heteroaryl including 1 to 3 heteroatoms each independently N, substituted with 0, 1, 2, or 3 R^{4a1}; and
each R^{4a1} is independently halogen, (C₁-C₆)haloalkyl, or —CN.

In some embodiments, the compound of Formula (I), (IA), (IA-1), (IB), (IB-1), (IB-2), (IB-3), (IB-4), or (IC), or pharmaceutically acceptable salt thereof, is the compound wherein
each R² is independently (C₁-C₃)alkyl; wherein each alkyl group is substituted with 1 or 2 R^{4a}.
each R^{4a} is independently 5- to 6-membered heteroaryl including 1 to 3 heteroatoms each independently N, substituted with 0, 1, or 2 R^{4a1}; and
each R^{4a1} is independently halogen, (C₁-C₃)haloalkyl, or —CN.

In some embodiments, the compound of Formula (I), (IA), (IA-1), (IB), (IB-1), (IB-2), (IB-3), (IB-4), or (IC), or pharmaceutically acceptable salt thereof, is the compound wherein
each R² is hydrogen, methyl, ethyl, n-propyl or iso-propyl; wherein each group is substituted with 1 or 2 R^{4a};
each R^{4a} is independently pyrrole, pyrazole, imidazole, or triazole, each substituted with 0, 1, or 2 R^{4a1}; and
each R^{4a1} is independently F, Cl, —CF₃ or —CN.

In some embodiments, the compound of Formula (I), (IA), (IA-1), (IB), (IB-1), (IB-2), (IB-3), (IB-4), or (IC), or pharmaceutically acceptable salt thereof, is the compound wherein each R² is independently —CH₂OMe,

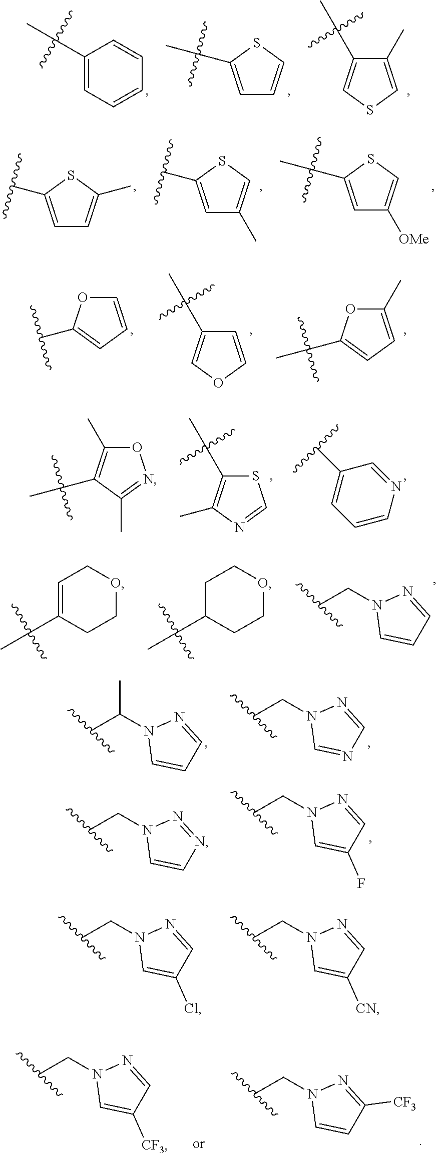

In some embodiments of Formula (IC), each R² is independently

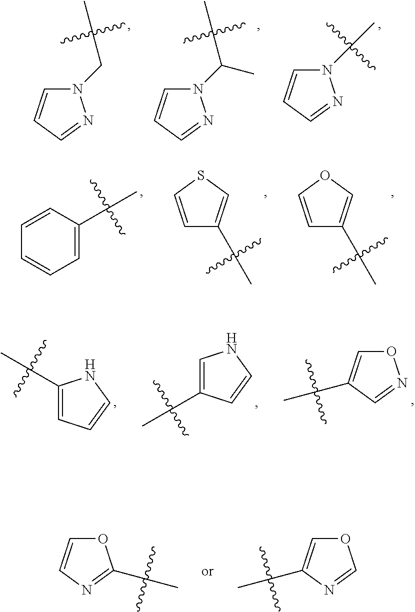

wherein each group is substituted with 0 or 1 (C₁-C₆)alkyl or (C₁-C₆)alkoxy.

In some embodiments, the compound of Formula (I), (IA), (IA-1), (IB), (IB-1), (IB-2), (IB-3), (IB-4), (IC), or (ID), or pharmaceutically acceptable salt thereof, is the compound wherein
each R³ is independently hydrogen or (C₁-C₆)alkyl; wherein each alkyl is substituted with 0, 1, 2, or 3 R^{3a}; and
each R^{3a} is independently (C₁-C₆)alkoxy or halogen.

In some embodiments, the compound of Formula (I), (IA), (IA-1), (IB), (IB-1), (IB-2), (IB-3), (IB-4), (IC), or (ID), or pharmaceutically acceptable salt thereof, is the compound wherein
each R³ is independently hydrogen or (C₁-C₃)alkyl; wherein each alkyl is substituted with 0 or 1 R^{3a}; and
each R^{3a} is independently (C₁-C₃)alkoxy.

In some embodiments, the compound of Formula (I), (IA), (IA-1), (IB), (IB-1), (IB-2), (IB-3), (IB-4), (IC), or (ID), or pharmaceutically acceptable salt thereof, is the compound wherein each R³ is independently hydrogen, Me, Et, CH₂OMe, CH₂OH, or CH₂F.

In some embodiments of Formula (I), (IA), (IA-1), (IB), (IB-1), (IB-2), (IB-3), (IB-4), and/or (IC), each R³ is independently hydrogen, Me or CH₂OMe. In some embodiments of Formula (I), (IA), (IA-1), (IB), (IB-1), (IB-2), (IB-3), (IB-4), and/or (IC), R³ is hydrogen.

In some embodiments of Formula (I), (IA), (IA-1), (IB), (IB-1), (IB-2), (IB-3), (IB-4), and/or (IC), R³ is Me. In some embodiments of Formula (I), (IA), (IA-1), (IB), (IB-1), (IB-2), (IB-3), (IB-4), and/or (IC), R³ is CH₂OMe.

In some embodiments, the compound of Formula (I), (IA), (IA-1), (IB), (IB-1), (IB-2), (IB-3), (IB-4), or (IC), or pharmaceutically acceptable salt thereof, is the compound wherein subscript ‘n’ is an integer of 1 or 2.

In some embodiments of Formula (IC), each R³ is independently hydrogen or (C₁-C₆)alkyl.

In some embodiments, the present invention provides a compound of Formula (IC), wherein
each R^{1a} and R^{1b} is independently (C₁-C₆)alkoxy;
each R² is independently

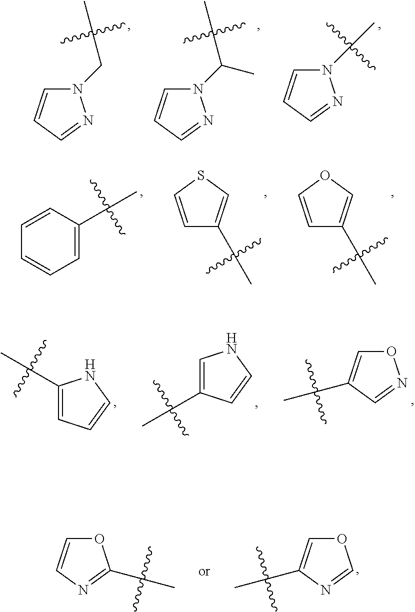

wherein each group is substituted with 0 or 1 (C₁-C₆)alkyl or (C₁-C₆)alkoxy; and
each R³ is independently hydrogen or (C₁-C₆)alkyl.

In some embodiments, the compound of Formula (I), (IA), (IA-1), (IB), (IB-1), (IB-2), (IB-3), (IB-4), or (IC), or pharmaceutically acceptable salt thereof, is the compound having the structure:

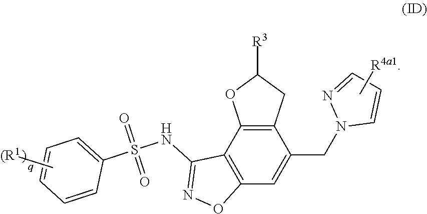

In some embodiments, the compound of Formula (ID), or pharmaceutically acceptable salt thereof, is the compound wherein
each R¹ is independently (C₁-C₄)alkyl, (C₁-C₃)alkoxy, halogen, (C₁-C₃)haloalkyl, (C₁-C₃)haloalkoxy, —C(O)OR^{1h}, (C₃-C₈)cycloalkyl, (C₆-C₁₀)aryl, or 5- to 6-membered heteroaryl having 1 to 3 heteroatoms each N, wherein each alkoxy is substituted with 0 or 1 R^{1f};
each R^{1h} is independently hydrogen or (C₁-C₃)alkyl; and
each R^{1f} is independently 5- to 6-membered heteroaryl having 1 to 3 heteroatoms each N; alternatively, two R¹ groups on adjacent ring atoms are combined to form (C₅-C₈)cycloalkyl;
R^{4a1} is hydrogen, halogen, (C₁-C₃)haloalkyl, or —CN;
each R³ is independently hydrogen or (C₁-C₃)alkyl; wherein each alkyl is substituted with 0 or 1 R^{3a};
each R^{3a} is independently (C₁-C₃)alkoxy; and subscript ‘q’ is 1, 2 or 3.

In some embodiments, the compound of Formula (ID), or pharmaceutically acceptable salt thereof, is the compound wherein
each R¹ is independently Et, t-Bu, OMe, OEt, OCH₂-pyrid-2-yl, F, Cl, CF₃, OCF₃, COOH, COOMe, cyclopropyl, phenyl, or

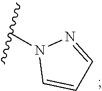 [SMILES: *n1cccn1]

alternatively, two R¹ groups on adjacent ring atoms are combined to form cyclopentyl or cyclohexyl;
R^{4a1} is hydrogen, Cl, F, CF₃, or CN;
R³ is hydrogen, methyl, or CH₂OMe; and subscript ‘q’ is 1, 2 or 3.

In some embodiments, the compound of Formula (I), (IA), (IA-1), (IB), (IB-1), (IB-2), (IB-3), (IB-4), or (IC), or pharmaceutically acceptable salt thereof, is the compound wherein
each R¹ is independently Et, t-Bu, OMe, OEt, OCH₂-pyrid-2-yl, F, Cl, CF₃, OCF₃, COOH, COOMe, cyclopropyl, phenyl, or

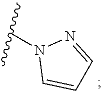 [SMILES: *n1cccn1]

alternatively, two R¹ groups on adjacent ring atoms are combined to form cyclopentyl or cyclohexyl;
R² is —CH₂OMe,

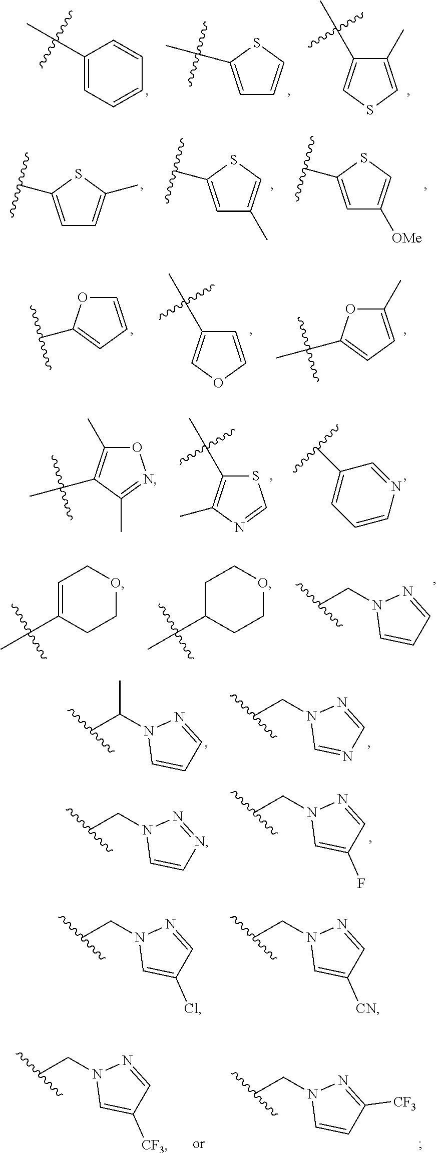

each R³ is independently hydrogen, Me or CH₂OMe;
R^{x} is hydrogen;
subscript ‘m’ is 1;
subscript ‘y’ is 1;
subscript ‘n’ is an integer of 1 or 2; and
subscript ‘q’ is an integer of from 1 to 3.

In some embodiments, the compound of Formula (I), (IA), (IA-1), (IB), (IB-1), (IB-2), (IB-3), (IB-4), or (IC), or pharmaceutically acceptable salt thereof, is the compound wherein
each R¹ is independently Et, t-Bu, OMe, OEt, OCH₂-pyrid-2-yl, F, Cl, CF₃, OCF₃, COOH, COOMe, cyclopropyl, phenyl, or

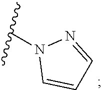 [SMILES: *n1cccn1]

alternatively, two R¹ groups on adjacent ring atoms are combined to form cyclopentyl or cyclohexyl;
R² is

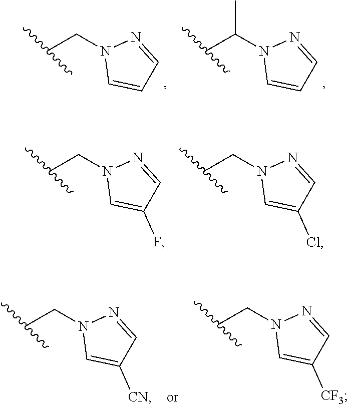

each R³ is independently hydrogen, Me or CH₂OMe;
R^{x} is hydrogen;
subscript ‘m’ is 1;
subscript ‘y’ is 1;
subscript ‘n’ is an integer of 1 or 2; and
subscript ‘q’ is an integer of from 1 to 3.

In some embodiments the present invention provides a compound or a pharmaceutically acceptable salt or a stereoisomer or a tautomer thereof, wherein the compound is selected from

| Compound | Structure |
|---|---|
| 1 | 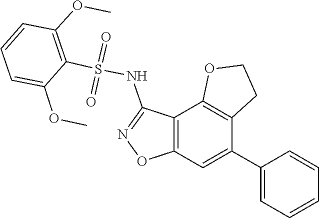 [SMILES: COc1cccc(OC)c1S(=O)(=O)Nc1noc2cc(-c3ccccc3)c3c(c12)OCC3] |
| 2 | 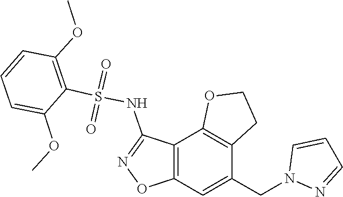 [SMILES: COc1cccc(OC)c1S(=O)(=O)Nc1noc2cc(Cn3cccn3)c3c(c12)OCC3] |
| 3 | 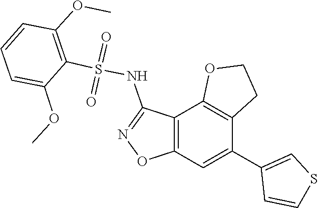 [SMILES: COc1cccc(OC)c1S(=O)(=O)Nc1noc2cc(-c3ccsc3)c3c(c12)OCC3] |
| 4 | 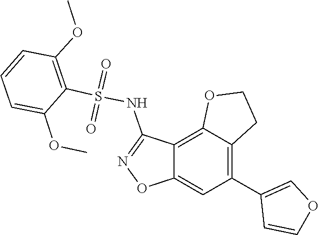 [SMILES: COc1cccc(OC)c1S(=O)(=O)Nc1noc2cc(-c3ccoc3)c3c(c12)OCC3] |
| 5 | 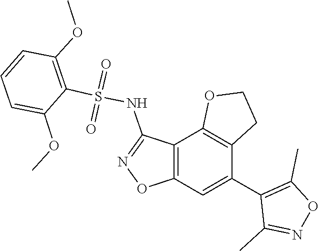 [SMILES: COc1cccc(OC)c1S(=O)(=O)Nc1noc2cc(-c3c(C)noc3C)c3c(c12)OCC3] |
| 6 | 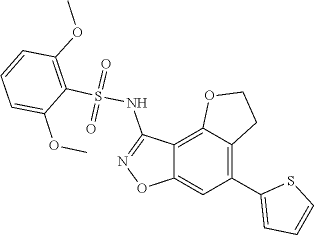 [SMILES: COc1cccc(OC)c1S(=O)(=O)Nc1noc2cc(-c3cccs3)c3c(c12)OCC3] |
| 7 | 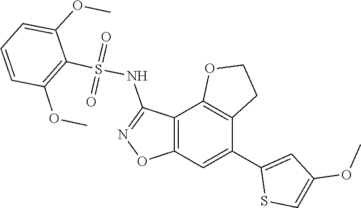 [SMILES: COc1csc(-c2cc3onc(NS(=O)(=O)c4c(OC)cccc4OC)c3c3c2CCO3)c1] |
| 8 | 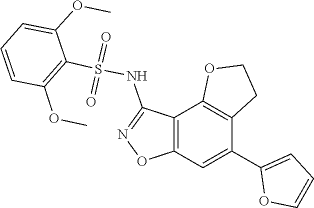 [SMILES: COc1cccc(OC)c1S(=O)(=O)Nc1noc2cc(-c3ccco3)c3c(c12)OCC3] |
| 9 | 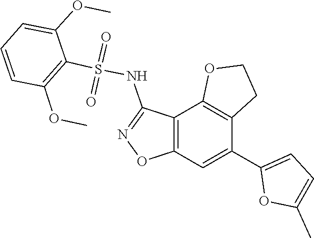 [SMILES: COc1cccc(OC)c1S(=O)(=O)Nc1noc2cc(-c3ccc(C)o3)c3c(c12)OCC3] |
| 10 | 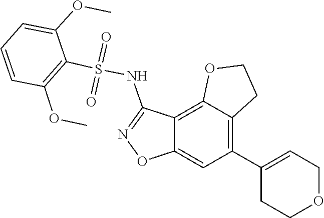 [SMILES: COc1cccc(OC)c1S(=O)(=O)Nc1noc2cc(C3=CCOCC3)c3c(c12)OCC3] |
| 11 | 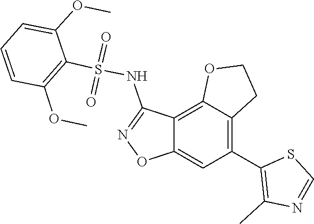 [SMILES: COc1cccc(OC)c1S(=O)(=O)Nc1noc2cc(-c3scnc3C)c3c(c12)OCC3] |
| 12 | 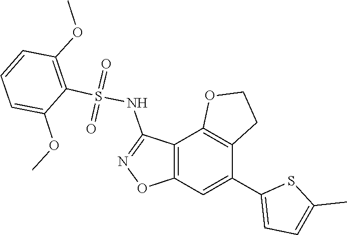 [SMILES: COc1cccc(OC)c1S(=O)(=O)Nc1noc2cc(-c3ccc(C)s3)c3c(c12)OCC3] |
| 13 | 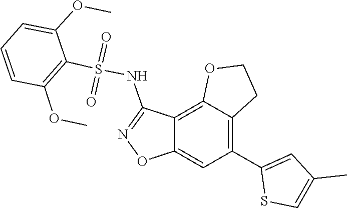 [SMILES: COc1cccc(OC)c1S(=O)(=O)Nc1noc2cc(-c3cc(C)cs3)c3c(c12)OCC3] |
| 14 | 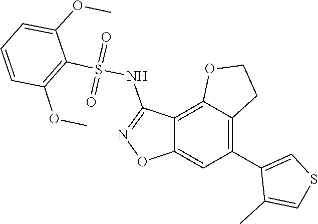 [SMILES: COc1cccc(OC)c1S(=O)(=O)Nc1noc2cc(-c3cscc3C)c3c(c12)OCC3] |
| 15 | 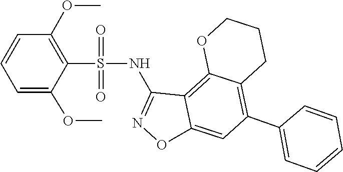 [SMILES: COc1cccc(OC)c1S(=O)(=O)Nc1noc2cc(-c3ccccc3)c3c(c12)OCCC3] |
| 16 | 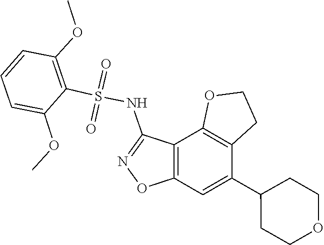 [SMILES: COc1cccc(OC)c1S(=O)(=O)Nc1noc2cc(C3CCOCC3)c3c(c12)OCC3] |
| 17 | 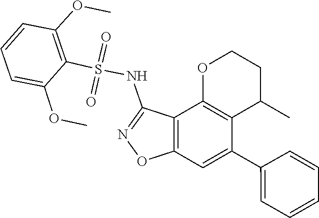 [SMILES: COc1cccc(OC)c1S(=O)(=O)Nc1noc2cc(-c3ccccc3)c3c(c12)OCCC3C] |
| 18 | 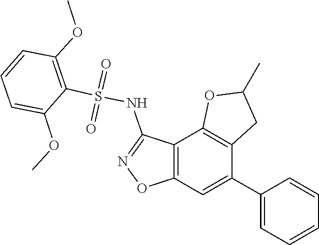 [SMILES: COc1cccc(OC)c1S(=O)(=O)Nc1noc2cc(-c3ccccc3)c3c(c12)OC(C)C3] |
| 19 | 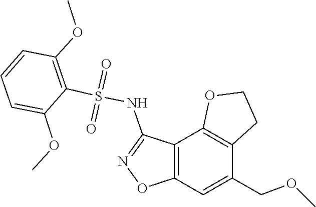 [SMILES: COCc1cc2onc(NS(=O)(=O)c3c(OC)cccc3OC)c2c2c1CCO2] |
| 20 | 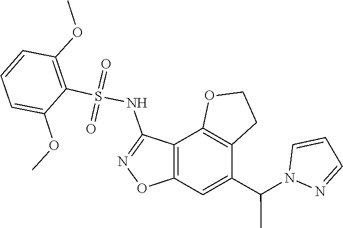 [SMILES: COc1cccc(OC)c1S(=O)(=O)Nc1noc2cc(C(C)n3cccn3)c3c(c12)OCC3] |
| 21 | 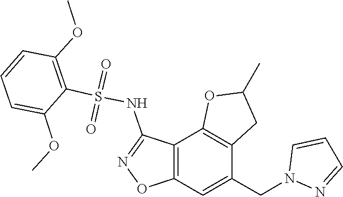 [SMILES: COc1cccc(OC)c1S(=O)(=O)Nc1noc2cc(Cn3cccn3)c3c(c12)OC(C)C3] |
| 22 | 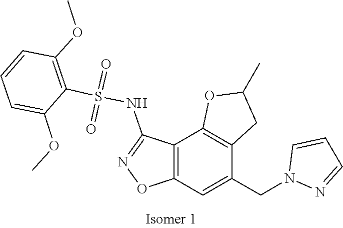 [SMILES: COc1cccc(OC)c1S(=O)(=O)Nc1noc2cc(Cn3cccn3)c3c(c12)OC(C)C3] |
| 23 | 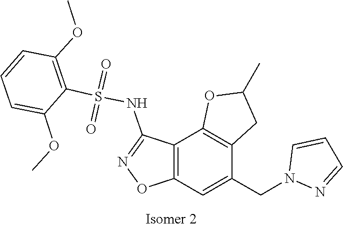 [SMILES: COc1cccc(OC)c1S(=O)(=O)Nc1noc2cc(Cn3cccn3)c3c(c12)OC(C)C3] |
| 24 | 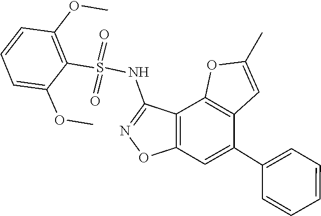 [SMILES: COc1cccc(OC)c1S(=O)(=O)Nc1noc2cc(-c3ccccc3)c3cc(C)oc3c12] |
| 25 | 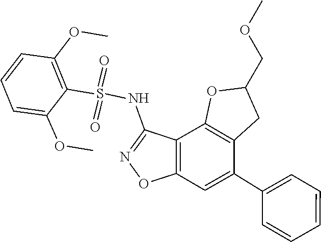 [SMILES: COCC1Cc2c(-c3ccccc3)cc3onc(NS(=O)(=O)c4c(OC)cccc4OC)c3c2O1] |
| 26 | 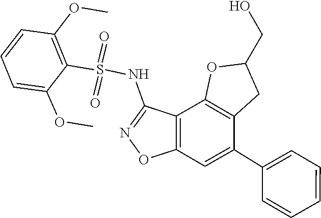 [SMILES: COc1cccc(OC)c1S(=O)(=O)Nc1noc2cc(-c3ccccc3)c3c(c12)OC(CO)C3] |
| 27 | 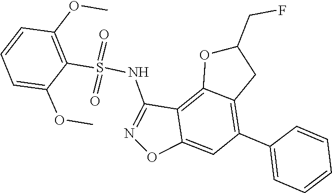 [SMILES: COc1cccc(OC)c1S(=O)(=O)Nc1noc2cc(-c3ccccc3)c3c(c12)OC(CF)C3] |
| 28 | 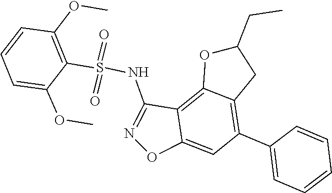 [SMILES: CCC1Cc2c(-c3ccccc3)cc3onc(NS(=O)(=O)c4c(OC)cccc4OC)c3c2O1] |
| 29 | 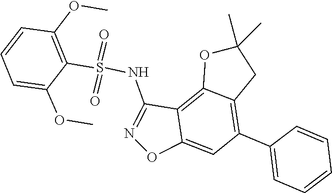 [SMILES: COc1cccc(OC)c1S(=O)(=O)Nc1noc2cc(-c3ccccc3)c3c(c12)OC(C)(C)C3] |
| 30 | 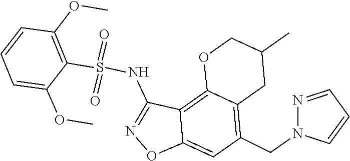 [SMILES: COc1cccc(OC)c1S(=O)(=O)Nc1noc2cc(Cn3cccn3)c3c(c12)OCC(C)C3] |

In some embodiments the present invention provides a compound or a pharmaceutically acceptable salt or a stereoisomer or a tautomer thereof, wherein the compound is selected from Table 1.

TABLE 1
1. Compounds

| Com- | |
|---|---|
| pound | Structure |
| 1 | 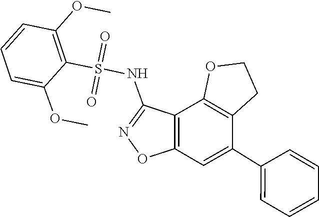 [SMILES: COc1cccc(OC)c1S(=O)(=O)Nc1noc2cc(-c3ccccc3)c3c(c12)OCC3] |
| 2 | 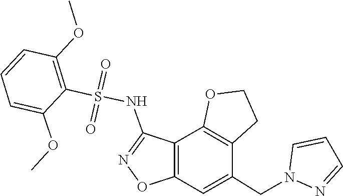 [SMILES: COc1cccc(OC)c1S(=O)(=O)Nc1noc2cc(Cn3cccn3)c3c(c12)OCC3] |
| 3 | 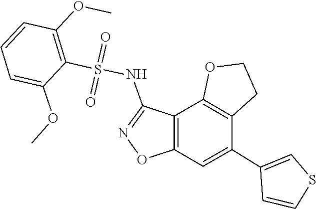 [SMILES: COc1cccc(OC)c1S(=O)(=O)Nc1noc2cc(-c3ccsc3)c3c(c12)OCC3] |
| 4 | 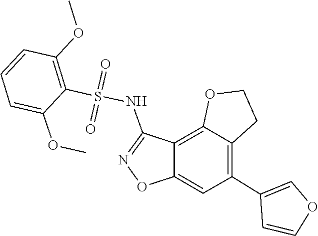 [SMILES: COc1cccc(OC)c1S(=O)(=O)Nc1noc2cc(-c3ccoc3)c3c(c12)OCC3] |
| 5 | 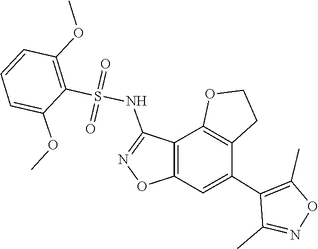 [SMILES: COc1cccc(OC)c1S(=O)(=O)Nc1noc2cc(-c3c(C)noc3C)c3c(c12)OCC3] |
| 6 | 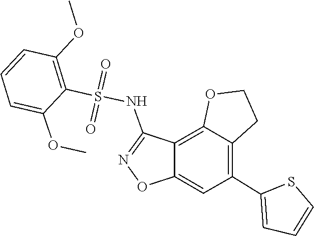 [SMILES: COc1cccc(OC)c1S(=O)(=O)Nc1noc2cc(-c3cccs3)c3c(c12)OCC3] |
| 7 | 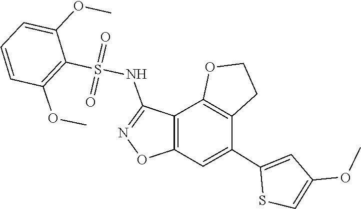 [SMILES: COc1csc(-c2cc3onc(NS(=O)(=O)c4c(OC)cccc4OC)c3c3c2CCO3)c1] |
| 8 | 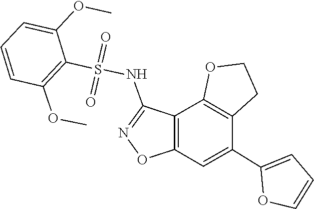 [SMILES: COc1cccc(OC)c1S(=O)(=O)Nc1noc2cc(-c3ccco3)c3c(c12)OCC3] |
| 9 | 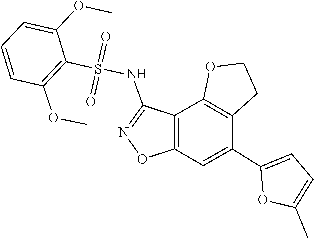 [SMILES: COc1cccc(OC)c1S(=O)(=O)Nc1noc2cc(-c3ccc(C)o3)c3c(c12)OCC3] |
| 10 | 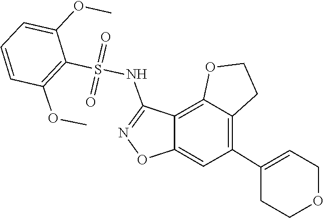 [SMILES: COc1cccc(OC)c1S(=O)(=O)Nc1noc2cc(C3=CCOCC3)c3c(c12)OCC3] |
| 11 | 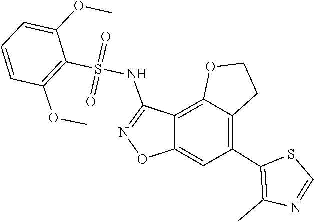 [SMILES: COc1cccc(OC)c1S(=O)(=O)Nc1noc2cc(-c3scnc3C)c3c(c12)OCC3] |
| 12 | 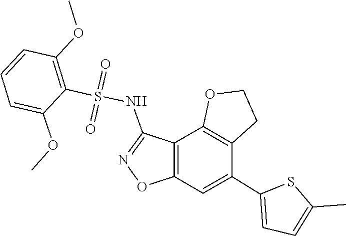 [SMILES: COc1cccc(OC)c1S(=O)(=O)Nc1noc2cc(-c3ccc(C)s3)c3c(c12)OCC3] |
| 13 | 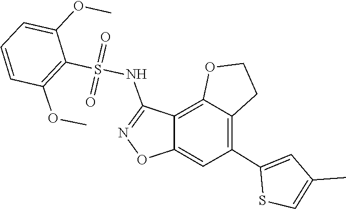 [SMILES: COc1cccc(OC)c1S(=O)(=O)Nc1noc2cc(-c3cc(C)cs3)c3c(c12)OCC3] |
| 14 | 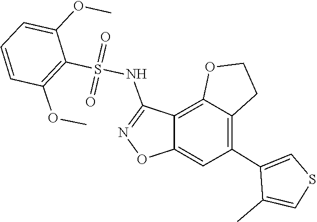 [SMILES: COc1cccc(OC)c1S(=O)(=O)Nc1noc2cc(-c3cscc3C)c3c(c12)OCC3] |
| 15 | 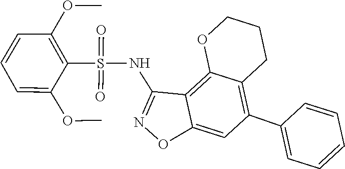 [SMILES: COc1cccc(OC)c1S(=O)(=O)Nc1noc2cc(-c3ccccc3)c3c(c12)OCCC3] |
| 16 | 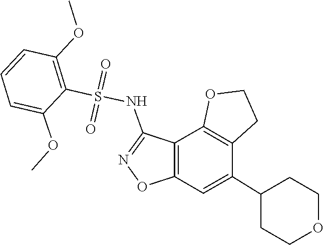 [SMILES: COc1cccc(OC)c1S(=O)(=O)Nc1noc2cc(C3CCOCC3)c3c(c12)OCC3] |
| 17 | 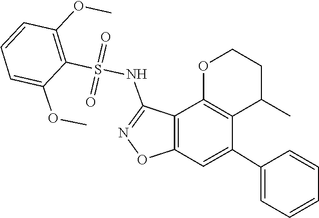 [SMILES: COc1cccc(OC)c1S(=O)(=O)Nc1noc2cc(-c3ccccc3)c3c(c12)OCCC3C] |
| 18 | 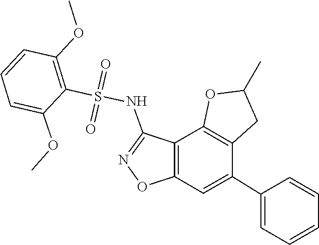 [SMILES: COc1cccc(OC)c1S(=O)(=O)Nc1noc2cc(-c3ccccc3)c3c(c12)OC(C)C3] |
| 19 | 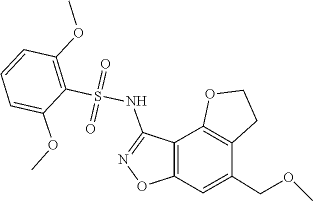 [SMILES: COCc1cc2onc(NS(=O)(=O)c3c(OC)cccc3OC)c2c2c1CCO2] |
| 20 | 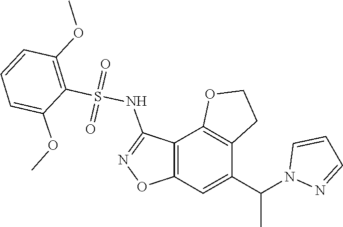 [SMILES: COc1cccc(OC)c1S(=O)(=O)Nc1noc2cc(C(C)n3cccn3)c3c(c12)OCC3] |
| 21 | 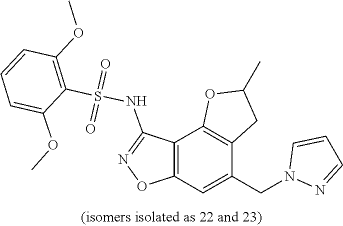 [SMILES: COc1cccc(OC)c1S(=O)(=O)Nc1noc2cc(Cn3cccn3)c3c(c12)OC(C)C3] |
| 22 | 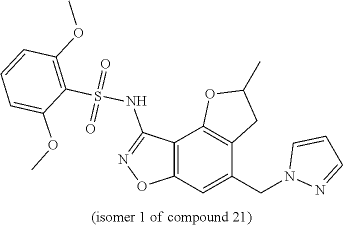 [SMILES: COc1cccc(OC)c1S(=O)(=O)Nc1noc2cc(Cn3cccn3)c3c(c12)OC(C)C3] |
| 23 | 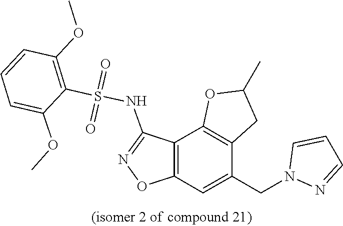 [SMILES: COc1cccc(OC)c1S(=O)(=O)Nc1noc2cc(Cn3cccn3)c3c(c12)OC(C)C3] |
| 31 | 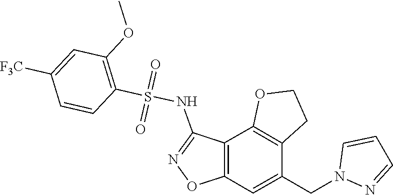 [SMILES: COc1cc(C(F)(F)F)ccc1S(=O)(=O)Nc1noc2cc(Cn3cccn3)c3c(c12)OCC3] |
| 32 | 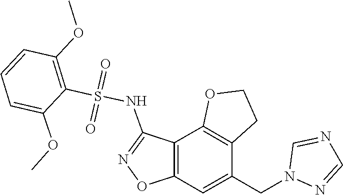 [SMILES: COc1cccc(OC)c1S(=O)(=O)Nc1noc2cc(Cn3cncn3)c3c(c12)OCC3] |
| 33 | 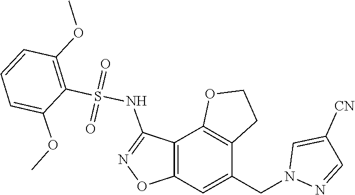 [SMILES: COc1cccc(OC)c1S(=O)(=O)Nc1noc2cc(Cn3cc(C#N)cn3)c3c(c12)OCC3] |
| 34 | 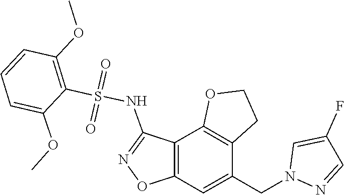 [SMILES: COc1cccc(OC)c1S(=O)(=O)Nc1noc2cc(Cn3cc(F)cn3)c3c(c12)OCC3] |
| 35 | 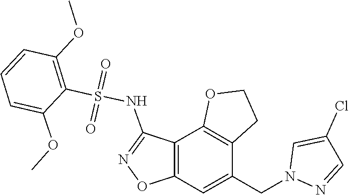 [SMILES: COc1cccc(OC)c1S(=O)(=O)Nc1noc2cc(Cn3cc(Cl)cn3)c3c(c12)OCC3] |
| 36 | 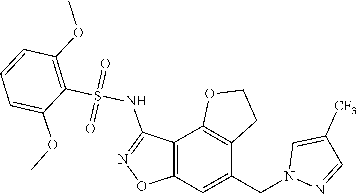 [SMILES: COc1cccc(OC)c1S(=O)(=O)Nc1noc2cc(Cn3cc(C(F)(F)F)cn3)c3c(c12)OCC3] |
| 37 | 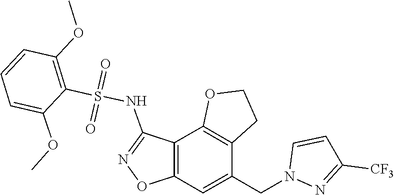 [SMILES: COc1cccc(OC)c1S(=O)(=O)Nc1noc2cc(Cn3ccc(C(F)(F)F)n3)c3c(c12)OCC3] |
| 38 | 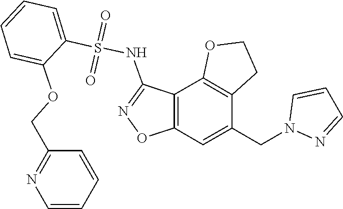 [SMILES: O=S(=O)(Nc1noc2cc(Cn3cccn3)c3c(c12)OCC3)c1ccccc1OCc1ccccn1] |
| 39 | 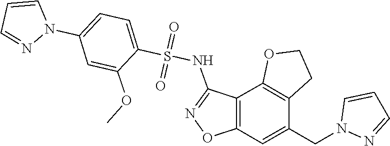 [SMILES: COc1cc(-n2cccn2)ccc1S(=O)(=O)Nc1noc2cc(Cn3cccn3)c3c(c12)OCC3] |
| 40 | 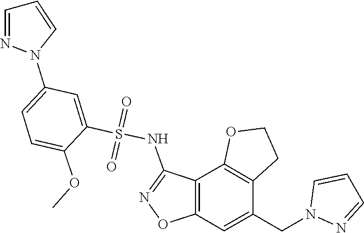 [SMILES: COc1ccc(-n2cccn2)cc1S(=O)(=O)Nc1noc2cc(Cn3cccn3)c3c(c12)OCC3] |
| 41 | 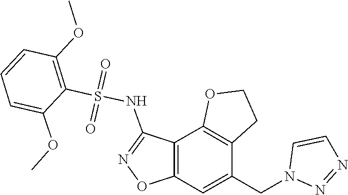 [SMILES: COc1cccc(OC)c1S(=O)(=O)Nc1noc2cc(Cn3ccnn3)c3c(c12)OCC3] |
| 42 | 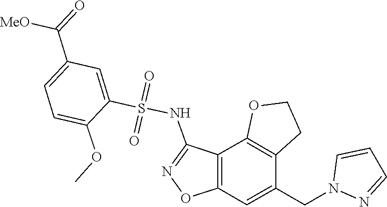 [SMILES: COC(=O)c1ccc(OC)c(S(=O)(=O)Nc2noc3cc(Cn4cccn4)c4c(c23)OCC4)c1] |
| 43 | 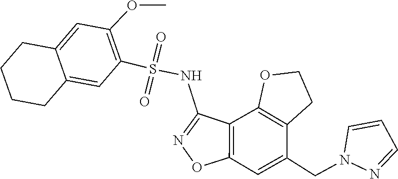 [SMILES: COc1cc2c(cc1S(=O)(=O)Nc1noc3cc(Cn4cccn4)c4c(c13)OCC4)CCCC2] |
| 44 | 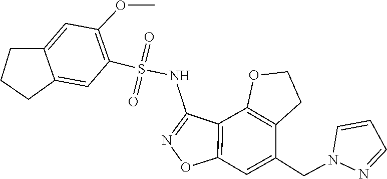 [SMILES: COc1cc2c(cc1S(=O)(=O)Nc1noc3cc(Cn4cccn4)c4c(c13)OCC4)CCC2] |
| 45 | 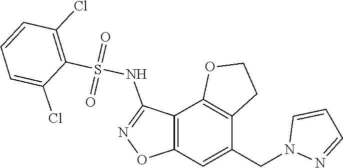 [SMILES: O=S(=O)(Nc1noc2cc(Cn3cccn3)c3c(c12)OCC3)c1c(Cl)cccc1Cl] |
| 46 | 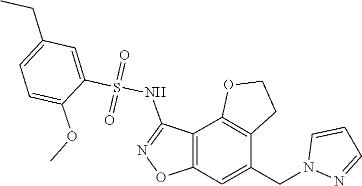 [SMILES: CCc1ccc(OC)c(S(=O)(=O)Nc2noc3cc(Cn4cccn4)c4c(c23)OCC4)c1] |
| 47 | 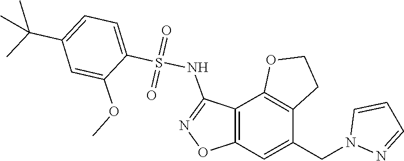 [SMILES: COc1cc(C(C)(C)C)ccc1S(=O)(=O)Nc1noc2cc(Cn3cccn3)c3c(c12)OCC3] |
| 48 | 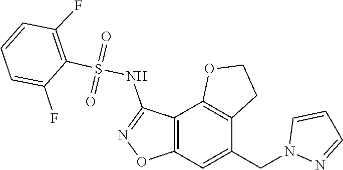 [SMILES: O=S(=O)(Nc1noc2cc(Cn3cccn3)c3c(c12)OCC3)c1c(F)cccc1F] |
| 49 | 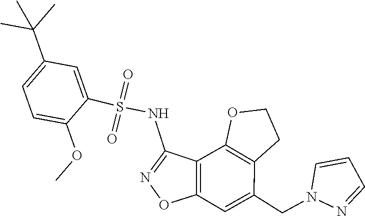 [SMILES: COc1ccc(C(C)(C)C)cc1S(=O)(=O)Nc1noc2cc(Cn3cccn3)c3c(c12)OCC3] |
| 50 | 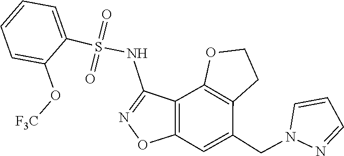 [SMILES: O=S(=O)(Nc1noc2cc(Cn3cccn3)c3c(c12)OCC3)c1ccccc1OC(F)(F)F] |
| 51 | 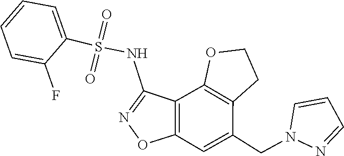 [SMILES: O=S(=O)(Nc1noc2cc(Cn3cccn3)c3c(c12)OCC3)c1ccccc1F] |
| 52 | 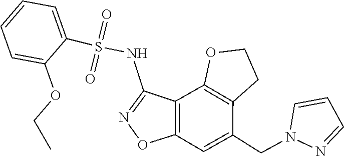 [SMILES: CCOc1ccccc1S(=O)(=O)Nc1noc2cc(Cn3cccn3)c3c(c12)OCC3] |
| 53 | 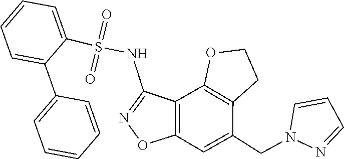 [SMILES: O=S(=O)(Nc1noc2cc(Cn3cccn3)c3c(c12)OCC3)c1ccccc1-c1ccccc1] |
| 54 | 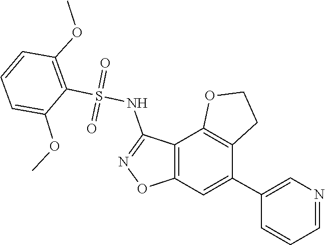 [SMILES: COc1cccc(OC)c1S(=O)(=O)Nc1noc2cc(-c3cccnc3)c3c(c12)OCC3] |
| 55 | 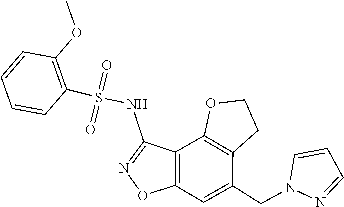 [SMILES: COc1ccccc1S(=O)(=O)Nc1noc2cc(Cn3cccn3)c3c(c12)OCC3] |
| 56 | 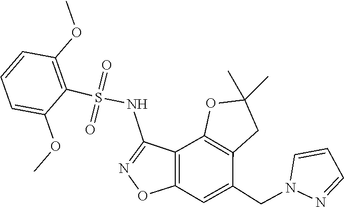 [SMILES: COc1cccc(OC)c1S(=O)(=O)Nc1noc2cc(Cn3cccn3)c3c(c12)OC(C)(C)C3] |
| 57 | 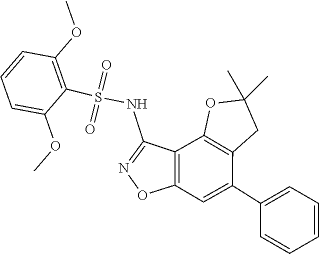 [SMILES: COc1cccc(OC)c1S(=O)(=O)Nc1noc2cc(-c3ccccc3)c3c(c12)OC(C)(C)C3] |
| 58 | 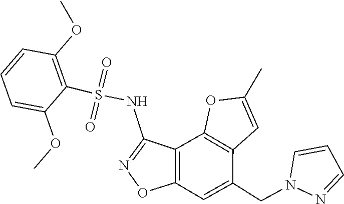 [SMILES: COc1cccc(OC)c1S(=O)(=O)Nc1noc2cc(Cn3cccn3)c3cc(C)oc3c12] |
| 59 | 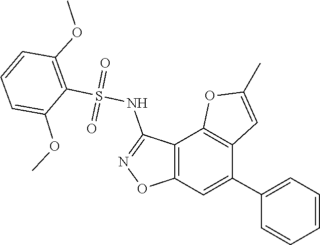 [SMILES: COc1cccc(OC)c1S(=O)(=O)Nc1noc2cc(-c3ccccc3)c3cc(C)oc3c12] |
| 60 | 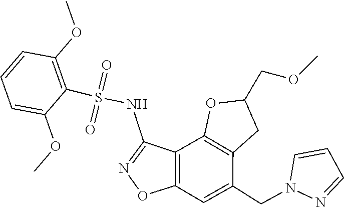 [SMILES: COCC1Cc2c(Cn3cccn3)cc3onc(NS(=O)(=O)c4c(OC)cccc4OC)c3c2O1] |
| 61 | 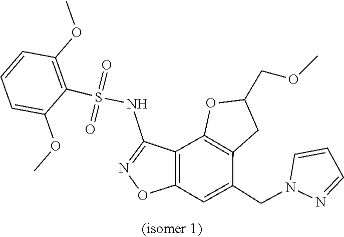 [SMILES: COCC1Cc2c(Cn3cccn3)cc3onc(NS(=O)(=O)c4c(OC)cccc4OC)c3c2O1] |
| 62 | 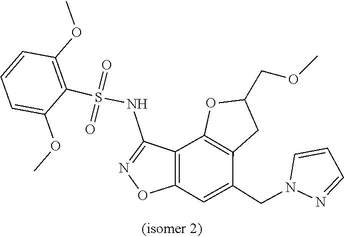 [SMILES: COCC1Cc2c(Cn3cccn3)cc3onc(NS(=O)(=O)c4c(OC)cccc4OC)c3c2O1] |
| 63 | 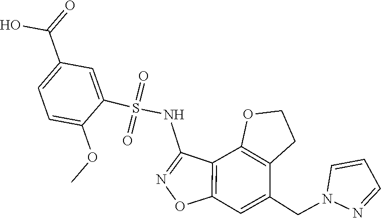 [SMILES: COc1ccc(C(=O)O)cc1S(=O)(=O)Nc1noc2cc(Cn3cccn3)c3c(c12)OCC3] |
| 64 | 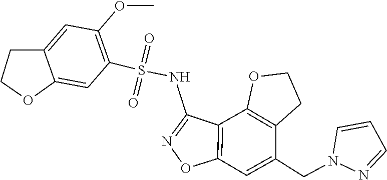 [SMILES: COc1cc2c(cc1S(=O)(=O)Nc1noc3cc(Cn4cccn4)c4c(c13)OCC4)OCC2] |
| 65 | 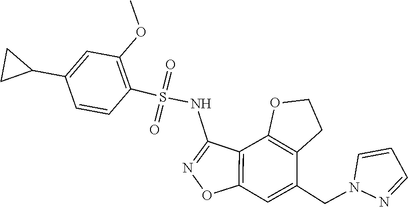 [SMILES: COc1cc(C2CC2)ccc1S(=O)(=O)Nc1noc2cc(Cn3cccn3)c3c(c12)OCC3] |

In some embodiments of the compound or pharmaceutically acceptable salt thereof, the compound has the structure:

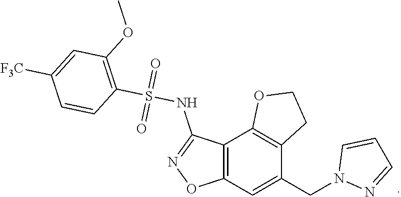 [SMILES: COc1cc(C(F)(F)F)ccc1S(=O)(=O)Nc1noc2cc(Cn3cccn3)c3c(c12)OCC3]

In some embodiments of the compound, the compound has the structure:

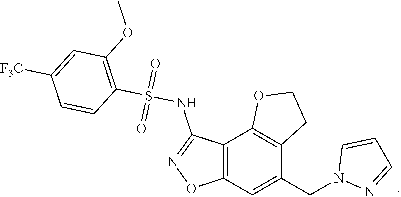 [SMILES: COc1cc(C(F)(F)F)ccc1S(=O)(=O)Nc1noc2cc(Cn3cccn3)c3c(c12)OCC3]

In some embodiments of the compound or pharmaceutically acceptable salt thereof, the compound has the structure:

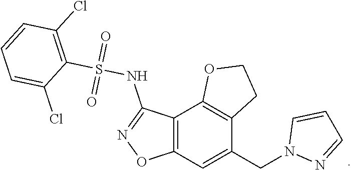 [SMILES: O=S(=O)(Nc1noc2cc(Cn3cccn3)c3c(c12)OCC3)c1c(Cl)cccc1Cl]

In some embodiments of the compound, the compound has the structure:

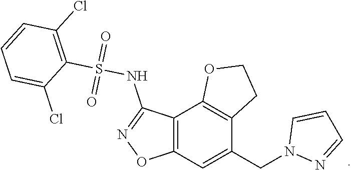 [SMILES: O=S(=O)(Nc1noc2cc(Cn3cccn3)c3c(c12)OCC3)c1c(Cl)cccc1Cl]

In some embodiments of the compound or pharmaceutically acceptable salt thereof, the compound has the structure:

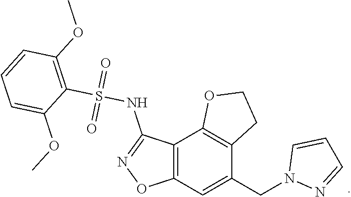 [SMILES: COc1cccc(OC)c1S(=O)(=O)Nc1noc2cc(Cn3cccn3)c3c(c12)OCC3]

In some embodiments of the compound, the compound has the structure:

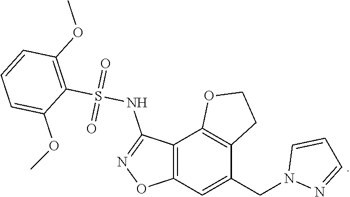 [SMILES: COc1cccc(OC)c1S(=O)(=O)Nc1noc2cc(Cn3cccn3)c3c(c12)OCC3]

In some embodiments of the compound or pharmaceutically acceptable salt thereof, the compound has the structure:

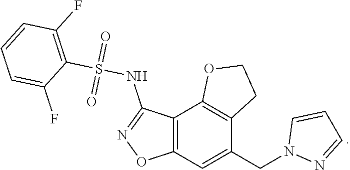 [SMILES: O=S(=O)(Nc1noc2cc(Cn3cccn3)c3c(c12)OCC3)c1c(F)cccc1F]

In some embodiments of the compound, the compound has the structure:

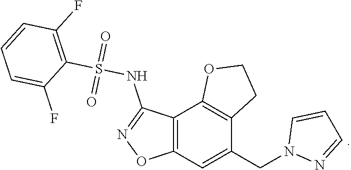 [SMILES: O=S(=O)(Nc1noc2cc(Cn3cccn3)c3c(c12)OCC3)c1c(F)cccc1F]

In some embodiments of the compound or pharmaceutically acceptable salt thereof, the compound has the structure:

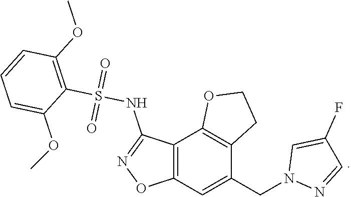 [SMILES: COc1cccc(OC)c1S(=O)(=O)Nc1noc2cc(Cn3cc(F)cn3)c3c(c12)OCC3]

In some embodiments of the compound, the compound has the structure:

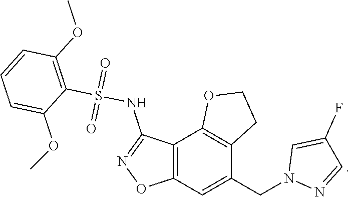 [SMILES: COc1cccc(OC)c1S(=O)(=O)Nc1noc2cc(Cn3cc(F)cn3)c3c(c12)OCC3]

In some embodiments of the compound or pharmaceutically acceptable salt thereof, the compound has the structure:

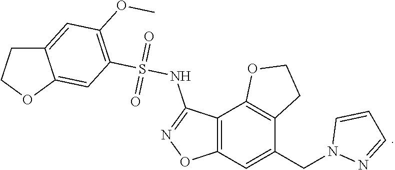 [SMILES: COc1cc2c(cc1S(=O)(=O)Nc1noc3cc(Cn4cccn4)c4c(c13)OCC4)OCC2]

In some embodiments of the compound, the compound has the structure:

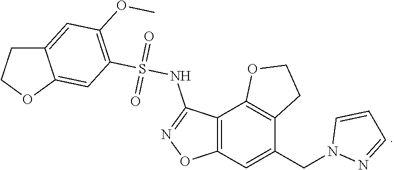 [SMILES: COc1cc2c(cc1S(=O)(=O)Nc1noc3cc(Cn4cccn4)c4c(c13)OCC4)OCC2]

In some embodiments of the compound or pharmaceutically acceptable salt thereof, the compound has the structure:

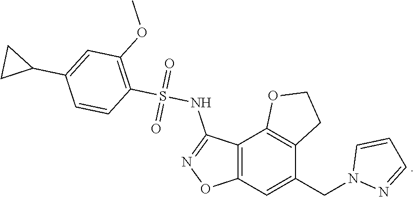 [SMILES: COc1cc(C2CC2)ccc1S(=O)(=O)Nc1noc2cc(Cn3cccn3)c3c(c12)OCC3]

In some embodiments of the compound, the compound has the structure:

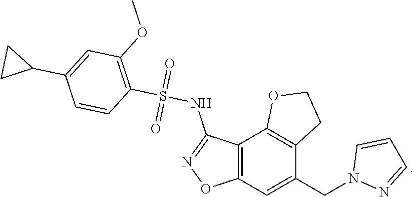 [SMILES: COc1cc(C2CC2)ccc1S(=O)(=O)Nc1noc2cc(Cn3cccn3)c3c(c12)OCC3]

The present invention also provides intermediates of the compounds of Formula (I), (IA), (IA-1), (IB), (IB-1), (IB-2), (IB-3), and/or (IB-4). In some embodiments, an intermediate is a compound of Formula (II) or salt thereof:

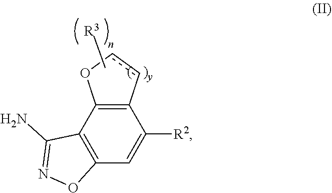

wherein dotted line, R², R³, subscript ‘n’, and subscript ‘y’ are as defined for the compound of Formula (I), (IA), and/or (IA-1) herein.

In some embodiments of the compound of Formula (II) or salt thereof, the compound has the structure of any one of the following:

| Intermediate | Structure |
|---|---|
| 1 | 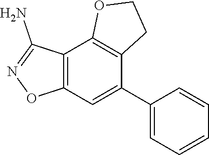 [SMILES: Nc1noc2cc(-c3ccccc3)c3c(c12)OCC3] |
| 2 | 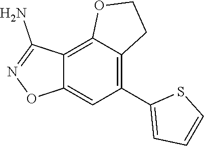 [SMILES: Nc1noc2cc(-c3cccs3)c3c(c12)OCC3] |
| 3 | 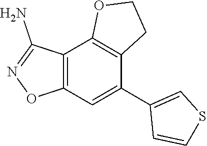 [SMILES: Nc1noc2cc(-c3ccsc3)c3c(c12)OCC3] |
| 4 | 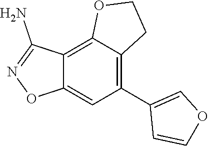 [SMILES: Nc1noc2cc(-c3ccoc3)c3c(c12)OCC3] |
| 5 | 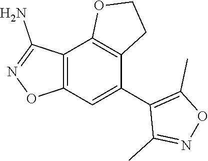 [SMILES: Cc1noc(C)c1-c1cc2onc(N)c2c2c1CCO2] |
| 6 | 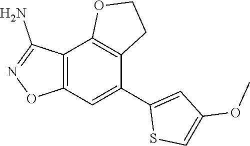 [SMILES: COc1csc(-c2cc3onc(N)c3c3c2CCO3)c1] |
| 7 | 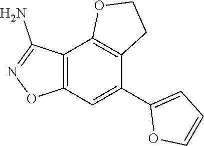 [SMILES: Nc1noc2cc(-c3ccco3)c3c(c12)OCC3] |
| 8 | 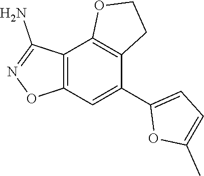 [SMILES: Cc1ccc(-c2cc3onc(N)c3c3c2CCO3)o1] |
| 9 | 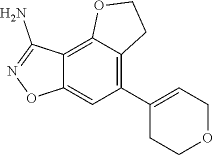 [SMILES: Nc1noc2cc(C3=CCOCC3)c3c(c12)OCC3] |
| 10 | 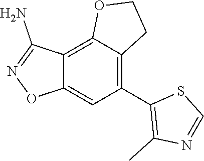 [SMILES: Cc1ncsc1-c1cc2onc(N)c2c2c1CCO2] |
| 11 | 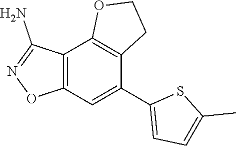 [SMILES: Cc1ccc(-c2cc3onc(N)c3c3c2CCO3)s1] |
| 12 | 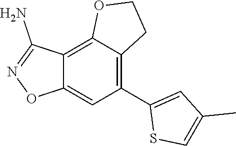 [SMILES: Cc1csc(-c2cc3onc(N)c3c3c2CCO3)c1] |
| 13 | 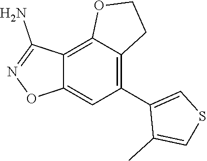 [SMILES: Cc1cscc1-c1cc2onc(N)c2c2c1CCO2] |
| 14 | 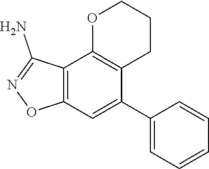 [SMILES: Nc1noc2cc(-c3ccccc3)c3c(c12)OCCC3] |
| 15 | 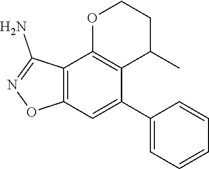 [SMILES: CC1CCOc2c1c(-c1ccccc1)cc1onc(N)c21] |
| 16 | 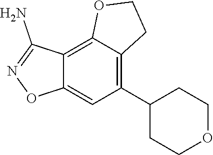 [SMILES: Nc1noc2cc(C3CCOCC3)c3c(c12)OCC3] |
| 18 | 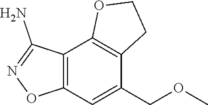 [SMILES: COCc1cc2onc(N)c2c2c1CCO2] |
| 19 | 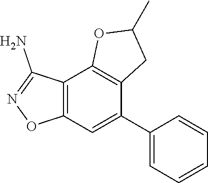 [SMILES: CC1Cc2c(-c3ccccc3)cc3onc(N)c3c2O1] |
| 20 | 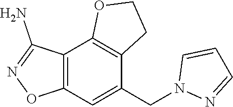 [SMILES: Nc1noc2cc(Cn3cccn3)c3c(c12)OCC3] |
| 21 | 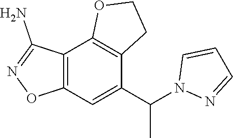 [SMILES: CC(c1cc2onc(N)c2c2c1CCO2)n1cccn1] |
| 22 | 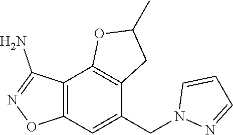 [SMILES: CC1Cc2c(Cn3cccn3)cc3onc(N)c3c2O1] |
| 23 | 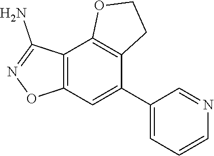 [SMILES: Nc1noc2cc(-c3cccnc3)c3c(c12)OCC3] |
| 24 | 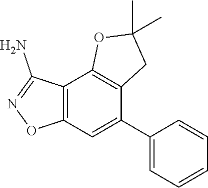 [SMILES: CC1(C)Cc2c(-c3ccccc3)cc3onc(N)c3c2O1] |
| 25 | 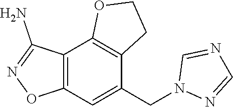 [SMILES: Nc1noc2cc(Cn3cncn3)c3c(c12)OCC3] |
| 26 | 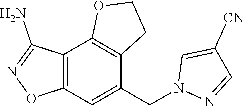 [SMILES: N#Cc1cnn(Cc2cc3onc(N)c3c3c2CCO3)c1] |
| 27 | 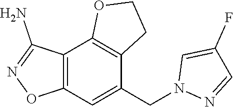 [SMILES: Nc1noc2cc(Cn3cc(F)cn3)c3c(c12)OCC3] |
| 28 | 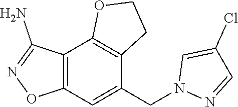 [SMILES: Nc1noc2cc(Cn3cc(Cl)cn3)c3c(c12)OCC3] |
| 29 | 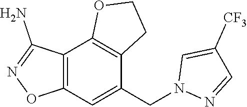 [SMILES: Nc1noc2cc(Cn3cc(C(F)(F)F)cn3)c3c(c12)OCC3] |
| 30 | 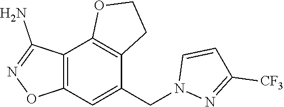 [SMILES: Nc1noc2cc(Cn3ccc(C(F)(F)F)n3)c3c(c12)OCC3] |
| 31 | 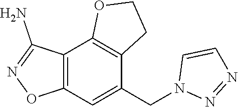 [SMILES: Nc1noc2cc(Cn3ccnn3)c3c(c12)OCC3] |
| 32 | 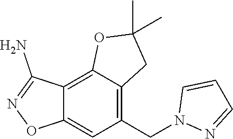 [SMILES: CC1(C)Cc2c(Cn3cccn3)cc3onc(N)c3c2O1] |
| 33 | 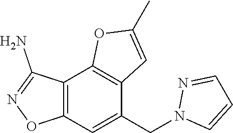 [SMILES: Cc1cc2c(Cn3cccn3)cc3onc(N)c3c2o1] |
| 34 | 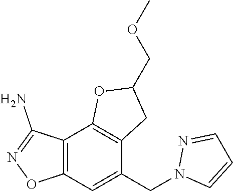 [SMILES: COCC1Cc2c(Cn3cccn3)cc3onc(N)c3c2O1] |

### IV. Compositions

In some embodiments, the pharmaceutical composition of the present invention comprises a compound of the present invention or pharmaceutically acceptable salt thereof, and a pharmaceutically acceptable carrier.

The compounds of the present invention may be used as single drug or as a pharmaceutical composition in which the compound is mixed with various pharmacologically acceptable excipients.

The compounds of the invention are typically administered in the form of a pharmaceutical composition. Such compositions can be prepared using procedures well known in the pharmaceutical art and comprise at least one compound of the invention. The pharmaceutical composition of the present patent application comprises one or more compounds described herein and one or more pharmaceutically acceptable excipients. Typically, the pharmaceutically acceptable excipients are approved by regulatory authorities or are generally regarded as safe for human or animal use. The pharmaceutically acceptable excipients include, but are not limited to, carriers, diluents, glidants and lubricants, preservatives, buffering agents, chelating agents, polymers, gelling agents, viscosifying agents and solvents.

The pharmaceutical composition can be administered by oral, parenteral or inhalation routes. Examples of the parenteral administration include administration by injection, percutaneous, transmucosal, transnasal and transpulmonary administrations.

Examples of suitable carriers include, but are not limited to, water, salt solutions, alcohols, polyethylene glycols, peanut oil, olive oil, gelatin, lactose, terra alba, sucrose, dextrin, magnesium carbonate, sugar, amylose, magnesium stearate, talc, gelatin, agar, pectin, acacia, stearic acid, lower alkyl ethers of cellulose, silicic acid, fatty acids, fatty acid amines, fatty acid monoglycerides and diglycerides, fatty acid esters and polyoxyethylene.

The pharmaceutical composition may also include one or more pharmaceutically acceptable auxiliary agents, wetting agents, suspending agents, preserving agents, buffers, sweetening agents, flavouring agents, colorants or any combination of the foregoing.

The pharmaceutical compositions may be in conventional forms, for example, tablets, capsules, solutions, suspensions, injectables or products for topical application. Further, the pharmaceutical composition of the present invention may be formulated so as to provide desired release profile.

Administration of the compounds of the invention, in pure form or in an appropriate pharmaceutical composition, can be carried out using any of the accepted routes of administration of pharmaceutical compositions. The route of administration may be any route which effectively transports the active compound of the patent application to the appropriate or desired site of action. Suitable routes of administration include, but are not limited to oral, nasal, buccal, dermal, intradermal, transdermal, parenteral, rectal, subcutaneous, intravenous, intraurethral, intramuscular or topical.

Solid oral formulations include, but are not limited to, tablets, capsules (soft or hard gelatin), dragees (containing the active ingredient in powder or pellet form), troches and lozenges.

Liquid formulations include, but are not limited to, syrups, emulsions and sterile injectable liquids, such as suspensions or solutions.

Topical dosage forms of the compounds include ointments, pastes, creams, lotions, powders, solutions, eye or ear drops, impregnated dressings and may contain appropriate conventional additives such as preservatives, solvents to assist drug penetration.

The pharmaceutical compositions of the present patent application may be prepared by conventional techniques known in literature.

Suitable doses of the compounds for use in treating the diseases or disorders described herein can be determined by those skilled in the relevant art. Therapeutic doses are generally identified through a dose ranging study in humans based on preliminary evidence derived from the animal studies. Doses must be sufficient to result in a desired therapeutic benefit without causing unwanted side effects. Mode of administration, dosage forms and suitable pharmaceutical excipients can also be well used and adjusted by those skilled in the art. All changes and modifications are envisioned within the scope of the present patent application.

In some embodiments, the present invention provides a pharmaceutical composition comprising a compound of Formula (I) or a pharmaceutically acceptable salt thereof or a stereoisomer or a tautomer thereof as described herein and at least one pharmaceutically acceptable excipient (such as a pharmaceutically acceptable carrier or diluent). Preferably, the pharmaceutical composition comprises a therapeutically effective amount of at least one compound described herein. The composition described herein the present invention may be associated with a pharmaceutically acceptable excipient (such as a carrier or a diluent) or be diluted by a carrier or enclosed within a carrier which can be in the form of a capsule, sachet, paper or other container.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof. Besides inert diluents, the oral compositions can also include adjuvants such as wetting agents, emulsifying and suspending agents, sweetening, flavoring and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are preferably suppositories which can be prepared by mixing the compounds of this application with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid compositions of a similar type may also be employed as fillers in soft and hard filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, including but not limited to tableting lubricants and other tableting aids such a magnesium stearate and microcrystalline cellulose. In the case of capsules, tablets and pills, the dosage forms may also comprise buffering agents.

Dosage forms for topical or transdermal administration of a compound of this application include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this application.

The ointments, pastes, creams and gels may contain, in addition to an active compound of this application, excipients such as animal and vegetable fats, oils, waxes, paraffins, starch, tragacanth, cellulose derivatives, polyethylene glycols, silicones, bentonites, silicic acid, talc and zinc oxide or mixtures thereof.

Powders and sprays can contain, in addition to the compounds of this application, excipients such as lactose, talc, silicic acid, aluminum hydroxide, calcium silicates and polyamide powder or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Administration of the disclosed compounds and pharmaceutical compositions can be accomplished via any mode of administration for therapeutic agents. These modes include systemic or local administration such as oral, nasal, parenteral, intravenous, transdermal, subcutaneous, vaginal, buccal, rectal or topical administration modes.

Depending on the intended mode of administration, the disclosed compounds or pharmaceutical compositions can be in solid, semi-solid or liquid dosage form, such as, for example, injectables, tablets, suppositories, pills, time-release capsules, elixirs, tinctures, emulsions, syrups, powders, liquids, suspensions or the like, sometimes in unit dosages and consistent with conventional pharmaceutical practices. Likewise, they can also be administered in intravenous (both bolus and infusion), intraperitoneal, subcutaneous or intramuscular form and all using forms well known to those skilled in the pharmaceutical arts.

Illustrative pharmaceutical compositions are tablets and gelatin capsules comprising one or more compounds of the present disclosure and a pharmaceutically acceptable carrier, such as, but not limited to, a) a diluent, e.g., purified water, triglyceride oils, such as hydrogenated or partially hydrogenated vegetable oil or mixtures thereof, corn oil, olive oil, sunflower oil, safflower oil, fish oils, such as EPA or DHA or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, alginic acid or its sodium salt or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG400, PEG200.

Liquid, particularly injectable, compositions can, for example, be prepared by dissolution, dispersion, etc. For example, one or more disclosed compound is dissolved in or mixed with a pharmaceutically acceptable solvent such as, for example, water, saline, aqueous dextrose, glycerol, ethanol and the like, to thereby form an injectable isotonic solution or suspension. Proteins such as albumin, chylomicron particles or serum proteins can be used to solubilize the disclosed compounds.

One or more disclosed compounds or compositions can be delivered by parental administration. The parental injectable administration is generally used for subcutaneous, intramuscular or intravenous injections and infusions. Injectables can be prepared in conventional forms, either as liquid solutions or suspensions or solid forms suitable for dissolving in liquid prior to injection.

In yet another aspect, the present invention provides a pharmaceutical composition comprising a compound of Formula (I) or a pharmaceutically acceptable salt or a stereoisomer or a tautomer thereof and at least one pharmaceutically acceptable excipient (such as a pharmaceutically acceptable carrier or diluent).

In another aspect, the present invention provides a pharmaceutical composition comprising a compound of Formula (I) or a pharmaceutically acceptable salt or a stereoisomer or a tautomer thereof for use as a medicament.

In another aspect, the present invention provides a pharmaceutical composition comprising a compound of Formula (I) or a pharmaceutically acceptable salt or a stereoisomer or a tautomer thereof for treating diseases or disorders that are dependent on or mediated by KAT6A.

### V. Administration

The compound of the present disclosure may be administered to an individual in accordance with an effective dosing regimen for a desired period of time or duration, such as at least about one week, at least about two weeks, at least about three weeks, one month, at least about 2 months, at least about 3 months, at least about 6 months, or at least about 12 months or longer. In one variation, the compound is administered on a daily or intermittent schedule for the required duration, up to the individual's life.

The dosage or dosing frequency of the compound of the present disclosure may be adjusted over the course of the treatment, based on the judgment of the administering physician.

The compound may be administered to an individual, such as a human in an effective amount.

The compound of the present invention can be administered by any useful route and means, such as by oral or parenteral (e.g., intravenous) administration. Therapeutically effective amounts of the compound of the present invention are from about 0.00001 mg/kg body weight per day to about 10 mg/kg body weight per day, such as from about 0.0001 mg/kg body weight per day to about 10 mg/kg body weight per day, or such as from about 0.001 mg/kg body weight per day to about 1 mg/kg body weight per day, or such as from about 0.01 mg/kg body weight per day to about 1 mg/kg body weight per day, or such as from about 0.05 mg/kg body weight per day to about 0.5 mg/kg body weight per day, or such as from about 0.3 μg to about 30 mg per day, or such as from about 30 μg to about 300 μg per day.

Therapeutically effective amounts of the compound of the present invention, are from about 0.01 mg per dose to about 1000 mg per dose, such as from about 0.01 mg per dose to about 100 mg per dose, or such as from about 0.1 mg per dose to about 100 mg per dose, or such as from about 1 mg per dose to about 100 mg per dose, or such as from about 1 mg per dose to about 10 mg per dose. Other therapeutically effective amounts of the compound of the present invention are about 1 mg per dose, or about 2, 3, 4, 5, 6, 7, 8, 9, 10, 15, 20, 25, 30, 35, 40, 45, 50, 55, 60, 65, 70, 75, 80, 85, 90, 95, or about 100 mg per dose. Other therapeutically effective amounts of the compound of the present invention are about 100 mg per dose, or about 125, 150, 175, 200, 225, 250, 275, 300, 350, 400, 450, or about 500 mg per dose. A single dose can be administered hourly, daily, or weekly. For example, a single dose can be administered once every 1 hour, 2, 3, 4, 6, 8, 12, 16 or once every 24 hours. A single dose can also be administered once every 1 day, 2, 3, 4, 5, 6, or once every 7 days. A single dose can also be administered once every 1 week, 2, 3, or once every 4 weeks. In certain embodiments, a single dose can be administered once every week. A single dose can also be administered once every month.

### VI. Methods and/or Uses

In another aspect, the present invention provides methods of treating a disease or a disorder comprising administering a therapeutically effective amount of a compound of Formula (I) or a pharmaceutically acceptable salt or a stereoisomer or a tautomer thereof to a subject, e.g., a human, in need thereof.

In another aspect, the present invention provides a use of a compound of Formula (I) or a pharmaceutically acceptable salt or a stereoisomer or a tautomer thereof for the manufacture of a medicament for treating a disease or condition, e.g., cancer.

In some embodiments, the present invention provides a compound of Formula (I) or a pharmaceutical acceptable salt or a stereoisomer or a tautomer thereof, for use in the treatment of diseases or disorders mediated by KAT6A.

In some embodiments, the present invention provides a method of inhibiting KAT6A in a subject comprising administering to the subject in need thereof, a therapeutically effective amount of a compound of Formula (I) or a pharmaceutically acceptable salt or a stereoisomer or a tautomer thereof.

In some embodiments, the present invention provides a method for treating a disease or disorder mediated by KAT6A, in a subject comprising administering to the subject in need thereof a therapeutically effective amount of a compound of Formula (I) or a pharmaceutically acceptable salt thereof or a stereoisomer or a tautomer thereof.

In some embodiments, the present invention provides a compound or a pharmaceutically acceptable salt or a stereoisomer or a tautomer thereof, for use as a medicament.

In some embodiments, the present invention provides a use of pharmaceutical composition comprising a compound of Formula (I), in the manufacture of a medicament for treating a disease or disorder mediated by KAT6A. In some embodiments, the present invention provides a use of pharmaceutical composition comprising a compound of Formula (I), in the manufacture of a medicament for treating cancer that is mediated by KAT6A.

In some embodiments, the present invention provides a use of compound of Formula (I) or a pharmaceutically acceptable salt or a stereoisomer or a tautomer thereof, in the manufacture of a medicament for treating or preventing a disease or disorder mediated by KAT6A.

In some embodiments, the disease or disorder dependent upon or mediated by KAT6A, is cancer.

In some embodiments, the cancer is selected from brain gliomas, glioblastomas, astrocytomas, multiforme, Bannayan-Zonana syndrome, Cowden disease, Lhermitte-Duclos disease, breast cancer, colon cancer, head and neck cancer, kidney, liver, lung cancer, bone cancer, colorectal cancer, germ cell cancer, melanoma, ovarian cancer, pancreatic cancer, adenocarcinoma, ductal adenocarcinoma, adenosquamous carcinoma, acinar cell carcinoma, glucagonoma, insulinoma, prostate, sarcoma and thyroid cancer, lymphoblastic T cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T cell leukemia, plasmacytoma, immunoblastic large cell leukemia, mantle cell leukemia, megakaryoblastic leukemia, multiple myeloma, acute megakaryocytic leukemia, promyelocytic leukemia, erythroleukemia, malignant lymphoma, Hodgkin's lymphoma, non-Hodgkin's lymphoma, lymphoblastic T cell lymphoma, Burkitt's lymphoma, follicular lymphoma, neuroblastoma, bladder cancer, urothelial cancer, vulval cancer, uterine/cervical cancer, endometrial cancer, renal cancer, mesothelioma, esophageal cancer, salivary gland cancer, hepatocellular cancer, gastric cancer, nasopharyngeal cancer, buccal cancer, cancer of the mouth, GIST (gastrointestinal stromal tumor), neuroendocrine cancers, testicular cancer and virus-related cancer.

In some embodiments, the present invention provides a use of compound of Formula (I) or a pharmaceutically acceptable salt or a stereoisomer or a tautomer thereof, in the manufacture of a medicament for treating cancer selected from brain gliomas, glioblastomas, astrocytomas, multiforme, bannayan-Zonana syndrome, Cowden disease, Lhermitte-Duclos disease, breast cancer, colon cancer, head and neck cancer, kidney, liver, lung cancer, bone cancer, colorectal cancer, germ cell cancer, melanoma, ovarian cancer, pancreatic cancer, adenocarcinoma, ductal adenocarcinoma, adenosquamous carcinoma, acinar cell carcinoma, glucagonoma, insulinoma, prostate, sarcoma and thyroid cancer, lymphoblastic T cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T cell leukemia, plasmacytoma, immunoblastic large cell leukemia, mantle cell leukemia, megakaryoblastic leukemia, multiple myeloma, acute megakaryocytic leukemia, promyelocytic leukemia, erythroleukemia, malignant lymphoma, Hodgkin's lymphoma, non-Hodgkin's lymphoma, lymphoblastic T cell lymphoma, Burkitt's lymphoma, follicular lymphoma, neuroblastoma, bladder cancer, urothelial cancer, vulval cancer, uterine/cervical cancer, endometrial cancer, renal cancer, mesothelioma, esophageal cancer, salivary gland cancer, hepatocellular cancer, gastric cancer, nasopharyngeal cancer, buccal cancer, cancer of the mouth, GIST (gastrointestinal stromal tumor), neuroendocrine cancers, testicular cancer and virus-related cancer.

In some embodiments, the present invention provides a compound of Formula (I), or a pharmaceutically acceptable salt or a stereoisomer or a tautomer thereof, for use in treating of a disease or disorder mediated by KAT6A.

In some embodiments, the present invention provides a compound of Formula (I), or a pharmaceutically acceptable salt or a stereoisomer or a tautomer thereof, for use in treating or preventing cancer selected from brain gliomas, glioblastomas, astrocytomas, multiforme, bannayan-Zonana syndrome, Cowden disease, Lhermitte-Duclos disease, breast cancer, colon cancer, head and neck cancer, kidney, liver, lung cancer, bone cancer, colorectal cancer, germ cell cancer, melanoma, ovarian cancer, pancreatic cancer, adenocarcinoma, ductal adenocarcinoma, adenosquamous carcinoma, acinar cell carcinoma, glucagonoma, insulinoma, prostate, sarcoma and thyroid cancer, lymphoblastic T cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T cell leukemia, plasmacytoma, immunoblastic large cell leukemia, mantle cell leukemia, megakaryoblastic leukemia, multiple myeloma, acute megakaryocytic leukemia, promyelocytic leukemia, erythroleukemia, malignant lymphoma, Hodgkin's lymphoma, non-Hodgkin's lymphoma, lymphoblastic T cell lymphoma, Burkitt's lymphoma, follicular lymphoma, neuroblastoma, bladder cancer, urothelial cancer, vulval cancer, uterine/cervical cancer, endometrial cancer, renal cancer, mesothelioma, esophageal cancer, salivary gland cancer, hepatocellular cancer, gastric cancer, nasopharyngeal cancer, buccal cancer, cancer of the mouth, GIST (gastrointestinal stromal tumor), neuroendocrine cancers, testicular cancer and virus-related cancer.

### VII. EXAMPLES

While specific embodiments of the subject invention have been discussed, the specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

The following abbreviations refer respectively to the definitions below: DMSO—dimethylsulfoxide; THF—tetrahydrofuran; DCM—dichloromethane; LiHMDS—lithium bis(trimethylsilyl)amide; Pd(Amphos)Cl₂—bis(di-tert-butyl(4-dimethylaminophenyl)phosphine)dichloropalladium(II); NH₄Cl—ammonium chloride; Na₂SO₄—sodium sulphate; br—broad; ° C.—degree Celsius; DMSO-d6—deuterated dimethylsulfoxide; DMF—N, N-dimethylformamide; g—gram; h—hours; 1H—proton; LC-MS—liquid chromatography-mass spectroscopy; MHz—megahertz (frequency); MS—mass spectroscopy; M—molar; mmol—millimole; mL—millilitre; min—minutes; mol—moles; M^{+/-}—molecular ion; m/z-mass to charge ratio; NMR—nuclear magnetic resonance; ppm—parts per million; rt or RT—room temperature; RM—reaction mixture; s—singlet; d—doublet, t—triplet; q—quartet; m—multiplet; dd—doublet of doublets; TLC—thin layer chromatography; %—percentage and 6—delta.

### Example 1. General Scheme

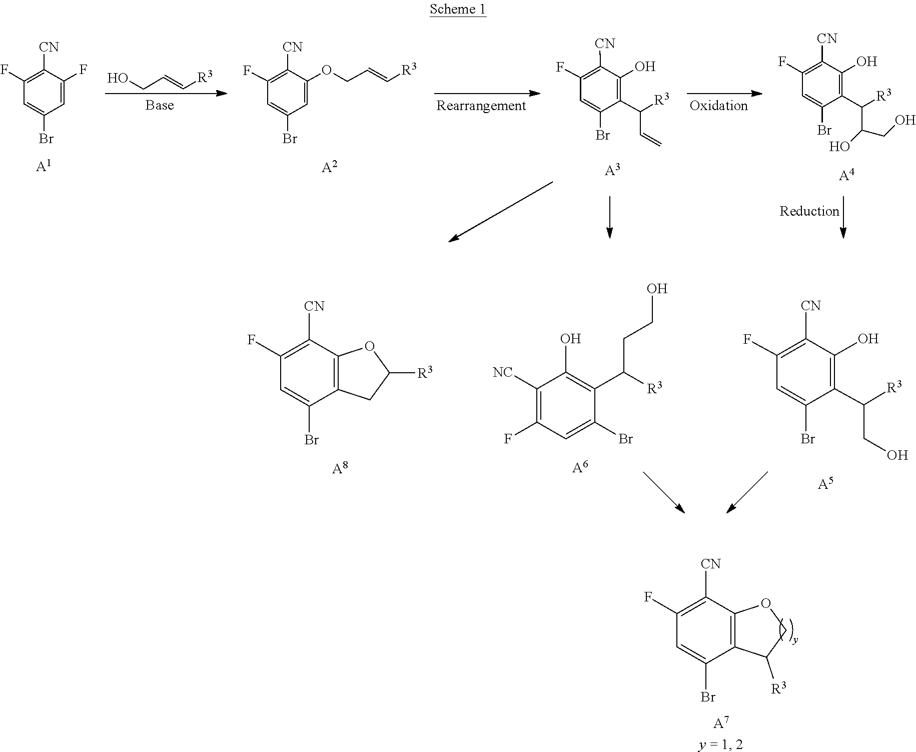

The general approach for the synthesis of compounds of general formula (A⁷ and A⁸) is depicted in above Scheme 1. Compound of formula (A²) can be obtained from compound of formula (A¹) by reacting with prop-2-en-1-ol (optionally substituted with R³) and a base in appropriate solvent at a room temperature. Compound of formula (A²) on rearrangement reaction at high temperature can give compound of formula (A³) which can be further oxidized in presence of oxidizing agents can yield compound of formula (A⁴). Compound of formula (A⁴) on reductive cleavage reaction can give compound of formula (A⁵). Compound of formula (A⁵) can undergo cyclization reaction to give compound of formula (A⁷). Further compound of formula (A³) react with boron derivatives can give compound of formula (A⁶) which can be cyclized with azodicarboxylate derivative to give compound of formula (A⁷). Further compound of formula (A³) can be reacted with an appropriate acid to give compound of formula (A⁸).

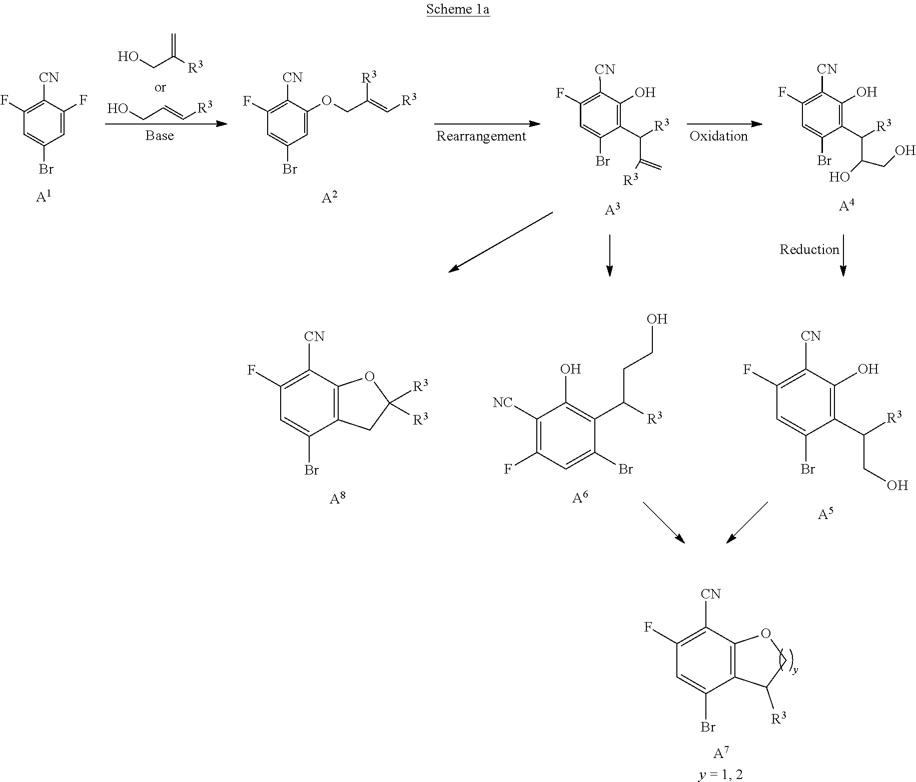

A general approach for the synthesis of compounds of general formula (A⁷ and A⁸) is depicted in above Scheme 1a. Compound of formula (A²) can be obtained from compound of formula (A¹) by reacting with prop-2-en-1-ol (optionally substituted with R³) and a base in appropriate solvent at room temperature. Compound of formula (A²) on rearrangement reaction at high temperature can give compound of formula (A³) which can be further oxidized in presence of oxidizing agents to yield compound of formula (A⁴). Compound of formula (A⁴) on reductive cleavage reaction can give compound of formula (A⁵). Compound of formula (A⁵) can undergo cyclization reaction to give compound of formula (A⁷). Further, a compound of formula (A³) can react with boron derivatives to give a compound of formula (A⁶), which can be cyclized, e.g., using an azodicarboxylate, to give a compound of formula (A⁷). Further, a compound of formula (A³) can be reacted with an appropriate acid to give compound of formula (A⁸).

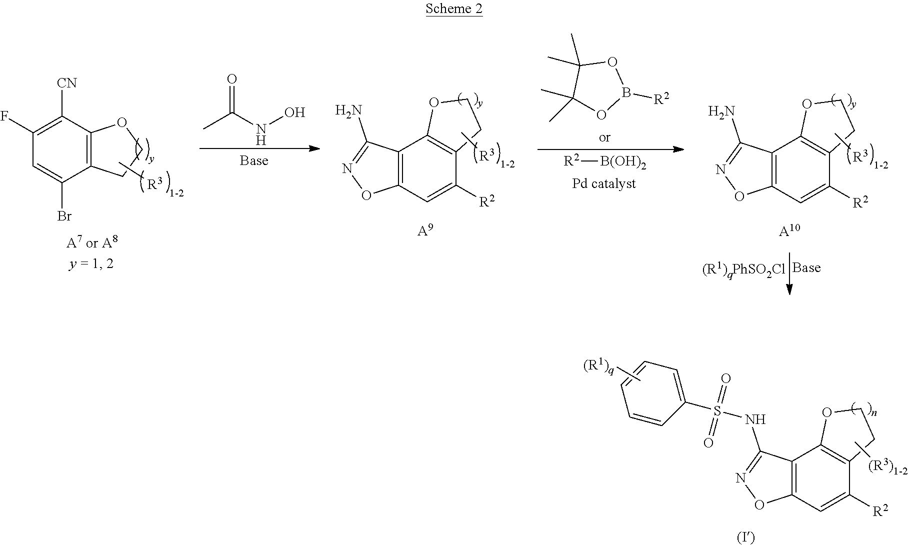

The general scheme for the synthesis of compounds of general Formula (I′) from the compound of formula (A⁷ and/or A⁸) is depicted in above scheme. Compound of formula (A⁹) can be obtained from (A⁷ and/or A⁸) by cyclization reaction in the presence of base. Compound of formula (A⁹) can be reacted with an appropriate boronates to give compound of formula (A¹⁰). Compound of formula (A¹⁰) upon reacting with appropriate sulfonyl chloride derivatives to give compound of Formula (I′).

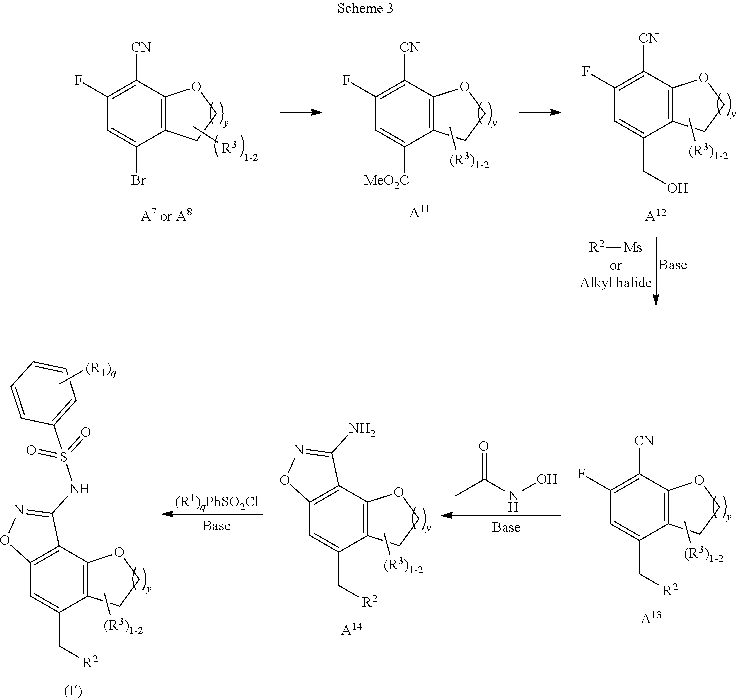

The general Scheme 3 for the synthesis of compounds of general Formula (I′) is depicted above. Compound of formula (A¹¹) can be obtained from (A⁷ and A⁸) by carbonylation. Compound of formula (A¹¹) can undergo reduction reaction in presence of suitable reducing agent and base to give compound of formula (A¹²). Compound of formula (A¹²) can be reacted with appropriate sulfonic acids or alkyl halides in the presence of base to gives compound of formula (A¹³) which upon cyclization in the presence of a base to give compound of formula (A¹⁴). Compound of formula (A¹⁴) upon reacting with appropriate sulfonyl chloride derivatives to give compound of formula (I′).

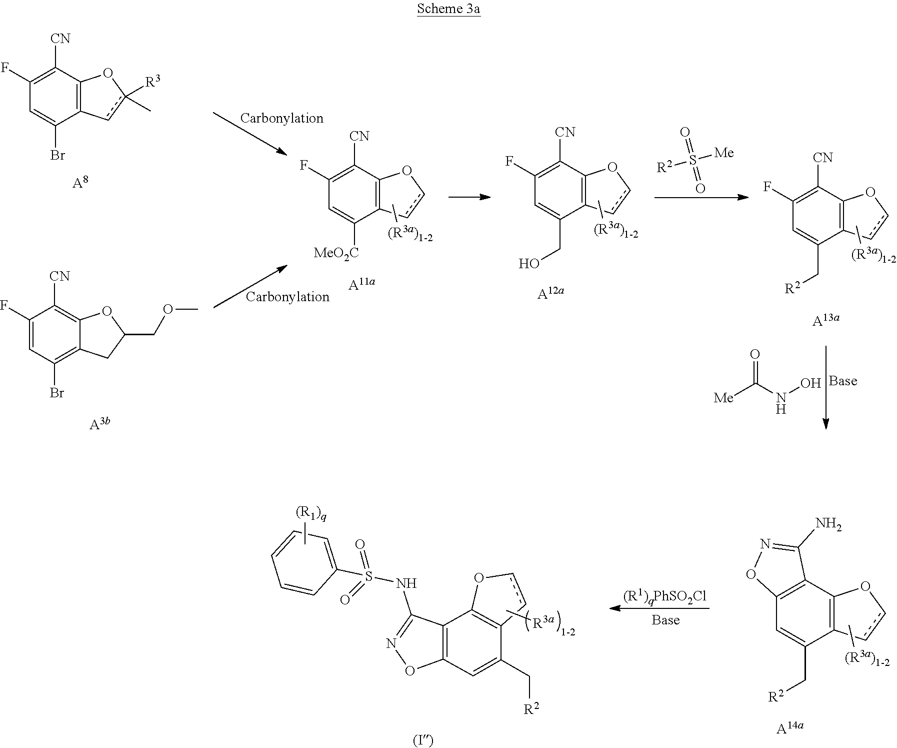

The general Scheme 3a for the synthesis of compounds of general formula (I″) is depicted above. Compound of formula (A^{11a}) can be obtained from either A^{3b} or A⁸ by carbonylation in the presence of suitable reagents. Compound of formula (A^{1a}) can undergo reduction reaction in presence of suitable reducing agent and base to give compound of formula (A^{12a}). Compound of formula (A^{12a}) can be reacted with appropriate sulphonic acids in the presence of a base to give compound of formula (A^{13a}) which upon cyclization in the presence of a base gives compound of formula (A^{14a}). Compound of formula (A^{14a}) upon reacting with appropriate sulfonyl chloride derivatives gives compound of Formula (I″).

### Example 2. Synthesis of Intermediates

### Intermediate-1: 4-Phenyl-2,3-dihydrobenzofuro[7,6-d]isoxazol-8-amine (Method-I)

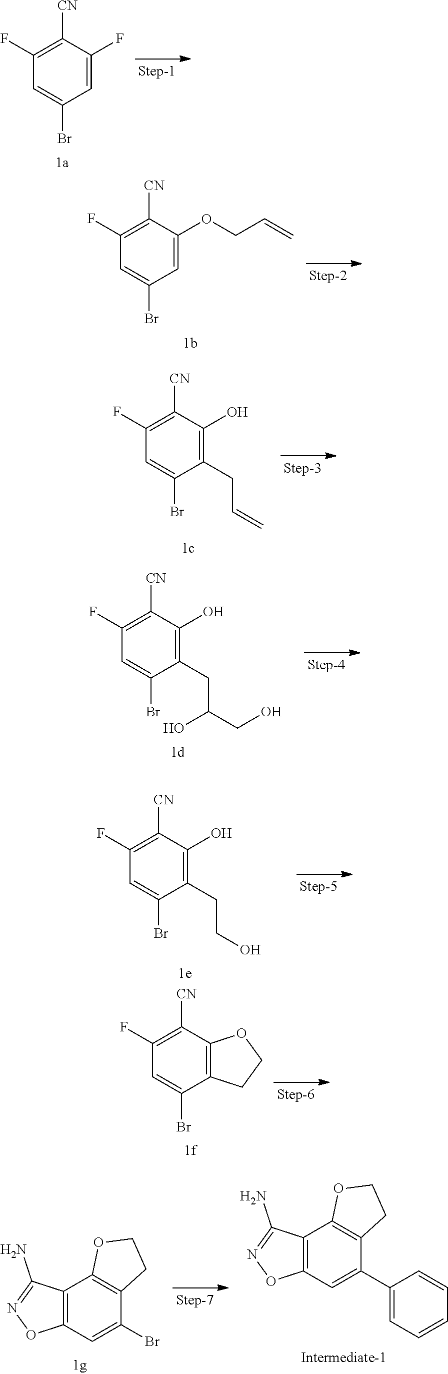

### Step-1: 2-(Allyloxy)-4-bromo-6-fluorobenzonitrile

A stirred solution of 4-bromo-2,6-difluorobenzonitrile (10 g, 45.8 mmol) and prop-2-en-1-ol (2.66 g, 45.8 mmol) in dioxane (50 mL) and THF (50 mL) was cooled to 0° C. and added sodium hydride (60% in mineral oil) (3.66 g, 91.74 mmol) in portions. The reaction mixture was gradually warmed to RT and stirred for 2 h. The mixture was quenched with ice and extracted with ethyl acetate, washed with brine solution, dried over Na₂SO₄ and concentrated under reduced pressure to get crude product which was purified by column using 100-200 silica and eluted with 0-10% ethyl acetate in hexane to afford title compound (8.2 g, 72.3%). LC-MS: 255.9 [M+H]⁺, LC-MS: 258.0 [M+2+H]⁺.

### Step-2: 3-Allyl-4-bromo-6-fluoro-2-hydroxybenzonitrile

A stirred solution of 2-(allyloxy)-4-bromo-6-fluorobenzonitrile (6.5 g, 25.3 mmol) in 1,2-dichlorobenzene (65 mL) was heated to 160° C. for 36 h. 1,2-Dichlorobenzene was concentrated off. The crude obtained was purified on silica gel (100-200 mesh) column using 30-40% ethyl acetate in hexane as eluent to afford the title compound (4 g, 61.5%). LC-MS: 255.9 [M+H]⁺.

### Step-3: 4-Bromo-3-(2,3-dihydroxypropyl)-6-fluoro-2-hydroxybenzonitrile

4-Methylmorpholine N-oxide (2.08 g, 17.7 mmol) was added to a stirred solution of 3-allyl-4-bromo-6-fluoro-2-hydroxybenzonitrile (2.6 g, 10.1 mmol) in acetone-water (20 mL, 2 mL) at RT. After stirring for 5 minutes, 4% Aq. solution of Osmium tetroxide (3.22 mL, 0.5 mmol) was added and the reaction mixture was stirred for 24 h. This was then quenched with saturated Na₂S₂O₃ solution and extracted with ethyl acetate. The organic portion was washed with NH₄Cl solution, dried over Na₂SO₄ and concentrated under reduced pressure to get crude product which was purified by Combiflash chromatograph using 10% methanol in DCM to afford the title compound (1.5 g, 50.9%). LC-MS: 289.9 [M+H]⁺.

### Step-4: 4-Bromo-6-fluoro-2-hydroxy-3-(2-hydroxyethyl)benzonitrile

A solution of 4-bromo-3-(2,3-dihydroxypropyl)-6-fluoro-2-hydroxybenzonitrile (1.5 g, 5.1 mmol) in water-acetone (15 mL and 0.5 mL respectively) was cooled to 0° C. Sodium periodate (1.1 g, 5.17 mmol) was added into the reaction mixture. After stirring the reaction mixture for 5 min, NaBH₄ was added in portions so that reaction temperature does not exceed 5° C. After complete addition, the reaction mixture was stirred at RT for an hour and acidified by adding 1N HCl slowly at 0° C. Then the mixture was extracted with ethyl acetate, washed with brine solution, dried over Na₂SO₄ and concentrated under reduced pressure to afford the title compound (0.5 g, 37.1%). LC-MS: 257.9 [M+H]⁺.

### Step-5: 4-Bromo-6-fluoro-2,3-dihydrobenzofuran-7-carbonitrile

A stirred solution of 4-bromo-6-fluoro-2-hydroxy-3-(2-hydroxyethyl)benzonitrile (0.4 g, 1.53 mmol) and triphenyl phosphine (0.403 g, 1.53 mmol) in dry THF (5 mL) was cooled 0° C. Diisopropyl azodicarboxylate (0.268 g, 1.53 mmol) was added slowly and the reaction mixture was gradually warmed to RT and stirred for 1 h. Then the mixture was quenched with water and extracted with ethyl acetate. The organic portion was dried over Na₂SO₄ and concentrated under reduced pressure to get crude product which was purified by Combi flash chromatograph by eluting with 30% ethyl acetate in hexane to afford pure title compound (0.26 g 69.8%). 1H-NMR (400 MHz, DMSO-D6) δ 7.28 (d, 1H), 4.82 (t, 2H), 3.18 (t, 2H).

### Step-6: 4-Bromo-2,3-dihydrobenzofuro[7,6-d]isoxazol-8-amine

To a stirred solution of 4-bromo-6-fluoro-2,3-dihydrobenzofuran-7-carbonitrile (0.15 g, 0.62 mmol) N-hydroxyacetamide (0.14 g, 1.86 mmol) in acetonitrile-water (9:1; 3 mL) was added potassium carbonate (0.68 g, 4.96 mmol). The mixture was heated to 70° C. for 12 h and then cooled to RT, extracted with ethyl acetate, washed with brine solution, dried over sodium sulphate and concentrated under reduced pressure to get crude which was purified in Combi flash chromatograph by eluting with 30% ethyl acetate in hexane to afford the pure title compound (0.07 g, 43.7%). LC-MS: 255.1 [M+H]⁺.

### Step-7: 4-Phenyl-2,3-dihydrobenzofuro[7,6-d]isoxazol-8-amine

A mixture of 4-bromo-2,3-dihydrobenzofuro[7,6-d]isoxazol-8-amine (0.1 g, 0.392 mmol), phenylboronic acid (0.072 g, 0.588 mmol) in dioxane-water (2 mL and 0.4 mL respectively) was degassed with nitrogen and Pd(amphos)Cl₂ (0.014 g, 0.02 mmol) was added and heated the mixture to 100° C. for 12 h. The reaction mixture was then extracted with ethyl acetate, washed with water, dried over Na₂SO₄ and concentrated under reduced pressure which was further purified using Combi flash chromatograph by eluting with 30% ethyl acetate in hexane to afford pure title compound (0.08 g, 80.9%). LC-MS: 253.05 [M+H]⁺.

### Intermediate-2: 4-(Thiophen-2-yl)-2,3-dihydrobenzofuro[7,6-d]isoxazol-8-amine (Method-II)

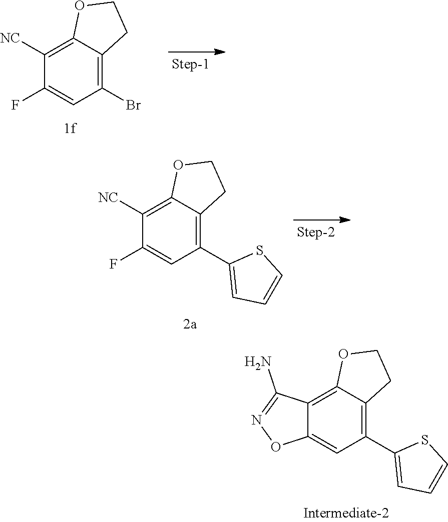 [SMILES: N#Cc1c(F)cc(Br)c2c1OCC2 ; N#Cc1c(F)cc(-c2cccs2)c2c1OCC2 ; Nc1noc2cc(-c3cccs3)c3c(c12)OCC3]

### Step-1:6-Fluoro-4-(thiophen-2-yl)-2,3-dihydrobenzofuran-7-carbonitrile

A solution of 4-bromo-6-fluoro-2,3-dihydrobenzofuran-7-carbonitrile (0.15 g, 0.62 mmol), thiophen-2-ylboronic acid (0.119 g, 0.93 mmol), K₃PO₄ (0.395 g, 1.86 mmol) in 1,4-dioxane (3.6 mL) and water (0.6 mL) was purged with nitrogen and added Pd(Amphos)Cl₂ (0.044 g, 0.06 mmol) and heated the mixture to 90° C. for 12 h. The reaction mass was cooled to RT, then diluted with 10% methanol in DCM and filtered through Celite. The filtrate was extracted with 10% methanol in DCM. The organic portion was washed with NaHCO₃ solution and concentrated under reduced pressure which was purified in Combi flash using 20% ethyl acetate in hexane to afford the title compound (0.14 g, 92.6%). LC-MS: 246.0 [M+H]⁺.

### Step-2: 4-(Thiophen-2-yl)-2,3-dihydrobenzofuro[7,6-d]isoxazol-8-amine

This intermediate was prepared according to the procedure described in Step-6 of Intermediate-1(1 g) with appropriate variations in reactants, quantities of reagents, and solvents. LC-MS: 259.1 [M+H]⁺.

The intermediates listed below in Table 2 were prepared by similar procedure described in the synthesis of Intermediate-1 & Intermediate-2 with appropriate variations in coupling methods, reactants, quantities of reagents and solvents, and reaction conditions. The characterization data of the compounds are summarized herein the below table.

TABLE 2
Intermediates 3 to 13

| | | Starting materials/ | |
|---|---|---|---|
| Intermediate | Structure | Coupling methods | Spectral data |
| 3 | 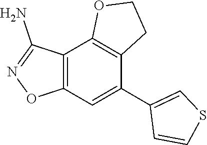 [SMILES: Nc1noc2cc(-c3ccsc3)c3c(c12)OCC3] | 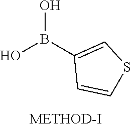 | LC-MS: 259.0 [M + H]⁺ |
| 4 | 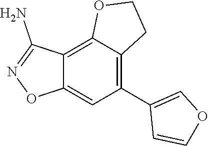 [SMILES: Nc1noc2cc(-c3ccoc3)c3c(c12)OCC3] | 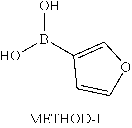 | LC-MS: 243.1 [M + H]⁺ |
| 5 | 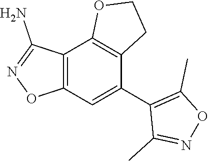 [SMILES: Cc1noc(C)c1-c1cc2onc(N)c2c2c1CCO2] | 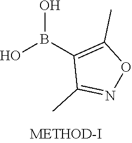 | LC-MS: 272.0 [M + H]⁺ |
| 6 | 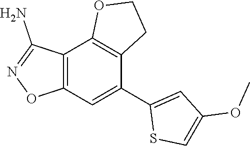 [SMILES: COc1csc(-c2cc3onc(N)c3c3c2CCO3)c1] | 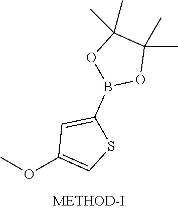 | LC-MS: 289.0 [M + H]⁺ |
| 7 | 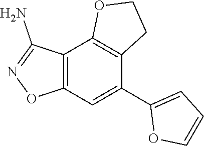 [SMILES: Nc1noc2cc(-c3ccco3)c3c(c12)OCC3] | 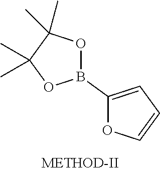 | LC-MS: 243.0 [M + H]⁺ |
| 8 | 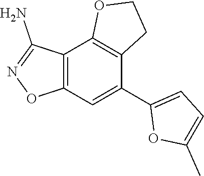 [SMILES: Cc1ccc(-c2cc3onc(N)c3c3c2CCO3)o1] | 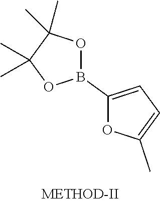 | LC-MS: 257.1 [M + H]⁺ |
| 9 | 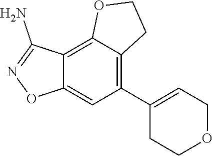 [SMILES: Nc1noc2cc(C3=CCOCC3)c3c(c12)OCC3] | 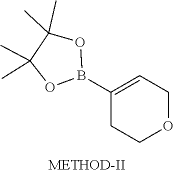 | LC-MS: 259.1 [M + H]⁺ |
| 10 | 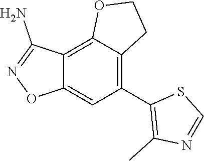 [SMILES: Cc1ncsc1-c1cc2onc(N)c2c2c1CCO2] | 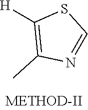 | LC-MS: 274.1 [M + H]⁺ |
| 11 | 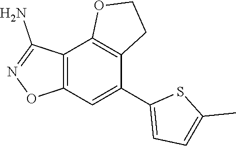 [SMILES: Cc1ccc(-c2cc3onc(N)c3c3c2CCO3)s1] | 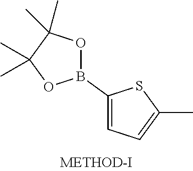 | LC-MS: 273.0 [M + H]⁺ |
| 12 | 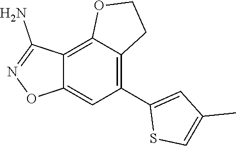 [SMILES: Cc1csc(-c2cc3onc(N)c3c3c2CCO3)c1] | 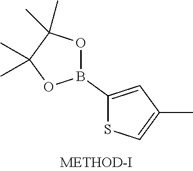 | LC-MS: 273.0 [M + H]⁺ |
| 13 | 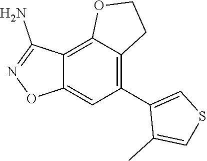 [SMILES: Cc1cscc1-c1cc2onc(N)c2c2c1CCO2] | 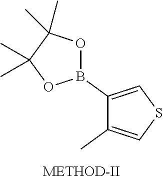 | LC-MS: 273.1 [M + H]⁺ |

### Intermediate-14: 5-Phenyl-3,4-dihydro-2H-chromeno[8,7-d]isoxazol-9-amine

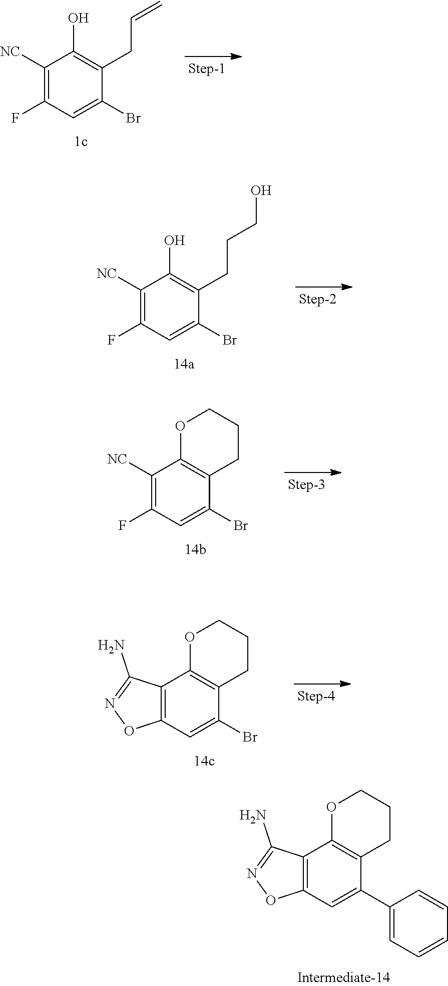 [SMILES: C=CCc1c(Br)cc(F)c(C#N)c1O ; N#Cc1c(F)cc(Br)c(CCCO)c1O ; N#Cc1c(F)cc(Br)c2c1OCCC2 ; Nc1noc2cc(Br)c3c(c12)OCCC3 ; Nc1noc2cc(-c3ccccc3)c3c(c12)OCCC3]

### Step-1: 4-Bromo-6-fluoro-2-hydroxy-3-(3-hydroxypropyl)benzonitrile

To a stirred solution of 3-allyl-4-bromo-6-fluoro-2-hydroxybenzonitrile (2.8 g, 10.9 mmol) in THF (30 mL) was added borane-dimethyl sulfide (0.99 g, 13.1 mmol) at 0° C. After stirring for 1 h at 0° C., 30% H₂O₂(6.2 mL, 54 mmol)) and NaHCO₃ (4.59 g, 54 mmol) were added slowly at 0° C. and stirred at RT for 2 h. Then the reaction mass was quenched with sodium thiosulphate solution, then acidified with 2N HCl, and extracted with ethyl acetate. The organic portion was washed with brine solution, then dried over Na₂SO₄ and concentrated under reduced pressure to get the crude product which was purified by silica gel flash column using 45-50% ethyl acetate in hexane to afford the title compound (1.2 g, 40.04%). LC-MS: 272.0 [M+H]⁺.

### Step-2: 5-Bromo-7-fluorochromone-8-carbonitrile

This intermediate was prepared according to the procedure described in the synthesis of Intermediate-1f with appropriate variations in reactants, quantities of reagents, and solvents. LC-MS: 256.1 [M+H]⁺; ¹H-NMR (400 MHz, DMSO-D6) δ 7.47 (d, 1H), 4.32 (t, 2H), 2.66 (t, 2H), 2.02-1.97 (m, 2H).

### Step-3: 5-Bromo-3,4-dihydro-2H-chromeno[8,7-d]isoxazol-9-amine

This intermediate was prepared according to the procedure described in Step-6 of Intermediate-1 with appropriate variations in reactants, quantities of reagents, and solvents. LC-MS: 270.9 [M+H+2]⁺.

### Step-4: 5-Phenyl-3,4-dihydro-2H-chromeno[8,7-d]isoxazol-9-amine

This intermediate was prepared according to the procedure described in Step-7 of Intermediate-1 with appropriate variations in reactants, quantities of reagents, and solvents. LC-MS: 267.2 [M+H]⁺.

The intermediate-15 in below Table 3 was prepared by the similar procedure described in the synthesis of Intermediate-14 with appropriate variations in coupling methods, reactants, quantities of reagents and solvents, and reaction conditions. The characterization data of the compound is summarized herein the below table.

TABLE 3
Intermediate 15

| | | Starting materials/ | |
|---|---|---|---|
| Intermediate | Structure | Coupling methods | Spectral data |
| 15 | 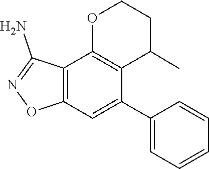 [SMILES: CC1CCOc2c1c(-c1ccccc1)cc1onc(N)c21] | 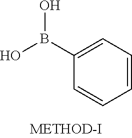 | LC-MS: 281.1 [M + H]⁺ |

### Intermediate-16: 4-(Tetrahydro-2H-pyran-4-yl)-2,3-dihydrobenzofuro[7,6-d]isoxazol-8-amine

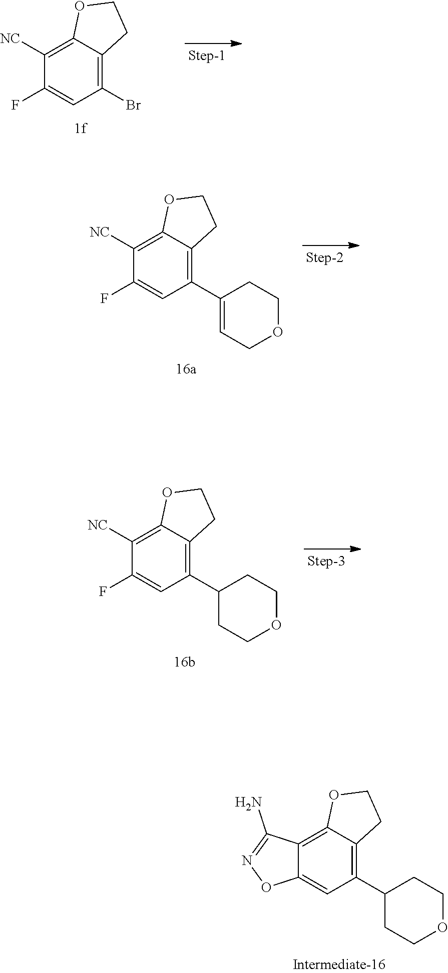 [SMILES: N#Cc1c(F)cc(Br)c2c1OCC2 ; N#Cc1c(F)cc(C2=CCOCC2)c2c1OCC2 ; N#Cc1c(F)cc(C2CCOCC2)c2c1OCC2 ; Nc1noc2cc(C3CCOCC3)c3c(c12)OCC3]

### Step-1:4-(3,6-Dihydro-2H-pyran-4-yl)-6-fluoro-2,3-dihydrobenzofuran-7-carbonitrile

This intermediate was prepared according to the procedure described in the synthesis of 2a with appropriate variations in reactants, quantities of reagents, and solvents.

### Step-2: 6-Fluoro-4-(tetrahydro-2H-pyran-4-yl)-2,3-dihydrobenzofuran-7-carbonitrile

To a solution of 4-(3,6-dihydro-2H-pyran-4-yl)-6-fluoro-2,3-dihydrobenzofuran-7-carbonitrile (0.15 g, 0.61 mmol) in ethanol (6 mL) was carefully added 10% Pd—C(0.16 g, ˜10% W/W) and stirred under positive pressure of hydrogen using a bladder. The resultant mixture was stirred for overnight, filtered through celite, filtrate concentrated under reduced pressure to get white solid. This was purified by Combi flash using 30% ethyl acetate in hexane to afford title compound (0.055 g, 36.3%).

### Step-3: 4-(Tetrahydro-2H-pyran-4-yl)-2,3-dihydrobenzofuro[7,6-d]isoxazol-8-amine

This intermediate was prepared according to the procedure described in Step-6 of Intermediate-1 with appropriate variations in reactants, quantities of reagents, and solvents. LC-MS: 261.2 [M+H]⁺.

### Intermediate-17: 6-Fluoro-4-(1-hydroxyethyl)-2,3-dihydrobenzofuran-7-carbonitrile

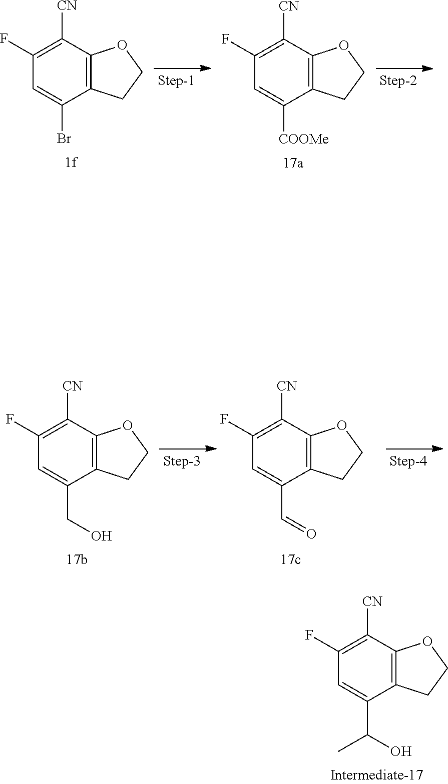 [SMILES: N#Cc1c(F)cc(Br)c2c1OCC2 ; COC(=O)c1cc(F)c(C#N)c2c1CCO2 ; N#Cc1c(F)cc(CO)c2c1OCC2 ; N#Cc1c(F)cc(C=O)c2c1OCC2 ; CC(O)c1cc(F)c(C#N)c2c1CCO2]

### Step-1: Methyl 7-cyano-6-fluoro-2,3-dihydrobenzofuran-4-carboxylate

A solution of 4-bromo-6-fluoro-2,3-dihydrobenzofuran-7-carbonitrile (2 g, 8.26 mmol), triethylamine (2.5 g, 24.7 mmol) in methanol (20 mL) was degassed and added Pd(DPPF)Cl₂ (0.67 g, 0.82 mmol). The mixture was heated in an autoclave at 80° C. under 80 PSI positive pressure of carbon monoxide. After 16 h, the mixture was cooled to RT, filtered through celite and concentrated under reduced pressure to get crude compound which was purified by Combi-flash using 20% ethyl acetate in hexane to afford pure title compound (1.2 g, 65.6%). LC-MS: 222.0 [M+H]⁺.

### Step-2: 6-Fluoro-4-(hydroxymethyl)-2,3-dihydrobenzofuran-7-carbonitrile

A solution of methyl 7-cyano-6-fluoro-2,3-dihydrobenzofuran-4-carboxylate (0.5 g, 2.26 mmol) in THF (5 mL) was cooled to 0° C. and added LiBH₄ (2.25 mL, 2 M in THF) dropwise. The mixture was warmed to RT and heated to reflux for 2 h. The reaction mass was quenched with water at RT, extracted with ethyl acetate, washed with water, brine solution, dried over Na₂SO₄, and concentrated under reduced pressure to get the title compound (0.4 g, 91.5%). LC-MS: 194.1 [M+H]⁺.

### Step-3: 6-Fluoro-4-formyl-2,3-dihydrobenzofuran-7-carbonitrile

To a stirred solution of (6-fluoro-4-(hydroxymethyl)-2,3-dihydrobenzofuran-7-carbonitrile (0.4 g, 2.07 mmol) in DCM (10 mL) was added MnO₂ (1.8 g, 20.7 mmol) at RT. The mixture was stirred at RT for 16 h. Then filtered through celite and the filtrate was concentrated under reduced pressure to get crude compound which was purified in Combi flash using 30% ethyl acetate in hexane to afford the pure title compound (0.25 g, 63.1%). LC-MS: 192.0 [M+H]⁺.

### Step-4: 6-Fluoro-4-(1-hydroxyethyl)-2,3-dihydrobenzofuran-7-carbonitrile

To a solution of 6-fluoro-4-formyl-2,3-dihydrobenzofuran-7-carbonitrile (0.2 g, 1.04 mmol) in THF (2 mL) was added methylmagnesium bromide (0.38 mL, 3 M) dropwise at −78° C. The reaction mixture was stirred at same temperature for 1 h and then quenched with saturated NH₄Cl and extracted with DCM. The organic portion was dried over Na₂SO₄ and concentrated under reduced pressure to get crude compound which was further purified by Combiflash using 30% ethyl acetate in hexane to afford the title compound (0.2 g, 92.2%). LC-MS: 205.95 [M−H]⁻.

### Intermediate-18: 4-(Methoxymethyl)-2,3-dihydrobenzofuro[7,6-d]isoxazol-8-amine

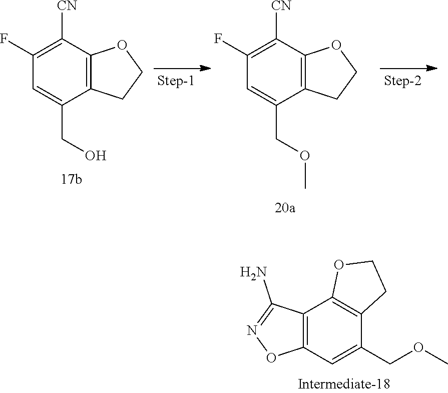 [SMILES: N#Cc1c(F)cc(CO)c2c1OCC2 ; COCc1cc(F)c(C#N)c2c1CCO2 ; COCc1cc2onc(N)c2c2c1CCO2]

### Step-1:6-Fluoro-4-(methoxymethyl)-2,3-dihydrobenzofuran-7-carbonitrile

A mixture of 6-fluoro-4-(hydroxymethyl)-2,3-dihydrobenzofuran-7-carbonitrile (0.2 g, 1.03 mmol), Cs₂CO₃ (1.01 g, 3.1 mmol) and methyl iodide (0.22 g, 1.55 mmol) was heated to 55° C. for 12 h. The mixture was then cooled to RT, diluted with water and extracted with ethyl acetate. The organic portion was washed with water, brine solution, dried over Na₂SO₄ and concentrated under reduced pressure to get crude compound which was purified in Combi flash using 20% ethyl acetate in hexane to afford pure title compound (0.125 g, 58.2%). LC-MS: 208.1 [M+H]⁺.

### Step-2: 4-(Methoxymethyl)-2,3-dihydrobenzofuro[7,6-d]isoxazol-8-amine

This intermediate was prepared according to the procedure described in Step-6 of Intermediate-1 with appropriate variations in reactants, quantities of reagents and solvents. LC-MS: 221.1 [M+H]⁺.

### Intermediate-19: 2-Methyl-4-phenyl-2,3-dihydrobenzofuro[7,6-d]isoxazol-8-amine

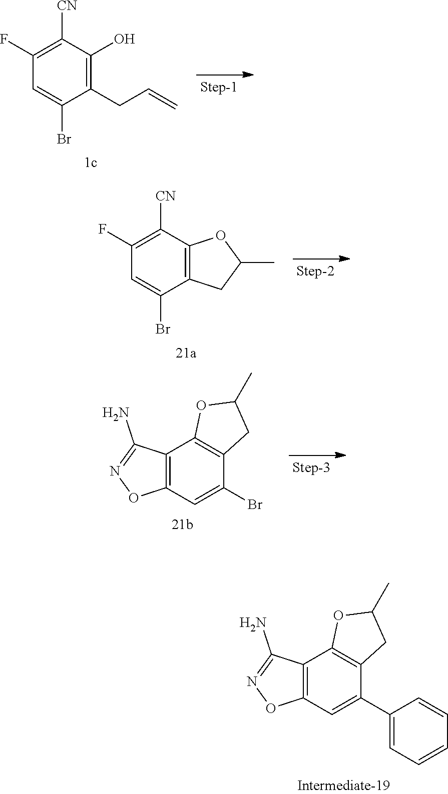 [SMILES: C=CCc1c(Br)cc(F)c(C#N)c1O ; CC1Cc2c(Br)cc(F)c(C#N)c2O1 ; CC1Cc2c(Br)cc3onc(N)c3c2O1 ; CC1Cc2c(-c3ccccc3)cc3onc(N)c3c2O1]

### Step-1: 4-Bromo-6-fluoro-2-methyl-2,3-dihydrobenzofuran-7-carbonitrile

To a solution of 3-allyl-4-bromo-6-fluoro-2-hydroxybenzonitrile (1 g, 3.09 mmol) in 1,2-DCE (30 mL) was added trifluoromethanesulfonic acid (0.293 g, 1.95 mmol) and heated the reaction mixture to 60° C. for 24 h. The mixture was concentrated under reduced pressure to get crude. The crude compound was purified in Combi flash using 5% ethyl acetate in hexane to afford pure title compound (0.7 g, 70%). LC-MS: 254.0 [M+H]⁺, 256.0 [M+2+H]⁺.

### Step-2: 4-Bromo-2-methyl-2,3-dihydrobenzofuro[7,6-d]isoxazol-8-amine

This intermediate was prepared according to the procedure described in the synthesis of step-6 of Intermediate-1 with appropriate variations in reactants, quantities of reagents, and solvents. LC-MS: 269.0 [M+H]⁺.

### Step-3: 2-Methyl-4-phenyl-2,3-dihydrobenzofuro[7,6-d]isoxazol-8-amine

This intermediate was prepared according to the procedure described in the synthesis of step-7 of Intermediate-1 with appropriate variations in reactants, quantities of reagents, and solvents. LC-MS: 267.1 [M+H]⁺.

### Intermediate-20: 4-((1H-pyrazol-1-yl)methyl)-2,3-dihydrobenzofuro[7,6-d]isoxazol-8-amine

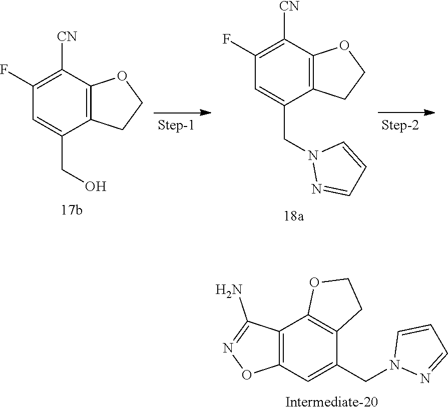 [SMILES: N#Cc1c(F)cc(CO)c2c1OCC2 ; N#Cc1c(F)cc(Cn2cccn2)c2c1OCC2 ; Nc1noc2cc(Cn3cccn3)c3c(c12)OCC3]

### Step-1:4-((1H-pyrazol-1-yl)methyl)-6-fluoro-2,3-dihydrobenzofuran-7-carbonitrile

A mixture of 6-fluoro-4-(hydroxymethyl)-2,3-dihydrobenzofuran-7-carbonitrile (0.9 g, 4.6 mmol), 1-(methylsulfonyl)-1H-pyrazole (0.81 g, 5.5 mmol) and Cs₂CO₃ (1.8 g, 5.5 mmol) in acetonitrile (15 mL) was heated to 70° C. for 1 h. The reaction mixture was cooled to RT, diluted with water, extracted with ethyl acetate, washed with brine solution, dried over Na₂SO₄ and concentrated under reduced pressure which was further purified in Combi flash using 40% ethyl acetate in hexane to afford pure title compound (0.9 g, 79.4%). LC-MS: 244.0 [M+H]⁺.

### Step-2: 4-((1H-pyrazol-1-yl)methyl)-2,3-dihydrobenzofuro[7,6-d]isoxazol-8-amine

A mixture of 4-((1H-pyrazol-1-yl)methyl)-6-fluoro-2,3-dihydrobenzofuran-7-carbonitrile, N-hydroxyacetamide (0.83 g, 11 mmol) and 1,1,3,3-tetramethylguanidine (2.5 g, 22.2 mmol) in acetonitrile-water mixture (10 mL and 1 mL respectively) was heated to 60° C. for 8 h. The reaction mixture was cooled to RT and concentrated under reduced pressure to get crude compound which was purified by Combi flash chromatograph using 5% ethyl acetate in hexane to afford pure title compound (0.33 g, 34.8%). LC-MS: 257.0 [M+H]⁺.

The intermediates listed below in Table 4 were prepared by the similar procedure described in the synthesis of Intermediates 19 and 20 with appropriate variations in coupling methods, reactants, quantities of reagents and solvents and reaction conditions.

TABLE 4
Intermediates 21 and 22

| Intermediate | Structure | Spectral data |
|---|---|---|
| 21 | 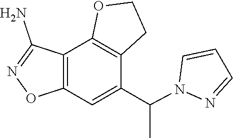 [SMILES: CC(c1cc2onc(N)c2c2c1CCO2)n1cccn1] | LC-MS: 271.2 [M + H]⁺. |
| 22 | 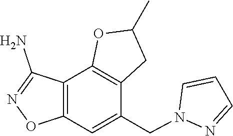 [SMILES: CC1Cc2c(Cn3cccn3)cc3onc(N)c3c2O1] | LC-MS: 271.0 [M + H]⁺. |

The intermediates listed below in Table 5 were prepared by similar procedure described in the synthesis of Intermediate-1 & Intermediate-2 with appropriate variations in coupling methods, reactants, quantities of reagents and solvents, and reaction conditions. The characterization data of the compounds are summarized herein the below table.

TABLE 5
Intermediates 23 and 24

| | | Starting materials/ | |
|---|---|---|---|
| Intermediate | Structure | Coupling methods | Spectral data |
| 23 | 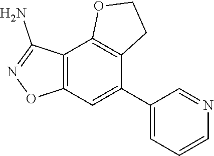 [SMILES: Nc1noc2cc(-c3cccnc3)c3c(c12)OCC3] | 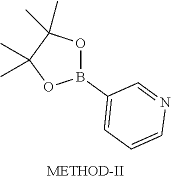 | LC-MS: 254.2 [M + H]⁺ |
| 24 | 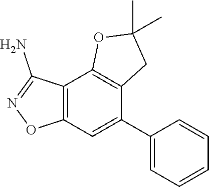 [SMILES: CC1(C)Cc2c(-c3ccccc3)cc3onc(N)c3c2O1] | 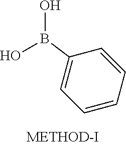 | LC-MS: 281.1 [M + H]⁺ |

The intermediates listed below in Table 6 were prepared by the similar procedure described in the synthesis of Intermediate 19 and 20 with appropriate variations in coupling methods, reactants, quantities of reagents and solvents and reaction conditions.

TABLE 6
Intermediates 25 to 31

| Intermediate | Structure | Starting materials | Spectral data |
|---|---|---|---|
| 25 | 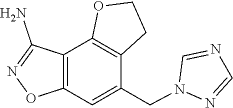 [SMILES: Nc1noc2cc(Cn3cncn3)c3c(c12)OCC3] | 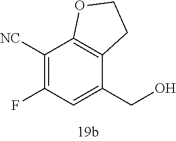 [SMILES: N#Cc1c(F)cc(CO)c2c1OCC2] | LC-MS: 258.1 [M + H]⁺. |
| 26 | 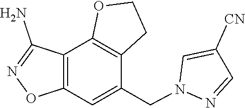 [SMILES: N#Cc1cnn(Cc2cc3onc(N)c3c3c2CCO3)c1] | 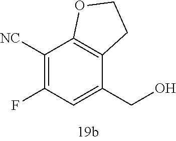 [SMILES: N#Cc1c(F)cc(CO)c2c1OCC2] | LC-MS: 282.1 [M + H]⁺. |
| 27 | 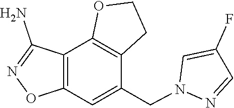 [SMILES: Nc1noc2cc(Cn3cc(F)cn3)c3c(c12)OCC3] | 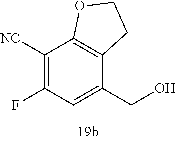 [SMILES: N#Cc1c(F)cc(CO)c2c1OCC2] | LC-MS: 275.1 [M + H]⁺. |
| 28 | 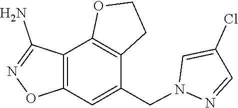 [SMILES: Nc1noc2cc(Cn3cc(Cl)cn3)c3c(c12)OCC3] | 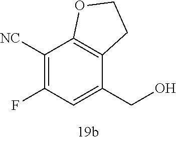 [SMILES: N#Cc1c(F)cc(CO)c2c1OCC2] | LC-MS: 291.0 [M + H]⁺. |
| 29 | 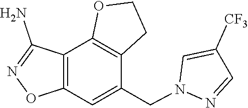 [SMILES: Nc1noc2cc(Cn3cc(C(F)(F)F)cn3)c3c(c12)OCC3] | 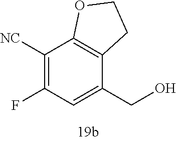 [SMILES: N#Cc1c(F)cc(CO)c2c1OCC2] | LC-MS: 325.1 [M + H]⁺. |
| 30 | 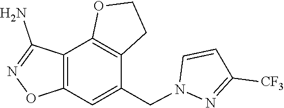 [SMILES: Nc1noc2cc(Cn3ccc(C(F)(F)F)n3)c3c(c12)OCC3] | 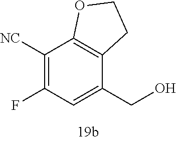 [SMILES: N#Cc1c(F)cc(CO)c2c1OCC2] | LC-MS: 325.1 [M + H]⁺. |
| 31 | 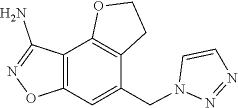 [SMILES: Nc1noc2cc(Cn3ccnn3)c3c(c12)OCC3] | 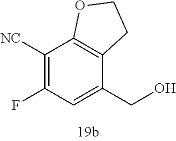 [SMILES: N#Cc1c(F)cc(CO)c2c1OCC2] | LC-MS: 258.1 [M + H]⁺. |

### Intermediate-32:4-((1H-pyrazol-1-yl)methyl)-2,2-dimethyl-2,3-dihydrobenzofuro[7,6-d]isoxazol-8-amine

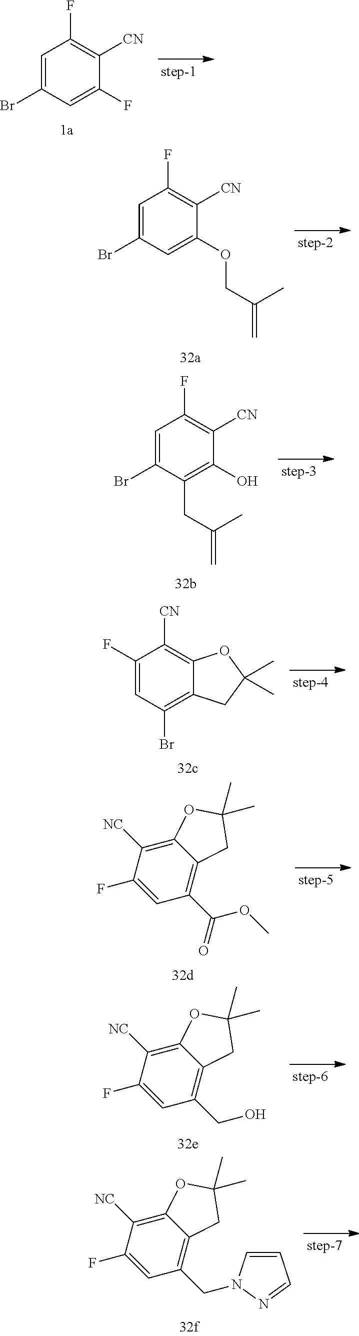

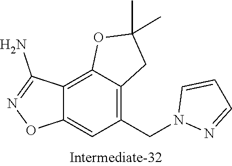 [SMILES: CC1(C)Cc2c(Cn3cccn3)cc3onc(N)c3c2O1]

Step-1: 4-Bromo-2-fluoro-6-((2-methylallyl)oxy)benzonitrile. To a stirred solution of the 4-bromo-2,6-difluorobenzonitrile (10.0 g, 45.87 mmol) and 2-methyl-2-propen-1-ol (3.30 g, 45.87 mmol) in a 1:1 mixture of dioxane (50 ml) and THF (50 ml) was added sodium hydride (2.10 g, 91.74 mmol) at 0° C. The reaction mixture was stirred for 1 h at 0° C. then quenched with ice cold water and extracted with ethyl acetate. The combined organic layer was washed with brine, dried over sodium sulphate, filtered and concentrated to afford the crude material. The crude material was purified by silica-gel flash chromatography using 5% ethyl acetate in hexane as eluent to afford the title compound (8.5 g, 68.60%).

Step-2: 4-Bomo-6-fluoro-2-hydroxy-3-(2-methylallyl)benzonitrile. A stirred solution of 4-bromo-2-fluoro-6-((2-methylallyl)oxy)benzonitrile (8.5 g, 31.46 mmol) in 1,2-dichlorobenzene (85 mL) was heated to 160° C. for 36 h. After reaction completion, reaction mixture was concentrated under vacuum and purified by silica-gel flash column chromatography using 3-5% ethyl acetate in hexane as eluent to afford the title compound (4 g, 61.5%).

Step-3: 4-Bromo-6-fluoro-2,2-dimethyl-2,3-dihydrobenzofuran-7-carbonitrile

To a stirred solution of 4-bromo-6-fluoro-2-hydroxy-3-(2-methylallyl)benzonitrile (0.60 g, 2.22 mmol) in DCE was added trifluoromethane sulfonic acid (0.17 g, 1.11 mmol) dropwise at 0° C. and then stirred at rt for 24 h. After the reaction completion, the reaction mixture was diluted with water extracted with ethyl acetate, washed with brine, dried over sodium sulphate, filtered and concentrated under reduced pressure to get crude which was purified by silica-gel flash column chromatograph using 5% ethyl acetate in hexane as eluent to afford the title compound (0.35 g, 58.34%).

Step-4: Methyl 7-cyano-6-fluoro-2,2-dimethyl-2,3-dihydrobenzofuran-4-carboxylate

This intermediate was prepared according to the procedure described in the synthesis of step-1 of Intermediate-19 with appropriate variations in reactants, quantities of reagents, and solvents.

Step-5: 6-Fluoro-4-(hydroxymethyl)-2,2-dimethyl-2,3-dihydrobenzofuran-7-carbonitrile. This intermediate was prepared according to the procedure described in the synthesis of step-2 of Intermediate-19 with appropriate variations in reactants, quantities of reagents, and solvents.

Step-6: 4-((1H-pyrazol-1-yl)methyl)-6-fluoro-2,2-dimethyl-2,3-dihydrobenzofuran-7-carbonitrile. This intermediate was prepared according to the procedure described in the synthesis of step-1 of Intermediate-20 with appropriate variations in reactants, quantities of reagents, and solvents. LC-MS: 272.1 [M+H]⁺.

Step-7: 4-((1H-pyrazol-1-yl)methyl)-2,2-dimethyl-2,3-dihydrobenzofuro[7,6-d]isoxazol-8-amine. This intermediate was prepared according to the procedure described in the synthesis of step-2 of Intermediate-20 with appropriate variations in reactants, quantities of reagents, and solvents. LC-MS: 285.2 [M+H]⁺.

Intermediate-33: 4-((1H-pyrazol-1-yl)methyl)-2-methylbenzofuro[7,6-d]isoxazol-8-amine

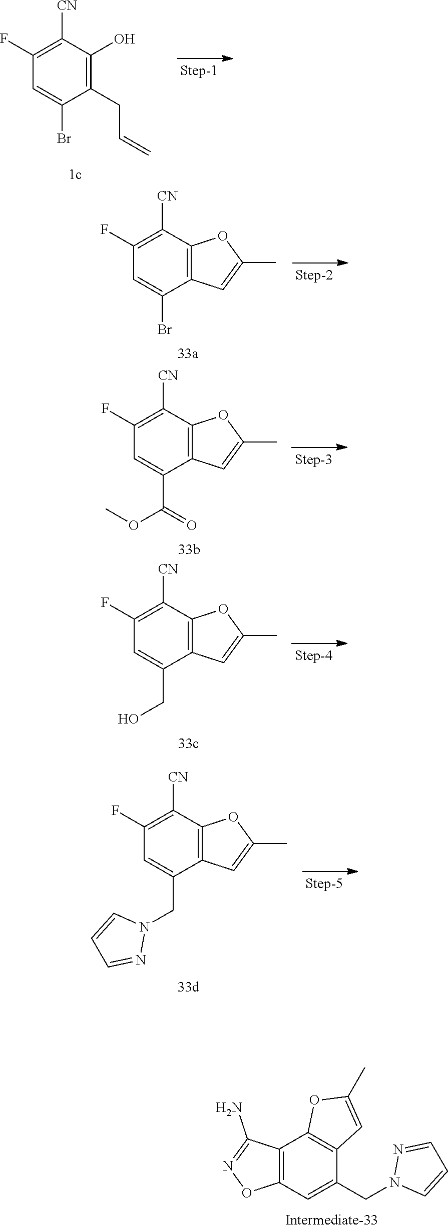

### Step-1:4-Bromo-6-fluoro-2-methylbenzofuran-7-carbonitrile

To a stirred solution of 3-allyl-4-bromo-6-fluoro-2-hydroxybenzonitrile (5.0 g, 19.52 mmol) in 1,4-dioxane (50 mL) were added PdCl₂(CH₃CN)₂ (0.25 g, 0.97 mmol), benzoquinone (2.11 g, 19.52 mmol) and stirred at 80° C. for 12 h. Then the reaction mixture was brought to rt, filtered through celite, and washed with ethyl acetate. The filtrate was diluted with water and extracted with ethyl acetate. The combined organic layer was dried over anhydrous sodium sulphate, filtered and concentrated under reduced pressure to obtain the crude. The crude was purified by silica-gel flash column chromatograph using 5% ethyl acetate in hexane as eluent to afford the title compound (2.0 g, 40.32%).

### Step-2: Methyl 7-cyano-6-fluoro-2-methylbenzofuran-4-carboxylate

This intermediate was prepared according to the procedure described in the synthesis of step-1 of Intermediate-19 with appropriate variations in reactants, quantities of reagents, and solvents.

### Step-3: 6-Fluoro-4-(hydroxymethyl)-2-methylbenzofuran-7-carbonitrile

This intermediate was prepared according to the procedure described in the synthesis of step-2 of Intermediate-19 with appropriate variations in reactants, quantities of reagents, and solvents.

### Step-4: 4-((1H-pyrazol-1-yl)methyl)-6-fluoro-2-methylbenzofuran-7-carbonitrile

This intermediate was prepared according to the procedure described in the synthesis of step-1 of Intermediate-20 with appropriate variations in reactants, quantities of reagents, and solvents. LC-MS: 256.0 [M+H]⁺.

### Step-5: 4-((1H-pyrazol-1-yl)methyl)-2-methylbenzofuro[7,6-d]isoxazol-8-amine

This intermediate was prepared according to the procedure described in the synthesis of step-2 of Intermediate-20 with appropriate variations in reactants, quantities of reagents, and solvents. LC-MS: 269.1 [M+H]⁺.

### Intermediate-34: 4-((1H-pyrazol-1-yl)methyl)-2-(methoxymethyl)-2,3-dihydrobenzofuro[7,6-d]isoxazol-8-amine

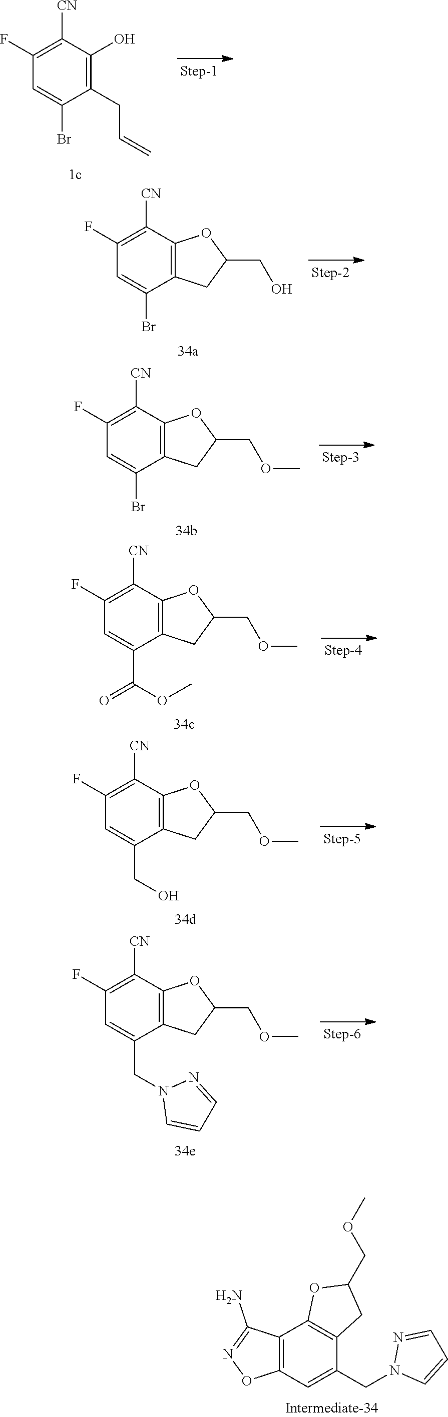

### Step-1: 4-Bromo-6-fluoro-2-(hydroxymethyl)-2,3-dihydrobenzofuran-7-carbonitrile

To a solution of 3-allyl-4-bromo-6-fluoro-2-hydroxybenzonitrile (6.0 g, 23.43 mmol) in dichloromethane (120 mL) was added 3-chloroperoxybenzoic acid (12.13 g, 70.30 mmol) at 0° C. and stirred at rt for 12 h. The reaction mixture was diluted with DCM and washed with a 1:1 solution of saturated sodium thiosulfate and saturated sodium bicarbonate. The combined organic layers were washed with brine, dried over sodium sulphate, filtered and concentrated to afford the crude material. The crude material was purified by silica-gel flash column chromatography using 15% ethyl acetate in hexane as eluent to afford pure title compound (5.5 g, 86.28%); LC-MS: 271.9 [M+H]⁺.

### Step-2: 4-Bromo-6-fluoro-2-(methoxymethyl)-2,3-dihydrobenzofuran-7-carbonitrile

To a stirred solution of the 4-bromo-6-fluoro-2-(hydroxymethyl)-2,3-dihydrobenzofuran-7-carbonitrile (2.0 g, 7.35 mmol) in THF (20 mL) was added sodium hydride (0.25 g, 11.02 mmol) at 0° C. and stirred for 1 h. Then Mel (2.08 g, 14.7 mmol) was added slowly at 0° C. After the reaction completion, the reaction mixture was quenched with ice-cold water, and extracted with ethyl acetate. The combined organic layer was washed with brine, dried over sodium sulphate, filtered and concentrated to afford the crude material. The crude material was purified by silica-gel flash column chromatography using 5% ethyl acetate in hexane as eluent to afford pure title compound (0.7 g, 33.28%).

### Step-3: Methyl 7-cyano-6-fluoro-2-(methoxymethyl)-2,3-dihydrobenzofuran-4-carboxylate

This intermediate was prepared according to the procedure described in the synthesis of step-1 of Intermediate-19 with appropriate variations in reactants, quantities of reagents, and solvents.

### Step-4: 6-Fluoro-4-(hydroxymethyl)-2-(methoxymethyl)-2,3-dihydrobenzofuran-7-carbonitrile

This intermediate was prepared according to the procedure described in the synthesis of step-2 of Intermediate-19 with appropriate variations in reactants, quantities of reagents, and solvents. LC-MS: 238.1 [M+H]⁺.

### Step-5: 4-((1H-pyrazol-1-yl)methyl)-6-fluoro-2-(methoxymethyl)-2,3-dihydrobenzofuran-7-carbonitrile

This intermediate was prepared according to the procedure described in the synthesis of step-1 of Intermediate-20 with appropriate variations in reactants, quantities of reagents, and solvents. LC-MS: 288.1 [M+H]⁺.

### Step-6: 4-((1H-pyrazol-1-yl)methyl)-2-(methoxymethyl)-2,3-dihydrobenzofuro[7,6-d]isoxazol-8-amine

This intermediate was prepared according to the procedure described in the synthesis of step-2 of Intermediate-20 with appropriate variations in reactants, quantities of reagents, and solvents. LC-MS: 301.1 [M+H]⁺.

### Intermediate-35: 2-Methoxy-4-(trifluoromethyl)benzenesulfonyl Chloride

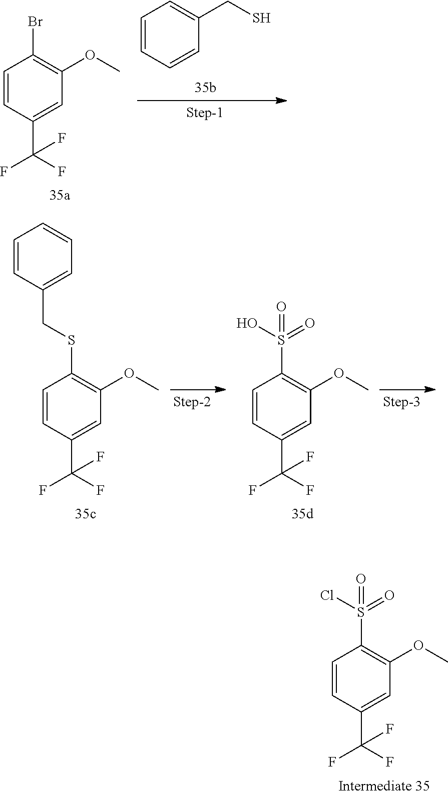

### Step-1: Benzyl(2-methoxy-4-(trifluoromethyl)phenyl)sulfane

To a degassed solution of 1-bromo-2-methoxy(trifluoromethyl)benzene (2 g, 7.84 mmol), phenylmethanethiol (0.97 g, 7.84 mmol) and N N-diisopropylethylamine (2.02 g, 15.68 mmol) in toluene (30 mL) was added Pd₂(dba)₃ (0.718 g, 0.78 mmol) and xantphos (0.907 g, 1.56 mmol) at rt. The reaction mixture was heated at 100° C. for 12 h then cooled to rt. The reaction mixture was passed through celite pad and washed with ethyl acetate. The filtrate was concentrated under reduced pressure to get crude product which was purified by flash column chromatography using 0-10% ethyl acetate in hexane as eluent to afford title compound (2.3 g, 98.3%). LC-MS: 297.0 [M−H]⁻.

### Step-2: 2-Methoxy-4-(trifluoromethyl)benzenesulfonic Acid

To a solution of benzyl(2-methoxy-4-(trifluoromethyl)phenyl)sulfane (1.5 g, 5.03 mmol) in acetonitrile (18 mL), acetic acid (12 mL), and water (6 mL) was added 1,3-dichloro-5,5-dimethylimidazolidine-2,4-dione (1.48 g, 7.54 mmol) at 0° C. and stirred at same temperature for 15 min. After completion of reaction, the reaction mixture was quenched with water and extracted with EtOAc. The combined organic layer was washed with aqueous sodium bicarbonate solution, brine, dried over sodium sulphate, filtered, and concentrated to afford the crude material. The Crude was purified by flash column chromatography using 3-5% EtOAc in hexane as eluent to get the title compound (0.85 g, 65.68%); LC-MS: 254.9[M−H]⁻.

### Step-3: 2-Methoxy-4-(trifluoromethyl)benzenesulfonyl Chloride

To a solution of 2-methoxy-4-(trifluoromethyl)benzenesulfonic acid (0.8 g, 3.12 mmol) in DCM (16 mL) was added PCl₅ (1.3 g, 6.24 mmol) portion-wise at 0° C. and refluxed for overnight. Then the reaction mixture was cooled to room temperature and concentrated under reduced pressure. The resulting crude was purified by silica gel flash column chromatography using 3-5% EtOAc in hexane as eluent to give the title compound (0.7 g 81.61%). ¹HNMR (DMSO-D6, 400 MHz): δ 8.12 (d, 1H), 7.41-7.28 (m, 2H), 4.14 (s, 3H).

### Intermediate-36: 2-(Pyridin-2-ylmethoxy)benzenesulfonyl Chloride

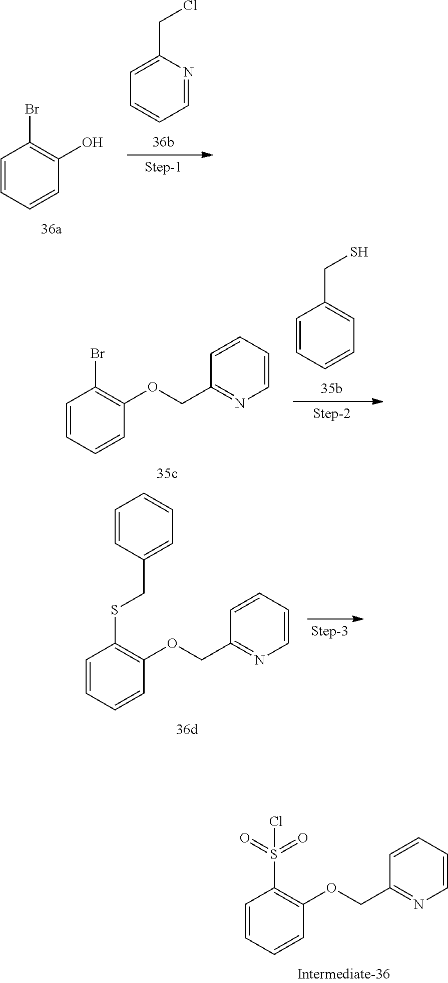

### Step-1: 2-((2-Bromophenoxy)methyl)pyridine

A mixture of 2-bromophenol (2 g, 11.56 mmol), 2-(chloromethyl)pyridine (1.17 g, 13.87 mmol) and K₂CO₃ (3.19 g, 23.12 mmol) in MeCN (30 mL), was heated at 90° C. for 12 h. The reaction mixture was cooled to room temperature, diluted with EtOAc and washed with 2N NaOH. The organic layer was dried over sodium sulfate, filtered and concentrated to afford the crude material. Crude was purified by silica gel flash column chromatography using 10-15% EtOAc in hexane as eluent to get the pure titled compound (2.8 g, 91.71%); LC-MS: 266.0[M+H]⁺.

### Step-2: 2-((2-(Benzylthio)phenoxy)methyl)pyridine

To a degassed solution of 2-((2-bromophenoxy)methyl)pyridine (2.8 g, 10.60 mmol) and phenylmethanethiol (1.31 g, 10.60 mmol) in 1,4-dioxane (30 mL) were added Pd₂(dba)3 (1.94 gm 2.12 mmol), N,N-diisopropylethylamine (2.74 g 21.20 mmol) and XantPhos (1.22 g 2.22 mmol) at rt. The reaction mixture was heated at 100° C. for 2 h. After completed reaction mixture passed through the celite and washed with ethyl acetate and concentrated under reduced pressure to get crude was purified by silica gel flash column chromatography using 10-15% EtOAc in hexane as eluent to get the pure titled compound (2.5 g, 76.71%); LC-MS: 308.1 [M+H]⁺.

### Step-3: 2-(Pyridin-2-ylmethoxy)benzenesulfonyl Chloride

To a stirred solution of 2-((2-(benzylthio)phenoxy)methyl)pyridine (1.5 g 4.87 mmol) in acetonitrile (12 mL) was added acetic acid (6 mL) and water (3 mL), followed sulfuryl chloride (1.31 g, 9.75 mmol) at a 0° C. The reaction mixture was stirred at same temperature for 15 min. After completion of reaction, the reaction mixture was quenched with water and extracted with EtOAc. The organic layer was washed with aqueous sodium bicarbonate solution, brine. The organic layer was dried over sodium sulphate, filtered, and concentrated to afford the crude material. Crude was purified by flash column chromatography using 5-10% EtOAc in hexane as eluent to get the title compound (0.35 g, 25.28%); LC-MS: 284.0 [M+H]⁺.

### Intermediate-37: 2-Methoxy-4-(1H-pyrazol-1-yl)benzenesulfonyl Chloride

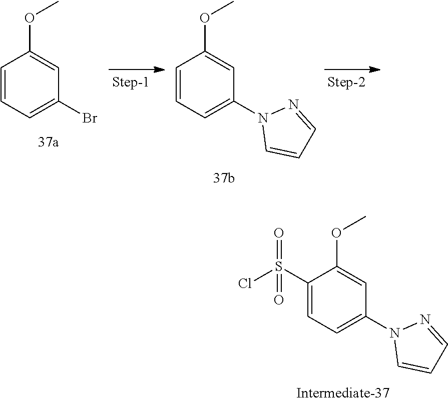

### Step-1:1-(3-Methoxyphenyl)-1H-pyrazole

To a degassed solution of 1-bromo-3-methoxybenzene (1 g, 5.34 mmol), 1H-pyrazole (0.73 g, 10.69 mmol), and K₃PO₄ (3.4 g, 16.03 mmol) in 1,4-dioxane was added CuI (0.10 g, 0.53 mmol) and N,N-dimethylethane-1,2-diamine (0.047 g, 0.53 mmol) at rt. The reaction mixture was heated at 100° C. for 12 h. Then reaction mixture was cooled to rt, passed through celite pad, and washed with ethyl acetate. The filtrate was concentrated under reduced pressure to get crude product which was purified by flash column chromatography using 0-10% ethyl acetate in hexane as eluent to afford title compound (0.400 g, 42.95%); LC-MS: 175.1 [M+H]⁺.

### Step-2: 2-Methoxy-4-(1H-pyrazol-1-yl)benzenesulfonyl Chloride

To a stirred solution of 1-(2-methoxyphenyl)-1H-pyrazole (0.4 g, 2.29 mmol) in DCM (10 mL) was added chlorosulfonic acid (1.5 mL, 22.95 mmol), and SOCl₂ (0.2 mL, 27.95 mmol) dropwise at 0° C. and stirred for 30 min. The reaction mixture was concentrated under reduced pressure, diluted with water and extracted with DCM. The organic layer was washed with aqueous sodium bicarbonate, and brine. The organic layer was dried over sodium sulphate, filtered, and concentrated to afford the crude material. Crude was purified by flash column chromatography using 5-10% EtOAc in hexane as eluent to get the title compound (0.4 g, 63.94%); LC-MS: 273.0[M+H]⁺.

### Intermediate 38: 2-Methoxy-5-(1H-pyrazol-1-yl)benzenesulfonyl Chloride

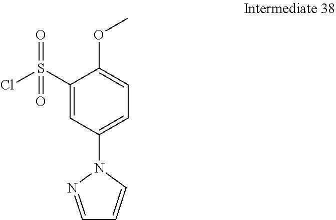 [SMILES: COc1ccc(-n2cccn2)cc1S(=O)(=O)Cl]

This intermediate was prepared by similar procedure described in Intermediate 36 with appropriate variations in coupling methods, reactants, quantities of reagents and solvents, and reaction conditions. LC-MS: 273.0 [M+H]⁺.

### Intermediate-39: 4-(Tert-butyl)-2-methoxybenzenesulfonyl Chloride

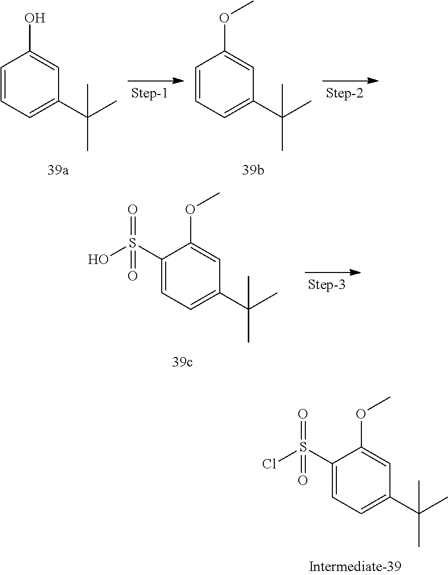 [SMILES: CC(C)(C)c1cccc(O)c1 ; COc1cccc(C(C)(C)C)c1 ; COc1cc(C(C)(C)C)ccc1S(=O)(=O)O ; COc1cc(C(C)(C)C)ccc1S(=O)(=O)Cl]

### Step-1:1-(Tert-Butyl)-3-methoxybenzene

To a stirred solution of 3-(tert-butyl)phenol (5 g, 33.28 mmol) in DMF (75 ml) was added NaH (1.14 g, 49.92 mmol) at 0° C. The reaction mixture was stirred at same temperature for 30 min. Mel (6.5 mL, 99.85) was added slowly to the reaction mixture at 0° C. and stirred at rt for 2 h. After completion of reaction, the reaction mixture was quenched with ice-cold water and extracted with EtOAc. The organic layer was washed with brine, dried over sodium sulphate, filtered, and concentrated to afford the crude material. The crude material was purified by flash column chromatography using 5-10% EtOAc in hexane as eluent to get the title compound (6.0 g, 88.60%).

### Step-2: 4-(Tert-butyl)-2-methoxybenzenesulfonic Acid

To a stirred solution of 1-(tert-butyl)-3-methoxybenzene (6 g, 36.53 mmol) in DCM (90 mL) was added chlorosulfonic acid (3 mL, 43.83 mmol) at 0° C. and stirred at rt for 30 min. After reaction completion the reaction was poured into sodium bicarbonate solution and extracted with DCM. The organic layer was dried over sodium sulphate, filtered, and concentrated to afford the crude material. Crude compound was purified by combi flash column chromatography using 0-15% EtOAc in hexane to get the title material (5 g, 84.48%).

### Step-3: 4-(Tert-butyl)-2-methoxybenzenesulfonyl Chloride

To a stirred solution of 4-(tert-butyl)-2-methoxybenzenesulfonic acid (5 g, 20.46 mmol) in DCM (75 mL) was added phosphorus pentachloride (8.52 g, 40.93 mmol) portion-wise and refluxed for overnight. The reaction mixture was cooled to room temperature and concentrated under reduced pressure. The resulting crude was purified by silica gel flash column chromatography using 3-5% EtOAc in hexane as eluent to give the title compound (4 g, 74.78%). ¹HNMR (DMSO-D6, 400 MHz): δ 7.58 (d, 1H), 6.94 (s, 1H), 6.88 (d, 1H), 3.78 (s, 3H), 1.44 (s, 9H).

### Intermediate-40: Methyl 3-(chlorosulfonyl)-4-methoxybenzoate

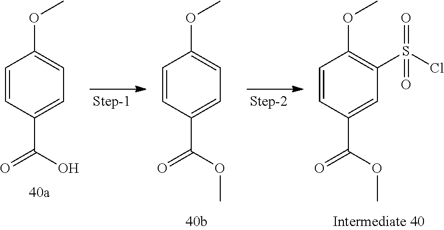 [SMILES: COc1ccc(C(=O)O)cc1 ; COC(=O)c1ccc(OC)cc1 ; COC(=O)c1ccc(OC)c(S(=O)(=O)Cl)c1]

### Step-1: Methyl 4-methoxybenzoate

To a stirred solution of 4-methoxybenzoic acid (2 g, 13.14 mmol) in methanol (30 mL) was added thionyl chloride (2.8 mL, 39.43 mmol) at 0° C. Then the reaction mixture was stirred at 80° C. for 1 h. After reaction completion, the reaction mixture was poured into sodium bicarbonate solution and extracted with ethyl acetate. The organic layer was washed with water, brine, dried over sodium sulphate, filtered and concentrated to afford the title compound (1.5 g, 68.68%); LC-MS: 166.9 [M+H]⁺.

### Step-2: Methyl 3-(chlorosulfonyl)-4-methoxybenzoate

To a stirred solution of methyl 4-methoxybenzoate (0.30 g, 1.80 mmol) in DCM (5 mL) was added chlorosulfonic acid (0.37 mL, 5.41 mmol) dropwise at 0° C. and stirred at rt for 12 h. After reaction completion the reaction was poured into sodium bicarbonate solution and extracted with DCM. The organic layer was dried over sodium sulphate, filtered, and concentrated to afford the title compound. (0.15 g, 68.68%). ¹HNMR (CDCl₃, 400 MHz): δ 8.66 (s, 1H), 8.38 (d, 1H), 7.20 (d, 1H), 4.18 (s, 3H), 3.96 (s, 3H).

The compounds listed in Table 7 were prepared by the similar procedure described in the step-2 of Intermediate-40 with appropriate variations in coupling methods, reactants, quantities of reagents, coupling methods and solvents. The characterization data of the compounds are summarized herein the below table.

TABLE 7
Intermediates 41 to 44

| Intermediate | Structure | Starting materials | ¹HNMR |
|---|---|---|---|
| 41 | 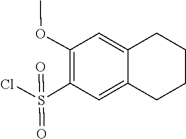 [SMILES: COc1cc2c(cc1S(=O)(=O)Cl)CCCC2] | 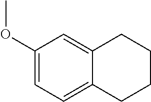 | ¹HNMR (DMSO-D6, 400 MHz): δ 7.35 (s, 1H), 6.65 (s, 1H), 3.70 (s, 3H), 2.69-2.61 (m, 4H), 1.70- 1.69 (m, 4H). |
| 42 | 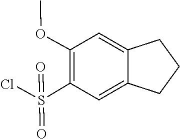 | 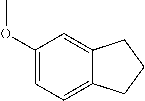 | ¹HNMR (CDCl₃, 400 MHz): δ 7.80 (s, 1H), 7.00 (s, 1H), 4.10 (s, 3H), 3.02 (t, 2H), 2.94 (t, 2H), 2.21-2.12 (m, 2H). |
| 43 | 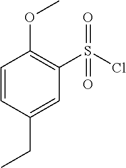 | 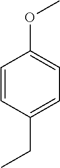 | ¹HNMR (DMSO-D₆, 400 MHz): δ 7.53 (s, 1H), 7.16 (d, 1H), 6.89 (d, 1H), 3.73 (s, 3H), 2.52 (q, 2H), 1.13 (t, 3H). |
| 44 | 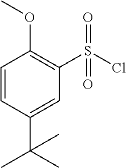 [SMILES: COc1ccc(C(C)(C)C)cc1S(=O)(=O)Cl] | 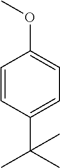 | ¹HNMR (DMSO-D₆, 400 MHz): δ 7.73 (s, 1H), 7.33 (d, 1H), 6.90 (d, 1H), 3.73 (s, 3H), 1.25 (s, 9H). |

### Intermediate-45: 2-Methyl-4-phenylbenzofuro[7,6-d]isoxazol-8-amine

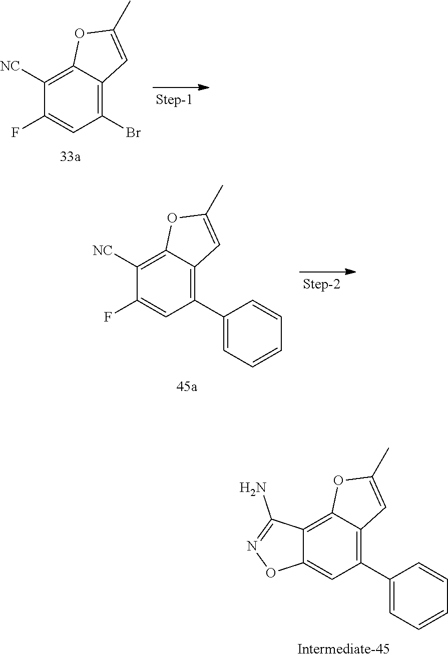 [SMILES: Cc1cc2c(Br)cc(F)c(C#N)c2o1 ; Cc1cc2c(-c3ccccc3)cc(F)c(C#N)c2o1 ; Cc1cc2c(-c3ccccc3)cc3onc(N)c3c2o1]

### Step-1:6-Fluoro-2-methyl-4-phenylbenzofuran-7-carbonitrile

This intermediate was prepared according to the procedure described in the synthesis of step-1 of Intermediate-2 with appropriate variations in reactants, quantities of reagents, and solvents. ¹H-NMR (400 MHz, DMSO-D₆) δ 7.61-7.50 (m, 5H), 7.12 (s, 1H), 6.65 (s, 1H), 1.58 (s, 3H).

### Step-2: 2-Methyl-4-phenylbenzofuro[7,6-d]isoxazol-8-amine

This intermediate was prepared according to the procedure described in the synthesis of step-2 of Intermediate-2 with appropriate variations in reactants, quantities of reagents, and solvents. LC-MS: 265.2 [M+H]⁺.

### Example 3. Synthesis of 2,6-dimethoxy-N-(4-phenyl-2,3-dihydrobenzofuro[7,6-d]isoxazol-8-yl)benzenesulfonamide (Compound-1)

### Method-A

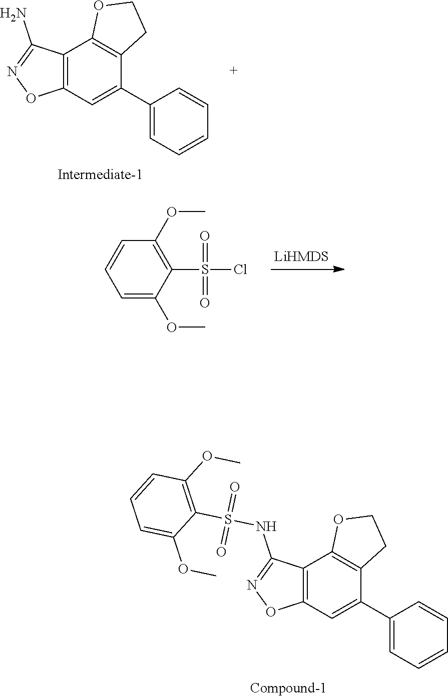 [SMILES: Nc1noc2cc(-c3ccccc3)c3c(c12)OCC3 ; COc1cccc(OC)c1S(=O)(=O)Cl ; COc1cccc(OC)c1S(=O)(=O)Nc1noc2cc(-c3ccccc3)c3c(c12)OCC3]

A stirred suspension of 4-phenyl-2,3-dihydrobenzofuro[7,6-d]isoxazol-8-amine (0.1 g, 0.396 mmol) and 2,6-dimethoxybenzenesulfonyl chloride (0.141 g, 0.594 mmol) in THF (5 mL) was cooled to 0° C. and added LiHMDS (0.59 mL, 2 M in THF) to the mixture and stirred the mixture for 10 min. The reaction mass was quenched with saturated NH₄Cl solution and extracted with ethyl acetate. The organic portion was washed with water, brine solution, dried over Na₂SO₄ and concentrated under reduced pressure to get the crude compound which was purified on preparative TLC (Silica gel) using 2% methanol in DCM as eluent to afford pure title compound (0.075 g, 41.8%): LC-MS: 451.2 [M−H]¹H-NMR (400 MHz, DMSO-D₆) δ 10.4 (s, 1H), 7.59-7.57 (m, 2H), 7.52-7.42 (m, 4H), 7.12 (s, 1H), 6.78 (d, 2H), 4.71 (t, 2H), 3.78 (s, 6H), 3.29 (t, 2H).

### Example 4. Synthesis of N-(4-((1H-pyrazol-1-yl)methyl)-2,3-dihydrobenzofuro[7,6-d]isoxazol-8-yl)-2,6-dimethoxybenzenesulfonamide (Compound-2)

### Method-B

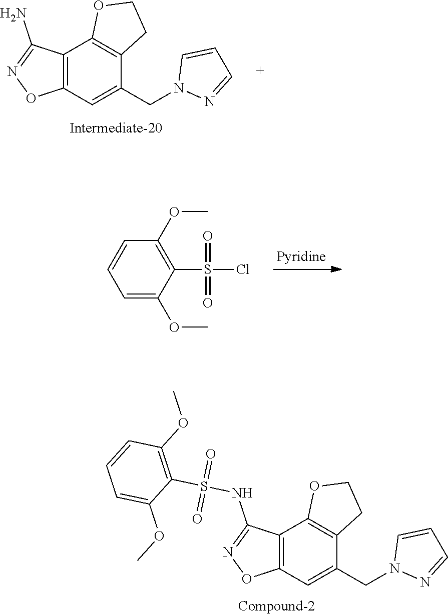 [SMILES: Nc1noc2cc(Cn3cccn3)c3c(c12)OCC3 ; COc1cccc(OC)c1S(=O)(=O)Cl ; COc1cccc(OC)c1S(=O)(=O)Nc1noc2cc(Cn3cccn3)c3c(c12)OCC3]

A mixture of 4-((1H-pyrazol-1-yl)methyl)-2,3-dihydrobenzofuro[7,6-d]isoxazol-8-amine (Intermediate 20, 0.1 g, 0.39 mmol) and 2,6-dimethoxybenzenesulfonyl chloride (0.277 g, 1.16 mmol) in pyridine (0.5 mL) was heated to 90° C. for 16 h. The reaction mass was then cooled to RT and concentrated under reduced pressure to get crude compound which was purified in preparative HPLC to afford pure title compound (0.015 g): LC-MS: 451.2 [M−H]⁻; ¹H-NMR (400 MHz, DMSO-D6) δ 10.41 (s, 1H), 7.82 (s, 1H), 7.51-7.49 (m, 2H), 6.75 (d, 2H), 6.67 (s, 1H), 6.29 (s, 1H), 5.40 (s, 2H), 4.71 (t, 2H), 3.74 (s, 6H), 3.09 (t, 2H).

### Example 5. Synthesis of Additional Compounds of Formula I

The compounds listed in Table 8 were prepared by the similar procedure described in the synthesis of the above Examples with appropriate variations in coupling methods, reactants, quantities of reagents, coupling methods and solvents. The characterization data of the compounds are summarized herein the below table.

TABLE 8
Compounds 3 to 21

| | | Coupling method/ | |
|---|---|---|---|
| Cpd | Structure | Intermediate used | Spectral data |
| 3 | 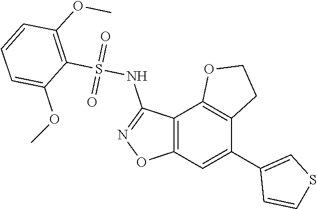 [SMILES: COc1cccc(OC)c1S(=O)(=O)Nc1noc2cc(-c3ccsc3)c3c(c12)OCC3] | Method-A/ Intermediate-3 | LC-MS: 459.2 [M + H]⁺; ¹H- NMR (400 MHz, DMSO-D₆) δ 10.38 (s, 1H), 7.87 (s, 1H), 7.69-7.67 (m, 1H), 7.55-7.47 (m, 2H), 7.30 (s, 1H), 6.76 (d, 2H), 4.74 (t, 2H), 3.76 (s, 6H), 3.40 (t, 2H). |
| 4 | 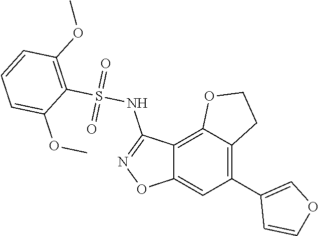 [SMILES: COc1cccc(OC)c1S(=O)(=O)Nc1noc2cc(-c3ccoc3)c3c(c12)OCC3] | Method-A/ Intermediate-4 | LC-MS: 443.1 [M + H]⁺; ¹H- NMR (400 MHz, DMSO-D₆) δ 10.3 (s, 1H), 8.01 (s, 1H), 8.77 (s, 1H), 7.46 (t, 1H), 7.30 (s, 1H), 7.04 (s, 1H), 6.73 (d, 2H), 4.73 (t, 2H), 3.72 (s, 6H), 3.4 (m, 2H). |
| 5 | 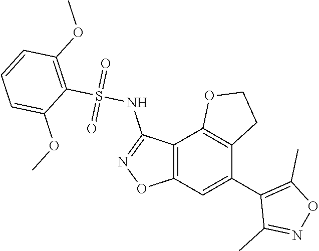 [SMILES: COc1cccc(OC)c1S(=O)(=O)Nc1noc2cc(-c3c(C)noc3C)c3c(c12)OCC3] | Method-A/ Intermediate-5 | LC-MS: 472.1 [M + H]⁺; ¹H- NMR (400 MHz, DMSO-D₆) δ 10.6 (s, 1H), 7.55 (t, 1H), 7.09 (s, 1H), 6.83 (d, 2H), 4.77 (t, 2H), 3.82 (s, 6H), 3.08-3.03 (m, 2H), 2.34 (s, 3H), 2.15 (s, 3H). |
| 6 | 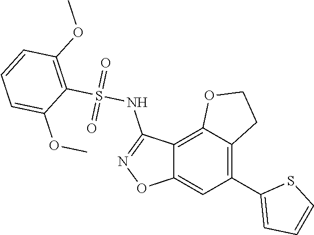 [SMILES: COc1cccc(OC)c1S(=O)(=O)Nc1noc2cc(-c3cccs3)c3c(c12)OCC3] | Method-A/ Intermediate-2 | LC-MS: 458.8 [M + H]⁺; ¹H- NMR (400 MHz, DMSO-D₆) δ 10.5 (s, 1H), 7.77 (d, 1H), 7.64 (brs, 1H), 7.54 (t, 1H), 7.41 (s, 1H), 7.26 (t, 1H), 6.82 (d, 2H), 4.82 (t, 2H), 3.81 (s, 6H), 3.49 (t, 2H). |
| 7 | 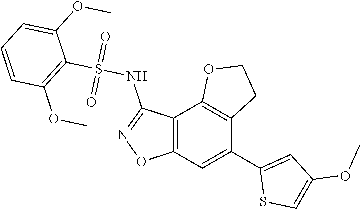 [SMILES: COc1csc(-c2cc3onc(NS(=O)(=O)c4c(OC)cccc4OC)c3c3c2CCO3)c1] | Method-A/ Intermediate-6 | LC-MS: 489.0 [M + H]⁺; ¹H- NMR (400 MHz, DMSO-D₆) δ 10.47 (s, 1H), 7.54 (t, 1H), 7.37 (s, 1H), 7.32 (s, 1H), 6.82-6.04 (m, 3H), 4.81 (t, 2H), 3.82 (s, 3H), 3.80 (s, 6H), 3.47 (t, 2H). |
| 8 | 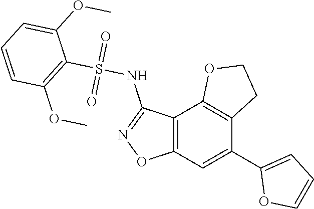 [SMILES: COc1cccc(OC)c1S(=O)(=O)Nc1noc2cc(-c3ccco3)c3c(c12)OCC3] | Method-A/ Intermediate-7 | LC-MS: 443.0 [M + H]⁺; ¹H- NMR (400 MHz, DMSO-D₆) δ 10.46 (s, 1H), 7.91 (d, 1H), 7.54 (t, 1H), 7.43 (s, 1H), 7.056 (d, 1H), 6.81 (d, 2H), 6.74-6.72 (m, 1H), 4.83 (t, 2H), 3.81 (s, 6H), 3.48 (t, 2H). |
| 9 | 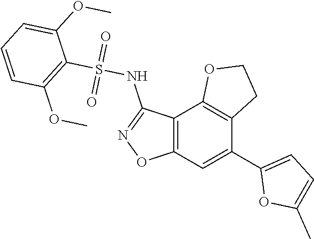 [SMILES: COc1cccc(OC)c1S(=O)(=O)Nc1noc2cc(-c3ccc(C)o3)c3c(c12)OCC3] | Method-A/ Intermediate-8 | LC-MS: 457.0 [M + H]⁺; ¹H- NMR (400 MHz, DMSO-D₆) δ 10.43 (s, 1H), 7.54 (t, 1H), 7.35 (s, 1H), 6.92 (d, 1H), 6.81 (d, 2H), 6.351 (d, 1H), 4.83 (t, 2H), 3.81 (s, 6H), 3.45 (t, 2H), 2.41 (s, 3H). |
| 10 | 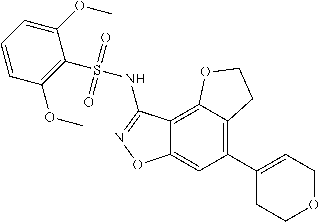 [SMILES: COc1cccc(OC)c1S(=O)(=O)Nc1noc2cc(C3=CCOCC3)c3c(c12)OCC3] | Method-A/ Intermediate-9 | LC-MS: 459.2 [M + H]⁺; ¹H- NMR (400 MHz, DMSO-D₆) δ 10.4 (s, 1H), 7.55 (t, 1H), 7.05 (s, 1H), 6.812 (d, 2H), 6.15 (s, 1H), 4.75 (t, 2H), 4.266 (d, 2H), 3.84 (m, 7H), 3.32 (t, 2H), 3.20 ((s, 1H), 2.5-2.4 (m, 2H). |
| 11 | 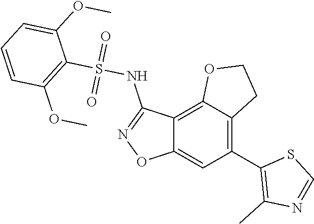 [SMILES: COc1cccc(OC)c1S(=O)(=O)Nc1noc2cc(-c3scnc3C)c3c(c12)OCC3] | Method-A/ Intermediate-10 | LC-MS: 474.1 [M + H]⁺; ¹H- NMR (400 MHz, DMSO-D₆) δ 10.5 (s, 1H), 9.1 (s, 1H), 7.5 (t, 1H), 7.15 (s, 1H), 6.77 (d, 2H), 4.74 (t, 2H), 3.77 (s, 6H), 3.14 (t, 2H), 2.36 (s, 3H). |
| 12 | 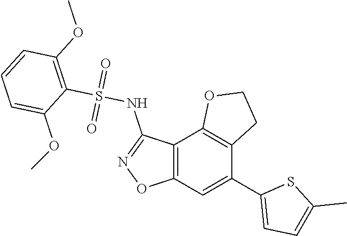 [SMILES: COc1cccc(OC)c1S(=O)(=O)Nc1noc2cc(-c3ccc(C)s3)c3c(c12)OCC3] | Method-A/ Intermediate-11 | LC-MS: 473.2 [M + H]⁺; ¹H- NMR (400 MHz, DMSO-D₆) δ 10.4 (s, 1H), 7.5 (t, 1H), 7.39 (d, 1H), 7.27 (s, 1H), 6.91 (d, 1H), 6.77 (d, 2H), 4.77 (t, 2H), 3.77 (s, 6H), 3.42 (t, 2H), 2.36 (s, 3H). |
| 13 | 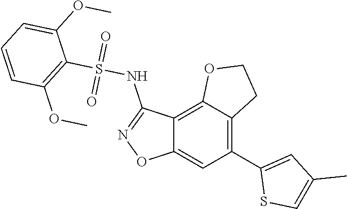 [SMILES: COc1cccc(OC)c1S(=O)(=O)Nc1noc2cc(-c3cc(C)cs3)c3c(c12)OCC3] | Method-A/ Intermediate-12 | LC-MS: 473.2 [M + H]⁺; ¹H- NMR (400 MHz, DMSO-D₆) δ 10.4 (s, 1H), 7.49 (t, 1H), 7.42 (s, 1H), 7.29 (s, 2H), 6.77 (d, 2H), 4.77 (t, 2H), 3.76 (s, 6H), 3.43 (t, 2H), 2.25 (s, 3H). |
| 14 | 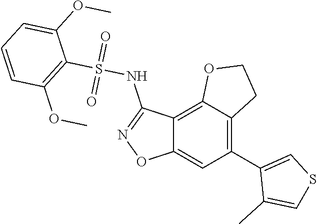 | Method-A/ Intermediate-13 | LC-MS: 443.0 [M + H]⁺; ¹H- NMR (400 MHz, DMSO-D₆) δ 8.18 (s, 1H), 7.53 (d, 1H), 7.35 (t, 1H), 7.28 (d, 1H), 6.76 (s, 1H), 6.68 (d, 2H), 4.65 (t, 2H), 3.69 (s, 6H), 3.06 (t, 2H), 2.12 (s, 3H). |
| 15 | 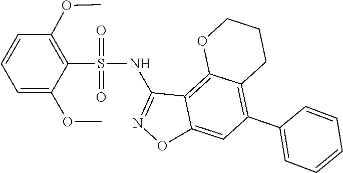 [SMILES: COc1cccc(OC)c1S(=O)(=O)Nc1noc2cc(-c3ccccc3)c3c(c12)OCCC3] | Method-A/ Intermediate-14 | LC-MS: 467.2 [M + H]⁺; ¹H- NMR (400 MHz, DMSO-D₆) δ 9.43 (s, 1H), 7.53-7.49 (m, 2H), 7.48-7.46 (m, 2H), 7.43- 7.37 (m, 2H), 6.96 (s, 1H), 6.80 (d, 2H), 4.31 (t, 2H), 3.82 (s, 6H), 2.54-2.52 (m, 2H), 1.91-1.88 (m, 2H). |
| 16 | 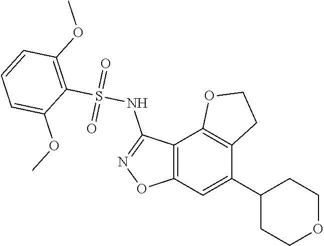 [SMILES: COc1cccc(OC)c1S(=O)(=O)Nc1noc2cc(C3CCOCC3)c3c(c12)OCC3] | Method-A/ Intermediate-16 | LC-MS: 461.1 [M + H]⁺; ¹H- NMR (400 MHz, CD₃OD) δ 7.45 (t, 1H), 6.82 (s, 1H), 6.71 (d, 2H), 4.78 (t, 2H), 4.03-4.0 (m, 2H), 3.82 (s, 6H), 3.57- 3.51 (m, 2H), 3.28-3.23 (m, 2H), 2.91-2.85 (m, 1H), 1.82- 1.70 (m, 4H). |
| 17 | 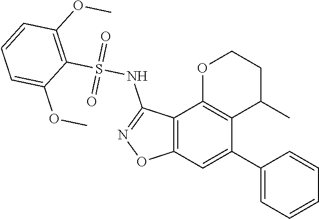 [SMILES: COc1cccc(OC)c1S(=O)(=O)Nc1noc2cc(-c3ccccc3)c3c(c12)OCCC3C] | Method-A/ Intermediate-15 | LC-MS: 481.2 [M + H]⁺; ¹H- NMR (400 MHz, DMSO-D₆) δ 9.43 (s, 1H) 7.53-7..39 (m, 4H), 7.36-7.34 (d, 2H), 6.86 (s, 1H), 6.81-6.78 (d, 2H), 4.42 (d, 1H), 4.22 (t, 1H), 3.81 (s, 6H), 3.15-3.05 (m, 1H), 2.13-2.09 (m, 1H), 2.73 (d, 1H), 0.79-0.77 (d, 3H). |
| 18 | 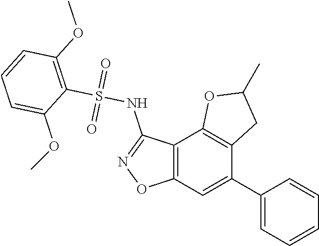 [SMILES: COc1cccc(OC)c1S(=O)(=O)Nc1noc2cc(-c3ccccc3)c3c(c12)OC(C)C3] | Method-A/ Intermediate-19 | LC-MS: 467.1 [M + H]⁺; ¹H- NMR (400 MHz, DMSO-D₆) δ 10.4 (s, 1H), 7.6-7.56 (m, 2H), 7.55-7.45 (m, 3H), 7.45- 7.35 (m, 1H), 7.10 (s, 1H), 6.78 (d, 2H), 5.15-5.05 (m, 1H), 3.78 (s, 6H), 3.45-3.35 (m, 1H), 2.95-2.85 (m, 1H), 1.42 (d, 3H). |
| 19 | 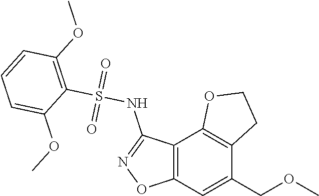 [SMILES: COCc1cc2onc(NS(=O)(=O)c3c(OC)cccc3OC)c2c2c1CCO2] | Method-A/ Intermediate-18 | LC-MS: 421.2 [M + H]⁺; ¹H- NMR (400 MHz, DMSO-D₆) δ 10.37 (s, 1H), 7.5 (t, 1H), 7.0 (s, 1H), 6.76 (d, 2H), 4.73 (t, 2H), 4.47 (s, 2H), 3.76 (s, 6H), 3.32 (s, 3H), 3.17 (t, 2H). |
| 20 | 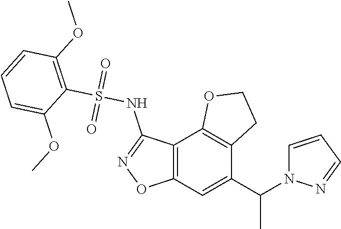 [SMILES: COc1cccc(OC)c1S(=O)(=O)Nc1noc2cc(C(C)n3cccn3)c3c(c12)OCC3] | Method-B/ Intermediate-21 | LC-MS: 471.2 [M + H]⁺; ¹H- NMR (400 MHz, DMSO-D₆) δ 10.40 (s, 1H), 7.86 (d, 1H), 7.49-7.47 (m, 2H), 6.83 (s, 1H), 6.76 (d, 2H), 6.28 (s, 1H), 5.71-5.70 (m, 1H), 4.69 (t, 2H), 3.75 (s, 6H), 3.05 (t, 2H), 1.79 (d, 3H). |
| 21 | 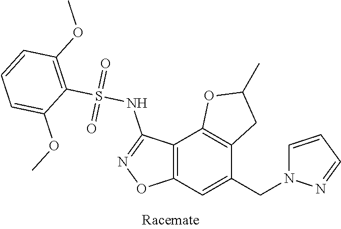 [SMILES: COc1cccc(OC)c1S(=O)(=O)Nc1noc2cc(Cn3cccn3)c3c(c12)OC(C)C3] | Method-B/ Intermediate-22 | LC-MS: 471.2 [M + H]⁺; ¹H- NMR (400 MHz, DMSO-D₆) δ 10.40 (s, 1H), 7.81 (d, 1H), 7.48 (d, 2H), 6.7 (brs, 2H), 6.64 (brs, 1H), 6.28 (m, 1H), 5.37 (s, 2H), 5.09-5.07 (m, 1H), 3.73 (s, 6H), 3.26-3.20 (m, 1H), 2.70-2.64 (m, 1H), 1.40 (d, 3H). |

### Example 6. Isomers separation of N-(4-((1H-pyrazol-1-yl)methyl)-2-methyl-2,3-dihydrobenzofuro[7,6-d]isoxazol-8-yl)-2,6-dimethoxybenzenesulfonamide (Compound

The enantiomers of N-(4-((1H-pyrazol-1-yl)methyl)-2-methyl-2,3-dihydrobenzofuro[7,6-d]isoxazol-8-yl)-2,6-dimethoxybenzenesulfonamide (Compound-21) are separated by Chiral preparative HPLC method to obtain Isomer-1 (Compound-22) and Isomer-2 (Compound-23). Separation method: Column used—CHIRAL PAK-IH (250×21.2 mm) 5p; Mobile phase: A-Hexane, B-Ethanol; Gradient (50% B at 0 min, 50% B at 30 min, flow rate 15 mL/min).

Compound-22: Spectral data of Isomer-1: LC-MS: 471.2 [M+H]⁺; ¹H-NMR (400 MHz, DMSO-D₆, VT-70° C.) δ 10.9 (brs, 1H), 7.73 (s, 1H), 7.46 (s, 1H), 7.3 (brs, 1H), 6.67 (d, 2H), 6.5 (brs, 1H), 6.27 (m, 1H), 5.32 (s, 2H), 5.06-5.04 (m, 1H), 3.68 (s, 6H), 3.26-3.21 (m, 1H), 2.69-2.63 (m, 1H), 1.43 (d, 3H).

Compound-23: Spectral data of Isomer-2: LC-MS: 471.2 [M+H]⁺; ¹H-NMR (400 MHz, DMSO-D₆, VT-70° C.) δ 10.9 (brs, 1H), 7.73 (s, 1H), 7.46 (s, 1H), 7.3 (brs, 1H), 6.67 (d, 2H), 6.5 (brs, 1H), 6.27 (m, 1H), 5.32 (s, 2H), 5.06-5.04 (m, 1H), 3.68 (s, 6H), 3.26-3.21 (m, 1H), 2.69-2.63 (m, 1H), 1.43 (d, 3H).

### Example 7. Compounds 31 to 60

The compounds listed in Table 9 were prepared by the similar procedure described in the synthesis of Example-1 & Example-2 with appropriate variations in reactants, quantities of reagents, coupling methods and solvents. The characterization data of the compounds are summarized herein the below table.

TABLE 9
Compounds 31 to 60

| | | Coupling method/ | |
|---|---|---|---|
| Cpd | Structure | Intermediate used | Spectral data |
| 31 | 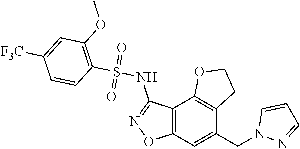 [SMILES: COc1cc(C(F)(F)F)ccc1S(=O)(=O)Nc1noc2cc(Cn3cccn3)c3c(c12)OCC3] | Method-A/ Intermediate-20 & Intermediate-35 | LC-MS: 495.0 [M + H]⁺; ¹HNMR (DMSO-D6, 400 MHz): δ 12.74 (s, 1H), 7.95 (dd, 1H), 7.79 (d, 1H), 7.48 (d, 1H), 7.34 (brs, 2H), 6.47 (brs, 1H), 6.29 (t, 1H), 5.36 (s, 2H), 4.67 (t, 2H), 3.84 (s, 3H), 3.06 (t, 2H). |
| 32 | 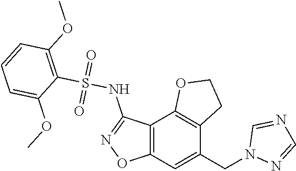 | Method-B/ Intermediate-25 | LC-MS: 428.02 [M + H]⁺; ¹HNMR (DMSO-D6, 400 MHz): δ 10.87 (s, 1H), 8.67 (s, 1H), 7.75 (dd, 1H), 7.65-7.60 (m, 1H), 7.21 (dd, 1H), 7.08 (t, 1H), 6.86 (s, 1H), 5.50 (s, 2H), 4.73 (t, 2H), 3.79 (s, 6H), 3.13 (t, 2H). |
| 33 | 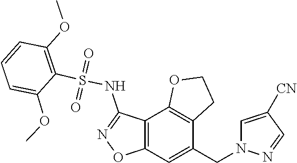 [SMILES: COc1cccc(OC)c1S(=O)(=O)Nc1noc2cc(Cn3cc(C#N)cn3)c3c(c12)OCC3] | Method-B/ Intermediate-26 | LC-MS: 482.2 [M + H]⁺; ¹HNMR (DMSO-D6, 400 MHz): δ 10.45 (s, 1H), 8.68 (s, 1H), 8.11 (s, 1H), 7.50 (t, 1H), 6.87 (s, 1H), 6.77 (d, 2H), 5.49 (s, 2H), 4.74 (t, 2H), 3.67 (s, 6H), 3.12 (t, 2H). |
| 34 | 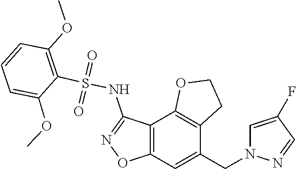 [SMILES: COc1cccc(OC)c1S(=O)(=O)Nc1noc2cc(Cn3cc(F)cn3)c3c(c12)OCC3] | Method-B/ Intermediate-27 | LC-MS: 475.2 [M + H]⁺; ¹HNMR (DMSO-D6, 400 MHz): δ 10.44 (s, 1H), 7.96 (d, 1H), 7.52-7.47 (m, 2H), 6.77-6.75 (m, 3H), 5.32 (s, 2H), 4.72 (t, 2H), 3.76 (s, 6H), 3.11 (t, 2H). |
| 35 | 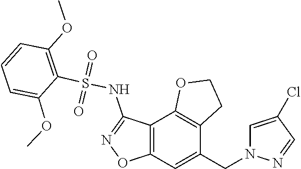 [SMILES: COc1cccc(OC)c1S(=O)(=O)Nc1noc2cc(Cn3cc(Cl)cn3)c3c(c12)OCC3] | Method-B/ Intermediate-28 | LC-MS: 491.2 [M + H]⁺; ¹HNMR (DMSO-D6, 400 MHz): δ 10.44 (s, 1H), 8.09 (s, 1H), 7.59 (s, 1H), 6.49 (t, 1H), 6.79 (s, 1H), 6.76 (d, 2H), 5.37 (s, 2H), 4.73 (t, 2H), 3.76 (s, 6H), 3.12 (t, 2H). |
| 36 | 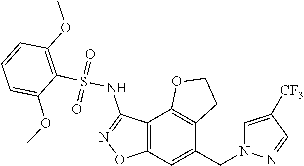 [SMILES: COc1cccc(OC)c1S(=O)(=O)Nc1noc2cc(Cn3cc(C(F)(F)F)cn3)c3c(c12)OCC3] | Method-B/ Intermediate-29 | LC-MS: 525.2[M + H]⁺; ¹HNMR (DMSO-D6, 400 MHz): δ 10.45 (s, 1H), 8.54 (s, 1H), 7.95 (s, 1H), 7.50 (t, 1H), 6.86 (s, 1H), 6.77 (d, 2H), 5.47 (s, 2H), 4.75 (t, 2H), 3.76 (s, 6H) 3.15 (t, 2H). |
| 37 | 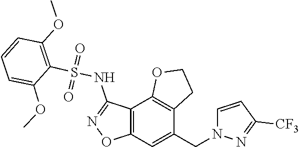 [SMILES: COc1cccc(OC)c1S(=O)(=O)Nc1noc2cc(Cn3ccc(C(F)(F)F)n3)c3c(c12)OCC3] | Method-B/ Intermediate-30 | LC-MS: 525.2 [M + H]⁺; ¹HNMR (DMSO-D6, 400 MHz): δ 10.45 (s, 1H), 8.09-8.08 (m, 1H), 7.50 (t, 1H), 6.83 (s, 1H), 6.79-6.75 (m, 3H), 5.53 (s, 2H), 4.74 (t, 2H), 3.76 (s, 6H), 3.12 (t, 2H). |
| 38 | 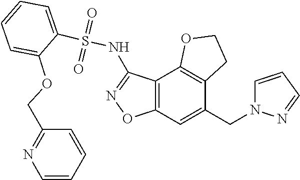 [SMILES: O=S(=O)(Nc1noc2cc(Cn3cccn3)c3c(c12)OCC3)c1ccccc1OCc1ccccn1] | Method-B/ Intermediate-36 & Intermediate-20 | LC-MS: 504.2 [M + H]⁺; ¹HNMR (DMSO-D6, 400 MHz): δ 11.33 (s, 1H), 8.55 (d, 1H), 8.54-7.80 (m, 2H), 7.71 (td, 1H), 7.64 (t, 1H), 7.49 (d, 1H), 7.46-7.44 (m, 1H), 7.33-7.28 (m, 2H), 7.14 (t, 1H) 6.62 (s, 1H), 6.30 (t, 1H), 5.38 (s, 2H), 5.32 (s, 2H), 4.70 (t, 2H), 3.09 (t, 2H). |
| 39 | 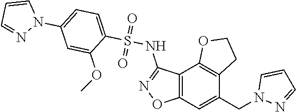 [SMILES: COc1cc(-n2cccn2)ccc1S(=O)(=O)Nc1noc2cc(Cn3cccn3)c3c(c12)OCC3] | Method-B/ Intermediate-37 & Intermediate-20 | LC-MS: 493.1[M + H]⁺; ¹HNMR (DMSO-D6, 400 MHz): δ 10.97 (s, 1H), 8.69 (d, 1H), 7.83-7.18 (m, 3H), 7.62 (d, 1H), 7.55 (dd, 1H), 7.49 (d, 1H), 6.70 (s, 1H), 6.64- 6.62 (m, 1H), 6.30 (t, 1H), 5.41 (s, 2H) 4.72 (t, 2H), 3.90 (s, 3H), 3.10 (t, 2H). |
| 40 | 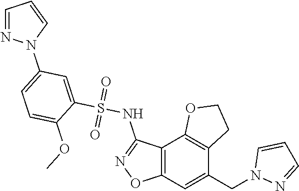 [SMILES: COc1ccc(-n2cccn2)cc1S(=O)(=O)Nc1noc2cc(Cn3cccn3)c3c(c12)OCC3] | Method-B/ Intermediate-38 & Intermediate-20 | LC-MS: 493.1 [M + H]⁺; ¹HNMR (DMSO-D6, 400 MHz): δ 11.15 (s, 1H), 8.50 (d, 1H), 8.19 (d, 1H), 8.08 (dd, 1H), 7.83 (d, 1H), 7.73 (d, 1H), 7.49 (d, 1H), 7.36 (d, 1H), 6.70 (s, 1H) 6.55-6.53 (m, 1H), 6.30 (t, 1H), 5.41 (s, 2H), 4.71 (t, 2H), 3.83 (s, 3H), 3.10 (t, 2H). |
| 41 | 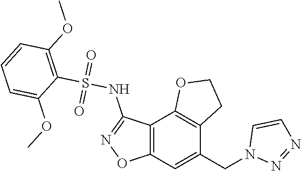 [SMILES: COc1cccc(OC)c1S(=O)(=O)Nc1noc2cc(Cn3ccnn3)c3c(c12)OCC3] | Method-B/ Intermediate-31 | LC-MS: 458.1 [M + H]⁺; ¹HNMR (DMSO-D6, 400 MHz): δ 10.47 (s, 1H), 8.19 (s, 1H), 7.78 (s, 1H), 7.50 (t, 1H), 6.86 (s, 1H), 6.76 (d, 2H), 5.70 (s, 2H), 4.73 (t, 2H), 3.75 (s, 6H) 3.12 (t, 2H). |
| 42 | 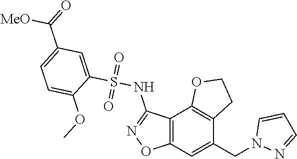 [SMILES: COC(=O)c1ccc(OC)c(S(=O)(=O)Nc2noc3cc(Cn4cccn4)c4c(c23)OCC4)c1] | Method-B/ Intermediate-40 & Intermediate-20 | LC-MS: 485.0 [M + H]⁺; ¹HNMR (DMSO-D6, 400 MHz): δ 11.20 (s, 1H), 8.32 (d, 1H), 8.20 (d, 1H), 7.83 (d, 1H), 7.50 (s, 1H), 7.36 (d, 1H), 6.70 (s, 1H), 6.30 (s, 1H), 5.41 (s, 2H) 4.69 (t, 2H), 3.89 (d, 3H), 3.85 (s, 3H), 3.10 (t, 2H). |
| 43 | 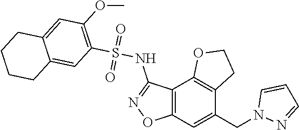 [SMILES: COc1cc2c(cc1S(=O)(=O)Nc1noc3cc(Cn4cccn4)c4c(c13)OCC4)CCCC2] | Method-B/ Intermediate-41 & Intermediate-20 | LC-MS: 481.2 [M + H]⁺; ¹HNMR (DMSO-D6, 400 MHz): δ 10.60 (s, 1H), 7.83 (d, 1H), 7.49 (dd, 1H), 7.42 (s, 1H), 6.88 (s, 1H), 6.70 (s, 1H), 6.31 (t, 1H), 5.42 (s, 2H), 4.73 (t, 2H), 3.73 (s, 3H), 3.11 (t, 2H), 2.77-2.72 (m, 2H), 2.68-2.65 (m, 2H), 1.72-1.70 (m, 4H). |
| 44 | 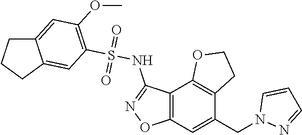 [SMILES: COc1cc2c(cc1S(=O)(=O)Nc1noc3cc(Cn4cccn4)c4c(c13)OCC4)CCC2] | Method-B/ Intermediate-42 & Intermediate-20 | LC-MS: 467.1 [M + H]⁺; ¹HNMR (DMSO-D6, 400 MHz): δ 8.18 (s, 1H), 7.77 (d, 1H), 7.59 (s, 1H), 7.47 (d, 1H), 6.83 (s, 1H), 6.35 (s, 1H), 6.27 (t, 1H), 5.32 (s, 2H), 4.65 (t, 2H), 3.66 (s, 3H), 3.04 (t, 2H), 2.82 (t, 2H), 2.76 (t, 2H), 2.03-1.95 (m, 2H). |
| 45 | 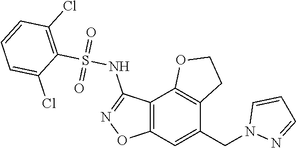 [SMILES: O=S(=O)(Nc1noc2cc(Cn3cccn3)c3c(c12)OCC3)c1c(Cl)cccc1Cl] | 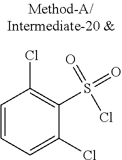 [SMILES: O=S(=O)(Cl)c1c(Cl)cccc1Cl] | LC-MS: 465.1 [M + H]⁺; ¹HNMR (DMSO-D6, 400 MHz): δ 7.83 (d, 1H), 7.67-7.65 (m, 2H), 7.62-7.57 (m, 1H), 7.50-7.49 (m, 1H), 6.73 (s, 1H), 6.31 (t, 1H), 5.42 (s, 2H), 4.67 (t, 2H), 3.11 (t, 2H). |
| 46 | 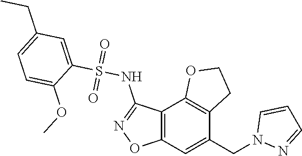 [SMILES: CCc1ccc(OC)c(S(=O)(=O)Nc2noc3cc(Cn4cccn4)c4c(c23)OCC4)c1] | Method-B/ Intermediate-43 & Intermediate-20 | LC-MS:455.3 [M + H]⁺; ¹HNMR (DMSO-D6, 400 MHz): δ 10.79 (s, 1H), 7.83 (d, 1H), 7.56 (d, 1H), 7.50-7.45 (m, 2H), 7.12 (d, 1H), 6.70 (s, 1H), 6.31 (t, 1H), 5.42 (s, 2H), 4.72 (t, 2H), 3.74 (s, 3H), 3.10 (t, 2H), 2.56 (q, 2H), 1.15 (t, 3H). |
| 47 | 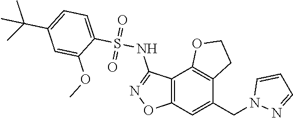 [SMILES: COc1cc(C(C)(C)C)ccc1S(=O)(=O)Nc1noc2cc(Cn3cccn3)c3c(c12)OCC3] | Method-B/ Intermediate-39 & Intermediate-20 | LC-MS: 483.1[M + H]⁺; ¹HNMR (DMSO-D6, 400 MHz): δ 10.78 (s, 1H), 7.83 (dd, 1H), 7.65 (d, 1H), 7.50 (dd, 1H), 7.12 (d, 1H), 7.09 (dd, 1H), 6.70 (s, 1H), 6.31 (t, 1H), 5.42 (s, 2H), 4.71 (t, 2H), 3.81 (s, 3H), 3.10 (t, 2H), 1.31 (s, 9H). |
| 48 | 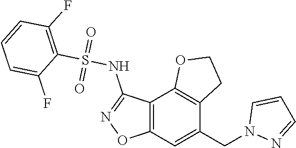 [SMILES: O=S(=O)(Nc1noc2cc(Cn3cccn3)c3c(c12)OCC3)c1c(F)cccc1F] | 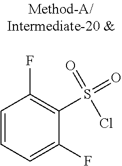 [SMILES: O=S(=O)(Cl)c1c(F)cccc1F] | LC-MS: 433.1 [M + H]⁺; ¹HNMR (DMSO-D6, 400 MHz): δ 7.80 (d, 1H), 7.78-7.68 (m, 1H), 7.46 (s, 1H), 7.29-7.24 (m, 2H), 6.69 (s, 1H), 6.27 (s, 1H), 5.39 (s, 2H), 4.65 (t, 2H), 3.07 (t, 2H). |
| 49 | 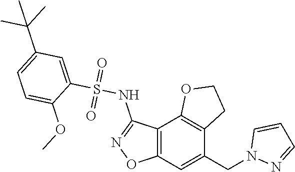 [SMILES: COc1ccc(C(C)(C)C)cc1S(=O)(=O)Nc1noc2cc(Cn3cccn3)c3c(c12)OCC3] | Method-B/ Intermediate-44 & Intermediate-20 | LC-MS: 483.2 [M + H]⁺; ¹HNMR (DMSO-D6, 400 MHz): δ 10.84 (s, 1H), 7.83 (d, 1H), 7.72 (d, 1H), 7.65 (d, 1H), 7.50 (s, 1H), 7.13 (d, 1H), 6.71 (s, 1H), 6.31 (s, 1H), 5.42 (s, 2H), 4.71 (t, 2H), 3.72 (s, 3H), 3.10 (t, 2H), 1.25 (s, 9H). |
| 50 | 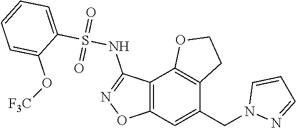 [SMILES: O=S(=O)(Nc1noc2cc(Cn3cccn3)c3c(c12)OCC3)c1ccccc1OC(F)(F)F] | 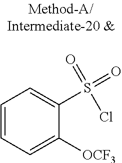 [SMILES: O=S(=O)(Cl)c1ccccc1OC(F)(F)F] | LC-MS: 481.0 [M + H]⁺; ¹HNMR (DMSO-D6, 400 MHz): δ 8.00 (dd, 1H), 7.84 (dd, 1H), 7.78 (t, 1H), 7.60-7.54 (m, 2H), 7.50 (dd, 1H), 6.70 (s, 1H), 6.31 (t, 1H), 5.42 (s, 2H), 4.71 (t, 2H), 3.11 (t, 2H). |
| 51 | 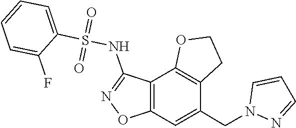 [SMILES: O=S(=O)(Nc1noc2cc(Cn3cccn3)c3c(c12)OCC3)c1ccccc1F] | 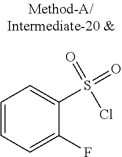 [SMILES: O=S(=O)(Cl)c1ccccc1F] | LC-MS: 415.0 [M + H]⁺; ¹HNMR (DMSO-D6, 400 MHz): δ 7.83- 7.78 (m, 2H), 7.72-7.67 (m, 1H), 7.46-7.33 (m, 3H), 6.67 (s, 1H), 6.27 (t, 1H), 5.38 (s, 2H), 4.68 (t, 2H), 3.07 (t, 2H). |
| 52 | 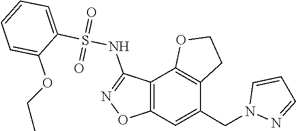 [SMILES: CCOc1ccccc1S(=O)(=O)Nc1noc2cc(Cn3cccn3)c3c(c12)OCC3] | 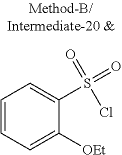 [SMILES: CCOc1ccccc1S(=O)(=O)Cl] | LC-MS: 441.2 [M + H]⁺; ¹HNMR (DMSO-D6, 400 MHz): δ 7.83 (d, 1H), 7.76 (d, 1H), 7.60 (t, 1H), 7.50 (s, 1H), 7.20 (d, 1H), 7.05 (t, 1H), 6.70 (s, 1H), 6.31 (t, 1H), 5.42 (s, 2H), 4.71 (t, 2H), 4.10 (q, 2H), 3.11 (t, 2H), 1.19 (t, 3H). s |
| 53 | 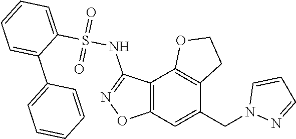 [SMILES: O=S(=O)(Nc1noc2cc(Cn3cccn3)c3c(c12)OCC3)c1ccccc1-c1ccccc1] | 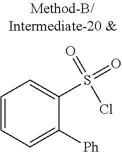 [SMILES: O=S(=O)(Cl)c1ccccc1-c1ccccc1] | LC-MS: 473.1 [M + H]⁺; ¹HNMR (DMSO-D6, 400 MHz): δ 11.20 (s, 1H), 8.09 (dd, 1H), 7.83 (d, 1H), 7.70-7.52 (m, 2H), 7.50 (dd, 1H), 7.40-7.30 (m, 6H), 6.69 (s, 1H), 6.31 (t, 1H), 5.41 (s, 2H), 4.73 (t, 2H), 3.11 (t, 2H). |
| 54 | 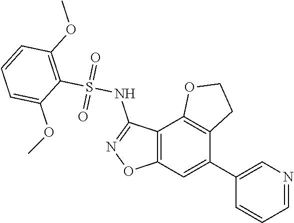 [SMILES: COc1cccc(OC)c1S(=O)(=O)Nc1noc2cc(-c3cccnc3)c3c(c12)OCC3] | Method-B/ Intermediate-23 | LC-MS: 454.2 [M + H]⁺; ¹HNMR (DMSO-D6, 400 MHz): δ 10.51 (s, 1H), 8.81 (dd, 1H), 8.64 (dd, 1H), 8.05-8.02 (m, 1H), 7.55-7.49 (m, 2H), 7.25 (s, 1H), 6.79 (d, 2H), 4.74 (t, 2H), 3.78 (s, 6H), 3.34 (t, 2H, overlapped with residual H₂O peak). |
| 55 | 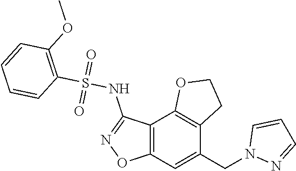 [SMILES: COc1ccccc1S(=O)(=O)Nc1noc2cc(Cn3cccn3)c3c(c12)OCC3] | 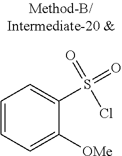 [SMILES: COc1ccccc1S(=O)(=O)Cl] | LC-MS: 427.2 [M + H]⁺; ¹HNMR (DMSO-D6, 400 MHz): δ 10.87 (s, 1H), 7.83 (d, 1H), 7.74 (dd, 1H), 7.62 (t, 1H), 7.49 (s, 1H), 7.21 (d, 1H), 7.06 (t, 1H), 6.69 (s, 1H), 6.30 (t, 1H), 5.41 (s, 2H), 4.72 (t, 2H), 3.79 (s, 3H), 3.10 (t, 2H). |
| 56 | 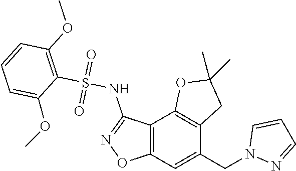 | Method-B/ Intermediate-32 | LC-MS: 427.2 [M + H]⁺; ¹HNMR (DMSO-D6, 400 MHz): δ 10.34 (s, 1H), 7.82 (d, 1H), 7.53-7.48 (m, 2H), 6.78 (d, 2H), 6.65 (s, 1H), 6.30 (t, 1H), 5.38 (s, 2H), 3.77 (s, 6H), 2.91 (s, 2H), 1.42 (s, 6H). |
| 57 | 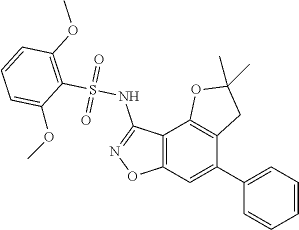 [SMILES: COc1cccc(OC)c1S(=O)(=O)Nc1noc2cc(-c3ccccc3)c3c(c12)OC(C)(C)C3] | Method-A/ Intermediate-24 | LC-MS: 481.2 [M + H]⁺; ¹HNMR (DMSO-D6, 400 MHz): δ 10.35 (s, 1H), 7.59-7.55 (m, 2H), 7.54-7.42 (m, 4H), 7.11 (s, 1H), 6.80 (d, 2H), 3.81 (s, 6H), 3.13 (s, 2H), 1.44 (s, 6H). |
| 58 | 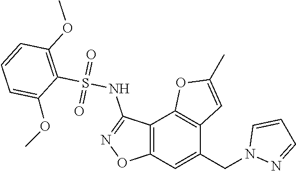 [SMILES: COc1cccc(OC)c1S(=O)(=O)Nc1noc2cc(Cn3cccn3)c3cc(C)oc3c12] | Method-B/ Intermediate-33 | LC-MS: 469.2 [M + H]⁺; ¹HNMR (DMSO-D6, 400 MHz): δ 10.91 (s, 1H), 7.90 (dd, 1H), 7. 52 (t, 1H), 7.49-7.48 (m, 1H), 7.26 (s, 1H), 6.79 (d, 2H), 6.71 (d, 1H), 6.30 (t, 1H), 5.66 (s, 2H), 3.75 (s, 6H), 2.44 (s, 3H). |
| 59 | 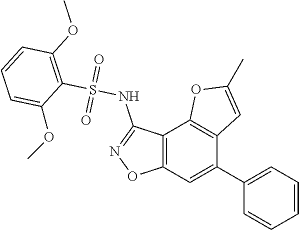 [SMILES: COc1cccc(OC)c1S(=O)(=O)Nc1noc2cc(-c3ccccc3)c3cc(C)oc3c12] | Method-A/ Intermediate-45 | LC-MS: 465.2 [M + H]⁺; ¹HNMR (DMSO-D6, 400 MHz): δ 10.92 (s, 1H), 7.69 (d, 2H), 7.54 (t, 2H), 7.48-7.34 (m, 3H), 6.75 (brs, 3H), 3.69 (s, 6H), 2.45 (s, 3H, over- lapped with residual solvent). |
| 60 | 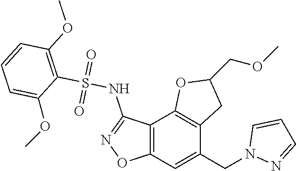 [SMILES: COCC1Cc2c(Cn3cccn3)cc3onc(NS(=O)(=O)c4c(OC)cccc4OC)c3c2O1] | Method-B/ Intermediate-34 | LC-MS: 501.1 [M + H]⁺; ¹HNMR (DMSO-D6, 400 MHz): δ 10.46 (s, 1H), 7.83 (d, 1H), 7.53-7.48 (m, 2H), 6.77 (d, 2H), 6.67 (s, 1H), 6.31 (t, 1H), 5.40 (s, 2H), 5.18- 5.13 (m, 1H), 3.76 (s, 6H), 3.57 (d, 2H), 3.32 (s, 3H), 3.16 (dd, 1H), 2.92 (dd, 1H). |

### Example 8. Isomers separation of N-(4-((1H-pyrazol-1-yl)methyl)-2-(methoxymethyl)-2,3-dihydrobenzofuro[7,6-d]isoxazol-8-yl)-2,6-dimethoxybenzenesulfonamide (Compound 60)

The enantiomers of N-(4-((1H-pyrazol-1-yl)methyl)-2-(methoxymethyl)-2,3-dihydrobenzofuro[7,6-d]isoxazol-8-yl)-2,6-dimethoxybenzenesulfonamide (Compound-60) are separated by chiral preparative HPLC method to obtain Isomer-1 (Compound-61) and Isomer-2 (Compound-62). Separation method: Column used—CHIRAL PAK-IH (250×21.2 mm) 5μ; Mobile phase: A-Hexane, B-0.1% DEA in EtOH:MeOH (70:30); Gradient (50% B at 0 min, 50% B at 30 min, flow rate 15 mL/min).

Compound-61: Spectral data of Isomer-1: LC-MS: 501.1 [M+H]⁺; ¹HNMR (DMSO-D6, 400 MHz): δ 10.45 (s, 1H), 7.83 (d, 1H), 7.53-7.48 (in, 2H), 6.77 (d, 2H), 6.66 (s, 1H), 6.31 (t, 1H), 5.40 (s, 2H), 5.20-5.10 (m, 1H), 3.76 (s, 6H), 3.57 (d, 2H), 3.32 (s, 3H), 3.16 (dd, 1H), 2.92 (dd, 1H).

Compound-62: Spectral data of Isomer-2: LC-MS: 501.1 [M+H]⁺; ¹HNMR (DMSO-D6, 400 MHz): δ 10.47 (s, 1H), 7.83 (dd, 1H), 7.51-7.48 (m, 2H), 6.77 (d, 2H), 6.66 (s, 1H), 6.31 (t, 1H), 5.40 (s, 2H), 5.19-5.15 (m, 1H), 3.76 (s, 6H), 3.57 (d, 2H), 3.32 (s, 3H), 3.16 (dd, 1H), 2.92 (dd, 1H).

### Example 9. 3—(N-(4-((1H-pyrazol-1-yl)methyl)-2,3-dihydrobenzofuro[7,6-d]isoxazol-8-yl)sulfamoyl)-4-methoxybenzoic Acid (Compound 63)

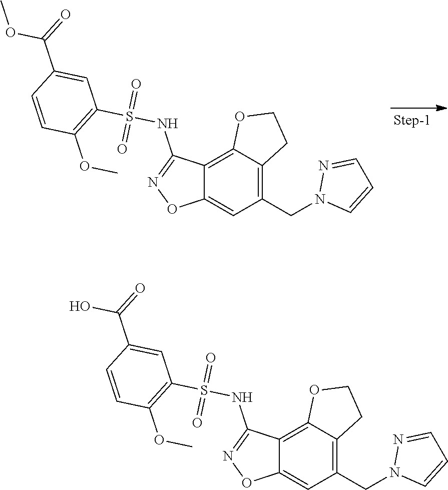 [SMILES: COC(=O)c1ccc(OC)c(S(=O)(=O)Nc2noc3cc(Cn4cccn4)c4c(c23)OCC4)c1 ; COc1ccc(C(=O)O)cc1S(=O)(=O)Nc1noc2cc(Cn3cccn3)c3c(c12)OCC3]

### Step-1:3—(N-(4-((1H-pyrazol-1-yl)methyl)-2,3-dihydrobenzofuro[7,6-d]isoxazol-8-yl)sulfamoyl)-4-methoxybenzoic Acid

To a stirred solution of methyl 3-(N-(4-((1H-pyrazol-1-yl)methyl)-2,3-dihydrobenzofuro[7,6-d]isoxazol-8-yl)sulfamoyl)-4-methoxybenzoate (0.025 g, 0.05 mmol) in THF:H₂O (2:0.2 mL) was added LiOH (0.002 g, 0.07 mmol) and stirred at rt for 2 h. The reaction mixture was concentrated and then diluted with water, acidified with 10% of citric acid to get the precipitate. The obtained precipitate was filtered and dried to get the title compound (0.010 g, 40.88 mmol). LC-MS: 470.85[M+H]⁺; ¹H NMR (DMSO-d₆, 400 MHz): δ 13.10 (s, 1H), 11.20 (s, 1H), 8.30 (d, 1H), 8.17 (d, 1H), 7.82 (d, 1H), 7.49 (s, 1H), 7.32 (d, 1H), 6.70 (s, 1H), 6.30 (t, 1H), 5.42 (s, 2H), 4.70 (t, 2H), 3.87 (s, 3H), 3.10 (t, 2H).

### Example 10. N-(4-((1H-pyrazol-1-yl)methyl)-2,3-dihydrobenzofuro[7,6-d]isoxazol-8-yl)-6-methoxy-2,3-dihydrobenzofuran-7-sulfonamide (Compound 64)

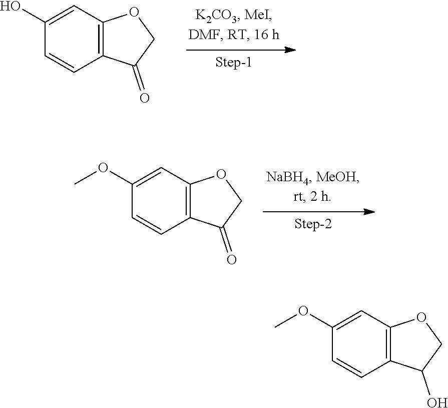

### Step-1: 6-Methoxybenzofuran-3(2H)-one

To a solution of 6-hydroxybenzofuran-3(2H)-one (7.2 g 48.62 mmol) and K₂CO₃ (13.44 g, 97.25 mmol) in DMF (80 mL) was added Mel (11.45 g, 80.71 mmol) at 0° C. The reaction mixture was warmed to room temperature and stirred for 16 h. The reaction mixture was diluted with ice water and extracted with EtOAc. The combined organic layer was washed with brine, dried over anhydrous Na₂SO₄, and concentrated to get the crude compound which was used for next step as such (6.10 g, 88.60%). ¹H NMR (DMSO-d₆, 400 MHz): δ 7.53 (d, 1H), 6.83 (d, 1H), 6.71 (dd, 1H), 4.77 (s, 2H), 3.87 (s, 3H).

### Step-2: 6-Methoxy-2,3-dihydrobenzofuran-3-ol

To a solution of 6-methoxybenzofuran-3(2H)-one (7.0 g, 42.64 mmol) in MeOH (80 mL) was added NaBH₄ (3.22 g, 85.27 mmol) at 0° C. The reaction mixture was warmed to room temperature and stirred for 1 h. The reaction mixture was diluted with ice water and extracted with EtOAc. The combined organic layer was washed with brine, dried over anhydrous Na₂SO₄, and concentrated to obtain the title compound. (6.20 g, 84.80%). 1H NMR (DMSO-d₆, 400 MHz): δ 7.23 (d, 1H), 6.46 (dd, 1H), 6.41 (s, 1H), 5.43 (d, 1H), 5.21-5.16 (m, 1H), 4.52-4.48 (m, 1H), 4.25-4.21 (m, 1H), 3.72 (s, 3H).

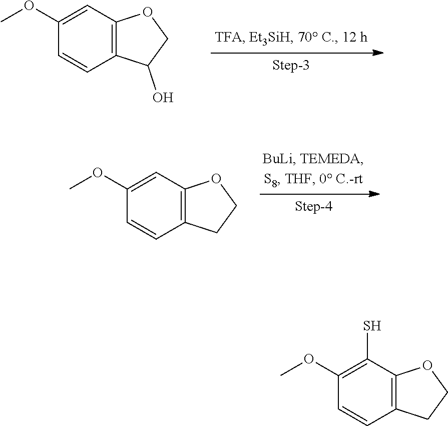

### Step-3: 6-Methoxy-2,3-dihydrobenzofuran

To a solution of 6-methoxy-2,3-dihydrobenzofuran-3-ol (6.0 g, 48.62 mmol) in TFA (8.22 g, 72.21 mmol) was added triethylsilylhydride (4.1 g, 36.10 mmol) at 0° C. and then reaction mixture heated at 70° C. for 12 h. The reaction mixture was cooled to rt and poured into sodium bicarbonate solution and extracted with ethyl acetate. The combined organic layer was washed with water, brine, and dried over anhydrous sodium sulphate, filtered, and concentrated to afford title compound (1.40 g, 25.82%). ¹H NMR (DMSO-d₆, 400 MHz): δ 7.11 (d, 1H), 6.39 (d, 1H), 6.37 (s, 1H), 4.55 (t, 2H), 3.72 (s, 3H), 3.08 (t, 2H).

### Step-4: 6-Methoxy-2,3-dihydrobenzofuran-7-thiol

To a solution of 6-methoxy-2,3-dihydrobenzofuran (1.40 g, 9.32 mmol) in THF (14 mL) was slowly added n-BuLi (5.59 mL, 13.99 mmol) dropwise and followed by TEMDA (0.10 g, 0.93 mmol) at 0° C. The reaction mixture was slowly warmed to rt and stirred at same temperature for 20 min. Sulphur powder (0.28 g, 8.39 mmol) in toluene was added to the reaction mixture at 0° C. Then reaction mixture was warmed to room temperature and stirred for 12 h. The reaction was quenched with 1N HCl and extracted with ethyl acetate. The combined organic layer was dried over solid sodium sulphate, filtered, and concentrated to afford crude. The crude was purified by silica-gel flash column chromatography using 15% of ethyl acetate in hexane as eluent to obtained pure compound (1.0 g, 58.86%). LC-MS: 181.0 [M−H]⁻.

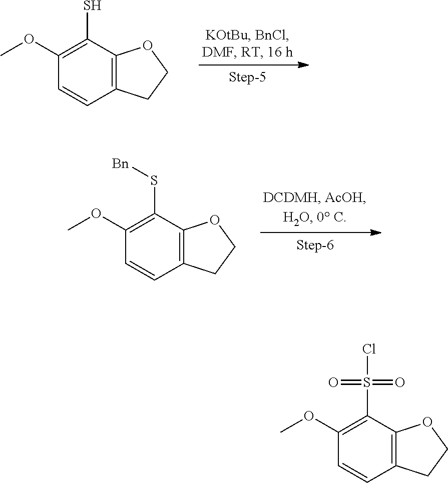 [SMILES: COc1ccc2c(c1S)OCC2 ; COc1ccc2c(c1SCc1ccccc1)OCC2 ; COc1ccc2c(c1S(=O)(=O)Cl)OCC2]

### Step-5: 7-(Benzylthio)-6-methoxy-2,3-dihydrobenzofuran

To a solution of 6-methoxy-2,3-dihydrobenzofuran-7-thiol (1.0 g, 5.48 mmol) and potassium tertiary butoxide (0.739 g, 6.58 mmol) in THF (10 mL) was added benzyl chloride (0.69 g, 5.48 mmol) at 0° C. The reaction mixture was slowly warmed to room temperature and stirred for 16 h. The reaction mixture was quenched with water and extracted with ethyl acetate. The combined organic layer was washed with brine, dried over anhydrous sodium sulphate, and concentrated under vacuum to afford the crude. The crude was purified by silica-gel flash column chromatography using 5-15% of ethyl acetate in hexane as eluent to obtained pure compound (0.45 g, 30.11%). LC-MS: 273.0 [M+H]⁺.

### Step-6: 6-Methoxy-2,3-dihydrobenzofuran-7-sulfonyl Chloride

To a solution of 7-(benzylthio)-6-methoxy-2,3-dihydrobenzofuran (0.25 g, 0.98 mmol) in acetonitrile (3 mL), acetic acid (2 mL) and water (1 mL), was added DCDMH (0.21 g, 1.10 mmol) at 0° C. and stirred for 15 min. The reaction mixture was quenched with water and extracted with ethyl acetate. The combined organic layer was washed with sodium bicarbonate solution, brine, dried over anhydrous sodium sulphate, and concentrated to afford the crude material. The crude compound was purified by silica-gel flash column chromatography using 15-20% ethyl acetate in hexane as eluent to obtained pure compound (0.17 g, 74.5%). ¹H NMR (DMSO-d₆, 400 MHz): δ 7.08 (d, 1H), 6.39 (d, 1H), 6.37 (s, 1H), 4.47 (t, 2H), 3.69 (s, 3H), 3.06 (t, 2H).

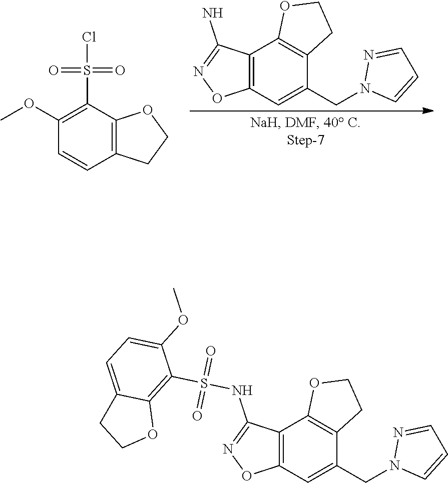 [SMILES: COc1ccc2c(c1S(=O)(=O)Cl)OCC2 ; Nc1noc2cc(Cn3cccn3)c3c(c12)OCC3 ; COc1ccc2c(c1S(=O)(=O)Nc1noc3cc(Cn4cccn4)c4c(c13)OCC4)OCC2]

This compound was synthesized according to step 5 for Compound 65: LCMS: 469.3 [M+H]⁺; ¹H NMR (DMSO-d₆, 400 MHz): δ 10.78 (brs, 1H), 7.81 (d, 1H), 7.49 (d, 1H), 7.24 (brs, 1H), 7.56-7.46 (m, 2H), 6.29 (d, 1H), 5.38 (s, 2H), 4.70 (t, 2H), 4.51 (t, 2H), 3.66 (s, 3H), 3.10-3.04 (m, 4H).

### Example 11. N-(4-((1H-pyrazol-1-yl)methyl)-2,3-dihydrobenzofuro[7,6-d]isoxazol-8-yl)-4-cyclopropyl-2-methoxybenzenesulfonamide (Compound 65)

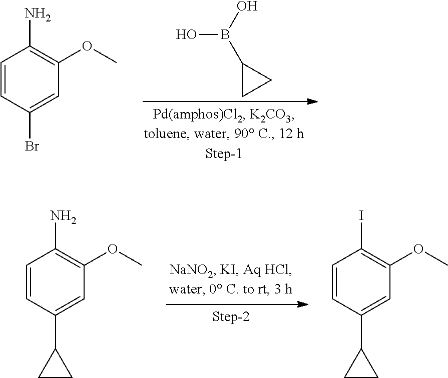

### Step 1: 4-Cyclopropyl-2-methoxyaniline

To a degassed solution of 4-bromo-2-methoxyaniline (3.0 g, 14.85 mmol), cyclopropylboronic acid (6.37 g, 76.24 mmol) and dipotassium carbonate (6.15 g, 138.21 mmol) in toluene (30 mL) and water (3 ml) was added Pd(amphos)Cl₂ (0.543 g, 0.74 mmol) and stirred at 90° C. for 12 h. Then reaction mixture was cooled to rt, passed through celite pad, and washed with ethyl acetate. The filtrate was concentrated under reduced pressure to get crude product which was purified by silica-gel flash column chromatography using 0-10% ethyl acetate in hexane as eluent to afford title compound (2.0 g, 82.53%). LC-MS: 164.1 [M+H]⁺.

### Step 2: 4-Cyclopropyl-1-iodo-2-methoxybenzene

To a solution of 4-cyclopropyl-2-methoxyaniline (1.3 g, 7.96 mmol) in 5N aqueous HCl (25 ml) was added a solution of NaNO₂ (0.824 g, 11.94 mmol) in water (19 ml) at 0° C. dropwise and stirred for 1 h. Then KI (2.909 g, 166 mmol) in water was added dropwise to the reaction mixture at 0° C. and stirred for 30 min. Then reaction mixture was slowly warmed to room temperature and stirred for 2 h. The reaction mixture was quenched with 6N NaOH solution and extracted with EtOAc. The combined organic layer was washed with brine, dried over sodium sulphate, filtered, and concentrated to afford the crude material. The crude material was purified by silica-gel flash column chromatography using 5-10% EtOAc in hexane as eluent to get the title compound (0.6 g, 27.48%). ¹H NMR (DMSO-d₆, 400 MHz): δ 7.58 (d, 1H), 6.73 (s, 1H), 6.48 (d, 1H), 3.82 (s, 3H), 1.95-1.88 (m, 1H), 0.98-0.94 (m, 2H), 0.74-0.69 (m, 2H).

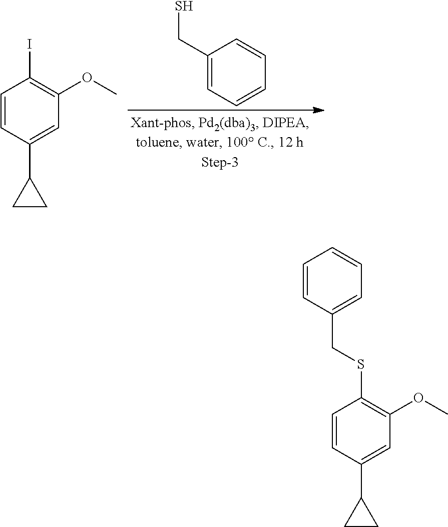 [SMILES: COc1cc(C2CC2)ccc1I ; SCc1ccccc1 ; COc1cc(C2CC2)ccc1SCc1ccccc1]

### Step 3: Benzyl(4-cyclopropyl-2-methoxyphenyl)sulfane

To a degassed solution of 4-cyclopropyl-1-iodo-2-methoxybenzene (1.3 g, 4.73 mmol), phenylmethanethiol (0.707 g, 5.69 mmol), N,N-diisopropylethylamine (1.26 g, 9.48 mmol), and XantPhos (0.55 g, 0.94 mmol) in toluene (13 mL) was added Pd₂(dba)₃ (0.1 g, 0.1 mmol), and stirred at 100° C. for 12 h. The reaction mixture was cooled to room temperature, passed through the celite, washed with ethyl acetate and concentrated the filtrate under reduced pressure to get crude. The crude was purified by silica-gel flash column chromatography using 10-15% EtOAc in hexane as eluent to get the pure titled compound (1.2 g, 93.57%). ¹H NMR (DMSO-d₆, 400 MHz) δ 7.37-7.22 (m, 5H), 7.09 (d, 1H), 6.81 (s, 1H), 6.58 (d, 1H), 4.10 (s, 2H), 3.82 (s, 3H), 1.92-1.85 (m, 1H), 0.96-0.91 (m, 2H), 0.69-0.67 (m, 2H).

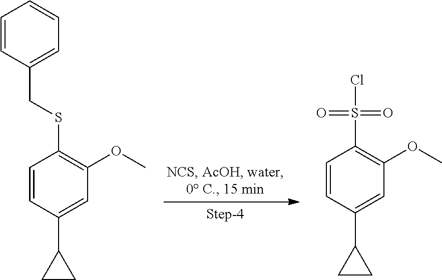 [SMILES: COc1cc(C2CC2)ccc1SCc1ccccc1 ; COc1cc(C2CC2)ccc1S(=O)(=O)Cl]

### Step 4: 4-Cyclopropyl-2-methoxybenzenesulfonyl Chloride

To a solution of benzyl(4-cyclopropyl-2-methoxyphenyl)sulfane (0.350 g, 1.30 mmol) in acetic acid (6 mL), and water (0.6 mL) was added 1-chloro-2,5-pyrrolidinedione (0.519 g, 3.88 mmol) at 0° C. and stirred at same temperature for 15 min. The reaction mixture was quenched with water and extracted with EtOAc. The combined organic layer was washed with aqueous sodium bicarbonate solution, and brine. The organic layer was dried over anhydrous sodium sulphate, filtered, and concentrated to afford the crude material. The crude was purified by silica-gel flash column chromatography using 3-5% EtOAc in hexane as eluent to get the title compound (0.29 g, 90.84%): ¹H NMR (DMSO-d₆, 400 MHz) δ 7.37-7.22 (m, 5H), 7.09 (d, 1H), 6.81 (s, 1H), 6.58 (d, 1H), 4.10 (s, 2H), 3.82 (s, 3H), 1.92-1.85 (m, 1H), 0.96-0.91 (m, 2H), 0.69-0.67 (m, 2H).

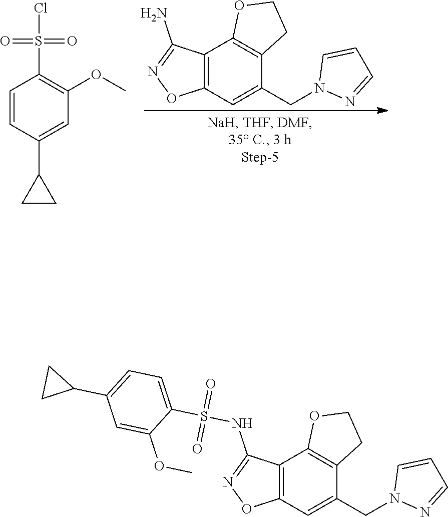

### Step 5: N-(4-((1H-pyrazol-1-yl)methyl)-2,3-dihydrobenzofuro[7,6-d]isoxazol-8-yl)-4-cyclopropyl-2-methoxybenzenesulfonamide

A stirred solution of 4-((1H-pyrazol-1-yl)methyl)-2,3-dihydrobenzofuro[7,6-d]isoxazol-8-amine (0.100 g, 0.39 mmol) in THF (2.5 mL) and DMF (1 mL) was cooled to 0° C. and was added sodium hydride (60% in mineral oil) (0.027 g, 1.170 mmol) in portions and stirred for 5 mins. 4-cyclopropyl-2-methoxybenzenesulfonyl chloride was dissolved in THF (0.5 ml) and slowly added to the reaction mixture and stirred at 35° C. for 3 h. The mixture was quenched with ice and extracted with ethyl acetate. The combined organic layer was washed with brine solution, dried over anhydrous Na₂SO₄, and concentrated under reduced pressure to get crude product which was purified by silica-gel flash column chromatography using 80% ethyl acetate in hexane as eluent to afford title compound (0.005 g, 2.75%). LC-MS: 467.2. [M+H]⁺; ¹H NMR (DMSO-d₆, 400 MHz) δ 10.68 (s, 1H), 7.84 (d, 1H), 7.58 (d, 1H), 7.49 (s, 1H), 6.86 (s, 1H), 6.75-6.68 (m, 2H), 6.31 (t, 1H), 5.42 (s, 2H), 4.72 (t, 2H), 3.77 (s, 3H), 3.10 (t, 2H), 2.03-1.96 (m, 1H) 1.08-1.02 (m, 2H), 0.84-0.79 (m, 2H).

### Example 12. KAT6A Biochemical Assay

TR-FRET based method was used for assaying compounds of the present invention for KAT6A enzyme inhibitory activity. TR-FRET is homogeneous proximity assay where Europium-labelled anti-acetyl lysine antibody binds to the acetylated substrate labelled with biotin which in turn binds to streptavidin labelled APC fluorescence acceptor. Europium can transfer energy to APC in the complex and the interaction of two dye-labelled binding partners is detected by the energy transfer between a donor and an acceptor dye, and the subsequent light emission by the acceptor dye. KAT6A transfer an acetyl group from acetyl CoA to lysine amino acids of histones/target proteins. Typically, 5 μL of human-KAT6A (MYST domain 507-778 aa) in assay buffer (100 mM Tris HCl (pH 7.8), 15 mM NaCl, 1 mM EDTA, 0.01% Tween-20, 0.02% BSA, 1 mM DTT) is added to 384-well plate containing 5 μL of selected test compound in final 1% DMSO, serially diluted in 1:3 in an 8-10-point titration. The selected compound of the present invention and enzyme are incubated for 30 min at room temperature. Next, 5 μL of substrate mix containing histone H4 peptide and acetyl-CoA in assay buffer is added to the plate. The final concentrations of H4 peptide and acetyl-CoA are 200 nM and 600 nM respectively. Following 30 min reaction at room temperature, 5 μL of detection mix containing Europium labelled anti-acetyl antibody and streptavidin-APC is added to the reaction wells. The plate is further incubated for 45 min at room temperature and is read in TR-FRET mode (Ex:340 nm; Em: 615 nm and 665 nm) on a plate reader. The percent inhibition was calculated from the ratio of the fluorescence (FL) intensities [(F665/F615)×10,000) using the formula (Control FL ratio—(Sample FL ratio/Control FL ratio))×100. IC₅₀ values of the compounds were determined by fitting the dose-response data to sigmoidal curve fitting equation using Graph pad prism software V8. The results of the aforesaid experiment are summarized in Table 10 below.

TABLE 10
KAT6A Activity

| | KAT6A IC₅₀ | Percent Inhibition |
|---|---|---|
| Compound | (nM) | (at 1 μM) |
| 1 | 57 | 100 |
| 2 | 20 | 100 |
| 3 | 43 | 100 |
| 4 | 29 | 100 |
| 5 | ND | 32 |
| 6 | 46 | 100 |
| 7 | 150 | 100 |
| 8 | 43 | 100 |
| 9 | 36 | 95 |
| 10 | ND | 93 |
| 11 | 88 | 100 |
| 12 | 291 | 95 |
| 13 | 71 | 100 |
| 14 | 156 | 100 |
| 15 | 45 | 100 |
| 16 | ND | 25 |
| 17 | 74 | 97 |
| 18 | 37 | 100 |
| 19 | 31 | 100 |
| 20 | 51 | 100 |
| 21 | 19 | 100 |
| 22 | 21 | 100 |
| 23 | 23 | 100 |
| 31 | 18 | 100 |
| 32 | 61 | 100 |
| 33 | 127 | 100 |
| 34 | 26 | 100 |
| 35 | 27 | 100 |
| 36 | 219 | 92 |
| 37 | 494 | 82 |
| 38 | 32 | 100 |
| 39 | 22 | 100 |
| 40 | 27 | 100 |
| 41 | 25 | 100 |
| 42 | 26.6 | 100 |
| 43 | 12 | 100 |
| 44 | 32.7 | 100 |
| 45 | 40 | 100 |
| 46 | 20 | 100 |
| 47 | 26 | 100 |
| 48 | 30 | 100 |
| 49 | 76 | 100 |
| 50 | 70 | 100 |
| 51 | 35 | 100 |
| 52 | 32 | 100 |
| 53 | 29 | 100 |
| 54 | 158 | 100 |
| 55 | 34 | 100 |
| 56 | 43 | 100 |
| 57 | 135 | 100 |
| 58 | 21 | 100 |
| 59 | 35 | 100 |
| 60 | 99 | 100 |
| 61 | 280 | 91 |
| 62 | 164 | 100 |
| 63 | 23 | 100 |
| 64 | 10 | 100 |
| 65 | 10 | 100 |

“ND” refers to ‘Not Determined’

Although the foregoing invention has been described in some detail by way of illustration and Example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A compound of Formula (I): 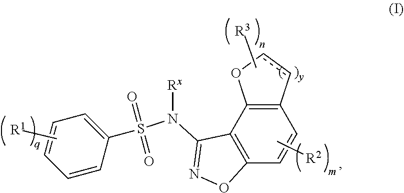
or a pharmaceutically acceptable salt or a stereoisomer or a tautomer thereof; wherein, dotted line [symbol] represents a single bond or is absent;
each R¹ is independently hydrogen, (C₁-C₆)alkyl, (C₂-C₆)alkenyl, (C₂-C₆)alkynyl, (C₁-C₆)alkoxy, (C₂-C₆)alkoxyalkyl, (C₁-C₆)hydroxyalkyl, (C₁-C₆)aminoalkyl, halogen, (C₁-C₆)haloalkyl, (C₁-C₆)haloalkoxy, —C(O)R^{1h}, —C(O)OR^{1h}, —OC(O)R^{1h}, —C(O)N(R^{1h})(R^{1j}), —N(R^{1h})C(O)(R^{1j}), —S(O)₂R^{1h}, —S(O)₂N(R^{1h})(R^{1j}), hydroxy, —CN, —NO₂, —SF₅, (C₃-C₈)cycloalkyl, 3- to 8-membered heterocycloalkyl, (C₆-C₁₀)aryl, or 5- to 10-membered heteroaryl, wherein each alkoxy is substituted with 0, 1, 2, or 3 R^{1f}, and each cycloalkyl, heterocycloalkyl, aryl, and heteroaryl is substituted with 0, 1, 2, or 3 R^{1g};
each R^{1h} and R¹ⁱ is independently hydrogen or (C₁-C₆)alkyl;
each R^{1f} is independently 5- to 10-membered heteroaryl;
each R^{1g} is independently (C₁-C₆)alkyl, (C₁-C₆)alkoxy, halogen, (C₁-C₆)haloalkyl, or (C₁-C₆)haloalkoxy;
alternatively, two R¹ groups on adjacent ring atoms are combined to form (C₅-C₈)cycloalkyl or a 5- to 8-membered heterocycloalkyl;
each R² is independently hydrogen, (C₁-C₆)alkyl, (C₂-C₆)alkenyl, (C₂-C₆)alkynyl, (C₁-C₆)alkoxy, (C₁-C₆)hydroxyalkyl, halogen, (C₁-C₆)haloalkoxy, hydroxy, —OR^{4c}, —NHR^{4c}, —SR^{4c}, —C(O)R^{4c}, —S(O)R^{4c}, —S(O)₂R^{4c}, (C₃-C₈)cycloalkyl, (C₆-C₁₀)aryl, 3- to 8-membered heterocycloalkyl, or 5- to 10-membered heteroaryl; wherein each alkyl group is substituted with 0, 1, 2 or 3 R^{4a}; and
wherein each cycloalkyl, heterocycloalkyl, aryl, and heteroaryl is substituted with 0, 1, 2 or 3 R^{4b};
each R³ is independently hydrogen, (C₁-C₆)alkyl, (C₁-C₆)alkoxy, halogen, (C₁-C₆)haloalkoxy, or hydroxy; wherein each alkyl is substituted with 0, 1, 2, or 3 R^{3a};
each R^{3a} is independently hydroxy, (C₁-C₆)alkoxy or halogen;
each R^{4a} is independently (C₁-C₆)alkoxy, halogen, C₁-C₆haloalkoxy, —NH₂, —CN, —NO₂, C₃-C₈ cycloalkyl, (C₆-C₁₀)aryl, 3- to 8-membered heterocycloalkyl, or 5- to 10-membered heteroaryl, wherein each cycloalkyl, heterocycloalkyl, aryl, and heteroaryl is substituted with 0, 1, 2, or 3 R^{4a1};
each R^{4a1} is independently (C₁-C₆)alkyl, (C₂-C₆)alkenyl, (C₂-C₆)alkynyl, (C₁-C₆)alkoxy, (C₂-C₆)alkoxyalkyl, (C₁-C₆)hydroxyalkyl, halogen, (C₁-C₆)haloalkyl, (C₁-C₆)haloalkoxy, hydroxy, —N(R^{4a2})(R^{4a3}), —C(O)R^{4a2}, —C(O)OR^{4a2}, —OC(O)R^{4a2}, —C(O)N(R^{4a2})(R^{4a3}), —N(R^{4a2})C(O)(R^{4a3}), —S(O)₂R^{4a2}, —S(O)₂N(R^{4a2})(R^{4a3}), —CN, or —NO₂;
each R^{4a2} and R^{4a3} is independently hydrogen or (C₁-C₆)alkyl;
each R^{4b} is independently (C₁-C₆)alkyl, (C₂-C₆)alkenyl, (C₂-C₆)alkynyl, (C₁-C₆)alkoxy, (C₁-C₆)hydroxyalkyl, halogen, (C₁-C₆)haloalkyl, (C₁-C₆)haloalkoxy, hydroxy, —NH₂, —CN, —NO₂, (C₃-C₈)cycloalkyl, (C₆-C₁₀)aryl, 3- to 8-membered heterocycloalkyl, or 5- to 10-membered heteroaryl;
each R^{4′} is (C₃-C₈)cycloalkyl, (C₆-C₁₀)aryl, 3- to 8-membered heterocycloalkyl, or 5- to 10-membered heteroaryl;
R^{x} is hydrogen or (C₁-C₆)alkyl;
subscript ‘m’ is an integer of 1 or 2;
subscript ‘y’ is an integer of 1 or 2;
subscript ‘n’ is an integer of 1, 2 or 3; and
subscript ‘q’ is an integer of from 1 to 5,
wherein each heterocycloalkyl and heteroaryl includes 1 to 4 heteroatoms each independently N, O or S.

2. The compound of claim 1, or a pharmaceutically acceptable salt or a stereoisomer or a tautomer thereof; wherein,
dotted line [symbol] represents a single bond or is absent;
each R¹ is independently hydrogen, halogen, (C₁-C₆)alkyl, (C₁-C₆)haloalkyl, (C₁-C₆)aminoalkyl, (C₁-C₆)alkoxy or (C₁-C₆)haloalkoxy;
each R² is independently hydrogen, (C₁-C₆)alkyl, (C₁-C₆)alkoxy, halogen, (C₁-C₆)haloalkoxy, (C₆-C₁₀)aryl, 3- to 8-membered heterocycloalkyl, or 5- to 10-membered heteroaryl; wherein the alkyl group is substituted with 0, 1, 2 or 3 R^{4a}; and the aryl, heterocycloalkyl and heteroaryl groups are substituted with 0, 1, 2 or 3 R^{4b};
each R³ is independently hydrogen, (C₁-C₆)alkyl, halogen, hydroxy or (C₁-C₆)alkoxy;
wherein the alkyl group is substituted with 0, 1, 2, or 3 substituent(s) selected from hydroxy, (C₁-C₆)alkoxy and halogen;
each R^{4a} is independently halogen, (C₁-C₆)haloalkoxy, amino, (C₁-C₆)alkoxy, (C₆-C₁₀)aryl, 3- to 8-membered heterocycloalkyl or 5- to 10-membered heteroaryl;
each R^{4b} is independently halogen, (C₁-C₆)haloalkoxy, amino, (C₁-C₆)alkyl, (C₁-C₆)alkoxy, (C₆-C₁₀)aryl, 3- to 8-membered heterocycloalkyl or 5- to 10-membered heteroaryl;
R^{x} is hydrogen or (C₁-C₆)alkyl;
subscript ‘m’ is an integer of 1 or 2;
subscript ‘y’ is an integer of 1 or 2;
subscript ‘n’ is an integer of 1, 2 or 3; and
subscript ‘q’ is an integer of from 1 to 5,
wherein each heterocycloalkyl and heteroaryl includes 1 to 4 heteroatoms each independently N, O or S.

3. The compound of claim 1 or 2, or a pharmaceutically acceptable salt thereof, wherein
each R¹ is independently (C₁-C₆)alkyl, (C₂-C₆)alkenyl, (C₂-C₆)alkynyl, (C₁-C₆)alkoxy, (C₂-C₆)alkoxyalkyl, (C₁-C₆)hydroxyalkyl, (C₁-C₆)aminoalkyl, halogen, (C₁-C₆)haloalkyl, (C₁-C₆)haloalkoxy, —C(O)R^{1h}, —C(O)OR^{1h}, (C₃-C₈)cycloalkyl, 3- to 8-membered heterocycloalkyl, (C₆-C₁₀)aryl, or 5- to 10-membered heteroaryl, wherein each alkoxy is substituted with 0, 1, 2, or 3 R^{1f}, and each cycloalkyl, heterocycloalkyl, aryl, and heteroaryl is substituted with 0, 1, 2, or 3 R^{1g};
each R^{1h} is independently hydrogen or (C₁-C₆)alkyl;
each R^{1f} is independently 5- to 10-membered heteroaryl; and
each R^{1g} is independently (C₁-C₆)alkyl, (C₁-C₆)alkoxy, or (C₁-C₆)haloalkyl;
alternatively, two R¹ groups on adjacent ring atoms are combined to form (C₅-C₈)cycloalkyl or a 5- to 8-membered heterocycloalkyl.

4. The compound of any one of claims 1 to 3, or a pharmaceutically acceptable salt thereof, wherein
each R¹ is independently (C₁-C₆)alkyl, (C₁-C₆)alkoxy, halogen, (C₁-C₆)haloalkyl, (C₁-C₆)haloalkoxy, —C(O)OR^{1h}, (C₃-C₈)cycloalkyl, (C₆-C₁₀)aryl, or 5- to 6-membered heteroaryl having 1 to 3 heteroatoms each N, wherein each alkoxy is substituted with 0, 1, or 2 R^{1f}, and each aryl, and heteroaryl is substituted with 0, 1, or 2 R^{1g};
each R^{1h} is independently hydrogen or (C₁-C₆)alkyl;
each R^{1f} is independently 5- to 6-membered heteroaryl having 1 to 3 heteroatoms each N; and
each R^{1g} is independently (C₁-C₆)haloalkyl;
alternatively, two R¹ groups on adjacent ring atoms are combined to form (C₅-C₈)cycloalkyl.

5. The compound of any one of claims 1 to 4, or a pharmaceutically acceptable salt thereof, wherein
each R¹ is independently (C₁-C₄)alkyl, (C₁-C₃)alkoxy, halogen, (C₁-C₃)haloalkyl, (C₁-C₃)haloalkoxy, —C(O)OR^{1h}, (C₃-C₈)cycloalkyl, (C₆-C₁₀)aryl, or 5- to 6-membered heteroaryl having 1 to 3 heteroatoms each N, wherein each alkoxy is substituted with 0 or 1 R^{1f};
each R^{1h} is independently hydrogen or (C₁-C₃)alkyl; and
each R^{1f} is independently 5- to 6-membered heteroaryl having 1 to 3 heteroatoms each N;
alternatively, two R¹ groups on adjacent ring atoms are combined to form (C₅-C₈)cycloalkyl.

6. The compound of any one of claims 1 to 5, or a pharmaceutically acceptable salt thereof, wherein
each R¹ is independently Et, t-Bu, OMe, OEt, OCH₂-pyrid-2-yl, F, Cl, CF₃, OCF₃, COOH, COOMe, cyclopropyl, phenyl, or 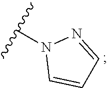 [SMILES: *n1cccn1]
alternatively, two R¹ on adjacent ring atoms are combined to form cyclopentyl or cyclohexyl.

7. The compound of any one of claims 1 to 6, or a pharmaceutically acceptable salt thereof, wherein
R^{x} is hydrogen.

8. The compound of any one of claims 1 to 7, or a pharmaceutically acceptable salt thereof, wherein
subscript ‘m’ is 1.

9. The compound of any one of claims 1 to 8, or a pharmaceutically acceptable salt thereof, having the structure of Formula IA-1:

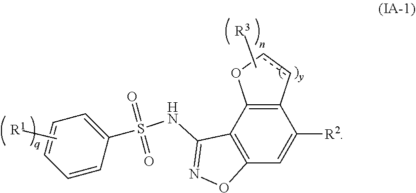

10. The compound of any one of claims 1 to 9, or a pharmaceutically acceptable salt thereof, wherein the dotted line [symbol] is absent.

11. The compound of any one of claims 1 to 10, or a pharmaceutically acceptable salt thereof, wherein
subscript ‘y’ is 1.

12. The compound of any one of claims 1 to 11, or a pharmaceutically acceptable salt thereof, having the structure of Formula IB-1:

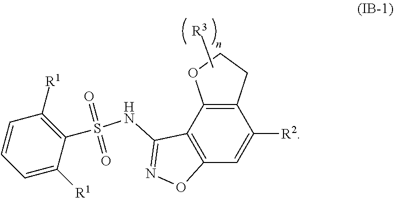

13. The compound of any one of claims 1 to 12, or a pharmaceutically acceptable salt thereof, wherein
each R¹ is independently (C₁-C₆)alkyl, (C₁-C₆)alkoxy, halogen, (C₁-C₆)haloalkyl, (C₁-C₆)haloalkoxy, —C(O)OR^{1h}, (C₃-C₈)cycloalkyl, (C₆-C₁₀)aryl, or 5- to 10-membered heteroaryl having 1 to 3 heteroatoms each N, wherein each alkoxy is substituted with 0, 1, 2, or 3 Rif, and each aryl, and heteroaryl is substituted with 0, 1, 2, or 3 R^{1g};
each R^{1h} is independently hydrogen or (C₁-C₆)alkyl;
each R^{1f} is independently 5- to 6-membered heteroaryl having 1 to 3 heteroatoms each N; and
each R^{1g} is independently (C₁-C₆)haloalkyl.

14. The compound of any one of claims 1 to 13, or a pharmaceutically acceptable salt thereof, wherein
each R¹ is independently (C₁-C₃)alkyl, (C₁-C₃)alkoxy, halogen, or (C₁-C₃)haloalkyl.

15. The compound of any one of claims 1 to 11, or a pharmaceutically acceptable salt thereof, having the structure of Formula IB-2:

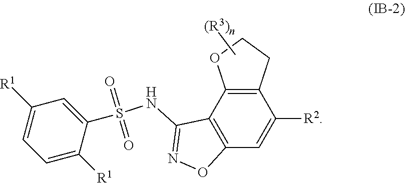

16. The compound of claim 15, or a pharmaceutically acceptable salt thereof, wherein
each R¹ is independently (C₁-C₆)alkyl, (C₁-C₆)alkoxy, halogen, (C₁-C₆)haloalkyl, (C₁-C₆)haloalkoxy, —C(O)OR^{1h}, (C₃-C₈)cycloalkyl, C₆-C₁₀ aryl, or 5- to 10-membered heteroaryl having 1 to 3 heteroatoms each N, wherein each alkoxy is substituted with 0, 1, 2, or 3 Rif, and each aryl, and heteroaryl is substituted with 0, 1, 2, or 3 R^{1g};
each R^{1h} is independently hydrogen or (C₁-C₆)alkyl;
each R^{1f} is independently 5- to 6-membered heteroaryl having 1 to 3 heteroatoms each N; and
each R^{1g} is independently (C₁-C₆)haloalkyl.

17. The compound of claim 16, or a pharmaceutically acceptable salt thereof, wherein
each R¹ is independently (C₁-C₃)alkyl, (C₁-C₃)alkoxy, halogen, or (C₁-C₃)haloalkyl.

18. The compound of any one of claims 1 to 11, or a pharmaceutically acceptable salt thereof, having the structure of Formula IB-3:

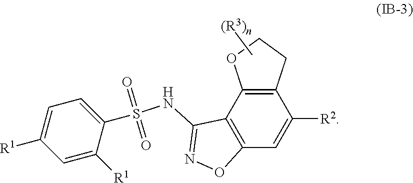

19. The compound of claim 18, or a pharmaceutically acceptable salt thereof, wherein
each R¹ is independently (C₁-C₆)alkyl, (C₁-C₆)alkoxy, halogen, (C₁-C₆)haloalkyl, (C₁-C₆)haloalkoxy, —C(O)OR^{1h}, (C₃-C₈)cycloalkyl, C₆-C₁₀ aryl, or 5- to 10-membered heteroaryl having 1 to 3 heteroatoms each N, wherein each alkoxy is substituted with 0, 1, 2, or 3 R^{1f}, and each aryl, and heteroaryl is substituted with 0, 1, 2, or 3 R^{1g};
each R^{1h} is independently hydrogen or (C₁-C₆)alkyl;
each R^{1f} is independently 5- to 6-membered heteroaryl having 1 to 3 heteroatoms each N; and
each R^{1g} is independently (C₁-C₆)haloalkyl.

20. The compound of claim 19, or a pharmaceutically acceptable salt thereof, wherein
each R¹ is independently (C₁-C₃)alkyl, (C₁-C₃)alkoxy, halogen, or (C₁-C₃)haloalkyl.

21. The compound of any one of claims 1 to 11, or a pharmaceutically acceptable salt thereof, having the structure of Formula IB-4:

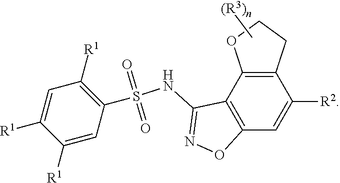

22. The compound of claim 21, or a pharmaceutically acceptable salt thereof, wherein
each R¹ is independently (C₁-C₆)alkyl, (C₁-C₆)alkoxy, halogen, (C₁-C₆)haloalkyl, (C₁-C₆)haloalkoxy, —C(O)OR^{1h}, (C₃-C₈)cycloalkyl, C₆-C₁₀ aryl, or 5- to 10-membered heteroaryl having 1 to 3 heteroatoms each N, wherein each alkoxy is substituted with 0, 1, 2, or 3 R^{1f}, and each aryl, and heteroaryl is substituted with 0, 1, 2, or 3 R^{1g};
each R^{1h} is independently hydrogen or (C₁-C₆)alkyl;
each R^{1f} is independently 5- to 6-membered heteroaryl having 1 to 3 heteroatoms each N; and
each R^{1g} is independently (C₁-C₆)haloalkyl;
alternatively, two R¹ groups on adjacent ring atoms are combined to form (C₅-C₈)cycloalkyl.

23. The compound of any one of claims 1 to 22, or a pharmaceutically acceptable salt thereof, wherein
each R² is independently (C₁-C₆)alkyl, (C₂-C₆)alkenyl, (C₂-C₆)alkynyl, (C₁-C₆)alkoxy, (C₁-C₆)hydroxyalkyl, halogen, (C₁-C₆)haloalkoxy, hydroxy, (C₃-C₈)cycloalkyl, (C₆-C₁₀)aryl, 3- to 8-membered heterocycloalkyl, or 5- to 10-membered heteroaryl; wherein each alkyl group is substituted with 0, 1, 2 or 3 R^{4a}; and each cycloalkyl, heterocycloalkyl, aryl, and heteroaryl is substituted with 0, 1, 2 or 3 R^{4b};
each R^{4a} is independently (C₁-C₆)alkoxy, halogen, C₁-C₆haloalkoxy, —NH₂, —CN, —NO₂, C₃-C₈ cycloalkyl, (C₆-C₁₀)aryl, 3- to 8-membered heterocycloalkyl, or 5- to 10-membered heteroaryl, wherein each cycloalkyl, heterocycloalkyl, aryl, and heteroaryl is substituted with 0, 1, 2, or 3 R^{4a1};
each R^{4a1} is independently (C₁-C₆)alkyl, (C₂-C₆)alkenyl, (C₂-C₆)alkynyl, (C₁-C₆)alkoxy, (C₂-C₆)alkoxyalkyl, (C₁-C₆)hydroxyalkyl, halogen, (C₁-C₆)haloalkyl, (C₁-C₆)haloalkoxy, or —CN; and
each R^{4b} is independently (C₁-C₆)alkyl, (C₂-C₆)alkenyl, (C₂-C₆)alkynyl, (C₁-C₆)alkoxy, (C₁-C₆)hydroxyalkyl, halogen, (C₁-C₆)haloalkyl, (C₁-C₆)haloalkoxy, (C₃-C₈)cycloalkyl, (C₆-C₁₀)aryl, 3- to 8-membered heterocycloalkyl, or 5- to 10-membered heteroaryl.

24. The compound of any one of claims 1 to 23, or a pharmaceutically acceptable salt thereof, wherein
each R² is independently (C₁-C₆)alkyl, (C₂-C₆)alkenyl, (C₂-C₆)alkynyl, (C₃-C₈)cycloalkyl, (C₆-C₁₀)aryl, 3- to 8-membered heterocycloalkyl, or 5- to 10-membered heteroaryl; wherein each alkyl group is substituted with 0, 1, 2 or 3 R^{4a}; and each cycloalkyl, heterocycloalkyl, aryl, and heteroaryl is substituted with 0, 1, 2 or 3 R^{4b};
each R^{4a} is independently C₃-C₈ cycloalkyl, (C₆-C₁₀)aryl, 3- to 8-membered heterocycloalkyl, or 5- to 10-membered heteroaryl, wherein each cycloalkyl, heterocycloalkyl, aryl, and heteroaryl is substituted with 0, 1, 2, or 3 R^{4a1};
each R^{4a1} is independently halogen, (C₁-C₆)haloalkyl, (C₁-C₆)haloalkoxy, or —CN; and
each R^{4b} is independently (C₁-C₆)alkyl, (C₂-C₆)alkenyl, (C₂-C₆)alkynyl, (C₁-C₆)alkoxy, or (C₁-C₆)hydroxyalkyl.

25. The compound of any one of claims 1 to 24, or a pharmaceutically acceptable salt thereof, wherein
each R² is independently (C₁-C₆)alkyl, (C₆-C₁₀)aryl, 5- to 6-membered heterocycloalkyl including 1 to 2 heteroatoms each independently O, or 5- to 6-membered heteroaryl including 1 to 3 heteroatoms each independently N, O or S; wherein each alkyl group is substituted with 0, 1, 2 or 3 R^{4a}; and each heterocycloalkyl, aryl, and heteroaryl is substituted with 0, 1, 2 or 3 R^{4b};
each R^{4a} is independently 5- to 6-membered heteroaryl including 1 to 3 heteroatoms each independently N, substituted with 0, 1, 2, or 3 R^{4a1};
each R^{4a1} is independently halogen, (C₁-C₆)haloalkyl, or —CN; and
each R^{4b} is independently (C₁-C₆)alkyl, or (C₁-C₆)alkoxy.

26. The compound of any one of claims 1 to 25, or a pharmaceutically acceptable salt thereof, wherein
each R² is independently (C₁-C₃)alkyl, phenyl, 5- to 6-membered heterocycloalkyl including 1 to 2 heteroatoms each independently O, or 5- to 6-membered heteroaryl including 1 to 3 heteroatoms each independently N, O or S; wherein
each alkyl group is substituted with 0, 1, 2 or 3 R^{4a}; and each heterocycloalkyl, aryl, and heteroaryl is substituted with 0, 1, 2 or 3 R^{4b};
each R^{4a} is independently 5- to 6-membered heteroaryl including 1 to 3 heteroatoms each independently N, substituted with 0, 1, 2, or 3 R^{4a1};
each R^{4a1} is independently halogen, (C₁-C₃)haloalkyl, or —CN; and
each R^{4b} is independently (C₁-C₃)alkyl, or (C₁-C₃)alkoxy.

27. The compound of any one of claims 1 to 26, or a pharmaceutically acceptable salt thereof, wherein
each R² is independently (C₁-C₆)alkyl; wherein each alkyl group is substituted with 1 or 2 R^{4a};
each R^{4a} is independently 5- to 6-membered heteroaryl including 1 to 3 heteroatoms each independently N, substituted with 0, 1, 2, or 3 R^{4a1}; and
each R^{4a1} is independently halogen, (C₁-C₆)haloalkyl, or —CN.

28. The compound of any one of claims 1 to 27, or a pharmaceutically acceptable salt thereof, wherein
each R² is independently (C₁-C₃)alkyl; wherein each alkyl group is substituted with 1 or 2 R^{4a};
each R^{4a} is independently 5- to 6-membered heteroaryl including 1 to 3 heteroatoms each independently N, substituted with 0, 1, or 2 R^{4a1}; and
each R^{4a1} is independently halogen, (C₁-C₃)haloalkyl, or —CN.

29. The compound of any one of claims 1 to 28, or a pharmaceutically acceptable salt thereof, wherein
each R² is hydrogen, methyl, ethyl, n-propyl or iso-propyl; wherein each group is substituted with 1 or 2 R^{4a};
each R^{4a} is independently pyrrole, pyrazole, imidazole, or triazole, each substituted with 0, 1, or 2 R^{4a1}; and
each R^{4a1} is independently F, Cl, —CF₃ or —CN.

30. The compound of any one of claims 1 to 26, or a pharmaceutically acceptable salt thereof, wherein
each R² is independently —CH₂OMe,

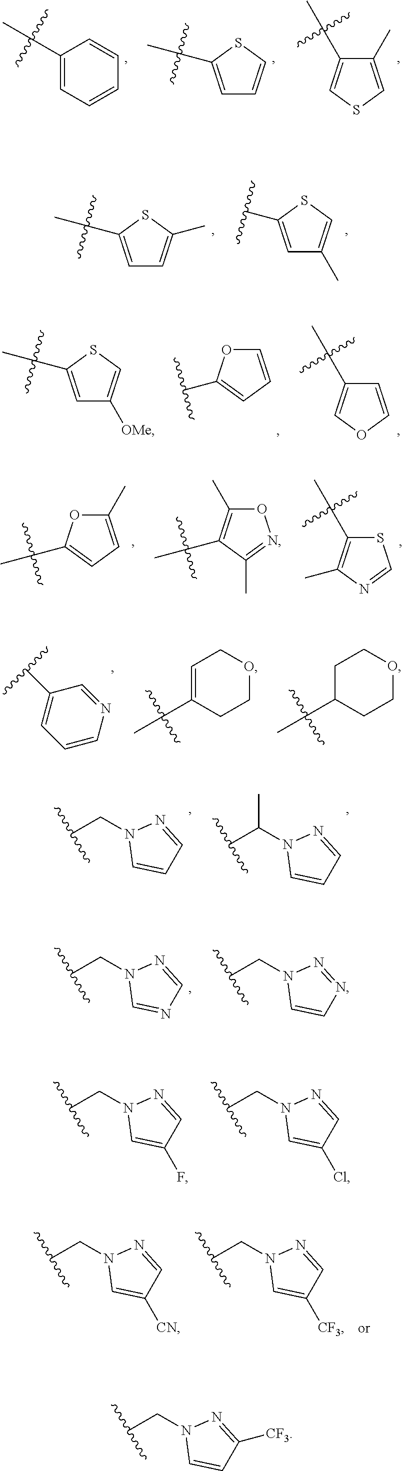

31. The compound of any one of claims 1 to 30, or a pharmaceutically acceptable salt thereof, wherein
each R³ is independently hydrogen or (C₁-C₆)alkyl; wherein each alkyl is substituted with 0, 1, 2, or 3 R^{3a}; and
each R^{3a} is independently (C₁-C₆)alkoxy or halogen.

32. The compound of any one of claims 1 to 31, or a pharmaceutically acceptable salt thereof, wherein
each R³ is independently hydrogen or (C₁-C₃)alkyl; wherein each alkyl is substituted with 0 or 1 R^{3a}; and
each R^{3a} is independently (C₁-C₃)alkoxy.

33. The compound of any one of claims 1 to 31, or a pharmaceutically acceptable salt thereof, wherein each R³ is independently hydrogen, Me, Et, CH₂OMe, CH₂OH, or CH₂F.

34. The compound of any one of claims 1 to 33, or a pharmaceutically acceptable salt thereof, wherein each R³ is independently hydrogen, Me or CH₂OMe.

35. The compound of any one of claims 1 to 34, or a pharmaceutically acceptable salt thereof, wherein subscript ‘n’ is an integer of 1 or 2.

36. The compound of any one of claims 1 to 35, or a pharmaceutically acceptable salt thereof, wherein subscript ‘q’ is an integer of from 1 to 3.

37. The compound of any one of claims 1 to 36, or a pharmaceutically acceptable salt thereof, wherein
each R¹ is independently Et, t-Bu, OMe, OEt, OCH₂-pyrid-2-yl, F, Cl, CF₃, OCF₃, COOH, COOMe, cyclopropyl, phenyl, or 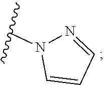 [SMILES: *n1cccn1]
alternatively, two R¹ groups on adjacent ring atoms are combined to form cyclopentyl or cyclohexyl;
R² is —CH₂OMe, 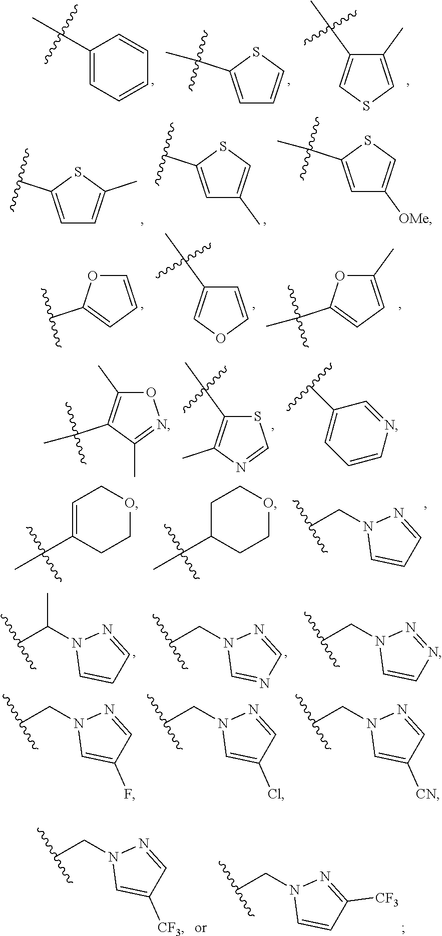
each R³ is independently hydrogen, Me or CH₂OMe;
R^{x} is hydrogen;
subscript ‘m’ is 1;
subscript ‘y’ is 1;
subscript ‘n’ is an integer of 1 or 2; and
subscript ‘q’ is an integer of from 1 to 3.

38. The compound of claim 1, wherein,
each R¹ is independently hydrogen, halogen, (C₁-C₆)alkyl or (C₁-C₆)alkoxy;
each R² is independently 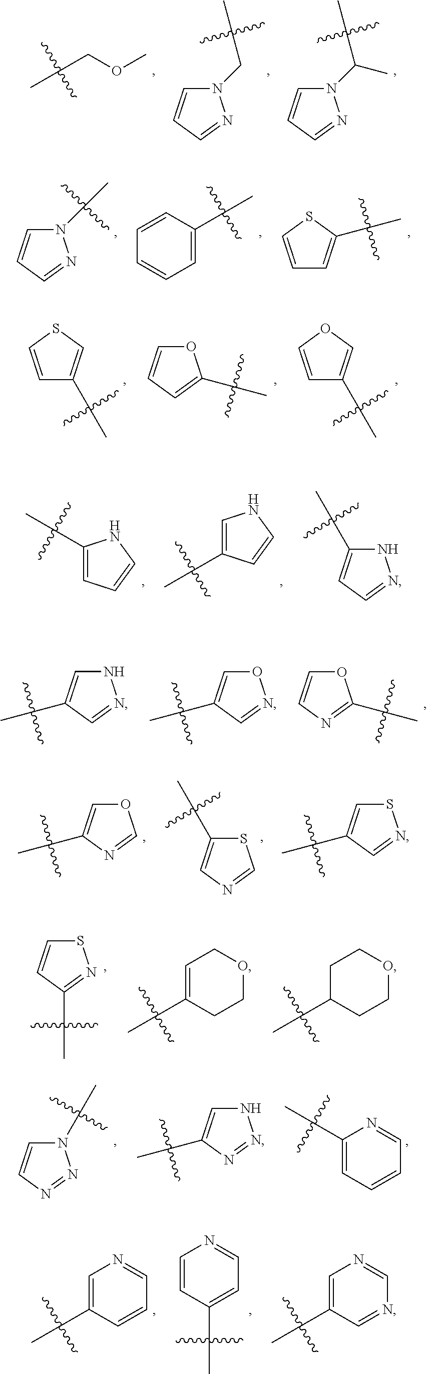 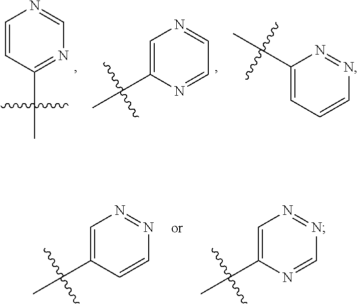 [SMILES: *c1ccncn1 ; *c1cnccn1 ; *c1cccnn1 ; *c1ccnnc1 ; *c1cnncn1]
wherein each group is substituted with 0 or 1 (C₁-C₆)alkyl or (C₁-C₆)alkoxy;
each R³ is independently hydrogen, (C₁-C₆)alkyl or halogen; wherein the alkyl group is substituted with 0 to 3 substituents selected from hydroxy, (C₁-C₆)alkoxy and halogen;
subscript ‘m’ is 1;
subscript ‘n’ is 1 or 2; and
subscript ‘q’ is 1 or 2.

39. The compound of claim 38, represented by compound of formula (IB): 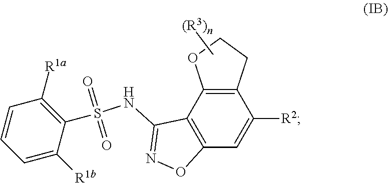
wherein,
each R^{1a} and R^{1b} is independently hydrogen, halogen, (C₁-C₆)alkyl or (C₁-C₆)alkoxy.

40. The compound of claim 38 or 39, wherein
each R^{1a} and R^{1b} is independently (C₁-C₆)alkyl or (C₁-C₆)alkoxy;
R² is 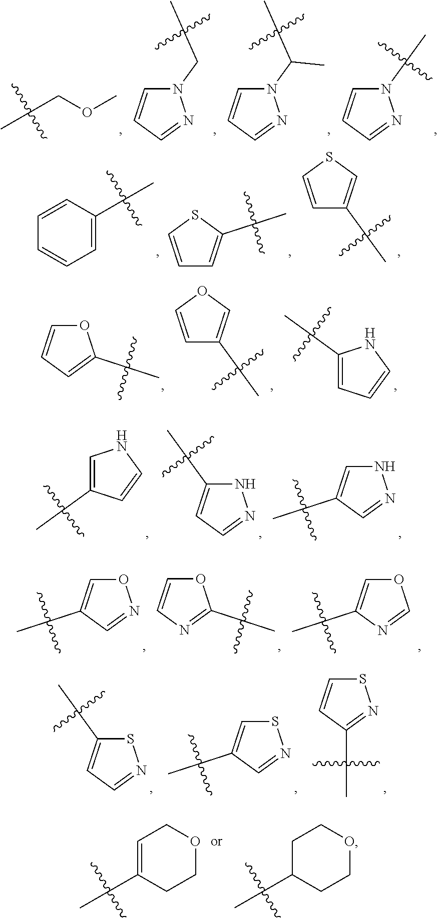
which is substituted with 0 or 1 (C₁-C₆)alkyl or (C₁-C₆)alkoxy; and
each R³ is independently hydrogen, (C₁-C₆)alkyl or halogen; wherein the alkyl group is substituted with 0 to 3 substituents selected from hydroxy, (C₁-C₆)alkoxy and halogen.

41. The compound of any one of claims 1 to 40, or a pharmaceutically acceptable salt thereof, wherein the compound has a structure as shown in the following table

| Compound | Structure |
|---|---|
| 1 | 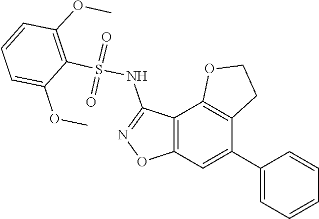 [SMILES: COc1cccc(OC)c1S(=O)(=O)Nc1noc2cc(-c3ccccc3)c3c(c12)OCC3] |
| 2 | 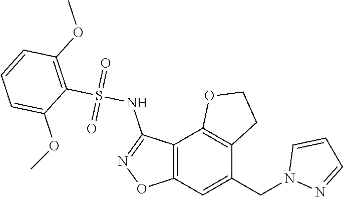 [SMILES: COc1cccc(OC)c1S(=O)(=O)Nc1noc2cc(Cn3cccn3)c3c(c12)OCC3] |
| 3 | 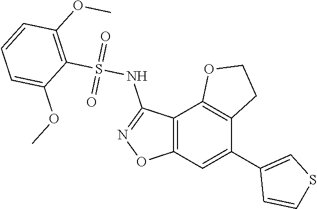 [SMILES: COc1cccc(OC)c1S(=O)(=O)Nc1noc2cc(-c3ccsc3)c3c(c12)OCC3] |
| 4 | 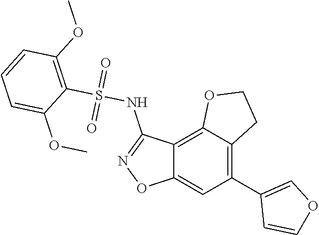 [SMILES: COc1cccc(OC)c1S(=O)(=O)Nc1noc2cc(-c3ccoc3)c3c(c12)OCC3] |
| 5 | 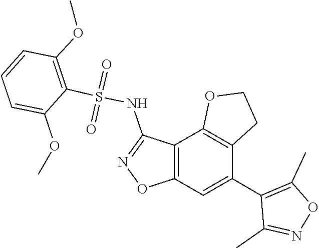 [SMILES: COc1cccc(OC)c1S(=O)(=O)Nc1noc2cc(-c3c(C)noc3C)c3c(c12)OCC3] |
| 6 | 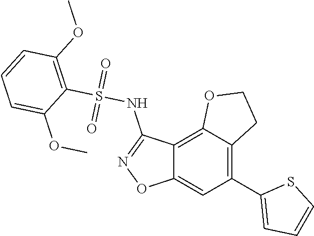 [SMILES: COc1cccc(OC)c1S(=O)(=O)Nc1noc2cc(-c3cccs3)c3c(c12)OCC3] |
| 7 | 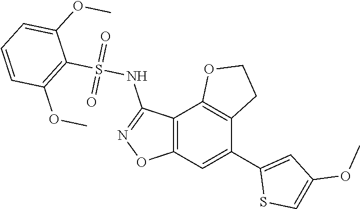 [SMILES: COc1csc(-c2cc3onc(NS(=O)(=O)c4c(OC)cccc4OC)c3c3c2CCO3)c1] |
| 8 | 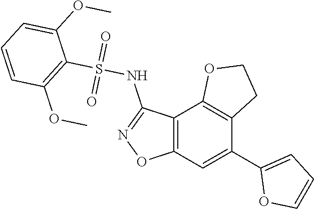 [SMILES: COc1cccc(OC)c1S(=O)(=O)Nc1noc2cc(-c3ccco3)c3c(c12)OCC3] |
| 9 | 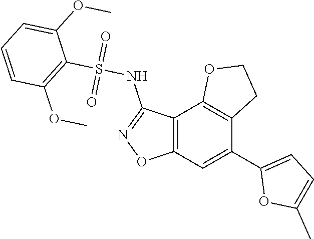 [SMILES: COc1cccc(OC)c1S(=O)(=O)Nc1noc2cc(-c3ccc(C)o3)c3c(c12)OCC3] |
| 10 | 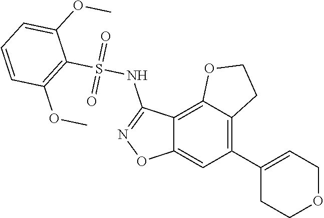 [SMILES: COc1cccc(OC)c1S(=O)(=O)Nc1noc2cc(C3=CCOCC3)c3c(c12)OCC3] |
| 11 | 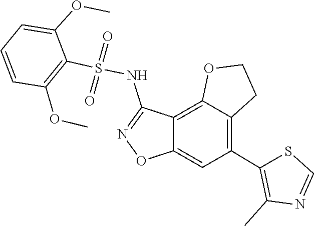 [SMILES: COc1cccc(OC)c1S(=O)(=O)Nc1noc2cc(-c3scnc3C)c3c(c12)OCC3] |
| 12 | 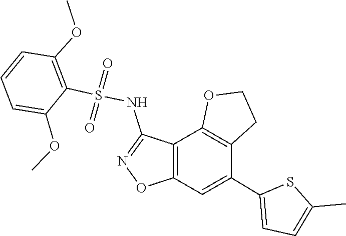 [SMILES: COc1cccc(OC)c1S(=O)(=O)Nc1noc2cc(-c3ccc(C)s3)c3c(c12)OCC3] |
| 13 | 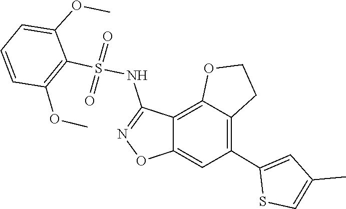 [SMILES: COc1cccc(OC)c1S(=O)(=O)Nc1noc2cc(-c3cc(C)cs3)c3c(c12)OCC3] |
| 14 | 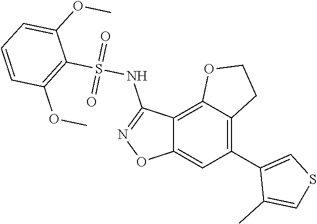 [SMILES: COc1cccc(OC)c1S(=O)(=O)Nc1noc2cc(-c3cscc3C)c3c(c12)OCC3] |
| 15 | 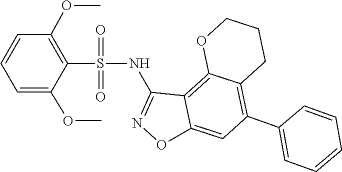 [SMILES: COc1cccc(OC)c1S(=O)(=O)Nc1noc2cc(-c3ccccc3)c3c(c12)OCCC3] |
| 16 | 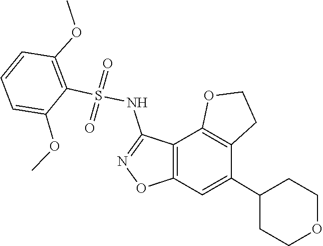 [SMILES: COc1cccc(OC)c1S(=O)(=O)Nc1noc2cc(C3CCOCC3)c3c(c12)OCC3] |
| 17 | 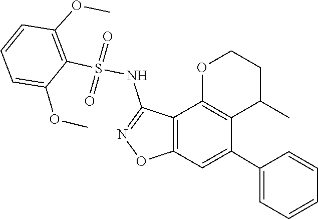 [SMILES: COc1cccc(OC)c1S(=O)(=O)Nc1noc2cc(-c3ccccc3)c3c(c12)OCCC3C] |
| 18 | 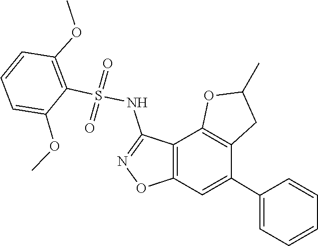 [SMILES: COc1cccc(OC)c1S(=O)(=O)Nc1noc2cc(-c3ccccc3)c3c(c12)OC(C)C3] |
| 19 | 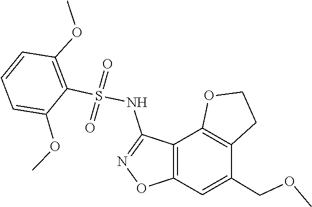 [SMILES: COCc1cc2onc(NS(=O)(=O)c3c(OC)cccc3OC)c2c2c1CCO2] |
| 20 | 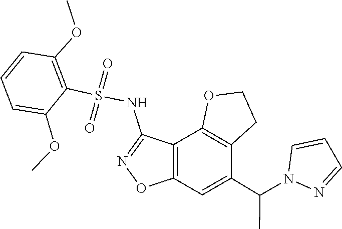 [SMILES: COc1cccc(OC)c1S(=O)(=O)Nc1noc2cc(C(C)n3cccn3)c3c(c12)OCC3] |
| 21 | 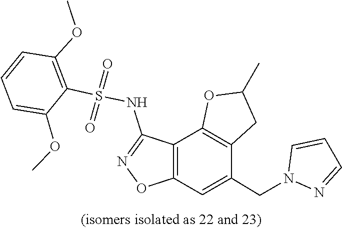 [SMILES: COc1cccc(OC)c1S(=O)(=O)Nc1noc2cc(Cn3cccn3)c3c(c12)OC(C)C3] |
| 22 | 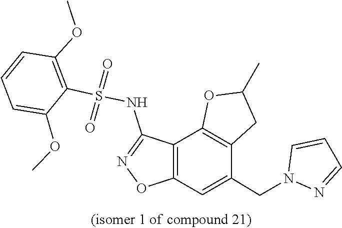 [SMILES: COc1cccc(OC)c1S(=O)(=O)Nc1noc2cc(Cn3cccn3)c3c(c12)OC(C)C3] |
| 23 | 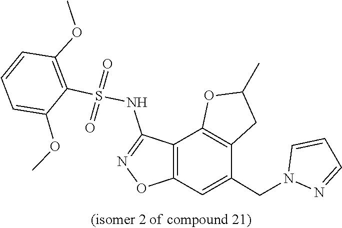 [SMILES: COc1cccc(OC)c1S(=O)(=O)Nc1noc2cc(Cn3cccn3)c3c(c12)OC(C)C3] |
| 31 | 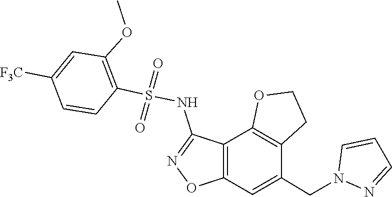 [SMILES: COc1cc(C(F)(F)F)ccc1S(=O)(=O)Nc1noc2cc(Cn3cccn3)c3c(c12)OCC3] |
| 32 | 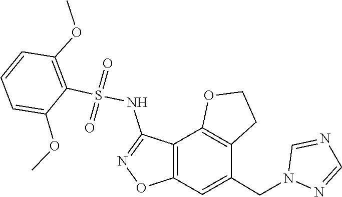 [SMILES: COc1cccc(OC)c1S(=O)(=O)Nc1noc2cc(Cn3cncn3)c3c(c12)OCC3] |
| 33 | 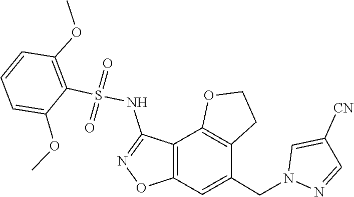 [SMILES: COc1cccc(OC)c1S(=O)(=O)Nc1noc2cc(Cn3cc(C#N)cn3)c3c(c12)OCC3] |
| 34 | 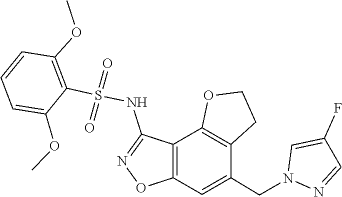 [SMILES: COc1cccc(OC)c1S(=O)(=O)Nc1noc2cc(Cn3cc(F)cn3)c3c(c12)OCC3] |
| 35 | 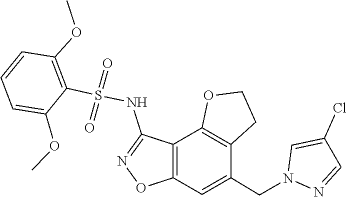 [SMILES: COc1cccc(OC)c1S(=O)(=O)Nc1noc2cc(Cn3cc(Cl)cn3)c3c(c12)OCC3] |
| 36 | 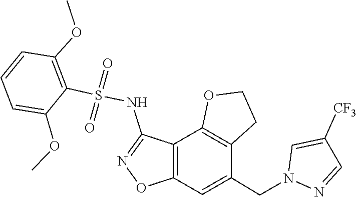 [SMILES: COc1cccc(OC)c1S(=O)(=O)Nc1noc2cc(Cn3cc(C(F)(F)F)cn3)c3c(c12)OCC3] |
| 37 | 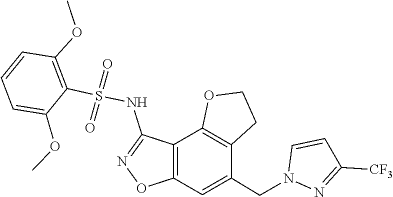 [SMILES: COc1cccc(OC)c1S(=O)(=O)Nc1noc2cc(Cn3ccc(C(F)(F)F)n3)c3c(c12)OCC3] |
| 38 | 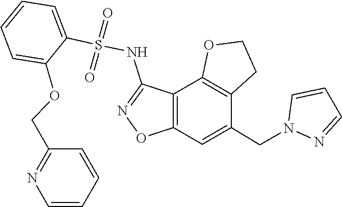 [SMILES: O=S(=O)(Nc1noc2cc(Cn3cccn3)c3c(c12)OCC3)c1ccccc1OCc1ccccn1] |
| 39 | 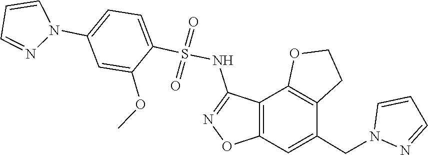 [SMILES: COc1cc(-n2cccn2)ccc1S(=O)(=O)Nc1noc2cc(Cn3cccn3)c3c(c12)OCC3] |
| 40 | 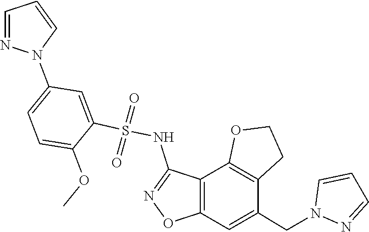 [SMILES: COc1ccc(-n2cccn2)cc1S(=O)(=O)Nc1noc2cc(Cn3cccn3)c3c(c12)OCC3] |
| 41 | 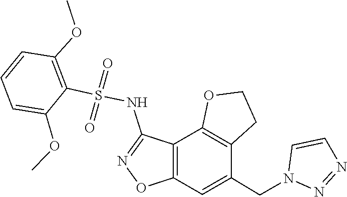 [SMILES: COc1cccc(OC)c1S(=O)(=O)Nc1noc2cc(Cn3ccnn3)c3c(c12)OCC3] |
| 42 | 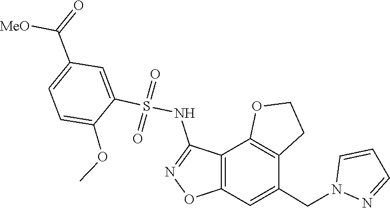 [SMILES: COC(=O)c1ccc(OC)c(S(=O)(=O)Nc2noc3cc(Cn4cccn4)c4c(c23)OCC4)c1] |
| 43 | 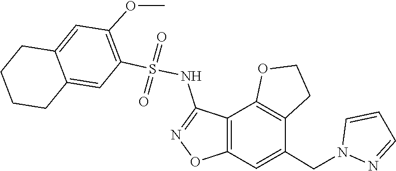 [SMILES: COc1cc2c(cc1S(=O)(=O)Nc1noc3cc(Cn4cccn4)c4c(c13)OCC4)CCCC2] |
| 44 | 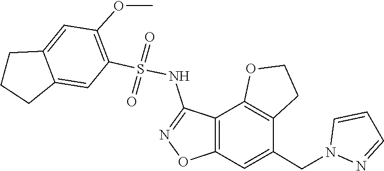 [SMILES: COc1cc2c(cc1S(=O)(=O)Nc1noc3cc(Cn4cccn4)c4c(c13)OCC4)CCC2] |
| 45 | 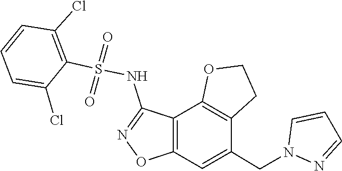 [SMILES: O=S(=O)(Nc1noc2cc(Cn3cccn3)c3c(c12)OCC3)c1c(Cl)cccc1Cl] |
| 46 | 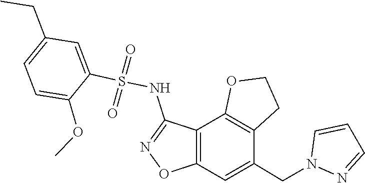 [SMILES: CCc1ccc(OC)c(S(=O)(=O)Nc2noc3cc(Cn4cccn4)c4c(c23)OCC4)c1] |
| 47 | 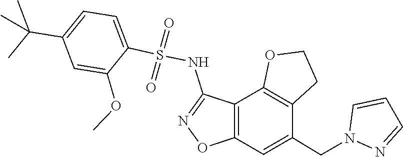 [SMILES: COc1cc(C(C)(C)C)ccc1S(=O)(=O)Nc1noc2cc(Cn3cccn3)c3c(c12)OCC3] |
| 48 | 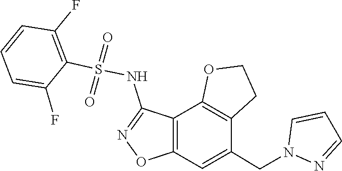 [SMILES: O=S(=O)(Nc1noc2cc(Cn3cccn3)c3c(c12)OCC3)c1c(F)cccc1F] |
| 49 | 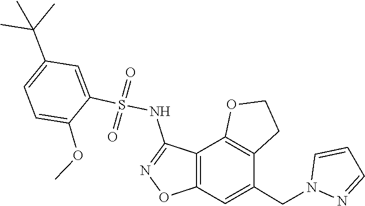 [SMILES: COc1ccc(C(C)(C)C)cc1S(=O)(=O)Nc1noc2cc(Cn3cccn3)c3c(c12)OCC3] |
| 50 | 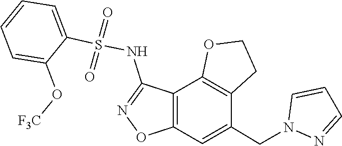 [SMILES: O=S(=O)(Nc1noc2cc(Cn3cccn3)c3c(c12)OCC3)c1ccccc1OC(F)(F)F] |
| 51 | 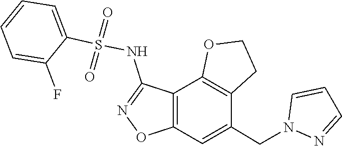 [SMILES: O=S(=O)(Nc1noc2cc(Cn3cccn3)c3c(c12)OCC3)c1ccccc1F] |
| 52 | 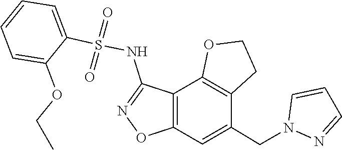 [SMILES: CCOc1ccccc1S(=O)(=O)Nc1noc2cc(Cn3cccn3)c3c(c12)OCC3] |
| 53 | 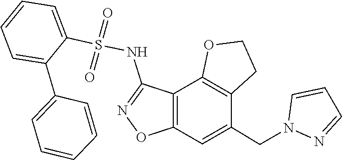 [SMILES: O=S(=O)(Nc1noc2cc(Cn3cccn3)c3c(c12)OCC3)c1ccccc1-c1ccccc1] |
| 54 | 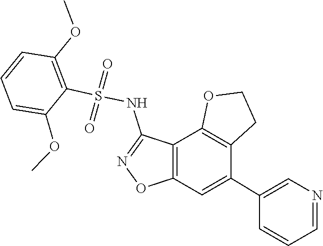 [SMILES: COc1cccc(OC)c1S(=O)(=O)Nc1noc2cc(-c3cccnc3)c3c(c12)OCC3] |
| 55 | 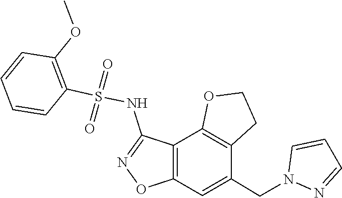 [SMILES: COc1ccccc1S(=O)(=O)Nc1noc2cc(Cn3cccn3)c3c(c12)OCC3] |
| 56 | 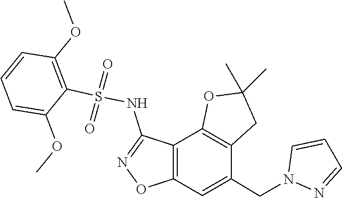 [SMILES: COc1cccc(OC)c1S(=O)(=O)Nc1noc2cc(Cn3cccn3)c3c(c12)OC(C)(C)C3] |
| 57 | 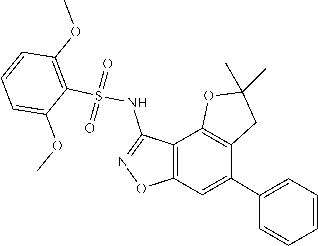 [SMILES: COc1cccc(OC)c1S(=O)(=O)Nc1noc2cc(-c3ccccc3)c3c(c12)OC(C)(C)C3] |
| 58 | 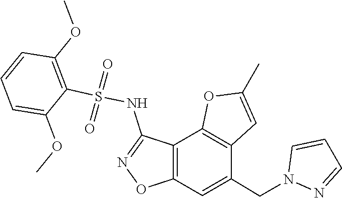 [SMILES: COc1cccc(OC)c1S(=O)(=O)Nc1noc2cc(Cn3cccn3)c3cc(C)oc3c12] |
| 59 | 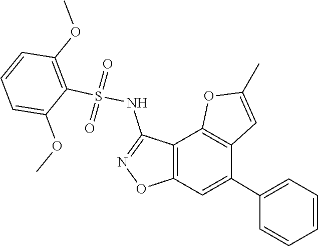 [SMILES: COc1cccc(OC)c1S(=O)(=O)Nc1noc2cc(-c3ccccc3)c3cc(C)oc3c12] |
| 60 | 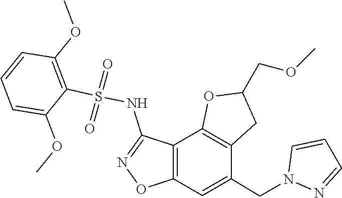 [SMILES: COCC1Cc2c(Cn3cccn3)cc3onc(NS(=O)(=O)c4c(OC)cccc4OC)c3c2O1] |
| 61 | 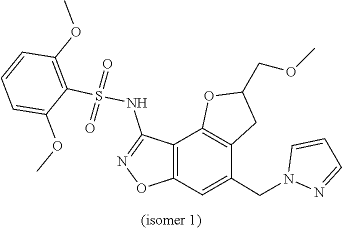 [SMILES: COCC1Cc2c(Cn3cccn3)cc3onc(NS(=O)(=O)c4c(OC)cccc4OC)c3c2O1] |
| 62 | 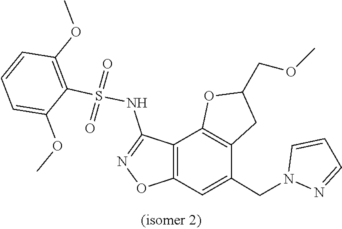 [SMILES: COCC1Cc2c(Cn3cccn3)cc3onc(NS(=O)(=O)c4c(OC)cccc4OC)c3c2O1] |
| 63 | 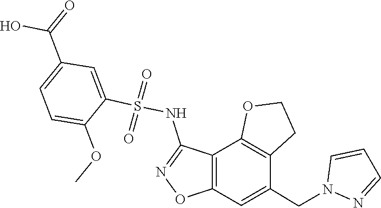 [SMILES: COc1ccc(C(=O)O)cc1S(=O)(=O)Nc1noc2cc(Cn3cccn3)c3c(c12)OCC3] |
| 64 | 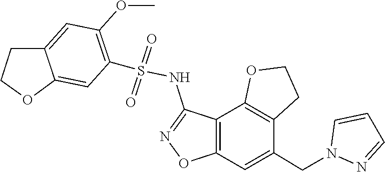 [SMILES: COc1cc2c(cc1S(=O)(=O)Nc1noc3cc(Cn4cccn4)c4c(c13)OCC4)OCC2] |
| 65 | 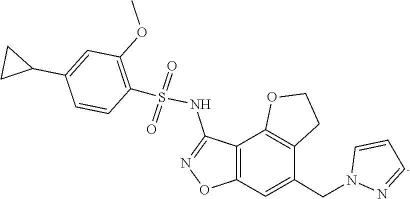 [SMILES: COc1cc(C2CC2)ccc1S(=O)(=O)Nc1noc2cc(Cn3cccn3)c3c(c12)OCC3] |

42. The compound of any one of claims 1 to 40, or a pharmaceutically acceptable salt thereof, wherein the compound has a structure

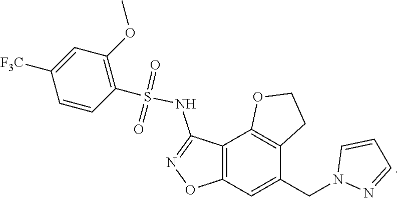 [SMILES: COc1cc(C(F)(F)F)ccc1S(=O)(=O)Nc1noc2cc(Cn3cccn3)c3c(c12)OCC3]

43. The compound of any one of claims 1 to 40, wherein the compound has a structure

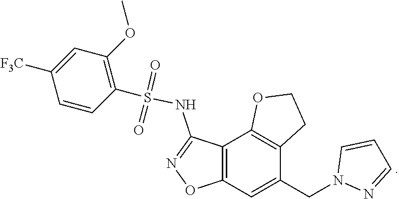 [SMILES: COc1cc(C(F)(F)F)ccc1S(=O)(=O)Nc1noc2cc(Cn3cccn3)c3c(c12)OCC3]

44. The compound of any one of claims 1 to 40, or a pharmaceutically acceptable salt thereof, wherein the compound has a structure

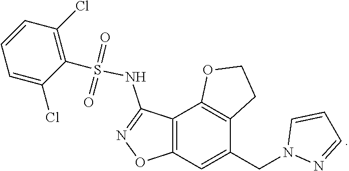 [SMILES: O=S(=O)(Nc1noc2cc(Cn3cccn3)c3c(c12)OCC3)c1c(Cl)cccc1Cl]

45. The compound of any one of claims 1 to 40, wherein the compound has a structure

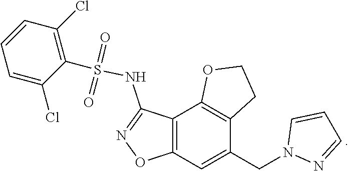 [SMILES: O=S(=O)(Nc1noc2cc(Cn3cccn3)c3c(c12)OCC3)c1c(Cl)cccc1Cl]

46. The compound of any one of claims 1 to 40, or a pharmaceutically acceptable salt thereof, wherein the compound has a structure

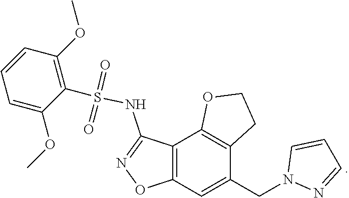 [SMILES: COc1cccc(OC)c1S(=O)(=O)Nc1noc2cc(Cn3cccn3)c3c(c12)OCC3]

47. The compound of any one of claims 1 to 40, wherein the compound has a structure

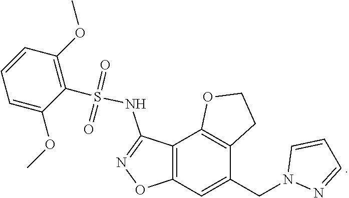 [SMILES: COc1cccc(OC)c1S(=O)(=O)Nc1noc2cc(Cn3cccn3)c3c(c12)OCC3]

48. The compound of any one of claims 1 to 40, or a pharmaceutically acceptable salt thereof, wherein the compound has a structure

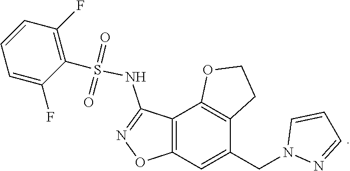 [SMILES: O=S(=O)(Nc1noc2cc(Cn3cccn3)c3c(c12)OCC3)c1c(F)cccc1F]

49. The compound of any one of claims 1 to 40, wherein the compound has a structure

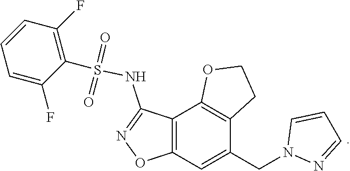 [SMILES: O=S(=O)(Nc1noc2cc(Cn3cccn3)c3c(c12)OCC3)c1c(F)cccc1F]

50. The compound of any one of claims 1 to 40, or a pharmaceutically acceptable salt thereof, wherein the compound has a structure

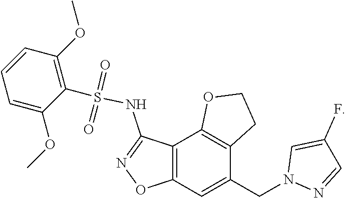 [SMILES: COc1cccc(OC)c1S(=O)(=O)Nc1noc2cc(Cn3cc(F)cn3)c3c(c12)OCC3]

51. The compound of any one of claims 1 to 40, wherein the compound has a structure

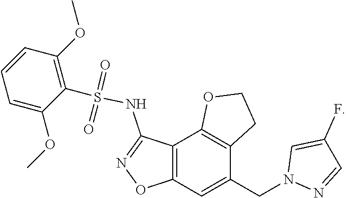 [SMILES: COc1cccc(OC)c1S(=O)(=O)Nc1noc2cc(Cn3cc(F)cn3)c3c(c12)OCC3]

52. The compound of any one of claims 1 to 40, or a pharmaceutically acceptable salt thereof, wherein the compound has a structure as shown in the following table

| Compound | Structure |
|---|---|
| 1 | 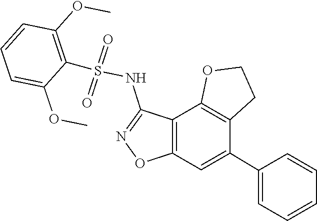 [SMILES: COc1cccc(OC)c1S(=O)(=O)Nc1noc2cc(-c3ccccc3)c3c(c12)OCC3] |
| 2 | 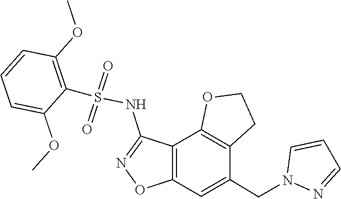 [SMILES: COc1cccc(OC)c1S(=O)(=O)Nc1noc2cc(Cn3cccn3)c3c(c12)OCC3] |
| 3 | 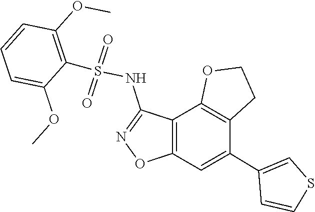 [SMILES: COc1cccc(OC)c1S(=O)(=O)Nc1noc2cc(-c3ccsc3)c3c(c12)OCC3] |
| 4 | 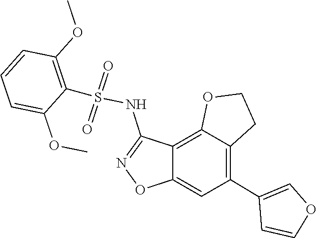 [SMILES: COc1cccc(OC)c1S(=O)(=O)Nc1noc2cc(-c3ccoc3)c3c(c12)OCC3] |
| 5 | 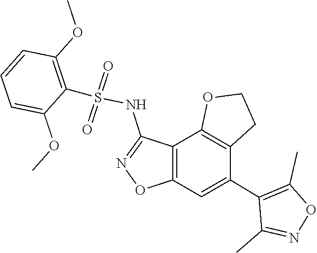 [SMILES: COc1cccc(OC)c1S(=O)(=O)Nc1noc2cc(-c3c(C)noc3C)c3c(c12)OCC3] |
| 6 | 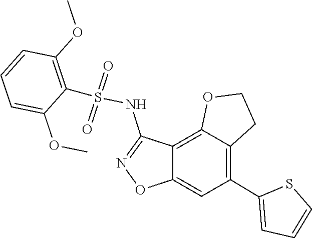 [SMILES: COc1cccc(OC)c1S(=O)(=O)Nc1noc2cc(-c3cccs3)c3c(c12)OCC3] |
| 7 | 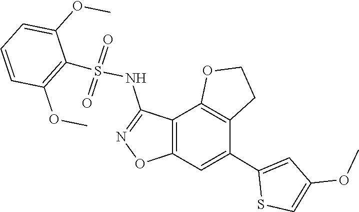 [SMILES: COc1csc(-c2cc3onc(NS(=O)(=O)c4c(OC)cccc4OC)c3c3c2CCO3)c1] |
| 8 | 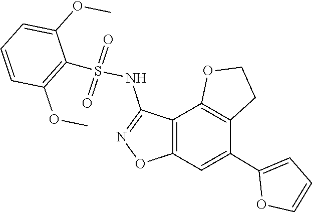 [SMILES: COc1cccc(OC)c1S(=O)(=O)Nc1noc2cc(-c3ccco3)c3c(c12)OCC3] |
| 9 | 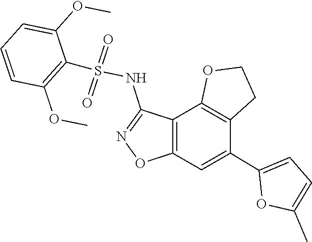 [SMILES: COc1cccc(OC)c1S(=O)(=O)Nc1noc2cc(-c3ccc(C)o3)c3c(c12)OCC3] |
| 10 | 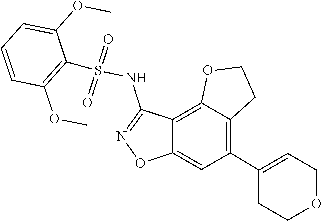 [SMILES: COc1cccc(OC)c1S(=O)(=O)Nc1noc2cc(C3=CCOCC3)c3c(c12)OCC3] |
| 11 | 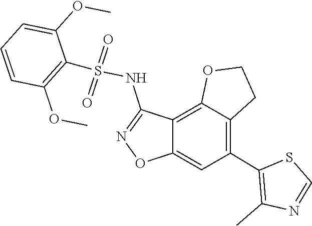 [SMILES: COc1cccc(OC)c1S(=O)(=O)Nc1noc2cc(-c3scnc3C)c3c(c12)OCC3] |
| 12 | 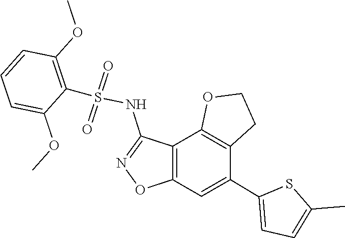 [SMILES: COc1cccc(OC)c1S(=O)(=O)Nc1noc2cc(-c3ccc(C)s3)c3c(c12)OCC3] |
| 13 | 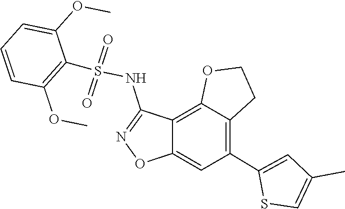 [SMILES: COc1cccc(OC)c1S(=O)(=O)Nc1noc2cc(-c3cc(C)cs3)c3c(c12)OCC3] |
| 14 | 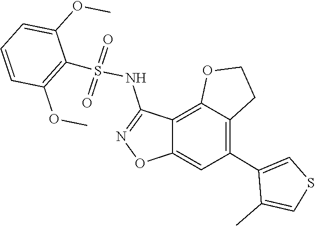 [SMILES: COc1cccc(OC)c1S(=O)(=O)Nc1noc2cc(-c3cscc3C)c3c(c12)OCC3] |
| 15 | 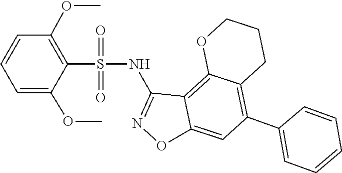 [SMILES: COc1cccc(OC)c1S(=O)(=O)Nc1noc2cc(-c3ccccc3)c3c(c12)OCCC3] |
| 16 | 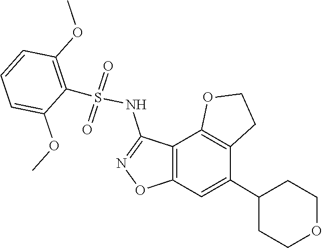 [SMILES: COc1cccc(OC)c1S(=O)(=O)Nc1noc2cc(C3CCOCC3)c3c(c12)OCC3] |
| 17 | 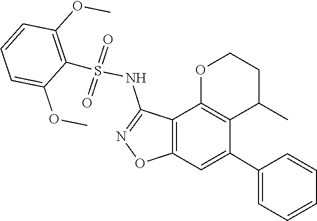 [SMILES: COc1cccc(OC)c1S(=O)(=O)Nc1noc2cc(-c3ccccc3)c3c(c12)OCCC3C] |
| 18 | 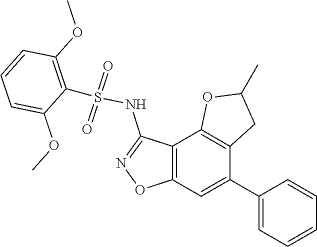 [SMILES: COc1cccc(OC)c1S(=O)(=O)Nc1noc2cc(-c3ccccc3)c3c(c12)OC(C)C3] |
| 19 | 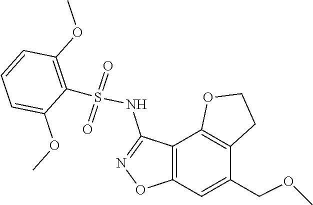 [SMILES: COCc1cc2onc(NS(=O)(=O)c3c(OC)cccc3OC)c2c2c1CCO2] |
| 20 | 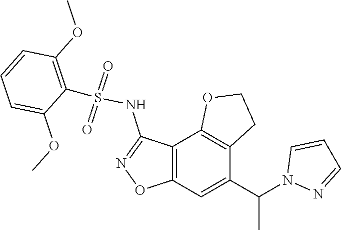 [SMILES: COc1cccc(OC)c1S(=O)(=O)Nc1noc2cc(C(C)n3cccn3)c3c(c12)OCC3] |
| 21 | 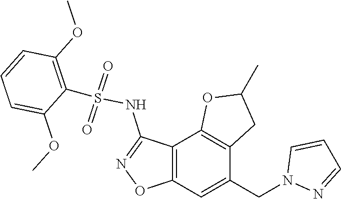 [SMILES: COc1cccc(OC)c1S(=O)(=O)Nc1noc2cc(Cn3cccn3)c3c(c12)OC(C)C3] |
| 22 | Isomer 1 of Compound 21 |
| 23 | Isomer 2 of Compound 21 |
| 24 | 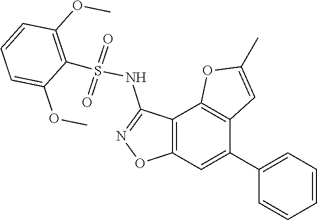 [SMILES: COc1cccc(OC)c1S(=O)(=O)Nc1noc2cc(-c3ccccc3)c3cc(C)oc3c12] |
| 25 | 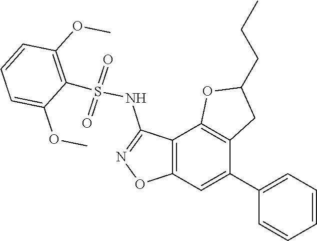 [SMILES: CCCC1Cc2c(-c3ccccc3)cc3onc(NS(=O)(=O)c4c(OC)cccc4OC)c3c2O1] |
| 26 | 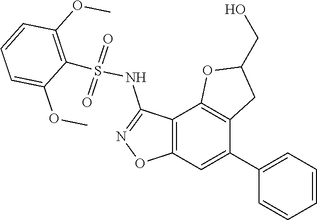 [SMILES: COc1cccc(OC)c1S(=O)(=O)Nc1noc2cc(-c3ccccc3)c3c(c12)OC(CO)C3] |
| 27 | 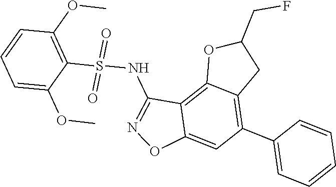 [SMILES: COc1cccc(OC)c1S(=O)(=O)Nc1noc2cc(-c3ccccc3)c3c(c12)OC(CF)C3] |
| 28 | 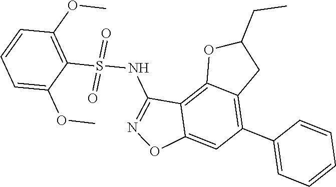 [SMILES: CCC1Cc2c(-c3ccccc3)cc3onc(NS(=O)(=O)c4c(OC)cccc4OC)c3c2O1] |
| 29 | 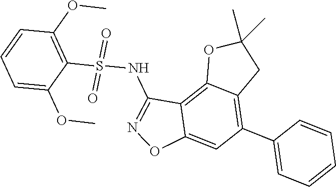 [SMILES: COc1cccc(OC)c1S(=O)(=O)Nc1noc2cc(-c3ccccc3)c3c(c12)OC(C)(C)C3] |
| 30 | 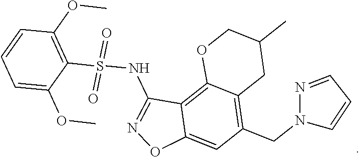 [SMILES: COc1cccc(OC)c1S(=O)(=O)Nc1noc2cc(Cn3cccn3)c3c(c12)OCC(C)C3] |

53. A pharmaceutical composition comprising a compound of any one of claims 1 to 52 or a pharmaceutically acceptable salt or stereoisomer or a tautomer thereof and a pharmaceutically acceptable carrier or excipient.

54. A compound according to any one of claims 1 to 52 or a pharmaceutically acceptable salt or a stereoisomer or a tautomer thereof, for use as a medicament.

55. A method of modulating KAT6A in a subject comprising administering to the subject in need thereof, a therapeutically effective amount of compound according to any one of claims 1 to 52 or a pharmaceutically acceptable salt or a stereoisomer or a tautomer thereof.

56. A method of treating a disease or disorder mediated by KAT6A in a subject comprising administering to the subject in need thereof a therapeutically effective amount of a compound according to any one of claims 1 to 52 or a pharmaceutically acceptable salt or a stereoisomer or a tautomer thereof.

57. The method of claim 56, wherein the disease or disorder is cancer.

58. The method of claim 57, wherein the cancer is selected from brain gliomas, glioblastomas, astrocytomas, multiforme, bannayan-Zonana syndrome, Cowden disease, Lhermitte-Duclos disease, breast cancer, colon cancer, head and neck cancer, kidney, liver, lung cancer, bone cancer, colorectal cancer, germ cell cancer, melanoma, ovarian cancer, pancreatic cancer, adenocarcinoma, ductal adenocarcinoma, adenosquamous carcinoma, acinar cell carcinoma, glucagonoma, insulinoma, prostate, sarcoma and thyroid cancer, lymphoblastic T cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T cell leukemia, plasmacytoma, immunoblastic large cell leukemia, mantle cell leukemia, megakaryoblastic leukemia, multiple myeloma, acute megakaryocytic leukemia, promyelocytic leukemia, erythroleukemia, malignant lymphoma, Hodgkin's lymphoma, non-Hodgkin's lymphoma, lymphoblastic T cell lymphoma, Burkitt's lymphoma, follicular lymphoma, neuroblastoma, bladder cancer, urothelial cancer, vulval cancer, uterine/cervical cancer, endometrial cancer, renal cancer, mesothelioma, esophageal cancer, salivary gland cancer, hepatocellular cancer, gastric cancer, nasopharyngeal cancer, buccal cancer, cancer of the mouth, GIST (gastrointestinal stromal tumor), neuroendocrine cancers, testicular cancer and virus-related cancer.

59. Compound according to any one of claims 1 to 41 for use in the treatment of a disease or disorder mediated by KAT6A.
